(19) United States
(12) Patent Application Publication
(10) Pub. No.: US 2025/0011401 A1
(43) Pub. Date: Jan. 9, 2025

(54) OPTIMIZED NUCLEIC ACID ANTIBODY CONSTRUCTS

(71) Applicants: The Wistar Institute of Anatomy and Biology, Philadelphia, PA (US); INOVIO PHARMACEUTICALS, INC., Plymouth Meeting, PA (US)

(72) Inventors: David Weiner, Merion, PA (US); Ghiabe Guibinga, La Mesa, CA (US); Neil Cooch, Oreland, PA (US); Charles Reed, Souderton, PA (US)

(21) Appl. No.: 18/765,882

(22) Filed: Jul. 8, 2024

Related U.S. Application Data
(63) Continuation of application No. 16/611,943, filed on Nov. 8, 2019, now Pat. No. 12,054,537.
(60) Provisional application No. 62/504,448, filed on May 10, 2017.
(60) Provisional application No. 62/624,367, filed on Jan. 31, 2018.
(60) Provisional application No. 62/624,320, filed on Jan. 31, 2018.
(60) Provisional application No. 62/504,460, filed on May 10, 2017.

(51) Int. Cl. C07K 16/10; A61K 39/00; A61K 48/00; C07K 16/00; C12N 15/10; G16B 15/20; G16B 30/10

(52) U.S. Cl. CPC C07K 16/1081; C07K 16/00; C07K 16/10; C07K 16/1027; A61K 2039/505; A61K 48/00; C07K 2317/522; C07K 2317/524; C07K 2317/53; C07K 2317/565; C07K 2317/567; C07K 2317/622; C07K 2317/66; C12N 15/1031; C12N 15/1079; G16B 15/20; G16B 30/10

(57) ABSTRACT

Disclosed herein are compositions comprising structurally modified DNA encoded antibodies (DMAbs), methods of structurally modifying DMAbs, and methods of using structurally modified DMAbs.

### CROSS REFERENCE TO RELATED APPLICATIONS

This application is a Continuation of U.S. patent application Ser. No. 16/611,943, filed Nov. 8, 2019, which is the U.S. national phase application filed under 35 U.S.C. § 371 claiming priority to International Patent Application No. PCT/US18/32023, filed May 10, 2018, which is entitled to priority under 35 U.S.C. § 119 (e) to U.S. Provisional Application No. 62/504,448, filed May 10, 2017, U.S. Provisional Application No. 62/504,460 filed May 10, 2017, U.S. Provisional Application No. 62/624,320 filed Jan. 31, 2018 and U.S. Provisional Application No. 62/624,367 filed Jan. 31, 2018, the contents of each of which is incorporated by reference herein in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. The Sequence Listing is written in the accompanying XML text file titled: “206194-0014-01US Sequence Listing.xml”; created on Jul. 7, 2024, and 339,376 bytes in size.

### TECHNICAL FIELD

The present invention relates to methods of optimizing a nucleic acid antibody construct and to optimized structurally modified nucleic acid antibody constructs. The compositions of the invention provide improved methods for inducing immune responses, and for prophylactically and/or therapeutically immunizing individuals against an antigen.

### BACKGROUND

The immunoglobulin molecule comprises two of each type of light (L) and heavy (H) chain, which are covalently linked by disulphide bonds (shown as S-S) between cysteine residues. The variable domains of the heavy chain (VH) and the light chain (VL) contribute to the binding site of the antibody molecule. The heavy-chain constant region is made up of three constant domains (CH1, CH2 and CH3) and the (flexible) hinge region. The light chain also has a constant domain (CL). The variable regions of the heavy and light chains comprise four framework regions (FRs; FR1, FR2, FR3 and FR4) and three complementarity-determining regions (CDRs; CDR1, CDR2 and CDR3). Accordingly, these are very complex genetic systems that have been difficult to assemble in vivo.

Targeted monoclonal antibodies (mAbs) represent one of the most important medical therapeutic advances of the last 25 years. This type of immune based therapy is now used routinely against a host of autoimmune diseases, treatment of cancer as well as infectious diseases. For malignancies, many of the immunoglobulin (Ig) based therapies currently used are in combination with cytotoxic chemotherapy regimens directed against tumors. This combination approach has significantly improved overall survival. Multiple mAb preparations are licensed for use against specific cancers, including Rituxan (Rituximab), a chimeric mAb targeting CD20 for the treatment of Non-Hodgkins lymphoma and Ipilimumab (Yervoy), a human mAb that blocks CTLA-4 and which has been used for the treatment of melanoma and other malignancies. Additionally, Bevacizumab (Avastin) is another prominent humanized mAb that targets VEGF and tumor neovascularization and has been used for the treatment of colorectal cancer. Perhaps the most high profile mAb for treatment of a malignancy is Trastuzumab (Herceptin), a humanized preparation targeting Her2/neu that has been demonstrated to have considerable efficacy against breast cancer in a subset of patients. Furthermore, a host of mAbs are in use for the treatment of autoimmune and specific blood disorders.

In addition to cancer treatments, passive transfer of polyclonal Igs mediate protective efficacy against a number of infectious diseases including diphtheria, hepatitis A and B, rabies, tetanus, chicken-pox and respiratory syncytial virus (RSV). In fact, several polyclonal Ig preparations provide temporary protection against specific infectious agents in individuals traveling to disease endemic areas in circumstances when there is insufficient time for protective Igs to be generated through active vaccination. Furthermore, in children with immune deficiency the Palivizumab (Synagis), a mAb, which targets RSV infection, has been demonstrated to clinically protect against RSV.

Currently available therapeutic antibodies that exist in the market are human IgG1 isotypes. These antibodies include glycoproteins bearing two N-linked biantennary complex-type oligosaccharides bound to the antibody constant region (Fc), in which a majority of the oligosaccharides are core-fucosylated. It exercises effector functions of antibody-dependent cellular toxicity (ADCC) and complement-dependent cytotoxicity (CDC) through the interaction of the Fc with either leukocyte receptors (FcγRs) or complement. There is a phenomena of reduced in vivo efficacy of therapeutic antibodies (versus in vitro), thus resulting in the need for large doses of therapeutic antibodies—sometimes weekly doses of several hundred milligrams. This is mainly due to the competition between serum IgG and therapeutic antibodies for binding to FcγRIIIa on natural killer (NK) cells. Endogenous human serum IgG inhibits ADCC induced by therapeutic antibodies. Thus, there can be enhanced efficacy of non-fucosylated therapeutic antibodies in humans. Non-fucosylated therapeutic antibodies have much higher binding affinity for FcγRIIIa than fucosylated human serum IgG, which is a preferable character to conquer the interference by human plasma IgG.

Antibody based treatments are not without risks. One such risk is antibody-dependent enhancement (ADE), which occurs when non-neutralizing antiviral proteins facilitate virus entry into host cells, leading to increased infectivity in the cells. Some cells do not have the usual receptors on their surfaces that viruses use to gain entry. The antiviral proteins (i.e., the antibodies) bind to antibody Fc receptors that some of these cells have in the plasma membrane. The viruses bind to the antigen binding site at the other end of the antibody. This virus can use this mechanism to infect human macrophages, causing a normally mild viral infection to become life-threatening. The most widely known example of ADE occurs in the setting of infection with the dengue virus (DENV). It is observed when a person who has previously been infected with one serotype of DENV becomes infected many months or years later with a different serotype. In such cases, the clinical course of the disease is more severe, and these people have higher viremia compared with those in whom ADE has not occurred. This explains the observation that while primary (first) infections cause mostly minor disease (DF) in children, secondary infection (re-infection at a later date) is more likely to be associated with severe disease (DHF and/or DSS) in both children and adults. There are four antigenically different serotypes of DENV (DENV-1-DENV-4). Infection with DENV induces the production of neutralizing homotypic immunoglobulin G (IgG) antibodies which provide lifelong immunity against the infecting serotype. Infection with DENV also produces some degree of cross-protective immunity against the other three serotypes. In addition to inducing neutralizing heterotypic antibodies, infection with DENV can also induce heterotypic antibodies which neutralize the virus only partially or not at all. The production of such cross-reactive but non-neutralizing antibodies could be the reason for more severe secondary infections. Once inside the white blood cell, the virus replicates undetected, eventually generating very high virus titers which cause severe disease.

The clinical impact of mAb therapy is impressive. However, issues remain that limit the use and dissemination of this therapeutic approach. Some of these include the high cost of production of these complex biologics that can limit their use in the broader population, particularly in the developing world where they could have a great impact. For example, mAbs targeting the Ebola virus glycoprotein (GP) represent an important treatment approach against Ebola virus disease (EVD). It has been shown that individual mAbs and mAb cocktails can successfully protect small animals and non-human primates against lethal Ebola virus infection. MAb-based therapy against EVD is further supported by favorable recovery in confirmed human EVD cases that received the anti-GP mAb cocktail, ZMapp. However, the dramatic cost, slow development, and requirement for several high-dose administrations (mg/kg) represent a significant challenge for protein mAb delivery, especially during a possible outbreak.

Furthermore, the frequent requirement for repeat administrations of the mAbs to attain and maintain efficacy can be an impediment in terms of logistics and patient compliance. New antibodies that would reduce or eliminate the low in vivo efficacy of therapeutic antibodies due to competition with serum IgGs are needed. New antibodies that can eliminate antibody dependent enhancement in viruses like EVD, Dengue, HIV, RSV and others are needed. Bispecific antibodies, bifunctional antibodies, and antibody cocktails are needed to perform several functions that could prove therapeutic or prophylactic. Combination therapies are needed as well that can utilize the synthetic antibodies described herein along with immunostimulating a host system through immunization with a vaccine, including a DNA based vaccine. Additionally, the long-term stability of these antibody formulations is frequently short and less than optimal.

Thus, there remains a need in the art for an optimized synthetic antibody molecule that can be delivered to a subject in a safe and cost effective manner.

### SUMMARY

In one embodiment, the invention relates to a method of generating a nucleic acid sequence encoding a structurally modified DNA encoded antibody (DMAb) comprising the steps of identifying one or more CDR region of a first DMAb, identifying one or more CDR region of a second DMAb, substituting the amino acid sequence of one or more CDR region of a second DMAb with the amino acid sequence of one or more CDR region of a first DMAb to generate an amino acid sequence of a structurally modified DMAb, and generating a nucleic acid sequence encoding a structurally modified DMAb. In one embodiment the method further comprises optimizing the nucleic acid sequence.

In one embodiment, the invention relates to a method of generating a nucleic acid sequence encoding a structurally modified DMAb comprising the steps of performing sequence alignment of an amino acid sequence or a fragment of an amino acid sequence of a first DMAb with the same from one or more additional DMAb sequences, substituting one or more amino acid residues of the amino acid sequence of the first DMAb with one or more amino acid residues of the amino acid sequence of one or more additional DMAb sequences, wherein the one or more amino acid residues are not residues in a CDR region, to generate an amino acid sequence of a structurally modified DMAb, and generating a nucleic acid sequence encoding a structurally modified DMAb. In one embodiment the method further comprises optimizing the nucleic acid sequence.

In one embodiment, the invention relates to a method of generating a nucleic acid sequence encoding a structurally modified DMAb comprising the steps of identifying amino acid residues of a DMAb that are predicted to participate in interaction at an interface of a variable heavy chain and variable light chain domain, making one or more amino acid substitutions of the identified amino acid residues, wherein the one or more substitutions is predicted to alter at least one of an isoelectric point and a surface charge at the interface, to generate an amino acid sequence of a structurally modified DMAb, and generating a nucleic acid sequence encoding a structurally modified DMAb. In one embodiment the method further comprises optimizing the nucleic acid sequence.

In one embodiment, the invention relates to a method of generating a nucleic acid sequence encoding a structurally modified DMAb comprising the steps of removing an amino acid sequence for a CH and CL domain from an amino acid sequence of a DMAb, adding an amino acid sequence of a linker between an amino acid sequence for a VH and VL domain to an amino acid sequence of a DMAb to generate an amino acid sequence of a structurally modified DMAb, performing at least one round of modeling on the generated amino acid sequence, and generating a nucleotide sequence encoding a structurally modified DMAb. In one embodiment the method further comprises optimizing the nucleic acid sequence.

In one embodiment, the method of modeling comprises at least one of linker modeling, hinge modification modeling, framework modeling, and CDR loop refinement.

In one embodiment, the linker is C-terminally linked to the VH domain and N-terminally linked to the VL domain. In one embodiment, the linker is N-terminally linked to the VH domain and C-terminally linked to the VL domain.

In one embodiment, the invention relates to a method of generating a nucleic acid sequence encoding a structurally modified DMAb comprising the steps of identifying one or more CDR region of a first DMAb, identifying one or more CDR region of a second DMAb, substituting the amino acid sequence of one or more CDR region of a second DMAb with the amino acid sequence of one or more CDR region of a first DMAb to generate an amino acid sequence of a structurally modified DMAb, removing an amino acid sequence for a CH and CL domain from an amino acid sequence of a DMAb, adding an amino acid sequence of a linker between an amino acid sequence for a VH and VL domain to an amino acid sequence of a DMAb to generate an amino acid sequence of a structurally modified DMAb, performing at least one round of modeling on the generated amino acid sequence, and generating a nucleotide sequence encoding a structurally modified DMAb. In one embodiment the method further comprises optimizing the nucleic acid sequence.

In one embodiment, the invention relates to a method of generating a nucleic acid sequence encoding a structurally modified DMAb comprising the steps of performing sequence alignment of an amino acid sequence or a fragment of an amino acid sequence of a first DMAb with the same from one or more additional DMAb sequences, substituting one or more amino acid residues of the amino acid sequence of the first DMAb with one or more amino acid residues of the amino acid sequence of one or more additional DMAb sequences, wherein the one or more amino acid residues are not residues in a CDR region, to generate an amino acid sequence of a structurally modified DMAb, removing an amino acid sequence for a CH and CL domain from an amino acid sequence of a DMAb, adding an amino acid sequence of a linker between an amino acid sequence for a VH and VL domain to an amino acid sequence of a DMAb to generate an amino acid sequence of a structurally modified DMAb, performing at least one round of modeling on the generated amino acid sequence, and generating a nucleic acid sequence encoding a structurally modified DMAb. In one embodiment the method further comprises optimizing the nucleic acid sequence.

In one embodiment, the invention relates to a method of generating a nucleic acid sequence encoding a structurally modified DMAb comprising the steps of identifying amino acid residues of a DMAb that are predicted to participate in interaction at an interface of a variable heavy chain and variable light chain domain, making one or more amino acid substitutions of the identified amino acid residues, wherein the one or more substitutions is predicted to alter at least one of an isoelectric point and a surface charge at the interface, to generate an amino acid sequence of a structurally modified DMAb, removing an amino acid sequence for a CH and CL domain from an amino acid sequence of a DMAb, adding an amino acid sequence of a linker between an amino acid sequence for a VH and VL domain to an amino acid sequence of a DMAb to generate an amino acid sequence of a structurally modified DMAb, performing at least one round of modeling on the generated amino acid sequence, and generating a nucleic acid sequence encoding a structurally modified DMAb. In one embodiment the method further comprises optimizing the nucleic acid sequence.

In one embodiment, the invention relates to a structurally modified DMAb encoded by the nucleic acid molecule generated using the methods of the invention.

In one embodiment, the invention relates to a structurally modified DMAb comprising one or more CDR region from a first DMAb and one or more constant region from a second DMAb, wherein the structurally modified DMAb exhibits at least one of higher in vivo expression and higher binding compared to a corresponding DMAb not so modified.

In one embodiment, the structurally modified DMAb exhibits at least the same or higher antigen binding specificity compared to the specificity of the structurally modified DMAb prior to having been so modified.

In one embodiment, the invention relates to a structurally modified DMAb comprising one or more amino acid substitutions, wherein the structurally modified DMAb exhibits at least one of higher in vivo expression and higher binding compared to a corresponding DMAb not so modified.

In one embodiment, the invention relates to a structurally modified DMAb comprising a CH1, CH2, hinge, VH, linker and VL domain, wherein the structurally modified DMAb exhibits at least one of higher in vivo expression and higher binding compared to a corresponding DMAb not so modified.

In one embodiment, the invention relates to a composition comprising a nucleic acid molecule comprising at least one nucleotide sequence encoding a structurally modified DMAb. In one embodiment, at least one structurally modified DMAb comprises an amino acid sequence selected from a) an amino acid sequence having at least 90% over an entire length of the encoded sequence to an amino acid sequence of: SEQ ID NO:2, SEQ ID NO: 4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO: 38, SEQ ID NO:40, SEQ ID NO:42, SEQ ID NO:44, SEQ ID NO:46, SEQ ID NO:64, SEQ ID NO:66, SEQ ID NO: 68, SEQ ID NO: 70, SEQ ID NO: 72, SEQ ID NO: 74, SEQ ID NO: 76, SEQ ID NO:78, SEQ ID NO:80, SEQ ID NO:82, SEQ ID NO:84, SEQ ID NO:86, SEQ ID NO:88, SEQ ID NO:90, SEQ ID NO: 92, SEQ ID NO:94, SEQ ID NO:96, SEQ ID NO: 98, SEQ ID NO: 100, SEQ ID NO: 102, SEQ ID NO: 104, SEQ ID NO: 106, SEQ ID NO: 108, SEQ ID NO:111, SEQ ID NO: 113, SEQ ID NO: 115 or SEQ ID NO: 119; or b) a fragment comprising at least 60% of an amino acid sequence of: SEQ ID NO:2, SEQ ID NO: 4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO: 38, SEQ ID NO:40, SEQ ID NO:42, SEQ ID NO:44, SEQ ID NO:46, SEQ ID NO:64, SEQ ID NO:66, SEQ ID NO:68, SEQ ID NO:70, SEQ ID NO:72, SEQ ID NO: 74, SEQ ID NO: 76, SEQ ID NO: 78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO: 98, SEQ ID NO: 100, SEQ ID NO: 102, SEQ ID NO: 104, SEQ ID NO: 106, SEQ ID NO: 108 SEQ ID NO:111, SEQ ID NO: 113, SEQ ID NO: 115 or SEQ ID NO: 119.

In one embodiment, the nucleic acid molecule comprises at least two nucleotide sequences wherein each nucleotide sequence encodes a structurally modified DMAb. In one embodiment, each of the at least two nucleotide sequence encodes a structurally modified DMAb comprising an amino acid sequence selected from a) an amino acid sequence having at least 90% over an entire length of the encoded sequence to an amino acid sequence of: SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO:42, SEQ ID NO:44, SEQ ID NO:46, SEQ ID NO:64, SEQ ID NO:66, SEQ ID NO:68, SEQ ID NO: 70, SEQ ID NO: 72, SEQ ID NO: 74, SEQ ID NO:76, SEQ ID NO: 78, SEQ ID NO:80, SEQ ID NO:82, SEQ ID NO:84, SEQ ID NO:86, SEQ ID NO:88, SEQ ID NO:90, SEQ ID NO: 92, SEQ ID NO:94, SEQ ID NO:96, SEQ ID NO:98, SEQ ID NO: 100, SEQ ID NO: 102, SEQ ID NO: 104, SEQ ID NO: 106, SEQ ID NO:108, SEQ ID NO:111, SEQ ID NO:113, SEQ ID NO:115 or SEQ ID NO: 119; or b) a fragment comprising at least 60% of an amino acid sequence of: SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO:42, SEQ ID NO:44, SEQ ID NO:46, SEQ ID NO:64, SEQ ID NO:66, SEQ ID NO:68, SEQ ID NO: 70, SEQ ID NO: 72, SEQ ID NO: 74, SEQ ID NO: 76, SEQ ID NO:78, SEQ ID NO:80, SEQ ID NO:82, SEQ ID NO:84, SEQ ID NO:86, SEQ ID NO:88, SEQ ID NO:90, SEQ ID NO: 92, SEQ ID NO:94, SEQ ID NO:96, SEQ ID NO:98, SEQ ID NO: 100, SEQ ID NO: 102, SEQ ID NO: 104, SEQ ID NO: 106, SEQ ID NO: 108 SEQ ID NO: 111, SEQ ID NO: 113, SEQ ID NO: 115 or SEQ ID NO:119.

In one embodiment, the nucleic acid molecule comprises a nucleotide sequence encoding a structurally modified DMAb comprising an amino acid sequence selected from SEQ ID NO:64, SEQ ID NO:66, SEQ ID NO:68, SEQ ID NO:70, SEQ ID NO: 72, SEQ ID NO:74, SEQ ID NO: 76, SEQ ID NO: 78, SEQ ID NO:80, SEQ ID NO:82, SEQ ID NO:84, SEQ ID NO: 86, SEQ ID NO: 88, SEQ ID NO:90, SEQ ID NO:92, SEQ ID NO: 94, SEQ ID NO:96, SEQ ID NO:98, SEQ ID NO: 100, SEQ ID NO: 102, SEQ ID NO: 104 or SEQ ID NO: 106 and a nucleotide sequence encoding a structurally modified DMAb comprising an amino acid sequence of SEQ ID NO: 108.

In one embodiment, the invention relates to a method of treating a subject having a disease or disorder comprising administering a composition comprising a nucleic acid molecule comprising a nucleotide sequence encoding a structurally modified DMAb to a subject in need thereof.

### BRIEF DESCRIPTION OF THE DRAWINGS

FIG. 1A through FIG. 1D depict exemplary models of various structurally modified DMAbs that can be generated. FIG. 1A depicts a diagram of a full graft DMAb. FIG. 1B depicts a diagram of partial grafting. Multiple modifications of amino acids in the FR1 and FR4 regions of the modified DMAb (SEQ ID NO:48) as compared to the parental DMAb (SEQ ID NO:47) are shown to the right of the modeled DMAb. FIG. 1C depicts a diagram of scaffold modification. Side chains of residues that participate in the VH VL interface are shown. Scaffold modifications of the light (upper) and heavy (lower) chains (SEQ ID NO:50 and SEQ ID NO:52 respectively) are shown with the modified residues highlighted. The parental DMAb sequences are SEQ ID NO:49 and SEQ ID NO: 51. FIG. 1D depicts protein ribbon images showing full and partial DMAb framework grafting. The VH-VL (yellow/blue) of a high expressing DMAb is shown in the upper left. The VH-VL (green/red) of a low expressing DMAb is shown in the upper right. The new DMAb molecule in the lower left is created by grafting the CDRs from the low expresser onto the framework of the high expresser. In the lower right is a new DMAb created by a partial graft, replacing the first 22 amino acids in the VL of the poorly expressing DMAb with those from the high expresser.

FIG. 2A through FIG. 2B depict exemplary experimental results demonstrating the effects of full and partial grafting of BDBV223. FIG. 2A depicts exemplary experimental results demonstrating expression of BDBV223 antibody and full and partial grafts. FIG. 2B depicts exemplary experimental results demonstrating EBOV antigen binding of BDBV223 antibody and full and partial grafts.

FIG. 3A through FIG. 3B depict exemplary experimental results demonstrating the effects of full and partial grafting and structural modification of Z5D2. FIG. 3A depicts exemplary experimental results demonstrating expression of Z5D2 antibody and full and partial grafts. FIG. 3B depicts exemplary experimental results demonstrating EBOV antigen binding of Z5D2 antibody and full and partial grafts.

FIG. 4 depicts an alignment of multiple light chains from low expressing DMAbs (SEQ ID NO:54 through SEQ ID NO:57) with light chains from two verified highly expressing DMAbs (SEQ ID NO:58 and SEQ ID NO:59).

FIG. 5 depicts an identity matrix comparing the full VL, the VL excluding the CDR regions, the FR1 and the FR4 for each parental light chain of FIG. 5 with each of the verified highly expressing DMAbs.

FIG. 6 depicts a diagram showing the structural differences between full length and scFv-Fc modified IgG DMAbs.

FIG. 7A through FIG. 7C depict protein modeling images demonstrating that the scFv-Fc linker does not interfere with CDRs. FIG. 7A depicts a stick and ribbon image of the predicted folding of an scFv-Fc antibody. The VL (red), VL CDRs (pink), VH (green), VH CDRs (purple), and linker (CPK) are shown. FIG. 7B and FIG. 7C depict two rotational views of a space filled model of a ScFv-Fc DMAb.

FIG. 8A through FIG. 8D depict exemplary experimental results demonstrating the effects of scFv-Fc conversion of DMAb BDBV223. FIG. 8A depicts the expression levels of the parental and ScFv-Fc modified DMAbs. FIG. 8B depicts the serum expression levels of the parental and ScFv-Fc modified DMAbs. FIG. 8C depicts the antigen binding of the parental and ScFv-Fc modified DMAbs to the glycoprotein antigen from the 1976 Ebola virus outbreak strain. FIG. 8D depicts the antigen binding of the parental and ScFv-Fc modified DMAbs to the glycoprotein antigen from the 2014 Ebola virus outbreak strain.

FIG. 9A through FIG. 9F depict exemplary experimental results demonstrating the effects of scFv-Fc conversion of DMAb Z5D2. FIG. 9A depicts the expression levels of the parental and ScFv-Fc modified DMAbs over 21 days. FIG. 9B depicts the serum expression levels of the parental and ScFv-Fc modified DMAbs. FIG. 9C depicts the antigen binding of the parental and ScFv-Fc modified DMAbs to the glycoprotein antigen from the 1976 Ebola virus outbreak strain. FIG. 9D depicts the antigen binding of the parental and ScFv-Fc modified DMAbs to the glycoprotein antigen from the 2014 Ebola virus outbreak strain. FIG. 9E depicts the expression levels of the parental and ScFv-Fc modified DMAbs over 35 days. FIG. 9F depicts the normalized expression levels of the parental and ScFv-Fc modified DMAbs over 35 days.

FIG. 10A through FIG. 10E depict exemplary experimental results demonstrating the effects of scFv-Fc conversion of DMAb Z1H3. FIG. 10A depicts the expression levels of the parental and ScFv-Fc modified DMAbs over 21 days. FIG. 10B depicts the antigen binding of the parental and ScFv-Fc modified DMAbs to the glycoprotein antigen from the 1976 Ebola virus outbreak strain. FIG. 10C depicts the antigen binding of the parental and ScFv-Fc modified DMAbs to the glycoprotein antigen from the 2014 Ebola virus outbreak strain. FIG. 10D depicts the expression levels of the parental and ScFv-Fc modified DMAbs over 35 days. FIG. 10E depicts the normalized expression levels of the parental and ScFv-Fc modified DMAbs over 35 days.

FIG. 11 depicts exemplary outputs from different steps in the ScFv-Fc modeling process.

FIG. 12 depicts exemplary images of Fv, VH-VL and VL-VH modeling of pGX9256.

FIG. 13 depicts exemplary images of Fv, VH-VL and VL-VH modeling of pGX9290.

FIG. 14 depicts a flow diagram showing engineered DMAbs are synthesized as DNA plasmids and then evaluated in vitro and in vivo for antigen binding and expression.

FIG. 15A through FIG. 15B depict exemplary experimental results demonstrating the neutralizing activity of structurally reformatted DMAbs. FIG. 15A depicts exemplary experimental results demonstrating the percent neutralization for Group 1 EBOMAb-10. FIG. 15B depicts exemplary experimental results demonstrating the percent neutralization for the Group 2 (EBOMAb-14) using lentivirus pseudotyped with EBOLA Zaire glycoprotein (EBOV-GP)

FIG. 16 depicts a quantification of the neutralizing activity, IC50 and IC90 of the Group 1 and Group 2 structurally modified DMAbs.

FIG. 17A through FIG. 17F depict exemplary experimental results demonstrating the expression and antigen binding of the Group 1 and Group 2 structurally modified DMAbs. FIG. 17A depicts an in vivo time course expression of Group 1 DMAbs. Immunosuppressed BALB/c mice were administrated with 170 μg of DNA-plasmid encoding DMAb through intramuscular delivery followed by electroporation (IM-EP) Serum levels of DMAb were assessed over 35 days. FIG. 17B depicts an antigen binding curve for the Group 1 DMAbs. DMAbs in the serum were evaluated for reactivity to Ebola antigen. FIG. 17C depicts a Group 1 DMAb neutralization curve as evaluated using EBOV-GP pseudotyped lentivirus vector. FIG. 17D depicts an in vivo time course expression of Group 2 DMAbs. Immunosuppressed BALB/c mice were administrated with 200 μg of DNA-plasmid encoding DMAb through intramuscular delivery followed by electroporation (IM-EP) Serum levels of DMAb were assessed over 35 days. FIG. 17E depicts an antigen binding curve for the Group 2 DMAbs. DMAbs in the serum were evaluated for reactivity to Ebola antigen. FIG. 17F depicts a Group 2 DMAb neutralization curve as evaluated using EBOV-GP pseudotyped lentivirus vector.

FIG. 18A and FIG. 18B depict an analysis of gene optimization of the codon optimized mouse Zika DMAb ZK190G1M3LALA. FIG. 18A depicts expression data for each gene optimized DMAb. FIG. 18B depicts antigen binding for each gene optimized DMAb. These experiments were performed in vitro, in HEK293 cells.

FIG. 19A and FIG. 19B depict an in vivo analysis of expression and binding of gene optimized mouse Zika DMAb ZK190G1M3LALA. FIG. 19A depicts in vivo day 7 expression data for each gene optimized DMAb. FIG. 19B depicts antigen binding for each gene optimized DMAb.

FIG. 20A and FIG. 20B depict an analysis of expression of gene optimized ZK185LALA FP2A codon optimized constructs. FIG. 20A depicts in vivo day 7 expression data for each gene optimized DMAb. FIG. 20B depicts in vitro expression data for each gene optimized DMAb, in HEK293 cells.

FIG. 21A and FIG. 21B depict an analysis of the binding capability of gene optimized ZK185LALA FP2A codon optimized constructs. FIG. 21A depicts in vivo day 7 binding activity for each gene optimized DMAb. FIG. 21B depicts in vitro binding activity for each gene optimized DMAb, in HEK293 cells.

FIG. 22A and FIG. 22B depict an analysis of ScFv-Fc conversion of the codon optimized mouse Zika DMAb ZK190G1M3LALA. FIG. 22A depicts expression data for each ScFv-Fc DMAb. FIG. 22B depicts antigen binding for each ScFv-Fc DMAb. These experiments were performed in vivo.

FIG. 23 depicts an analysis of the in vivo expression of ScFv-Fc conversion constructs of the ZK185LALA FP2A codon optimized DMAb.

FIG. 24 depicts an analysis of the in vivo binding capability of ScFv-Fc conversion constructs of the ZK185LALA FP2A codon optimized DMAb.

FIG. 25A and FIG. 25B depict a schematic diagram of an overview of DMAb in-vivo delivery and protein-engineering. FIG. 25A depicts a schematic diagram demonstrating that anti-RSV antibodies were engineered for scFv-Fc expression. FIG. 25B depicts a schematic diagram demonstrating that DMAbs are delivered in-vivo by facilitated Electroporation using the CELLECTRA-3P® device. Transfected myocytes express and secret the protein MAb. Protein-MAb enters blood circulation.

FIG. 26A through FIG. 26D depict expression of scFv-Fc anti-RSV DMAbs. FIG. 26A depicts experimental results demonstrating the expression kinetics of RSV DMAbs. FIG. 26B depicts experimental results demonstrating the peak expression of RSV-DMAbs. FIG. 26C depicts experimental results demonstrating amount of DMAbs in Bronchoalveolar lavage (BAL) samples. FIG. 26D depicts experimental results demonstrating RSV-F binding.

FIG. 27 depicts experimental results demonstrating neutralization of the anti-RSV DMAbs

FIG. 28 depicts experimental results demonstrating anti-RSV-DMAbs in the cotton rat.

FIG. 29A through FIG. 29D depict exemplary experimental results demonstrating the peak expression and functionality of human sc-Fv anti RSV in immunocompetent mice. Mice were dosed with 200 μg human sc-Fv anti RSV dMAb delivered into leg muscles of balb/c mice. Delivery was assisted by CELLECTRA-3P®. FIG. 29A depicts exemplary experimental results demonstrating that average serum-level expression of 13200 ng/ml of protein human sc-Fv was achieved 7 days after treatment. FIG. 29B depicts exemplary experimental results demonstrating in-vivo expressed human sc-Fv binds to RSV-F antigen. FIG. 29C depicts exemplary experimental results demonstrating that the serum of treated mice exhibits live RSV-A virus-neutralizing activity as demonstrated by the in-vitro plaque reduction assay and results in average of 6.9 log recip. Neut. titer. FIG. 29D depicts exemplary experimental results demonstrating human sc-Fv is present in the lung of treated mice with an average concentration of 1.1 ng of human sc-Fv per μg of total protein in Bronchioalveolar-lavage (BAL).

FIG. 30 depicts exemplary experimental results demonstrating the maintained expression of human sc-Fv in cotton rats. 100 μg and 800 μg of human sc-Fv was delivered into TA muscle of cotton rats. Delivery was assisted with CELLECTRA-3P®. Peak expression in serum is reached after 7 days (226 ng/ml and 1353 ng/ml respectively).

FIG. 31A and FIG. 31B depict exemplary experimental results demonstrating the peak expression and functionality of human sc-Fv in cotton rats. FIG. 31A depicts exemplary experimental results demonstrating that CELLECTRA-3P® assisted delivery of 2.4 mg human sc-Fv in leg muscles of cotton rat results in average serum-expression of 7030 ng/ml at day 7. FIG. 31B depicts exemplary experimental results demonstrating that the serum of treated cotton rats is neutralizing in in-vitro plaque-reduction assay resulting in average of 5.4 log Recip. Neut. titer.

FIG. 32A through FIG. 32C depict schematic diagrams of the molecular and experimental design Dengue and Zika scFv-Fc were structurally-reformatted. FIG. 32A depicts a schematic representations of design approaches used. Human dMAb design strategies for ScFv-Fc. The VH and VL regions were connected by a (G4S)3 linker and fused to a prototypical human IgG1 hinge and Fc. FIG. 32B depicts a diagram of the molecular organization of scFv-Fc Zika and Dengue dMAb encoding plasmids (Z-dMAb1-sc and D-dMAb1-sc) as well as the multivalent bi-directional promoter construct (Z/D-dMAb1-sc). FIG. 32C depicts a flow chart of the Experimental design, various dMAb constructs in combination or within a multivalent cassette were delivered in each tibialis anterior (TA) muscle C57BL/6 mice followed by electroporation dMAb expression and antigen binding were evaluated at Day 7 post-DNA delivery.

FIG. 33 depicts exemplary experimental results demonstrating an evaluation of the cross-reactivity between Zika and Dengue virus. Zika dMAb react specifically with Zika antigen. Dengue dMAb reacts specifically to DENV1 and DENV2 antigens. Plasmid encoding scFv-FC-dMAbs were transfected in 293 T cells. Day 2 post transfection supernatant containing the scFv-Fc protein were submitted to antigen binding. 96 well plates were coated with 100 μl/well 1 μg/ml of Zika or Dengue antigen (DENV1-4). Reciprocal serum dilutions one in third (⅓) serial dilutions were performed with pre-diluted serum samples on 96 well plate. Antigen binding was assessed by ELISA. Data are expressed as mean OD450±SEM

FIG. 34A through FIG. 34C depict exemplary experimental results demonstrating in vitro expression and antigen binding of scFV-Fc dMAbs. Plasmid encoding scFv-FC-dMAbs were transfected in 293 T cells. FIG. 34A depicts exemplary experimental results demonstrating day 2 post transfection expression of scFv-Fc dMAbs and their respective contribution in co-transfection and in the multivalent Z/D-dMAb1-sc construct was assessed. FIG. 34B depicts exemplary experimental results demonstrating the ability of the scFv-Fc dMAbs to bind Dengue (DENV1). FIG. 34C depicts exemplary experimental results demonstrating the ability of the scFv-Fc dMAbs to bind Zika antigen.

FIG. 35A through FIG. 35C depict exemplary experimental results demonstrating in vivo expression and antigen binding of scFv-Fc dMAbs. FIG. 35A depicts exemplary experimental results demonstrating scFv-Fc dMAb expression day 7 post electroporation (EP) mediated-dMAb transfection. Data show individual dMAb expression for Z-dMAb1-sc & D-dMAb1-sc, as well as their respective contribution when delivered in cocktail in the same animals or when individually delivered at two distinct intramuscular sites in the same animals. Data also show the contribution of Dengue and Zika scFv-Fc dMAbs when expressed in a single multivalent plasmid construct. A total of 100 μg of dMAb encoding plasmid were delivered for the Z-dMAb1-sc, D-dMAb1-sc as well as Z/D-dMAb1-sc groups, and 200 μg for the cocktail delivery and individual delivery groups, followed by EP. Sera were collected Day 7 post-treatment for quantitative ELISA assessing scFv-Fc dMAb expression in C57BL/6 mice. FIG. 35B depicts ab exemplary antigen binding assay evaluating the ability for the various constructs to recognize Zika antigen. FIG. 35C depicts an exemplary antigen binding assay evaluating the ability for the various constructs to recognize Dengue antigen.

### DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV promoter, EF1alpha promoter, ACTA1 promoter, SV40 early promoter or SV 40 late promoter and the CMV IE promoter.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, including a nanoplasmid or mini-circle plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Structurally Modified DMAbs

The present invention relates to compositions comprising structurally modified DNA encoded synthetic antibody (DMAb), compositions comprising a nucleic acid molecules encoding structurally modified DMAbs, methods of generating structurally modified DMAbs, and methods of use of structurally modified DMAbs.

In one embodiment, a structurally modified DMAb, comprises at least one modification to increase expression, antigen binding, stability, or a combination thereof in vivo. In one embodiment, at least one modification is made on the basis of increasing the in vivo expression of a DMAb that has been designated as a low expressing DMab. In one embodiment, at least one modification is made on the basis of increasing in vivo antigen binding of a DMAb.

In one embodiment, a candidate DMAb for being structurally modified according to the present invention is a DMAb to exhibits desirable antigen binding in vivo, but low expression. Accordingly, the structural modification to generate a desirable DMAb is to increase the expression of that DMAb in order to generate a DMAb that exhibits both desirable antigen binding and higher expression level in vivo. In one embodiment, a structurally modified DMAb comprises at least one modification that results in the increased expression over the expression level of the unmodified DMAb.

In one embodiment, a structurally modified DMAb comprises one or more modification that increases the expression of a corresponding DMAb that has not be so modified. In one embodiment, the modification includes but is not limited to full graft, partial graft, scaffold modification, ScFv-Fc conversion, and the like. However, the invention should not be limited to these types of modifications. Rather, the invention includes any type of modification that is able to increase the in vivo expression or antigen binding of a DMAb. In one embodiment, the invention relates to a nucleic acid molecule encoding a structurally modified DMAb.

### Full Graft

In one embodiment, the structurally modified DMAb of the invention is a full graft DMAb. In one embodiment, full grafting relates to a method of transferring the sequence encoding at least one CDR region of a DMAb onto the backbone of a different DMAb. For example, in one embodiment, full grafting includes identifying at least one CDR region of a DMAb having low in vivo expression and modifying at least one CDR region of a DMAb having high in vivo expression levels to have the at least one CDR sequence of the low expressing DMAb.

In one embodiment, a full graft DMAb comprises a DMAb wherein at least one, at least two or all three CDRs from the variable heavy chain of one DMAb have been modified to be identical to at least one, at least two or all three CDRs from the variable heavy chain of a second DMAb. In one embodiment, a full graft DMAb comprises a DMAb wherein at least one, at least two or all three CDRs from the variable heavy chain of one DMAb have been modified to be at least 80%, 85%, 90%, 95%, 96%, 97%, 98%, or 99% identical to at least one, at least two or all three CDRs from the variable heavy chain of a second DMAb. In one embodiment, a full graft DMAb comprises a DMAb wherein at least one, at least two or all three CDRs from the variable light chain of one DMAb have been modified to be identical to at least one, at least two or all three CDRs from the variable light chain of a second DMAb.

The immunoglobulin scaffold for use in generating the full graft DMAb of the invention can be from any immunoglobulin isotype. Heavy chain immunoglobulin isotypes include, but are not limited to, IgA, including IgA1 and IgA2, IgD, IgE, IgG, including IgG1, IgG2, IgG3 and IgG4, and IgM. Light chain immunoglobulin isotypes include, but are not limited to kappa and lambda. In one embodiment, the DMAbs that serve as a basis for generation of a full graft DMAb are of the same immunoglobulin isotype. In one embodiment, the DMAbs that serve as a basis for generation of the full graft DMAb are from different immunoglobulin isotypes.

In one embodiment, the full graft DMAb of the invention has modified expression, stability, half-life, antigen binding, heavy chain-light chain pairing, tissue penetration or a combination thereof as compared to a parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the full graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

### Partial Graft

In one embodiment, the structurally modified DMAb of the invention is a partial graft DMAb. In one embodiment, partial grafting relates to a method of modifying one or more FR region, or fragment thereof, of a DMAb to contain one or more FR region, or fragment thereof, of a different DMAb. For example, in one embodiment, partial grafting includes modifying a FR region, or fragment thereof, of a DMAb having low in vivo expression to be similar to that of a second DMAb having high in vivo expression. In one embodiment, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more than 50 residues of at least one FR region are altered. In one embodiment, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more than 50 residues of multiple FR regions are altered. In one embodiment, at least 1, 2, 3, 4, 5, 6, 7, 8, 9, 10, 11, 12, 13, 14, 15, 16, 17, 18, 19, 20 or more than 20 residues are altered within close proximity to each other (e.g., at least 1, 2, 3, 4, 5, 6, 7, 8, 9, 10, 11, 12, 13, 14, 15, 16, 17, 18, 19, 20 or more than 20 residues are altered within a consecutive 30 residue region.) In one embodiment, at least 1, 2, 3, 4, 5, 6, 7, 8, 9, 10, 11, 12, 13, 14, 15, 16, 17, 18, 19, 20 or more than 20 residues are altered within a single FR (i.e., within FR1, FR2, FR3 or FR4.) In one embodiment, at least 1, 2, 3, 4, 5, 6, 7, 8, 9, 10, 11, 12, 13, 14, 15, 16, 17, 18, 19, 20 or more than 20 residues are altered within multiple FRs (e.g., within FR1 and FR4.) In an exemplary embodiment, at least 1, 2, 3, 4, 5, 6, 7, 8, 9, 10, 11, 12, 13, 14, 15, 16, 17, 18, 19 or 20 residues are altered at the N-terminus of the variable light chain.

The immunoglobulin scaffold of the DMAbs that serve as a basis for generating a partial graft DMAb of the invention can be from any immunoglobulin isotype including, but not limited to IgA, including IgA1 and IgA2, IgD, IgE, IgG, including IgG1, IgG2, IgG3 and IgG4, IgM, kappa and lambda. In one embodiment, the DMAbs that serve as a basis for generation of the partial graft DMAb are the same immunoglobulin isotype. In one embodiment, the DMAbs that serve as a basis for generation of the partial graft DMAb are different immunoglobulin isotypes.

In one embodiment, the partial graft DMAb of the invention has modified expression, stability, half-life, antigen binding, heavy chain-light chain pairing, tissue penetration or a combination thereof as compared to a parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the partial graft DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

### Scaffold Modification

In one embodiment, the structurally modified DMAb of the invention is a scaffold modified DMAb. In one embodiment, scaffold modification relates to a method of modifying at least one amino acid residue of a DMAb to increase stabilizing interactions at the V_{H}-V_{L} interface or to favorably alter isoelectric point. Residues that can be modified according to the method of the invention can be identified using any method known in the art for predicting residues involved in V_{H}-V_{L} interactions (e.g., using bioinformatics methods for predicting residues involved in the V_{H}-V_{L} interface as described in Abhinandan et al., 2010, Protein Eng Des Sel, 23(9):689-697.) In various embodiments, a scaffold modified DMAb comprises one or more modifications to alter non-aromatic side chain residues to aromatic side chain residues. In one embodiment, a scaffold modified DMAb comprises one or more modifications to alter a V_{H}-V_{L} interface amino acid with an electrically charged side chain to an amino acid with an uncharged side chain (e.g., a lysine (K) to glutamine (Q) modification). In one embodiment, a scaffold modified DMAb comprises one or more modifications to alter a V_{H}-V_{L} interface amino acid with an uncharged side chain to an amino acid with a hydrophobic side chain (e.g., a Q to tyrosine (Y) modification). In one embodiment, a scaffold modified DMAb comprises one or more modifications to alter a V_{H}-V_{interface} amino acid to an amino acid with an aromatic side chain (e.g., a Q to tyrosine (Y) modification). In various embodiments, a scaffold modified DMAb comprises one or more modifications within FR2 or FR3.

The immunoglobulin scaffold of the structurally modified DMAbs of the invention can be from any immunoglobulin isotype including, but not limited to IgA, including IgA1 and IgA2, IgD, IgE, IgG, including IgG1, IgG2, IgG3 and IgG4, IgM, kappa and lambda.

In one embodiment, the scaffold modified DMAb of the invention has modified expression, stability, half-life, antigen binding, heavy chain-light chain pairing, tissue penetration or a combination thereof as compared to a parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the scaffold modified DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

### Single Chain Fv-Fc (ScFv-Fc) Conversion

In one embodiment, the structurally modified DMAb of the invention is a ScFv-Fc DMAb. In one embodiment, ScFv-Fc conversion relates to the removal of CH1 and CL regions, and the addition of a linker between VH and VL. Therefore, in one embodiment, the structurally modified DMAb of the invention comprises a DMAb encoded by nucleic acid molecule lacking an encoding sequence for the CH1 and CL domain and comprising an encoding sequence for a linker between the sequence encoding the VH domain and the sequence encoding the VL domain. In one embodiment, a ScFv-Fc DMAb is in VH-VL orientation, comprising a linker attaching the C terminus of the VH region to the N terminus of the VL region. In one embodiment, a ScFv-Fc DMAb is in VL-VH orientation, comprising a linker attaching the C terminus of the VL region to the N terminus of the VH region. In one embodiment, a linker comprises at least 10, 11, 12, 13, 14, 15, 16, 17, 18, 19, 20, or more than 20 amino acid residues. In one embodiment, a linker comprises a (G4S)3 linker, having a sequence according to GGGGSGGGGSGGGGS (SEQ ID NO:53). In another embodiment, the linker is the Whitlow linker, having an amino acid sequence GSTSGSGKPGSGEGSTKG (SEQ ID NO:109).

The immunoglobulin scaffold of the ScFv-Fc DMAb of the invention can be from any immunoglobulin isotype including, but not limited to IgA, including IgA1 and IgA2, IgD, IgE, IgG, including IgG1, IgG2, IgG3 and IgG4, IgM, kappa and lambda.

In one embodiment, the ScFv-Fc converted antibody of the invention has modified expression, stability, half-life, antigen binding, heavy chain-light chain pairing, tissue penetration or a combination thereof as compared to a parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater tissue penetration than the parental DMAb.

In one embodiment, the ScFv-Fc DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold greater heavy chain-light chain pairing than the parental DMAb.

### Gene Optimization

In one embodiment, the structurally modified DMAb of the invention is a gene optimized (GO) DMAb. In one embodiment, gene optimization relates to a method in which multiple parameters affecting transcription and translation, such as codon usage, GC content, cryptic splice sites and mRNA secondary structure are weighted in multivariate regression algorithms to generate a sequence having modified expression, stability, half-life, antigen binding, or a combination thereof as compared to a parental DMAb.

In one embodiment, the gene optimized DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher expression than the parental DMAb.

In one embodiment, the gene optimized DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher antigen binding than the parental DMAb.

In one embodiment, the gene optimized DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold longer half-life than the parental DMAb.

In one embodiment, the gene optimized DMAb of the invention has at least 1.1 fold, at least 1.2 fold, fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 20 fold, at least 30 fold, at least 40 fold, at least 50 fold or greater than 50 fold higher stability than the parental DMAb.

Nucleic acid molecules encoding structurally modified DMAbs

In one embodiment, the invention provides compositions comprising a nucleic acid molecule encoding a structurally modified DMAb. In various embodiments, the nucleic acid sequence encodes a structurally modified DMAb designed to have increased expression, stability, half-life, antigen binding, or a combination thereof over a parental DMAb. In one embodiment, the nucleic acid sequence encodes a full graft DMAb, a partial graft DMA, a scaffold modified DMAb, a gene optimized DMAb, or a ScFv-Fc conversion DMAb.

### Anti-Ebola DMAb

In one embodiment, the structurally modified DMAb is an anti-Ebola DMAb.

In one embodiment, a nucleic acid molecule encoding a full graft structurally modified anti-Ebola DMAb encodes a DMAb having an amino acid sequence of SEQ ID NO:6, SEQ ID NO: 8, SEQ ID NO:10, SEQ ID NO: 16 or SEQ ID NO:18. In one embodiment, a nucleic acid molecule encoding a full graft structurally modified anti-Ebola DMAb comprises an nucleotide sequence of SEQ ID NO:5, SEQ ID NO:7, SEQ ID NO:9, SEQ ID NO: 15 or SEQ ID NO: 17.

In one embodiment, a nucleic acid molecule encoding a partial graft structurally modified anti-Ebola DMAb encodes a DMAb having an amino acid sequence of SEQ ID NO:4, SEQ ID NO: 14, SEQ ID NO:30 or SEQ ID NO:34. In one embodiment, a nucleic acid molecule encoding a partial graft structurally modified anti-Ebola DMAb comprises an nucleotide sequence of SEQ ID NO:3, SEQ ID NO: 13, SEQ ID NO:29 or SEQ ID NO: 33.

In one embodiment, a nucleic acid molecule encoding a scaffold modified structurally modified anti-Ebola DMAb encodes a DMAb having an amino acid sequence of SEQ ID NO:2 or SEQ ID NO:12. In one embodiment, a nucleic acid molecule encoding a scaffold modified structurally modified anti-Ebola DMAb comprises an nucleotide sequence of SEQ ID NO:2 or SEQ ID NO: 12.

In one embodiment, a nucleic acid molecule encoding a ScFv-Fc modified anti-Ebola DMAb encodes a DMAb having an amino acid sequence of SEQ ID NO:20, SEQ ID NO: 22, SEQ ID NO:24, SEQ ID NO:26, SEQ ID NO:28, SEQ ID NO:32 or SEQ ID NO: 36. In one embodiment, a nucleic acid molecule encoding a ScFv-Fc modified anti-Ebola DMAb comprises an nucleotide sequence of SEQ ID NO:20, SEQ ID NO:22, SEQ ID NO: 24, SEQ ID NO:26, SEQ ID NO:28, SEQ ID NO:32 or SEQ ID NO:36.

In one embodiment, a nucleic acid molecule comprises a sequence encoding a fragment of a structurally modified anti-Ebola DMAb. In one embodiment, a fragment of a nucleic acid molecule encoding a structurally modified DMAb is encodes a variable light chain region or a variable heavy chain region of a structurally modified DMAb.

In one embodiment, a nucleic acid molecule encoding one or more structurally modified anti-Ebola DMAb encodes a DMAb having an amino acid sequence having at least 90%, at least 91%, at least 92%, at least 93%, at least 94%, at least 95%, at least 96%, at least 97%, at least 98%, at least 99%, or 100% identity over an entire length of the encoded sequence to an amino acid sequence of SEQ ID NO:2, SEQ ID NO:4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO: 40, SEQ ID NO:42, SEQ ID NO:44 or SEQ ID NO:46.

In one embodiment, a nucleic acid molecule encoding one or more structurally modified anti-Ebola DMAb comprises a nucleotide sequence having at least 90%, at least 91%, at least 92%, at least 93%, at least 94%, at least 95%, at least 96%, at least 97%, at least 98%, at least 99%, or 100% identity over an entire length of the nucleic acid sequence to a nucleic acid sequence of SEQ ID NO: 1, SEQ ID NO:3, SEQ ID NO:5, SEQ ID NO:7, SEQ ID NO:9, SEQ ID NO: 11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO: 41, SEQ ID NO:43 or SEQ ID NO:45.

In one embodiment, a nucleic acid molecule comprises a sequence encoding a fragment of a structurally modified anti-Ebola DMAb. In one embodiment, a fragment of a nucleic acid molecule encoding a structurally modified anti-Ebola DMAb is encodes a variable light chain region or a variable heavy chain region of a structurally modified anti-Ebola DMAb.

In one embodiment, a nucleic acid molecule encoding one or more structurally modified anti-Ebola DMAbs comprises a nucleotide sequence encoding a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of an amino acid sequence of SEQ ID NO: 2, SEQ ID NO:4, SEQ ID NO:6, SEQ ID NO:8, SEQ ID NO: 10, SEQ ID NO: 12, SEQ ID NO: 14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO: 36, SEQ ID NO:38, SEQ ID NO:40, SEQ ID NO:42, SEQ ID NO:44 or SEQ ID NO:46.

In one embodiment, a nucleic acid molecule encoding one or more structurally modified anti-Ebola DMAbs comprises a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a nucleotide sequence of SEQ ID NO:1, SEQ ID NO:3, SEQ ID NO:5, SEQ ID NO: 7, SEQ ID NO:9, SEQ ID NO:11, SEQ ID NO:13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO: 29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45.

In one embodiment, a nucleic acid molecule encoding one or more structurally modified anti-Ebola DMAbs comprises a nucleotide sequence encoding an amino acid sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the encoded sequence to an amino acid sequence of SEQ ID NO: 2, SEQ ID NO:4, SEQ ID NO:6, SEQ ID NO:8, SEQ ID NO: 10, SEQ ID NO: 12, SEQ ID NO: 14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO: 36, SEQ ID NO:38, SEQ ID NO:40, SEQ ID NO:42, SEQ ID NO:44 or SEQ ID NO:46.

In one embodiment, a nucleic acid molecule encoding one or more structurally modified anti-Ebola DMAbs comprises a nucleotide sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the nucleic acid sequence to a nucleic acid sequence of SEQ ID NO:1, SEQ ID NO:3, SEQ ID NO: 5, SEQ ID NO:7, SEQ ID NO:9, SEQ ID NO: 11, SEQ ID NO: 13, SEQ ID NO: 15, SEQ ID NO: 17, SEQ ID NO:19, SEQ ID NO:21, SEQ ID NO:23, SEQ ID NO:25, SEQ ID NO: 27, SEQ ID NO:29, SEQ ID NO:31, SEQ ID NO:33, SEQ ID NO:35, SEQ ID NO:37, SEQ ID NO:39, SEQ ID NO:41, SEQ ID NO:43 or SEQ ID NO:45.

In one embodiment, the nucleotide sequence encoding one or more structurally modified anti-Ebola DMAbs comprises one or more RNA sequences transcribed from one or more DNA sequences encoding an amino acid sequence at least 90%, at least 91%, at least 92%, at least 93%, at least 94%, at least 95%, at least 96%, at least 97%, at least 98%, or at least 99% identical to SEQ ID NO:2, SEQ ID NO:4, SEQ ID NO:6, SEQ ID NO: 8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO: 30, SEQ ID NO:32, SEQ ID NO:34, SEQ ID NO:36, SEQ ID NO:38, SEQ ID NO:40, SEQ ID NO:42, SEQ ID NO:44 or SEQ ID NO:46 or a fragment of an amino acid sequence at least 90%, at least 91%, at least 92%, at least 93%, at least 94%, at least 95%, at least 96%, at least 97%, at least 98%, or at least 99% identical to SEQ ID NO:2, SEQ ID NO:4, SEQ ID NO: 6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO: 40, SEQ ID NO:42, SEQ ID NO:44 or SEQ ID NO:46.

In one embodiment, the nucleotide sequence encoding an anti-Ebola DMAb comprises one or more RNA sequences transcribed from one or more DNA sequences encoding an amino acid sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO: 40, SEQ ID NO:42, SEQ ID NO:44 or SEQ ID NO:46, or a fragment of an amino acid sequence as set forth in SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO: 32, SEQ ID NO:34, SEQ ID NO:36, SEQ ID NO:38, SEQ ID NO:40, SEQ ID NO:42, SEQ ID NO:44 or SEQ ID NO:46.

In one embodiment, the nucleotide sequence encoding an anti-Ebola DMAb comprises one or more RNA sequences transcribed from one or more DNA sequences at least 90%, at least 91%, at least 92%, at least 93%, at least 94%, at least 95%, at least 96%, at least 97%, at least 98%, or at least 99% identical to SEQ ID NO:1, SEQ ID NO:3, SEQ ID NO: 5, SEQ ID NO:7, SEQ ID NO:9, SEQ ID NO:11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO: 39, SEQ ID NO:41, SEQ ID NO:43 or SEQ ID NO:45, or a fragment of a DNA sequence at least 90%, at least 91%, at least 92%, at least 93%, at least 94%, at least 95%, at least 96%, at least 97%, at least 98%, or at least 99% identical to SEQ ID NO:1, SEQ ID NO:3, SEQ ID NO: 5, SEQ ID NO: 7, SEQ ID NO:9, SEQ ID NO:11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO: 39, SEQ ID NO:41, SEQ ID NO:43 or SEQ ID NO:45.

In one embodiment, the nucleotide sequence encoding an anti-Ebola DMAb comprises one or more RNA sequence transcribed from one or more DNA sequences as set forth in SEQ ID NO:1, SEQ ID NO:3, SEQ ID NO:5, SEQ ID NO:7, SEQ ID NO:9, SEQ ID NO:11, SEQ ID NO: 13, SEQ ID NO: 15, SEQ ID NO: 17, SEQ ID NO: 19, SEQ ID NO: 21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO: 43 or SEQ ID NO:45, or a fragment of a DNA sequence as set forth in SEQ ID NO:1, SEQ ID NO:3, SEQ ID NO:5, SEQ ID NO:7, SEQ ID NO:9, SEQ ID NO: 11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO: 35, SEQ ID NO:37, SEQ ID NO:39, SEQ ID NO:41, SEQ ID NO:43 or SEQ ID NO:45.

The composition of the invention can treat, prevent and/or protect against any disease, disorder, or condition associated with Ebola virus infection. In certain embodiments, the composition can treat, prevent, and or/protect against viral infection. In certain embodiments, the composition can treat, prevent, and or/protect against a condition associated with Ebola virus infection.

### Anti-Zika DMAb

In one embodiment, the structurally modified DMAb is an anti-ZIKV DMAb.

In one embodiment, a nucleic acid molecule encoding a gene optimized anti-ZIKV DMAb encodes a DMAb having an amino acid sequence of SEQ ID NO:64, SEQ ID NO: 66, SEQ ID NO:68, SEQ ID NO: 70, SEQ ID NO:72, SEQ ID NO: 74, SEQ ID NO: 76, SEQ ID NO: 78, SEQ ID NO:80, SEQ ID NO:82, SEQ ID NO: 84 or SEQ ID NO:86. In one embodiment, a nucleic acid molecule encoding a gene optimized anti-ZIKV DMAb comprises an nucleotide sequence of SEQ ID NO:63, SEQ ID NO:65, SEQ ID NO:67, SEQ ID NO: 69, SEQ ID NO: 71, SEQ ID NO: 73, SEQ ID NO: 75, SEQ ID NO: 77, SEQ ID NO: 79, SEQ ID NO:81, SEQ ID NO:83 or SEQ ID NO:85.

In one embodiment, a nucleic acid molecule encoding a ScFv-Fc modified anti-ZIKV DMAb encodes a DMAb having an amino acid sequence of SEQ ID NO:90, SEQ ID NO: 92, SEQ ID NO:94, SEQ ID NO:96, SEQ ID NO: 100, SEQ ID NO: 102, SEQ ID NO: 104 or SEQ ID NO:106. In one embodiment, a nucleic acid molecule encoding a ScFv-Fc modified DMAb comprises an nucleotide sequence of SEQ ID NO:89, SEQ ID NO:91, SEQ ID NO:93, SEQ ID NO:95, SEQ ID NO: 99, SEQ ID NO: 101, SEQ ID NO: 103 or SEQ ID NO: 105.

In one embodiment, a nucleic acid molecule encoding one or more structurally modified anti-ZIKV DMAb encodes a DMAb having an amino acid sequence having at least 90%, at least 91%, at least 92%, at least 93%, at least 94%, at least 95%, at least 96%, at least 97%, at least 98%, at least 99%, or 100% identity over an entire length of the encoded sequence to an amino acid sequence of SEQ ID NO:64, SEQ ID NO:66, SEQ ID NO: 68, SEQ ID NO: 70, SEQ ID NO: 72, SEQ ID NO: 74, SEQ ID NO: 76, SEQ ID NO: 78, SEQ ID NO:80, SEQ ID NO:82, SEQ ID NO:84, SEQ ID NO:86, SEQ ID NO:88, SEQ ID NO: 90, SEQ ID NO:92, SEQ ID NO:94, SEQ ID NO:96, SEQ ID NO:98, SEQ ID NO: 100, SEQ ID NO:102, SEQ ID NO: 104 or SEQ ID NO:106.

In one embodiment, a nucleic acid molecule encoding one or more structurally modified anti-ZIKV DMAb comprises a nucleotide sequence having at least 90%, at least 91%, at least 92%, at least 93%, at least 94%, at least 95%, at least 96%, at least 97%, at least 98%, at least 99%, or 100% identity over an entire length of the nucleic acid sequence to a nucleic acid sequence of SEQ ID NO:63, SEQ ID NO:65, SEQ ID NO:67, SEQ ID NO: 69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO: 91, SEQ ID NO:93, SEQ ID NO:95, SEQ ID NO:97, SEQ ID NO:99, SEQ ID NO: 101, SEQ ID NO: 103 or SEQ ID NO:105.

In one embodiment, a nucleic acid molecule comprises a sequence encoding a fragment of a structurally modified anti-ZIKV DMAb. In one embodiment, a fragment of a nucleic acid molecule encoding a structurally modified anti-ZIKV DMAb is encodes a variable light chain region or a variable heavy chain region of a structurally modified anti-ZIKV DMAb.

In one embodiment, a nucleic acid molecule encoding one or more structurally modified anti-ZIKV DMAbs comprises a nucleotide sequence encoding a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of an amino acid sequence of SEQ ID NO: 64, SEQ ID NO: 66, SEQ ID NO: 68, SEQ ID NO: 70, SEQ ID NO: 72, SEQ ID NO: 74, SEQ ID NO:76, SEQ ID NO:78, SEQ ID NO:80, SEQ ID NO:82, SEQ ID NO:84, SEQ ID NO: 86, SEQ ID NO:88, SEQ ID NO:90, SEQ ID NO:92, SEQ ID NO:94, SEQ ID NO:96, SEQ ID NO:98, SEQ ID NO: 100, SEQ ID NO:102, SEQ ID NO:104 or SEQ ID NO:106.

In one embodiment, a nucleic acid molecule encoding one or more structurally modified anti-ZIKV DMAbs comprises a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a nucleotide sequence of SEQ ID NO:63, SEQ ID NO:65, SEQ ID NO:67, SEQ ID NO:69, SEQ ID NO: 71, SEQ ID NO:73, SEQ ID NO: 75, SEQ ID NO: 77, SEQ ID NO: 79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO: 99, SEQ ID NO: 101, SEQ ID NO:103 or SEQ ID NO:105.

In one embodiment, a nucleic acid molecule encoding one or more structurally modified anti-ZIKV DMAbs comprises a nucleotide sequence encoding an amino acid sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the encoded sequence to an amino acid sequence of SEQ ID NO: 64, SEQ ID NO:66, SEQ ID NO: 68, SEQ ID NO: 70, SEQ ID NO: 72, SEQ ID NO: 74, SEQ ID NO:76, SEQ ID NO:78, SEQ ID NO:80, SEQ ID NO:82, SEQ ID NO:84, SEQ ID NO: 86, SEQ ID NO: 88, SEQ ID NO:90, SEQ ID NO:92, SEQ ID NO:94, SEQ ID NO:96, SEQ ID NO:98, SEQ ID NO: 100, SEQ ID NO: 102, SEQ ID NO: 104 or SEQ ID NO:106.

In one embodiment, a nucleic acid molecule encoding one or more structurally modified anti-ZIKV DMAbs comprises a nucleotide sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the nucleic acid sequence to a nucleic acid sequence of SEQ ID NO:63, SEQ ID NO:65, SEQ ID NO: 67, SEQ ID NO:69, SEQ ID NO: 71, SEQ ID NO: 73, SEQ ID NO: 75, SEQ ID NO: 77, SEQ ID NO: 79, SEQ ID NO:81, SEQ ID NO:83, SEQ ID NO:85, SEQ ID NO:87, SEQ ID NO: 89, SEQ ID NO:91, SEQ ID NO: 93, SEQ ID NO: 95, SEQ ID NO:97, SEQ ID NO:99, SEQ ID NO: 101, SEQ ID NO: 103 or SEQ ID NO:105.

In one embodiment, the nucleotide sequence encoding one or more structurally modified anti-ZIKV DMAbs comprises one or more RNA sequences transcribed from one or more DNA sequences encoding an amino acid sequence at least 90%, at least 91%, at least 92%, at least 93%, at least 94%, at least 95%, at least 96%, at least 97%, at least 98%, or at least 99% identical to SEQ ID NO:64, SEQ ID NO:66, SEQ ID NO:68, SEQ ID NO: 70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO: 92, SEQ ID NO:94, SEQ ID NO:96, SEQ ID NO:98, SEQ ID NO: 100, SEQ ID NO: 102, SEQ ID NO:104 or SEQ ID NO:106 or a fragment of an amino acid sequence at least 90%, at least 91%, at least 92%, at least 93%, at least 94%, at least 95%, at least 96%, at least 97%, at least 98%, or at least 99% identical to SEQ ID NO:64, SEQ ID NO:66, SEQ ID NO:68, SEQ ID NO: 70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO: 92, SEQ ID NO:94, SEQ ID NO:96, SEQ ID NO:98, SEQ ID NO: 100, SEQ ID NO: 102, SEQ ID NO: 104 or SEQ ID NO:106.

In one embodiment, the nucleotide sequence encoding an anti-ZIKV DMAb comprises one or more RNA sequences transcribed from one or more DNA sequences encoding an amino acid sequence as set forth in SEQ ID NO:64, SEQ ID NO:66, SEQ ID NO: 68, SEQ ID NO: 70, SEQ ID NO: 72, SEQ ID NO: 74, SEQ ID NO: 76, SEQ ID NO: 78, SEQ ID NO:80, SEQ ID NO:82, SEQ ID NO:84, SEQ ID NO:86, SEQ ID NO:88, SEQ ID NO: 90, SEQ ID NO: 92, SEQ ID NO:94, SEQ ID NO:96, SEQ ID NO:98, SEQ ID NO: 100, SEQ ID NO:102, SEQ ID NO:104 or SEQ ID NO:106, or a fragment of an amino acid sequence as set forth in SEQ ID NO:64, SEQ ID NO:66, SEQ ID NO:68, SEQ ID NO: 70, SEQ ID NO:72, SEQ ID NO:74, SEQ ID NO:76, SEQ ID NO: 78, SEQ ID NO:80, SEQ ID NO: 82, SEQ ID NO: 84, SEQ ID NO: 86, SEQ ID NO: 88, SEQ ID NO:90, SEQ ID NO:92, SEQ ID NO:94, SEQ ID NO:96, SEQ ID NO:98, SEQ ID NO:100, SEQ ID NO: 102, SEQ ID NO: 104 or SEQ ID NO:106.

In one embodiment, the nucleotide sequence encoding an anti-ZIKV DMAb comprises one or more RNA sequences transcribed from one or more DNA sequences at least 90%, at least 91%, at least 92%, at least 93%, at least 94%, at least 95%, at least 96%, at least 97%, at least 98%, or at least 99% identical to SEQ ID NO:63, SEQ ID NO:65, SEQ ID NO: 67, SEQ ID NO:69, SEQ ID NO:71, SEQ ID NO:73, SEQ ID NO: 75, SEQ ID NO: 77, SEQ ID NO: 79, SEQ ID NO:81, SEQ ID NO:83, SEQ ID NO:85, SEQ ID NO:87, SEQ ID NO: 89, SEQ ID NO: 91, SEQ ID NO: 93, SEQ ID NO: 95, SEQ ID NO: 97, SEQ ID NO:99, SEQ ID NO: 101, SEQ ID NO: 103 or SEQ ID NO:105 or a fragment of a DNA sequence at least 90%, at least 91%, at least 92%, at least 93%, at least 94%, at least 95%, at least 96%, at least 97%, at least 98%, or at least 99% identical to SEQ ID NO:63, SEQ ID NO:65, SEQ ID NO: 67, SEQ ID NO:69, SEQ ID NO: 71, SEQ ID NO: 73, SEQ ID NO: 75, SEQ ID NO: 77, SEQ ID NO: 79, SEQ ID NO:81, SEQ ID NO:83, SEQ ID NO:85, SEQ ID NO:87, SEQ ID NO: 89, SEQ ID NO:91, SEQ ID NO: 93, SEQ ID NO:95, SEQ ID NO:97, SEQ ID NO:99, SEQ ID NO: 101, SEQ ID NO:103 or SEQ ID NO:105.

In one embodiment, the nucleotide sequence encoding an anti-ZIKV DMAb comprises one or more RNA sequence transcribed from one or more DNA sequences as set forth in SEQ ID NO:63, SEQ ID NO:65, SEQ ID NO:67, SEQ ID NO:69, SEQ ID NO: 71, SEQ ID NO: 73, SEQ ID NO: 75, SEQ ID NO: 77, SEQ ID NO: 79, SEQ ID NO:81, SEQ ID NO:83, SEQ ID NO:85, SEQ ID NO:87, SEQ ID NO:89, SEQ ID NO:91, SEQ ID NO: 93, SEQ ID NO:95, SEQ ID NO:97, SEQ ID NO:99, SEQ ID NO: 101, SEQ ID NO: 103 or SEQ ID NO: 105, or a fragment of a DNA sequence as set forth in SEQ ID NO:63, SEQ ID NO: 65, SEQ ID NO:67, SEQ ID NO:69, SEQ ID NO: 71, SEQ ID NO: 73, SEQ ID NO: 75, SEQ ID NO:77, SEQ ID NO:79, SEQ ID NO:81, SEQ ID NO:83, SEQ ID NO:85, SEQ ID NO: 87, SEQ ID NO: 89, SEQ ID NO:91, SEQ ID NO:93, SEQ ID NO:95, SEQ ID NO:97, SEQ ID NO:99, SEQ ID NO: 101, SEQ ID NO: 103 or SEQ ID NO: 105.

The composition of the invention can treat, prevent and/or protect against any disease, disorder, or condition associated with Zika virus infection. In certain embodiments, the composition can treat, prevent, and or/protect against viral infection. In certain embodiments, the composition can treat, prevent, and or/protect against a condition associated with Zika virus infection.

### Anti-DENV DMAb

In one embodiment, the structurally modified DMAb is an anti-DENV DMAb.

In one embodiment, a nucleic acid molecule encoding a ScFv-Fc structurally modified anti-DENV DMAb encodes a DMAb having an amino acid sequence of SEQ ID NO:108. In one embodiment, a nucleic acid molecule encoding a ScFv-Fc structurally modified DMAb comprises an nucleotide sequence of SEQ ID NO:107.

In one embodiment, a nucleic acid molecule encoding one or more structurally modified anti-DENV DMAb encodes a DMAb having an amino acid sequence having at least 90%, at least 91%, at least 92%, at least 93%, at least 94%, at least 95%, at least 96%, at least 97%, at least 98%, at least 99%, or 100% identity over an entire length of the encoded sequence to an amino acid sequence of SEQ ID NO:108.

In one embodiment, a nucleic acid molecule encoding one or more structurally modified anti-DENV DMAb comprises a nucleotide sequence having at least 90%, at least 91%, at least 92%, at least 93%, at least 94%, at least 95%, at least 96%, at least 97%, at least 98%, at least 99%, or 100% identity over an entire length of the nucleic acid sequence to a nucleic acid sequence of SEQ ID NO:107.

In one embodiment, a nucleic acid molecule comprises a sequence encoding a fragment of a structurally modified anti-DENV DMAb. In one embodiment, a fragment of a nucleic acid molecule encoding a structurally modified anti-DENV DMAb is encodes a variable light chain region or a variable heavy chain region of a structurally modified anti-DENV DMAb.

In one embodiment, a nucleic acid molecule encoding one or more structurally modified anti-DENV DMAbs comprises a nucleotide sequence encoding a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of an amino acid sequence of SEQ ID NO: 108.

In one embodiment, a nucleic acid molecule encoding one or more structurally modified anti-DENV DMAbs comprises a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a nucleotide sequence of SEQ ID NO:107.

In one embodiment, a nucleic acid molecule encoding one or more structurally modified anti-DENV DMAbs comprises a nucleotide sequence encoding an amino acid sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the encoded sequence to an amino acid sequence of SEQ ID NO: 108.

In one embodiment, a nucleic acid molecule encoding one or more structurally modified anti-DENV DMAbs comprises a nucleotide sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the nucleic acid sequence to a nucleic acid sequence of SEQ ID NO:107.

In one embodiment, the nucleotide sequence encoding one or more structurally modified anti-DENV DMAbs comprises one or more RNA sequences transcribed from one or more DNA sequences encoding an amino acid sequence at least 90%, at least 91%, at least 92%, at least 93%, at least 94%, at least 95%, at least 96%, at least 97%, at least 98%, or at least 99% identical to SEQ ID NO:108 or a fragment of an amino acid sequence at least 90%, at least 91%, at least 92%, at least 93%, at least 94%, at least 95%, at least 96%, at least 97%, at least 98%, or at least 99% identical to SEQ ID NO: 108.

In one embodiment, the nucleotide sequence encoding an anti-DENV DMAb comprises one or more RNA sequences transcribed from one or more DNA sequences encoding an amino acid sequence as set forth in SEQ ID NO:108 or a fragment of an amino acid sequence as set forth in SEQ ID NO:108.

In one embodiment, the nucleotide sequence encoding an anti-DENV DMAb comprises one or more RNA sequences transcribed from one or more DNA sequences at least 90%, at least 91%, at least 92%, at least 93%, at least 94%, at least 95%, at least 96%, at least 97%, at least 98%, or at least 99% identical to SEQ ID NO:107 or a fragment of a DNA sequence at least 90%, at least 91%, at least 92%, at least 93%, at least 94%, at least 95%, at least 96%, at least 97%, at least 98%, or at least 99% identical to SEQ ID NO:107.

In one embodiment, the nucleotide sequence encoding an anti-DENV DMAb comprises one or more RNA sequence transcribed from one or more DNA sequences as set forth in SEQ ID NO: 107 or a fragment of a DNA sequence as set forth in SEQ ID NO: 107.

The composition of the invention can treat, prevent and/or protect against any disease, disorder, or condition associated with DENV virus infection. In certain embodiments, the composition can treat, prevent, and or/protect against viral infection. In certain embodiments, the composition can treat, prevent, and or/protect against a condition associated with DENV virus infection.

### Anti-RSV DMAb

In one embodiment, the structurally modified DMAb is an anti-RSV DMAb.

In one embodiment, a nucleic acid molecule encoding a ScFv-Fc structurally modified anti-RSV DMAb encodes a DMAb having an amino acid sequence of SEQ ID NO:113 or SEQ ID NO:117. In one embodiment, a nucleic acid molecule encoding a ScFv-Fc structurally modified DMAb comprises an nucleotide sequence of SEQ ID NO:112 or SEQ ID NO:116.

In one embodiment, a nucleic acid molecule encoding a gene optimized anti-RSV DMAb encodes a DMAb having an amino acid sequence of SEQ ID NO:111, SEQ ID NO: 115 or SEQ ID NO:119. In one embodiment, a nucleic acid molecule encoding a gene optimized anti-RSV DMAb comprises an nucleotide sequence of SEQ ID NO:110, SEQ ID NO: 114 or SEQ ID NO:118.

In one embodiment, a nucleic acid molecule encoding one or more structurally modified anti-RSV DMAb encodes a DMAb having an amino acid sequence having at least 90%, at least 91%, at least 92%, at least 93%, at least 94%, at least 95%, at least 96%, at least 97%, at least 98%, at least 99%, or 100% identity over an entire length of the encoded sequence to an amino acid sequence of SEQ ID NO:111, SEQ ID NO:113, SEQ ID NO: 115, SEQ ID NO: 117 or SEQ ID NO:119.

In one embodiment, a nucleic acid molecule encoding one or more structurally modified anti-RSV DMAb comprises a nucleotide sequence having at least 90%, at least 91%, at least 92%, at least 93%, at least 94%, at least 95%, at least 96%, at least 97%, at least 98%, at least 99%, or 100% identity over an entire length of the nucleic acid sequence to a nucleic acid sequence of SEQ ID NO:110, SEQ ID NO: 112, SEQ ID NO:114, SEQ ID NO: 116 or SEQ ID NO:118.

In one embodiment, a nucleic acid molecule comprises a sequence encoding a fragment of a structurally modified anti-RSV DMAb. In one embodiment, a fragment of a nucleic acid molecule encoding a structurally modified anti-RSV DMAb is encodes a variable light chain region or a variable heavy chain region of a structurally modified anti-RSV DMAb.

In one embodiment, a nucleic acid molecule encoding one or more structurally modified anti-RSV DMAbs comprises a nucleotide sequence encoding a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of an amino acid sequence of SEQ ID NO: 111, SEQ ID NO:113, SEQ ID NO:115, SEQ ID NO:117 or SEQ ID NO:119.

In one embodiment, a nucleic acid molecule encoding one or more structurally modified anti-RSV DMAbs comprises a fragment comprising at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of a nucleotide sequence of SEQ ID NO: 110, SEQ ID NO: 112, SEQ ID NO:114, SEQ ID NO:116 or SEQ ID NO:118.

In one embodiment, a nucleic acid molecule encoding one or more structurally modified anti-RSV DMAbs comprises a nucleotide sequence encoding an amino acid sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the encoded sequence to an amino acid sequence of SEQ ID NO: 111, SEQ ID NO:113, SEQ ID NO:115, SEQ ID NO: 117 or SEQ ID NO:119.

In one embodiment, a nucleic acid molecule encoding one or more structurally modified anti-RSV DMAbs comprises a nucleotide sequence having at least 90%, at least 91%, at least 92%, at least 93%, at least 94%, at least 95%, at least 96%, at least 97%, at least 98%, at least 99%, or 100% identity over at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the nucleic acid sequence to a nucleic acid sequence of SEQ ID NO:110, SEQ ID NO: 112, SEQ ID NO: 114, SEQ ID NO:116 or SEQ ID NO:118.

In one embodiment, the nucleotide sequence encoding one or more structurally modified anti-RSV DMAbs comprises one or more RNA sequences transcribed from one or more DNA sequences encoding an amino acid sequence at least 90%, at least 91%, at least 92%, at least 93%, at least 94%, at least 95%, at least 96%, at least 97%, at least 98%, or at least 99% identical to SEQ ID NO:111, SEQ ID NO:113, SEQ ID NO: 115, SEQ ID NO: 117 or SEQ ID NO:119, or a fragment of an amino acid sequence at least 90%, at least 91%, at least 92%, at least 93%, at least 94%, at least 95%, at least 96%, at least 97%, at least 98%, or at least 99% identical to SEQ ID NO:111, SEQ ID NO: 113, SEQ ID NO: 115, SEQ ID NO: 117 or SEQ ID NO:119.

In one embodiment, the nucleotide sequence encoding an anti-RSV DMAb comprises one or more RNA sequences transcribed from one or more DNA sequences encoding an amino acid sequence as set forth in SEQ ID NO:111, SEQ ID NO:113, SEQ ID NO: 115, SEQ ID NO:117 or SEQ ID NO:119, or a fragment of an amino acid sequence as set forth in SEQ ID NO:111, SEQ ID NO:113, SEQ ID NO:115, SEQ ID NO: 117 or SEQ ID NO: 119.

In one embodiment, the nucleotide sequence encoding an anti-RSV DMAb comprises one or more RNA sequences transcribed from one or more DNA sequences at least 90%, at least 91%, at least 92%, at least 93%, at least 94%, at least 95%, at least 96%, at least 97%, at least 98%, or at least 99% identical to SEQ ID NO:110, SEQ ID NO:112, SEQ ID NO: 114, SEQ ID NO:116 or SEQ ID NO:118 or a fragment of a DNA sequence at least 90%, at least 91%, at least 92%, at least 93%, at least 94%, at least 95%, at least 96%, at least 97%, at least 98%, or at least 99% identical to SEQ ID NO: 110, SEQ ID NO: 112, SEQ ID NO: 114, SEQ ID NO:116 or SEQ ID NO:118.

In one embodiment, the nucleotide sequence encoding an anti-RSV DMAb comprises one or more RNA sequence transcribed from one or more DNA sequences as set forth in SEQ ID NO:110, SEQ ID NO: 112, SEQ ID NO: 114, SEQ ID NO: 116 or SEQ ID NO: 118, or a fragment of a DNA sequence as set forth in SEQ ID NO:110, SEQ ID NO:112, SEQ ID NO:114, SEQ ID NO:116 or SEQ ID NO:118.

The composition of the invention can treat, prevent and/or protect against any disease, disorder, or condition associated with RSV virus infection. In certain embodiments, the composition can treat, prevent, and or/protect against viral infection. In certain embodiments, the composition can treat, prevent, and or/protect against a condition associated with RSV virus infection.

### DNA Encoded Antibody

As described above, the composition can comprise a recombinant nucleic acid sequence. The recombinant nucleic acid sequence can encode the structurally modified DMAb, a fragment thereof, a variant thereof, or a combination thereof. The antibody is described in more detail below.

The recombinant nucleic acid sequence can be a heterologous nucleic acid sequence. The recombinant nucleic acid sequence can include at least one heterologous nucleic acid sequence or one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides. In one embodiment, the linker sequence is a (G4S), linker, including but not limited to, the (G4S)₃ linker having an amino acid sequence GGGGSGGGGSGGGGS (SEQ ID NO:53). In another embodiment, the linker is the Whitlow linker, having an amino acid sequence GSTSGSGKPGSGEGSTKG (SEQ ID NO:109).

### Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### Intron

The recombinant nucleic acid sequence construct can include one or more introns. Each intron can include functional splice donor and acceptor sites. The intron can include an enhancer of splicing. The intron can include one or more signals required for efficient splicing.

### Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

### Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

### ScFv-Fc Arrangement

In a ScFv-Fc arrangement, the recombinant nucleic acid sequence can include a sequence encoding the VH domain of the heavy chain polypeptide, and the VL domain of the light chain polypeptide, and further a linker sequence positioned between the heterologous nucleic acid sequence encoding the VH domain and VL domain.

An example of a ScFv-Fc arrangement can include the vector (and thus recombinant nucleic acid sequence construct) encoding the VH, linker, VL, hinge region, CH2, and CH3. The VH region can be N-terminally or C-terminally linked to a VL region via a linker.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAXI, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be p YES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### RNA

The one or more vectors may be an RNA molecule. In one embodiment, the RNA molecule is transcribed from a DNA sequence described herein. For example, in some embodiments, the RNA molecule encodes a structurally modified DMAb or a variant thereof or a fragment thereof. In one embodiment, an RNA molecule is a transcript generated from a DNA molecule encoding a structurally modified DMAb or a variant thereof or a fragment thereof. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA.

### Circular and Linear Vector

The one or more vectors may be circular plasmid, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### Bidirectional Expression Vector

The one or more vectors may be a bidirectional expression vector. The bidirectional vector may designed to express a protein or polypeptide of interest and a reporter protein, or alternatively to express two proteins or polypeptides of interest from a single promoter. The expression may be driven by a constitutively active bidirectional human cytomegalovirus promoter (PminCM). In one embodiment, a first polypeptide of interest is a DMAb and a second polypeptide of interest is an antigen. In one embodiment, a first polypeptide of interest is a first DMAb and a second polypeptide of interest is a second DMAb. In one embodiment, one or more of a first and second DMAb may be a structurally modified DMAb. A second DMAb may target the same antigen as a first DMAb, a different antigen from the same virus as a first DMAb, or an antigen of a different virus. For example, in one embodiment, the invention provides multivalent bidirectional expression vectors encoding a combination of an anti-ZIKV structurally modified DMAb and an anti-DENV structurally modified DMAb. In one embodiment, a bidirectional expression vector comprises a nucleotide sequence encoding a structurally modified DMAb comprising an amino acid sequence selected from SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO: 82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO: 100, SEQ ID NO: 102, SEQ ID NO: 104 or SEQ ID NO:106 and a nucleotide sequence encoding a structurally modified DMAb comprising an amino acid sequence of SEQ ID NO:108.

### Viral Vectors

Viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in WO/2008/148010, published Dec. 4, 2008. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### Antibody

As described above, the recombinant nucleic acid sequence can encode the structurally modified DMAb, a fragment thereof, a variant thereof, or a combination thereof. The structurally modified DMAb can bind or react with the antigen, which is described in more detail below.

The structurally modified DMAb may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The structurally modified DMAb can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The structurally modified DMAb may lack the CH1 and CL region of the heavy and light chain respectively. In such an embodiment, the structurally modified DMAb may be a single chain DMAb and comprise a flexible amino acid linker sequence which serves to tether the VL region to the VH region. The structurally modified DMAb may comprise a single chain including a VL region, a linker, a VH region, a hinge region, a CH2 region, and a CH3 region. The VH region can be N-terminally or C-terminally linked to a VL region via a linker.

The structurally modified DMAb can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The structurally modified DMAb can be an IgG1 antibody, an IgG2 antibody, an IgG3 antibody, an IgG4 antibody, an IgA1 antibody, an IgA2 antibody, an IgD antibody, an IgE antibody, or an IgM antibody. The structurally modified DMAb can be a chimera of any of an IgG1 antibody, an IgG2 antibody, an IgG3 antibody, an IgG4 antibody, an IgA1 antibody, an IgA2 antibody, an IgD antibody, an IgE antibody, or an IgM antibody. In some embodiments, the antibody hinge domain is modified. For example, in one embodiment, the structurally modified DMAb includes a Serine to Proline amino acid substitution in the hinge domain.

The structurally modified DMAb can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

The antibody may be modified to reduce or prevent antibody-dependent enhancement (ADE) of disease associated with the antigen as described in more detail below.

### Bispecific Antibody

The recombinant nucleic acid sequence can encode a bispecific structurally modified DMAb, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker, including a cancer marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments, one of the binding sites of an antibody molecule according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing .alpha.- and .beta.-chains, in some embodiments it encompasses .gamma.-chains and .delta.-chains (supra). Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments the first binding site of the antibody molecule binds a target antigen and the second binding site binds a T cell specific receptor molecule and/or a natural killer (NK) cell specific receptor molecule.

In some embodiments the first binding site of the antibody molecule binds a target antigen including, but not limited to, a viral antigen (e.g., Ebolavirus GP glycan cap, Ebolavirus GP fusion loop, or Ebolavirus GP chalice base) or a self antigen (e.g., tumor antigens), and the second binding site binds a T cell specific receptor molecule and/or a natural killer (NK) cell specific receptor molecule. In some embodiments the first binding site of the antibody molecule binds a target antigen and the second binding site binds one of CD3, the T cell receptor (TCR), CD28, CD16, NKG2D, Ox40, 4-1BB, CD2, CD5 and CD95.

In some embodiments the first binding site of the antibody molecule binds a T cell specific receptor molecule and/or a natural killer (NK) cell specific receptor molecule and the second binding site binds a target antigen including, but not limited to, viral antigens (e.g., Ebolavirus GP glycan cap, Ebolavirus GP fusion loop, or Ebolavirus GP chalice base) and self antigens (e.g., tumor antigens). In some embodiments the first binding site of the antibody binds a T cell specific receptor molecule and/or a natural killer (NK) cell specific receptor molecule and the second binding site binds a target antigen. In some embodiments the first binding site of the antibody binds one of CD3, the T cell receptor (TCR), CD28, CD16, NKG2D, Ox40, 4-1BB, CD2, CD5 and CD95, and the second binding site binds a target antigen.

### Bifunctional Antibody

The recombinant nucleic acid sequence can encode a bifunctional structurally modified DMAb, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

### Extension of Antibody Half-Life

As described above, the structurally modified DMAb may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode a structurally modified DMAb that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The structurally modified DMAb may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Reduced ADE Response

The structurally modified DMAb may be modified to reduce or prevent antibody-dependent enhancement (ADE) of disease associated with the antigen, but still neutralize the antigen. For example, the antibody may be modified to reduce or prevent ADE of disease associated with DENV, which is described below in more detail, but still neutralize DENV.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### Antigen

The structurally modified DMAb of the invention is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

The antigen can be from any number of organisms, for example, a virus, a parasite, a bacterium, a fungus, or a mammal. The antigen can be associated with an autoimmune disease, allergy, or asthma. In other embodiments, the antigen can be associated with cancer, herpes, influenza, hepatitis B, hepatitis C, human papilloma virus (HPV), or human immunodeficiency virus (HIV).

In some embodiments, the antigen is foreign. In some embodiments, the antigen is a self-antigen.

### Foreign Antigens

In some embodiments, the antigen is foreign. A foreign antigen is any non-self substance (i.e., originates external to the subject) that, when introduced into the body, is capable of stimulating an immune response.

### Viral Antigens

The foreign antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Polyomaviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papilloma virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Merkel cell polyoma virus (MCV) or cancer causing virus.

### Human Immunodeficiency Virus (HIV) Antigen

The viral antigen may be from Human Immunodeficiency Virus (HIV) virus. In some embodiments, the HIV antigen can be a subtype A envelope protein, subtype B envelope protein, subtype C envelope protein, subtype D envelope protein, subtype B Nef-Rev protein, Gag subtype A, B, C, or D protein, MPol protein, a nucleic acid or amino acid sequences of Env A, Env B, Env C, Env D, B Nef-Rev, Gag, or any combination thereof.

### Chikungunya Virus

The viral antigen may be from Chikungunya virus. Chikungunya virus belongs to the alphavirus genus of the Togaviridae family. Chikungunya virus is transmitted to humans by the bite of infected mosquitoes, such as the genus Aedes.

### Dengue Virus

The viral antigen may be from Dengue virus. The Dengue virus antigen may be one of three proteins or polypeptides (C, prM, and E) that form the virus particle. The Dengue virus antigen may be one of seven other proteins or polypeptides (NS1, NS2a, NS2b, NS3, NS4a, NS4b, NS5) which are involved in replication of the virus. The Dengue virus may be one of five strains or serotypes of the virus, including DENV-1, DENV-2, DENV-3 and DENV-4. The antigen may be any combination of a plurality of Dengue virus antigens.

Exemplary structurally modified DMAbs specific for Dengue virus include, but are not limited to, the structurally modified DMAb listed in SEQ ID NO:107, which encodes SEQ ID NO: 108.

### Hepatitis Antigen

The viral antigen may include a hepatitis virus antigen (i.e., hepatitis antigen), or a fragment thereof, or a variant thereof. The hepatitis antigen can be an antigen or immunogen from one or more of hepatitis A virus (HAV), hepatitis B virus (HBV), hepatitis C virus (HCV), hepatitis D virus (HDV), and/or hepatitis E virus (HEV).

The hepatitis antigen can be an antigen from HAV. The hepatitis antigen can be a HAV capsid protein, a HAV non-structural protein, a fragment thereof, a variant thereof, or a combination thereof.

The hepatitis antigen can be an antigen from HCV. The hepatitis antigen can be a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

The hepatitis antigen can be an antigen from HDV. The hepatitis antigen can be a HDV delta antigen, fragment thereof, or variant thereof.

The hepatitis antigen can be an antigen from HEV. The hepatitis antigen can be a HEV capsid protein, fragment thereof, or variant thereof.

The hepatitis antigen can be an antigen from HBV. The hepatitis antigen can be a HBV core protein, a HBV surface protein, a HBV DNA polymerase, a HBV protein encoded by gene X, fragment thereof, variant thereof, or combination thereof. The hepatitis antigen can be a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof.

In some embodiments, the hepatitis antigen can be an antigen from HBV genotype A, HBV genotype B, HBV genotype C, HBV genotype D, HBV genotype E, HBV genotype F, HBV genotype G, or HBV genotype H.

### Human Papilloma Virus (HPV) Antigen

The viral antigen may comprise an antigen from HPV. The HPV antigen can be from HPV types 16, 18, 31, 33, 35, 45, 52, and 58 which cause cervical cancer, rectal cancer, and/or other cancers. The HPV antigen can be from HPV types 6 and 11, which cause genital warts, and are known to be causes of head and neck cancer.

The HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof.

### RSV Antigen

The viral antigen may comprise a RSV antigen. The RSV antigen can be a human RSV fusion protein (also referred to herein as “RSV F,” “RSV F protein,” and “F protein”), or fragment or variant thereof. The human RSV fusion protein can be conserved between RSV subtypes A and B. The RSV antigen can be a RSV F protein, or fragment or variant thereof, from the RSV Long strain (GenBank AAX23994.1). The RSV antigen can be a RSV F protein from the RSV A2 strain (GenBank AAB59858.1), or a fragment or variant thereof. The RSV antigen can be a monomer, a dimer, or trimer of the RSV F protein, or a fragment or variant thereof.

The RSV F protein can be in a prefusion form or a postfusion form. The postfusion form of RSV F elicits high titer neutralizing antibodies in immunized animals and protects the animals from RSV challenge.

The RSV antigen can also be human RSV attachment glycoprotein (also referred to herein as “RSV G,” “RSV G protein,” and “G protein”), or fragment or variant thereof. The human RSV G protein differs between RSV subtypes A and B. The antigen can be RSV G protein, or fragment or variant thereof, from the RSV Long strain (GenBank AAX23993). The RSV antigen can be RSV G protein from the RSV subtype B isolate H5601, the RSV subtype B isolate H1068, the RSV subtype B isolate H5598, the RSV subtype B isolate H1123, or a fragment or variant thereof.

In other embodiments, the RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1). The RSV antigen human can also be RSV non-structural protein 2 (“NS2 protein”), or fragment or variant thereof. For example, the RSV antigen can be RSV NS2 protein, or fragment or variant thereof, from the RSV Long strain (GenBank AAX23988.1). The RSV antigen can further be human RSV nucleocapsid (“N”) protein, or fragment or variant thereof. For example, the RSV antigen can be RSV N protein, or fragment or variant thereof, from the RSV Long strain (GenBank AAX23989.1). The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1). The RSV antigen also can be human RSV Matrix protein (“M”) protein, or fragment or variant thereof. For example, the RSV antigen can be RSV M protein, or fragment or variant thereof, from the RSV Long strain (GenBank AAX23991.1).

In still other embodiments, the RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1). The RSV antigen can also be human RSV Matrix protein2-1 (“M2-1”) protein, or fragment or variant thereof. For example, the RSV antigen can be RSV M2-1 protein, or fragment or variant thereof, from the RSV Long strain (GenBank AAX23995.1). The RSV antigen can further be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1). The RSV antigen human can be RSV Polymerase L (“L”) protein, or fragment or variant thereof. For example, the RSV antigen can be RSV L protein, or fragment or variant thereof, from the RSV Long strain (GenBank AAX23996.1).

In further embodiments, the RSV antigen can have an optimized amino acid sequence of NS1, NS2, N, P, M, SH, M2-1, M2-2, or L protein. The RSV antigen can be a human RSV protein or recombinant antigen, such as any one of the proteins encoded by the human RSV genome.

In other embodiments, the RSV antigen can be, but is not limited to, the RSV F protein from the RSV Long strain, the RSV G protein from the RSV Long strain, the optimized amino acid RSV G amino acid sequence, the human RSV genome of the RSV Long strain, the optimized amino acid RSV F amino acid sequence, the RSV NS1 protein from the RSV Long strain, the RSV NS2 protein from the RSV Long strain, the RSV N protein from the RSV Long strain, the RSV P protein from the RSV Long strain, the RSV M protein from the RSV Long strain, the RSV SH protein from the RSV Long strain, the RSV M2-1 protein from the RSV Long strain, the RSV M2-2 protein from the RSV Long strain, the RSV L protein from the RSV Long strain, the RSV G protein from the RSV subtype B isolate H5601, the RSV G protein from the RSV subtype B isolate H1068, the RSV G protein from the RSV subtype B isolate H5598, the RSV G protein from the RSV subtype B isolate H1123, or fragment thereof, or variant thereof.

Exemplary structurally modified DMAbs specific for RSV include, but are not limited to, the structurally modified DMAbs listed in SEQ ID NO:110 through SEQ ID NO: 119.

### Influenza Antigen

The viral antigen may comprise an antigen from influenza virus. The influenza antigens are those capable of eliciting an immune response in a mammal against one or more influenza serotypes. The antigen can comprise the full length translation product HA0, subunit HA1, subunit HA2, a variant thereof, a fragment thereof or a combination thereof. The influenza hemagglutinin antigen can be derived from multiple strains of influenza A serotype H1, serotype H2, a hybrid sequence derived from different sets of multiple strains of influenza A serotype H1, or derived from multiple strains of influenza B. The influenza hemagglutinin antigen can be from influenza B.

The influenza antigen can also contain at least one antigenic epitope that can be effective against particular influenza immunogens against which an immune response can be induced. The antigen may provide an entire repertoire of immunogenic sites and epitopes present in an intact influenza virus. The antigen may be derived from hemagglutinin antigen sequences from a plurality of influenza A virus strains of one serotype such as a plurality of influenza A virus strains of serotype H1 or of serotype H2. The antigen may be a hybrid hemagglutinin antigen sequence derived from combining two different hemagglutinin antigen sequences or portions thereof. Each of two different hemagglutinin antigen sequences may be derived from a different set of a plurality of influenza A virus strains of one serotype such as a plurality of influenza A virus strains of serotype H1. The antigen may be a hemagglutinin antigen sequence derived from hemagglutinin antigen sequences from a plurality of influenza B virus strains.

In some embodiments, the influenza antigen can be H1 HA, H2 HA, H3 HA, H5 HA, or a BHA antigen.

### Ebola Virus

The viral antigen may be from Ebola virus. Ebola virus disease (EVD) or Ebola hemorrhagic fever (EHF) includes any of four of the five known Ebola viruses including Bundibugyo virus (BDBV), Ebola virus (EBOV), Sudan virus (SUDV), and Taï Forest virus (TAFV, also referred to as Côthe d'Ivoire Ebola virus (Ivory Coast Ebolavirus, CIEBOV).

A structurally modified DMAb may be specific for an Ebola virus antigen. Exemplary structurally modified DMAbs specific for Ebola virus include, but are not limited to, the structurally modified DMAbs listed in SEQ ID NO: 1 through SEQ ID NO:46.

### Marburg Marburgvirus

The viral antigen may be from Marburg marburgvirus. Marburg virus disease (MVD) or Marburg hemorrhagic fever (EHF) includes any of four of the five known Marburg marburgviruses including Marburg virus (MARV) and Ravn virus (RAVV). Marburgvirus immunogens that can be used to induce broad immunity against multiple subtypes or serotypes of Marburgvirus. The antigen may be derived from a Marburg virus envelope glycoprotein.

### Zika Virus

The viral antigen may be from Zika virus. Zika disease is caused by infection with the Zika virus and can be transmitted to humans through the bite of infected mosquitoes or sexually transmitted between humans. The Zika antigen can include a Zika Virus Envelope protein, Zika Virus NS1 protein, or a Zika Virus Capsid protein.

A structurally modified DMAb may be specific for a Zika virus antigen. Exemplary structurally modified DMAbs specific for Zika virus include, but are not limited to, the structurally modified DMAbs listed in SEQ ID NO:63 through SEQ ID NO:106.

### Bacterial Antigens

The foreign antigen can be a bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile. The bacterium can be Mycobacterium tuberculosis.

### Pseudomonas Antigens

The bacterial antigen may be a Pseudomonas antigen. For example, in one embodiment, the antigen may be a Pseudomonas aeruginosa antigen, or fragment thereof, or variant thereof. The Pseudomonas aeruginosa antigen can be from a virulence factor. Virulence factors associated with Pseudomonas aeruginosa include, but are not limited to structural components, enzymes and toxins. A Pseudomonas aeruginosa virulence factor can be one of exopolysaccharide, Adhesin, lipopolysaccharide, Pyocyanin, Exotoxin A, Exotoxin S, Cytotoxin, Elastase, Alkaline protease, Phospholipase C, Rhamnolipid, and components of a bacterial secretion system.

In one embodiment, an Pseudomonas antigen is an extracellular polysaccharide (e.g. Alginate, Pel and Psl). In one embodiment, an antigen is one of polysaccharide synthesis locus (psl), a gene contained therein (e.g. pslA, pslB, psIC, pslD, pslE, pslF, pslG, pslH, pslI, pslJ, pslK, pslL, pslM, psIN and pslO), a protein or enzyme encoded therein (e.g. a glycosyl transferase, phosphomannose isomerase/GDP-D-mannose pyrophosphorylase, a transporter, a hydrolase, a polymerase, an acetylase, a dehydrogenase and a topoisomerase) or a product produced therefrom (e.g. Psl exopolysaccharide, referred to as “Psl”).

In one embodiment, a Pseudomonas antigen is a component of a bacterial secretion system. Six different classes of secretion systems (types I through VI) have been described in bacteria, five of which (types I, II, II, V and VI) are found in gram negative bacteria, including Pseudomonas aeruginosa. In one embodiment, an antigen is one of a gene (e.g. an apr or has gene) or protein (e.g. AprD, AprE, AprF, HasD, HasE, HasF and HasR) or a secreted protein (e.g. AprA, AprX and HasAp) of a type I secretion system. In one embodiment, an antigen is one of a gene (e.g. xcpA/pilD, xphA, xqhA, xcpP to Q and xcpR to Z) or protein (e.g. GspC to M, GspAB, GspN, GspO, GspS, XcpT to XcpX, FppA,) or a secreted protein (e.g. LasB, LasA, PlcH, PlcN, PlcB, CbpD, ToxA, PmpA, PrpL, LipA, LipC, PhoA, PsAP, LapA) of a type II secretion system. In one embodiment, an antigen is one of a gene (e.g. a psc, pcr, pop or exs gene) or protein (e.g. PscC, PscE to PscF, PscJ, PscN, PscP, PscW, PopB, PopD, PcrH and PcrV) or a secreted protein (e.g. ExoS, ExoT, ExoU and ExoY) of a type III secretion system. In one embodiment, an antigen is a regulator of a type III secretion system (e.g. ExsA and ExsC). In one embodiment, an antigen is one of a gene (e.g. estA) or protein (e.g. EstA, CupB3, CupB5 and LepB) or a secreted protein (e.g. EstA, LepA, and CupB5) of a type V secretion system. In one embodiment, an antigen is one of a gene (e.g. a HSI-I, HSI-II and HSI-III gene) or protein (e.g. Fhal, ClpV1, a VgrG protein or a Hcp protein) or a secreted protein (e.g. Hcp1) of a type VI secretion system.

### Borrelia Antigens

The bacterial antigen may be a Borrelia spp antigen, or fragment thereof, or variant thereof. The Borrelia spp antigen can be from any one of Borrelia burgdorferi, Borrelia lusitaniae, Borrelia afzelii, Borrelia bissettii, Borreliella bavariensis, Borrelia chilensis, Borrelia garinii, Borrelia valaisiana, Borrelia spielmanii, and Borrelia finlandensis.

The bacterial antigen may be a Borrelia spp antigen, or fragment thereof, or variant thereof. The Borrelia spp antigen can be from a bacterial product that allows a Borrelia spp to replicate or survive. Bacterial products that allow a Borrelia spp to replicate or survive include, but are not limited to structural components, enzymes and toxins. Such a product can be one of a lipoprotein, an outer surface protein, a product required for infectivity or persistence within vertebrate hosts, and a product involved in motility and chemotaxis.

In one embodiment, an antigen is a lipoprotein (e.g. BptA). In one embodiment, an antigen is an outer surface protein (e.g. OspA, OspB, and OspC). In one embodiment, an antigen is a product required for infectivity or persistence within vertebrate hosts (e.g. PncA, DbpA, DbpB, Bgp, P66 and VlsE).

Mycobacterium tuberculosis Antigens

The bacterial antigen may be a Mycobacterium tuberculosis antigen (i.e., TB antigen or TB immunogen), or fragment thereof, or variant thereof. The TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, and EsxW.

### Parasitic Antigens

The foreign antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Lyme Antigen

The foreign antigen may be a Lyme disease antigen. The antigen may be an outer-surface protein A antigen (OspA antigen), or fragment thereof, or variant thereof. The antigen can be from a parasite causing malaria. The Lyme disease is caused by the bacterium Borrelia burgdorferi and is transmitted to humans through the bite of infected Ixodes scapularis (Blacklegged tick or Deer tick).

### Malaria Antigen

The foreign antigen may be a malaria antigen (i.e., PF antigen or PF immunogen), or fragment thereof, or variant thereof. The antigen can be from a parasite causing malaria. The malaria causing parasite can be Plasmodium falciparum. The Plasmodium falciparum antigen can include the circumsporozoite (CS) antigen.

In some embodiments, the malaria antigen can be one of P. falciparum immunogens CS; LSA1; TRAP; CelTOS; and Ama1. The immunogens may be full length or immunogenic fragments of full length proteins.

In other embodiments, the malaria antigen can be TRAP, which is also referred to as SSP2. In still other embodiments, the malaria antigen can be CelTOS, which is also referred to as Ag2 and is a highly conserved Plasmodium antigen. In further embodiments, the malaria antigen can be Ama1, which is a highly conserved Plasmodium antigen. In some embodiments, the malaria antigen can be a CS antigen.

In other embodiments, the malaria antigen can be a fusion protein comprising a combination of two or more of the PF proteins set forth herein. For example, fusion proteins may comprise two or more of CS immunogen, ConLSA1 immunogen, ConTRAP immunogen, ConCelTOS immunogen, and ConAma1 immunogen linked directly adjacent to each other or linked with a spacer or one or more amino acids in between. In some embodiments, the fusion protein comprises two PF immunogens; in some embodiments the fusion protein comprises three PF immunogens, in some embodiments the fusion protein comprises four PF immunogens, and in some embodiments the fusion protein comprises five PF immunogens. Fusion proteins with two PF immunogens may comprise: CS and LSA1; CS and TRAP; CS and CelTOS; CS and Ama1; LSA1 and TRAP; LSA1 and CelTOS; LSA1 and Ama1; TRAP and CelTOS; TRAP and Ama1; or CelTOS and Ama1. Fusion proteins with three PF immunogens may comprise: CS, LSA1 and TRAP; CS, LSA1 and CelTOS; CS, LSA1 and Ama1; LSA1, TRAP and CelTOS; LSA1, TRAP and Ama1; or TRAP, CelTOS and Ama1. Fusion proteins with four PF immunogens may comprise: CS, LSA1, TRAP and CelTOS; CS, LSA1, TRAP and Ama1; CS, LSA1, CelTOS and Ama1; CS, TRAP, CelTOS and Ama1; or LSA1, TRAP, CelTOS and Ama1. Fusion proteins with five PF immunogens may comprise CS or CS-alt, LSA1, TRAP, CelTOS and Ama1.

### Fungal Antigens

The foreign antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

### Self Antigens

In some embodiments, the antigen is a self antigen. A self antigen may be a constituent of the subject's own body that is capable of stimulating an immune response. In some embodiments, a self antigen does not provoke an immune response unless the subject is in a disease state, e.g., an autoimmune disease.

Self antigens may include, but are not limited to, cytokines, antibodies against viruses such as those listed above including HIV and Dengue, antigens affecting cancer progression or development, and cell surface receptors or transmembrane proteins.

### WT-1

The self-antigen antigen can be Wilm's tumor suppressor gene 1 (WT1), a fragment thereof, a variant thereof, or a combination thereof. WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug. Mutation of WT1 can lead to tumor or cancer formation, for example, Wilm's tumor or tumors expressing WT1.

### EGFR

The self-antigen may include an epidermal growth factor receptor (EGFR) or a fragment or variation thereof. EGFR (also referred to as ErbB-1 and HER1) is the cell-surface receptor for members of the epidermal growth factor family (EGF-family) of extracellular protein ligands. EGFR is a member of the ErbB family of receptors, which includes four closely related receptor tyrosine kinases: EGFR (ErbB-1), HER2/c-neu (ErbB-2), Her 3 (ErbB-3), and Her 4 (ErbB-4). Mutations affecting EGFR expression or activity could result in cancer.

The antigen may include an ErbB-2 antigen. Erb-2 (human epidermal growth factor receptor 2) is also known as Neu, HER2, CD340 (cluster of differentiation 340), or p185 and is encoded by the ERBB2 gene. Amplification or over-expression of this gene has been shown to play a role in the development and progression of certain aggressive types of breast cancer. In approximately 25-30% of women with breast cancer, a genetic alteration occurs in the ERBB2 gene, resulting in the production of an increased amount of HER2 on the surface of tumor cells. This overexpression of HER2 promotes rapid cell division and thus, HER2 marks tumor cells.

### Cocaine

The self-antigen may be a cocaine receptor antigen. Cocaine receptors include dopamine transporters.

### PD-1

The self-antigen may include programmed death 1 (PD-1). Programmed death 1 (PD-1) and its ligands, PD-L1 and PD-L2, deliver inhibitory signals that regulate the balance between T cell activation, tolerance, and immunopathology. PD-1 is a 288 amino acid cell surface protein molecule including an extracellular IgV domain followed by a transmembrane region and an intracellular tail.

### LAG-3

The self-antigen may include lymphocyte activation gene 3 (Lag-3 also known as CD223). LAG-3 is a member of the Ig superfamily that is expressed only on activated and tolerized T cells that binds MHC-II molecules and which is known to transduce inhibitory signals. LAG-3 is markedly upregulated on exhausted T cells compared to effector or memory T cells. LAG-3 negatively regulates T cell expansion by inhibiting T cell receptor induced calcium fluxes, thus controlling the size of the T cell memory pool in the context of cancer, LAG3 is upregulated on TILs and blockade of LAG-3 can enhance antitumor T cell immune responses. Blockage of LAG-3 in a viral chronic model that evokes CD8 T cells exhaustion, can invigorate the CD8 T cell responses.

### GITR

The self-antigen may include glucocorticoid-induced TNFR-related protein (GITR), also referred to as TNF receptor superfamily 18 (TNFRSF 18). GITR activation sends a co-activating signal to CD4+ and CD8+ T cells and prevents suppression of an immune response by regulatory T cells. Additionally, GITR-expressing effector T cells and regulatory T cells infiltrate multiple tumor types, yet there is little or no expression of GITR on non-hematopoetic cells. This distribution profile means that GITR-expressing cells can become concentrated at tumors. This combination of activities and distribution collectively makes GITR activation an attractive approach for treating a variety of cancers. The antigen binding proteins can be used to treat both solid tumors, as well as hematological cancers, including leukemia. In addition to use in treating cancer, in another embodiment, the antigen binding proteins that are provided can be used to induce or enhance an immune response against foreign antigens, such as those present on various infectious agents. Examples of antigens present on infectious agents against which an immune response can be generated include, but are not limited to proteins, glycoproteins, lipoproteins and glycolipids present on viruses, parasites, bacteria, and other microorganisms.

### CD40

The self-antigen may include CD40. CD40 is a 55 kDa cell-surface antigen present on the surface of normal and neoplastic human B cells, dendritic cells, other antigen presenting cells (APCs), endothelial cells, monocytic cells, CD8+ T cells, epithelial cells, some epithelial carcinomas, and many solid tumors, including lung, breast, ovary, and colon cancers. Malignant B cells from several tumors of B-cell lineage express a high level of CD40 and appear to depend on CD40 signaling for survival and proliferation. Thus, transformed cells from patients with low- and high-grade B-cell lymphomas, B-cell acute lymphoblastic leukemia, multiple myeloma, chronic lymphocytic leukemia, and Hodgkin's disease express CD40. CD40 expression is also detected in two-thirds of acute myeloblastic leukemia cases and 50% of AIDS-related lymphomas.

### OX40

The self-antigen may include OX40. OX40 (also referred to as CD134) is a 50 kilodalton (KDa) glycoprotein and a member of the tumor necrosis factor receptor superfamily (TNFRSF). The ligand for OX40, OX40L (also referred to as TNFSF4, CD252), has been reported to be expressed on endothelial cells, activated antigen presenting cells including macrophages, dendritic cells, B cells and natural killer cells. Although not wishing to be bound by theory, binding between CD40 on antigen presenting cells increases OX40L expression as can lipopolysaccharide (LPS). Expression of OX40 on T cells can be induced following signaling via the T cell antigen receptor. For example, OX40 is expressed on recently activated T cells at the site of inflammation. CD4 and CD8 T cells can upregulate OX40 under inflammatory conditions.

### TIM-3

The self-antigen may include TIM-3. TIM-3 is a transmembrane receptor that is expressed on Th1 (T helper 1) CD4-T cells and cytotoxic CD8-T cells that secrete IFN-.gamma. TIM-3 is generally not expressed on naive T cells but rather upregulated on activated, effector T cells. TIM-3 has a role in regulating immunity and tolerance in vivo.

### 4-1BB

The self-antigen may include 4-1BB ligand. 4-1BB ligand is a type 2 transmembrane glycoprotein belonging to the TNF superfamily. 4-1BB ligand may be expressed on activated T Lymphocytes. 4-1BB is an activation-induced T-cell costimulatory molecule. Signaling via 4-1BB upregulates survival genes, enhances cell division, induces cytokine production, and prevents activation-induced cell death in T cells.

### CTLA4

The self-antigen may include CTLA-4 (Cytotoxic T-Lymphocyte Antigen 4), also known as CD152 (Cluster of differentiation 152). CTLA-4 is a protein receptor found on the surface of T cells, which lead the cellular immune attack on antigens. The antigen may be a fragment of CTLA-4, such as an extracellular V domain, a transmembrane domain, and a cytoplasmic tail, or combination thereof.

### IL-6

The self-antigen may include interleukin 6 (IL-6). IL-6 stimulates the inflammatory and auto-immune processes in many diseases including, but not limited to, diabetes, atherosclerosis, depression, Alzheimer's Disease, systemic lupus erythematosus, multiple myeloma, cancer, Behçet's disease, rheumatoid arthritis, sepsis, bacterial infection, viral infection, fungal infection, and the like.

### CD126

The self-antigen may include CD126. CD126 is the receptor for IL-6 and stimulates the inflammatory and auto-immune processes in many diseases including, but not limited to, diabetes, atherosclerosis, depression, Alzheimer's Disease, systemic lupus erythematosus, multiple myeloma, cancer, Behçet's disease, rheumatoid arthritis, sepsis, bacterial infection, viral infection, fungal infection, and the like.

### MCP-1

The self-antigen may include monocyte chemotactic protein-1 (MCP-1). MCP-1 is also referred to as chemokine (C-C motif) ligand 2 (CCL2) or small inducible cytokine A2. MCP-1 is a cytokine that belongs to the CC chemokine family. MCP-1 recruits monocytes, memory T cells, and dendritic cells to the sites of inflammation produced by either tissue injury or infection.

### Amyloid Beta

The self-antigen may include amyloid beta (Aβ) or a fragment or a variant thereof. The Aβ antigen can comprise an Aβ(X-Y) peptide, wherein the amino acid sequence from amino acid position X to amino acid Y of the human sequence Aβ protein including both X and Y, in particular to the amino acid sequence from amino acid position X to amino acid position Y of the amino acid sequence corresponding to amino acid positions 1 to 47; the human query sequence (SEQ ID NO:62) or variants thereof. The Aβ antigen can comprise an Aβ polypeptide of Aβ(X-Y) polypeptide wherein X can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 and Y can be 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, or 15. The Aβ polypeptide can comprise a fragment that is at least 15, at least 16, at least 17, at least 18, at least 19, at least 20, at least 21, at least 22, at least 23, at least 24, at least 25, at least 30, at least 35, at least 36, at least 37, at least 38, at least 39, at least 40, at least 41, at least 42, at least 43, at least 44, at least 45, or at least 46 amino acids.

### IP-10

The self-antigen may include interferon (IFN)-gamma-induced protein 10 (IP-10). IP-10 is also known as small-inducible cytokine B10 or C-X-C motif chemokine 10 (CXCL10). CXCL10 is secreted by several cell types, such as monocytes, endothelial cells and fibroblasts, in response to IFN-γ.

### TERT

The self-antigen may include TERT (telomerase reverse transcriptase). TERT is a telomerase reverse transcriptase that synthesizes a TTAGGG tag on the end of telomeres to prevent cell death due to chromosomal shortening. Hyperproliferative cells with abnormally high expression of TERT may be targeted by immunotherapy. Recent studies demonstrate that TERT expression in dendritic cells transfected with TERT genes can induce CD8+ cytotoxic T cells and elicit a CD4+ T cells in an antigen-specific fashion.

### Tyrosinase

The self-antigen may include tyrosinase (Tyr). Tyr is an important target for immune mediated clearance by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8+ (CTL) to attack and kill tumor cells; (3) increase T helper cell responses; (4) and increase inflammatory responses via IFN-γ and TFN-α or all of the aforementioned.

Tyrosinase is a copper-containing enzyme that can be found in plant and animal tissues. Tyrosinase catalyzes the production of melanin and other pigments by the oxidation of phenols such as tyrosine. In melanoma, tyrosinase can become unregulated, resulting in increased melanin synthesis. Tyrosinase is also a target of cytotoxic T cell recognition in subjects suffering from melanoma. Accordingly, tyrosinase can be an antigen associated with melanoma.

The antigen can comprise protein epitopes that make them particularly effective as immunogens against which anti-Tyr immune responses can be induced. The Tyr antigen can comprise the full length translation product, a variant thereof, a fragment thereof or a combination thereof.

The Tyr antigen can comprise a consensus protein. The Tyr antigen induces antigen-specific T-cell and high titer antibody responses both systemically against all cancer and tumor related cells. As such, a protective immune response is provided against tumor formation by vaccines comprising the Tyr consensus antigen. Accordingly, any user can design an immunogenic composition of the present invention to include a Tyr antigen to provide broad immunity against tumor formation, metastasis of tumors, and tumor growth. Proteins may comprise sequences homologous to the Tyr antigens, fragments of the Tyr antigens and proteins with sequences homologous to fragments of the Tyr antigens.

### NY-ESO-1

The self-antigen may include NY-ESO-1. NY-ESO-1 is a cancer-testis antigen expressed in various cancers where it can induce both cellular and humoral immunity. Gene expression studies have shown upregulation of the gene for NY-ESO-1, CTAG1B, in myxoid and round cell liposarcomas.

### MAGE

The self-antigen may include MAGE (Melanoma-associated Antigen). The MAGE antigen may include MAGE-A4 (melanoma associated antigen 4). NY-ESO-1 is a cancer-testis antigen expressed in various cancers where it can induce both cellular and humoral immunity. Gene expression studies have shown upregulation of the gene for NY-ESO-1, CTAG1B, in myxoid and round cell liposarcomas.

MAGE-A4 is expressed in male germ cells and tumor cells of various histological types such as gastrointestinal, esophageal and pulmonary carcinomas. MAGE-A4 binds the oncoprotein, Gankyrin. This MAGE-A4 specific binding is mediated by its C-terminus. Studies have shown that exogenous MAGE-A4 can partly inhibit the adhesion-independent growth of Gankyrin-overexpressing cells in vitro and suppress the formation of migrated tumors from these cells in nude mice. This inhibition is dependent upon binding between MAGE-A4 and Gankyrin, suggesting that interactions between Gankyrin and MAGE-A4 inhibit Gankyrin-mediated carcinogenesis. It is likely that MAGE expression in tumor tissue is not a cause, but a result of tumorgenesis, and MAGE genes take part in the immune process by targeting early tumor cells for destruction.

Melanoma-associated antigen 4 protein (MAGEA4) can be involved in embryonic development and tumor transformation and/or progression. MAGEA4 is normally expressed in testes and placenta. MAGEA4, however, can be expressed in many different types of tumors, for example, melanoma, head and neck squamous cell carcinoma, lung carcinoma, and breast carcinoma. Accordingly, MAGEA4 can be antigen associated with a variety of tumors.

The MAGEA4 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune responses. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts.

The MAGEA4 antigen can comprise protein epitopes that make them particularly effective as immunogens against which anti-MAGEA4 immune responses can be induced. The MAGEA4 antigen can comprise the full length translation product, a variant thereof, a fragment thereof or a combination thereof.

### FSHR

The self-antigen may include FSHR (Follicle stimulating hormone receptor). FSHR is an antigen that is selectively expressed in women in the ovarian granulosa cells (Simoni et al., Endocr Rev. 1997, 18:739-773) and at low levels in the ovarian endothelium (Vannier et al., Biochemistry, 1996, 35:1358-1366). Most importantly, this surface antigen is expressed in 50-70% of ovarian carcinomas.

### Tumor Microenvironment Antigens

The self-antigen may include Tumor microenvironment antigen. Several proteins are overexpressed in the tumor microenvironment including, but not limited to, Fibroblast Activation Protein (FAP), Platelet Derived Growth Factor Receptor Beta (PDGFR-β), and Glypican-1 (GPC1). FAP is a membrane-bound enzyme with gelatinase and peptidase activity that is up-regulated in cancer-associated fibroblasts in over 90% of human carcinomas. PDGFR-β is a cell surface tyrosine kinase receptor that has roles in the regulation of many biological processes including embryonic development, angiogenesis, cell proliferation and differentiation. GPC1 is a cell surface proteoglycan that is enriched in cancer cells.

### PRAME

The self-antigen may include PRAME (Melanoma antigen preferentially expressed in tumors). PRAME is a protein that in humans is encoded by the PRAME gene. This gene encodes an antigen that is predominantly expressed in human melanomas and that is recognized by cytolytic T lymphocytes. It is not expressed in normal tissues, except testis. The gene is also expressed in acute leukemias. Five alternatively spliced transcript variants encoding the same protein have been observed for this gene. Proteins may comprise sequences homologous to the PRAME antigens, fragments of the PRAME antigens and proteins with sequences homologous to fragments of the PRAME antigens.

### Prostate Antigen

The self-antigen may include prostate antigens such as prostate-specific membrane antigen (PSMA), PSA antigen, STEAP antigen, PSCA antigen, Prostatic acid phosphatase (PAP) antigen, and other known prostate tumor antigens. PSMA is also known as glutamate carboxypeptidase II (GCPII), N-acetyl-L-aspartyl-L-glutamate peptidase I (NAALADase I), NAAG peptidase, or folate hydrolase (FOLH). PMSA is an integral membrane protein highly expressed by prostate cancer cells.

In some embodiments, the recombinant nucleic acid sequence encoding an antibody directed against PSMA (anti-PSMA antibody) may be a recombinant nucleic acid sequence including a recombinant nucleic acid sequence construct in arrangement 2.

In still other embodiments, the anti-PSMA antibody encoded by the recombinant nucleic acid sequence may be modified as described herein. One such modification is a defucosylated antibody, which as demonstrated in the Examples, exhibited increased ADCC activity as compared to commercial antibodies. The modification may be in the heavy chain, light chain, or a combination thereof. The modification may be one or more amino acid substitutions in the heavy chain, one or more amino acid substitutions in the light chain, or a combination thereof.

### Tumor Antigen

The self-antigen may include a tumor antigen. In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

### Other Antigens

In some embodiments, the antigen is an antigen other than a foreign antigen and/or a self-antigen. Exemplary other antigens include, but are not limited to:

### HIV-1 VRC01

The other antigen can be HIV-1 VRC01. HIV-1 VCR01 is a neutralizing CD4-binding site-antibody for HIV. HIV-1 VCR01 contacts portions of HIV-1 including within the gp120 loop D, the CD4 binding loop, and the V5 region of HIV-1.

### HIV-1 PG9

The other antigen can be HIV-1 PG9. HIV-1 PG9 is the founder member of an expanding family of glycan-dependent human antibodies that preferentially bind the HIV (HIV-1) envelope (Env) glycoprotein (gp) trimer and broadly neutralize the virus.

### HIV-1 4E10

The other antigen can be HIV-1 4E10. HIV-1 4E10 is a neutralizing anti-HIV antibody. HIV-1 4E10 is directed against linear epitopes mapped to the membrane-proximal external region (MPER) of HIV-1, which is located at the C terminus of the gp41 ectodomain.

### DV-SF1

The other antigen can be DV-SF1. DV-SF1 is a neutralizing antibody that binds the envelope protein of the four Dengue virus serotypes.

### DV-SF2

The other antigen can be DV-SF2. DV-SF2 is a neutralizing antibody that binds an epitope of the Dengue virus. DV-SF2 can be specific for the DENV4 serotype.

### DV-SF3

The other antigen can be DV-SF3. DV-SF3 is a neutralizing antibody that binds the EDIII A strand of the Dengue virus envelope protein.

### Nucleic Acid Vaccine

A composition comprising a nucleic acid molecule comprising a nucleotide sequence encoding a structurally modified DMAb, a fragment thereof, a variant thereof, or a combination thereof can be administered alone or in combination to a subject in need thereof to facilitate in vivo expression and formation of an engineered DNA encoded synthetic antibody.

In one embodiment, the composition of the invention can be administered in combination with a composition that elicits an immune response in a mammal against an antigen. In one embodiment, the composition of the invention can be administered in combination with a nucleic acid encoding one or more antigens. In one embodiment, the first composition comprises a DNA vaccine.

In one embodiment, the combination of the invention comprises at least two nucleic acid molecules encoding at least two structurally modified dMAbs, wherein each dMAb targets a different antigen. For example, in one embodiment, each dMAb targets a different viral antigen of a single virus. In another embodiment, each dMAb targets a viral antigen of a different virus. In yet another embodiment, each dMAb targets a different self antigen.

In one embodiment, the combination vaccine of the invention comprises at least 2, at least 3, at least 4, at least 5, at least 6, or more than 6 nucleic acid molecules encoding at least two structurally modified dMAbs, wherein each dMAb targets a different antigen. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

In one embodiment, the immunogenic composition of the invention comprises at least two nucleic acid molecules encoding at least two structurally modified dMAbs, wherein each dMAb targets a different antigen, wherein each antigen is an antigen of a different virus. In one embodiment, the combination vaccine of the invention comprises at least 3, at least 4, at least 5, at least 6, or more than 6 nucleic acid molecules encoding at least 2, at least 3, at least 4, at least 5, at least 6 or more than 6 dMAbs, wherein the encoded dMABs target antigens from at least 2, at least 3, at least 4, at least 5, at least 6, or more than 6 different viruses.

The present invention relates to a composition comprising a recombinant nucleic acid sequence encoding a structurally modified DMAb, a fragment thereof, a variant thereof, or a combination thereof. The composition, when administered to a subject in need thereof, can result in the generation of a structurally modified DMAb in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

The structurally modified DMAb can treat, prevent, and/or protect against disease in the subject administered the composition. The structurally modified DMAb, by binding the antigen, can treat, prevent, and/or protect against disease in the subject administered the composition. The structurally modified DMAb can promote survival of the disease in the subject administered the composition. In one embodiment, the structurally modified DMAb can provide increased survival of the disease in the subject over the expected survival of a subject having the disease who has not been administered the structurally modified DMAb. In various embodiments, the structurally modified DMAb can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or a greater than 100% increase in survival of the disease in subjects administered the composition over the expected survival in the absence of the composition. In one embodiment, the structurally modified DMAb can provide increased protection against the disease in the subject over the expected protection of a subject who has not been administered the structurally modified DMAb. In various embodiments, the structurally modified DMAb can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition can result in the generation of the structurally modified DMAb in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the structurally modified DMAb in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the structurally modified DMAb in the subject more quickly than the generation of an endogenous antibody in a subject who is administered an antigen to induce a humoral immune response. The composition can result in the generation of the structurally modified DMAb at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

In one embodiment, the method relates to administration of a first composition comprising a nucleic acid molecule encoding a structurally modified DMAb in combination with a second composition comprising a nucleic acid molecule encoding a second structurally modified DMAb. A first composition and a second composition may be administered concurrently or in any order. In one embodiment, a first composition and second composition are administered concurrently at different injection sites.

In one embodiment, the method relates to administration of a single composition comprising one or more nucleic acid molecules encoding two or more structurally modified DMAb. In such an embodiment, the two or more DMAbs may be encoded on a single nucleic acid molecule, or on separate nucleic acid molecules which are combined into a single composition for administration.

In one embodiment, the method relates to administration of one or more nucleic acid molecules encoding one or more structurally modified DMAb in combination with a nucleic acid vaccines that is capable of generating in a mammal an immune response against an antigen. In one embodiment, the nucleic acid vaccine comprises at least one nucleic acid molecule capable of expressing a consensus antigen in the mammal and a pharmaceutically acceptable excipient. In one embodiment, the nucleic acid molecule comprises a promoter operably linked to a coding sequence that encodes the consensus antigen.

In some embodiments, the nucleic acid molecule comprises an encoding sequence that encodes for an antigen. In some embodiments, the nucleic acid molecule includes an encoding sequence that encodes for an antigen operably linked to an IgE leader sequence on the N-terminal end of the coding sequence.

The nucleic acid molecule can further include a polyadenylation sequence attached to the C-terminal end of the coding sequence. In one embodiment, the nucleic acid molecule is codon optimized.

In some embodiments, the pharmaceutically acceptable excipient is an adjuvant. In one embodiment, the adjuvant is selected from the group consisting of: IL-12 and IL-15. In some embodiments, the pharmaceutically acceptable excipient is a transfection facilitating agent. In one embodiment, the transfection facilitating agent is a polyanion, polycation, or lipid, and more preferably poly-L-glutamate. In one embodiment, the poly-L-glutamate is at a concentration less than 6 mg/ml. In one embodiment, the nucleic acid vaccine has a concentration of total nucleic acid of 1 mg/ml or greater.

In some embodiments, the nucleic acid vaccine comprises a plurality of unique DNA plasmids, wherein each of the plurality of unique DNA plasmids encodes a polypeptide comprising a consensus antigen.

In some embodiments of the present invention, the nucleic acid vaccine can further include an adjuvant. In some embodiments, the adjuvant is selected from the group consisting of: alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80,CD86 including IL-15 having the signal sequence deleted and optionally including the signal peptide from IgE. Other genes which may be useful adjuvants include those encoding: MCP-1, MIP-1-alpha, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof. In some preferred embodiments, the adjuvant is selected from IL-12, IL-15, CTACK, TECK, or MEC.

In some embodiments, methods of eliciting an immune response in mammals against a consensus antigen include methods of inducing mucosal immune responses. Such methods include administering to the mammal one or more of CTACK protein, TECK protein, MEC protein and functional fragments thereof or expressible coding sequences thereof in combination with a DNA plasmid including a consensus antigen, described above. The one or more of CTACK protein, TECK protein, MEC protein and functional fragments thereof may be administered prior to, simultaneously with or after administration of the nucleic acid vaccine provided herein. In some embodiments, an isolated nucleic acid molecule that encodes one or more proteins of selected from the group consisting of: CTACK, TECK, MEC and functional fragments thereof is administered to the mammal.

The composition of the present invention can have features required of effective compositions such as being safe so that the composition does not cause illness or death; being protective against illness; and providing ease of administration, few side effects, biological stability and low cost per dose.

As described above, the composition can comprise immunogenic compositions, such as vaccines, comprising one or more antigens. The vaccine can be used to protect against any number of antigens, thereby treating, preventing, and/or protecting against antigen based pathologies. The vaccine can significantly induce an immune response of a subject administered the vaccine, thereby protecting against and treating infection by the antigen.

The vaccine can be a DNA vaccine, a peptide vaccine, or a combination DNA and peptide vaccine. The DNA vaccine can include a nucleic acid sequence encoding the antigen. The nucleic acid sequence can be DNA, RNA, cDNA, a variant thereof, a fragment thereof, or a combination thereof. The nucleic acid sequence can also include additional sequences that encode linker, leader, or tag sequences that are linked to the antigen by a peptide bond. The peptide vaccine can include a antigenic peptide, a antigenic protein, a variant thereof, a fragment thereof, or a combination thereof. The combination DNA and peptide vaccine can include the above described nucleic acid sequence encoding the antigen and the antigenic peptide or protein, in which the antigenic peptide or protein and the encoded antigen have the same amino acid sequence.

The vaccine can induce a humoral immune response in the subject administered the vaccine. The induced humoral immune response can be specific for the antigen. The induced humoral immune response can be reactive with the antigen. The humoral immune response can be induced in the subject administered the vaccine by about 1.5-fold to about 16-fold, about 2-fold to about 12-fold, or about 3-fold to about 10-fold. The humoral immune response can be induced in the subject administered the vaccine by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold.

The humoral immune response induced by the vaccine can include an increased level of neutralizing antibodies associated with the subject administered the vaccine as compared to a subject not administered the vaccine. The neutralizing antibodies can be specific for the antigen. The neutralizing antibodies can be reactive with the antigen. The neutralizing antibodies can provide protection against and/or treatment of infection and its associated pathologies in the subject administered the vaccine.

The humoral immune response induced by the vaccine can include an increased level of IgG antibodies associated with the subject administered the vaccine as compared to a subject not administered the vaccine. These IgG antibodies can be specific for the antigen. These IgG antibodies can be reactive with the antigen. Preferably, the humoral response is cross-reactive against two or more strains of the antigen. The level of IgG antibody associated with the subject administered the vaccine can be increased by about 1.5-fold to about 16-fold, about 2-fold to about 12-fold, or about 3-fold to about 10-fold as compared to the subject not administered the vaccine. The level of IgG antibody associated with the subject administered the vaccine can be increased by at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 15.5-fold, or at least about 16.0-fold as compared to the subject not administered the vaccine.

The vaccine can induce a cellular immune response in the subject administered the vaccine. The induced cellular immune response can be specific for the antigen. The induced cellular immune response can be reactive to the antigen. Preferably, the cellular response is cross-reactive against two or more strains of the antigen. The induced cellular immune response can include eliciting a CD8⁺ T cell response. The elicited CD8⁺ T cell response can be reactive with the antigen. The elicited CD8⁺ T cell response can be polyfunctional. The induced cellular immune response can include eliciting a CD8⁺ T cell response, in which the CD8⁺ T cells produce interferon-gamma (IFN-γ), tumor necrosis factor alpha (TNF-α), interleukin-2 (IL-2), or a combination of IFN-γ and TNF-α.

The induced cellular immune response can include an increased CD8⁺ T cell response associated with the subject administered the vaccine as compared to the subject not administered the vaccine. The CD8⁺ T cell response associated with the subject administered the vaccine can be increased by about 2-fold to about 30-fold, about 3-fold to about 25-fold, or about 4-fold to about 20-fold as compared to the subject not administered the vaccine. The CD8⁺ T cell response associated with the subject administered the vaccine can be increased by at least about 1.5-fold, at least about 2.0-fold, at least about 3.0-fold, at least about 4.0-fold, at least about 5.0-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, at least about 15.0-fold, at least about 16.0-fold, at least about 17.0-fold, at least about 18.0-fold, at least about 19.0-fold, at least about 20.0-fold, at least about 21.0-fold, at least about 22.0-fold, at least about 23.0-fold, at least about 24.0-fold, at least about 25.0-fold, at least about 26.0-fold, at least about 27.0-fold, at least about 28.0-fold, at least about 29.0-fold, or at least about 30.0-fold as compared to the subject not administered the vaccine.

The induced cellular immune response can include an increased frequency of CD3⁺CD8⁺ T cells that produce IFN-γ. The frequency of CD3⁺CD8 IFN-γ⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD8⁺ T cells that produce TNF-α. The frequency of CD3⁺CD8⁺ TNF-α⁺ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, or 14-fold as compared to the subject not administered the vaccine.

The induced cellular immune response can include an increased frequency of CD3⁺CD8⁺ T cells that produce IL-2. The frequency of CD3⁺CD8 IL-2⁺ T cells associated with the subject administered the vaccine can be increased by at least about 0.5-fold, 1.0-fold, 1.5-fold, 2.0-fold, 2.5-fold, 3.0-fold, 3.5-fold, 4.0-fold, 4.5-fold, or 5.0-fold as compared to the subject not administered the vaccine.

The induced cellular immune response can include an increased frequency of CD3⁺CD8⁺ T cells that produce both IFN-γ and TNF-α. The frequency of CD3⁺CD8⁺IFN-γ⁺ TNF-α⁺ T cells associated with the subject administered the vaccine can be increased by at least about 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 160-fold, 170-fold, or 180-fold as compared to the subject not administered the vaccine.

The cellular immune response induced by the vaccine can include eliciting a CD4⁺ T cell response. The elicited CD4⁺ T cell response can be reactive with the desired antigen. The elicited CD4⁺ T cell response can be polyfunctional. The induced cellular immune response can include eliciting a CD4⁺ T cell response, in which the CD4⁺ T cells produce IFN-γ, TNF-α, IL-2, or a combination of IFN-γ and TNF-α.

The induced cellular immune response can include an increased frequency of CD3⁺CD4⁺ T cells that produce IFN-γ. The frequency of CD3⁺CD4 IFN-γ T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold as compared to the subject not administered the vaccine.

The induced cellular immune response can include an increased frequency of CD3⁺CD4⁺ T cells that produce TNF-α. The frequency of CD3⁺CD4⁺ TNF-α T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, or 22-fold as compared to the subject not administered the vaccine.

The induced cellular immune response can include an increased frequency of CD3⁺CD4⁺ T cells that produce IL-2. The frequency of CD3⁺CD4 IL-2 T cells associated with the subject administered the vaccine can be increased by at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 45-fold, 50-fold, 55-fold, or 60-fold as compared to the subject not administered the vaccine.

The induced cellular immune response can include an increased frequency of CD3⁺CD4⁺ T cells that produce both IFN-γ and TNF-α. The frequency of CD3⁺CD4 IFN-γ⁺ TNF-α⁺ associated with the subject administered the vaccine can be increased by at least about 2-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 22-fold, 23-fold 24-fold, 25-fold, 26-fold, 27-fold, 28-fold, 29-fold, 30-fold, 31-fold, 32-fold, 33-fold, 34-fold, or 35-fold as compared to the subject not administered the vaccine.

The vaccine of the present invention can have features required of effective vaccines such as being safe so the vaccine itself does not cause illness or death; is protective against illness resulting from exposure to live pathogens such as viruses or bacteria; induces neutralizing antibody to prevent invention of cells; induces protective T cells against intracellular pathogens; and provides ease of administration, few side effects, biological stability, and low cost per dose.

The vaccine can further induce an immune response when administered to different tissues such as the muscle or skin. The vaccine can further induce an immune response when administered via electroporation, or injection, or subcutaneously, or intramuscularly.

### Vaccine Constructs and Plasmids

The vaccine can comprise nucleic acid constructs or plasmids that encode the one or more antigens. The nucleic acid constructs or plasmids can include or contain one or more heterologous nucleic acid sequences. Provided herein are genetic constructs that can comprise a nucleic acid sequence that encodes the antigens. The genetic construct can be present in the cell as a functioning extrachromosomal molecule. The genetic construct can be a linear minichromosome including centromere, telomeres or plasmids or cosmids. The genetic constructs can include or contain one or more heterologous nucleic acid sequences.

The genetic constructs can be in the form of plasmids expressing the antigen in any order.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells.

The genetic constructs can comprise regulatory elements for gene expression of the coding sequences of the nucleic acid. The regulatory elements can be a promoter, an enhancer an initiation codon, a stop codon, or a polyadenylation signal.

The nucleic acid sequences can make up a genetic construct that can be a vector. The vector can be capable of expressing the antigen in the cell of a mammal in a quantity effective to elicit an immune response in the mammal. The vector can be recombinant. The vector can comprise heterologous nucleic acid encoding the antigen. The vector can be a plasmid. The vector can be useful for transfecting cells with nucleic acid encoding the antigen, which the transformed host cell is cultured and maintained under conditions wherein expression of the antigen takes place.

Coding sequences can be optimized for stability and high levels of expression. In some instances, codons are selected to reduce secondary structure formation of the RNA such as that formed due to intramolecular bonding.

The vector can comprise heterologous nucleic acid encoding the antigens and can further comprise an initiation codon, which can be upstream of the one or more cancer antigen coding sequence(s), and a stop codon, which can be downstream of the coding sequence(s) of the antigen. The initiation and termination codon can be in frame with the coding sequence(s) of the antigen. The vector can also comprise a promoter that is operably linked to the coding sequence(s) of the antigen. The promoter operably linked to the coding sequence(s) of the antigen can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the coding sequence(s) of the antigen. The polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, CA).

The vector can also comprise an enhancer upstream of the antigen. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. The vector can be pVAX1, pCEP4 or pREP4 from Invitrogen (San Diego, CA), which can comprise the Epstein Barr virus origin of replication and nuclear antigen EBNA-1 coding region, which can produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad CA). The CMV promoter is located at bases 137-724. The T7 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993.

Based upon the sequence of pVAX1 available from Invitrogen, the following mutations were found in the sequence of pVAX1 that was used as the backbone for plasmids 1-6 set forth herein:
C>G241 in CMV promoter
C>T 1942 backbone, downstream of the bovine growth hormone polyadenylation signal (bGHpolyA)
A>-2876 backbone, downstream of the Kanamycin gene
C>T 3277 in pUC origin of replication (Ori) high copy number mutation (see Nucleic Acid Research 1985)
G>C 3753 in very end of pUC Ori upstream of RNASeH site

Base pairs 2, 3 and 4 are changed from ACT to CTG in backbone, upstream of CMV promoter.

The backbone of the vector can be pAV0242. The vector can be a replication defective adenovirus type 5 (Ad5) vector.

The vector can also comprise a regulatory sequence, which can be well suited for gene expression in a mammalian or human cell into which the vector is administered. The one or more cancer antigen sequences disclosed herein can comprise a codon, which can allow more efficient transcription of the coding sequence in the host cell.

The vector can be pSE420 (Invitrogen, San Diego, Calif.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which maybe used for protein production in mammalian cells such as Chinese hamster ovary (CHO) cells. The vector can be expression vectors or systems to produce protein by routine techniques and readily available starting materials including Sambrook et al., Molecular Cloning and Laboratory Manual, Second Ed., Cold Spring Harbor (1989), which is incorporated fully by reference.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligram. In some preferred embodiments, composition according to the present invention comprises about 5 nanogram to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligram, from about 5 nanogram to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Methods of Generating a Nucleic Acid Molecule Encoding a Structurally Modified DMAb

There are multiple engineering strategies that can be used to arrive at an engineered DMAb of the invention.

Full grafting of DMAbs

In one embodiment, a structurally modified DMAb is generated by full grafting. In one embodiment, the method of full grafting comprises transposing the CDR regions of the variable light and variable heavy chain of the parental DMAb onto the framework of higher expressing DMAb. In one embodiment, a nucleic acid sequence encoding a structurally modified DMAb is generated from an amino acid sequence of a full graft DMAb.

In one embodiment, the nucleic acid sequence is optimized. Optimization can include one or more of the following: addition of a low GC content leader sequence to increase transcription; mRNA stability and codon optimization; addition of a kozak sequence (e.g., GCC ACC) for increased translation; addition of an IRES sequence for increased translation; addition of a WPRE sequence for increased transcription; addition of an immunoglobulin (Ig) leader sequence encoding a signal peptide; and eliminating to the extent possible cis-acting sequence motifs (i.e., internal TATA boxes).

In one embodiment, a nucleic acid molecule is generated comprising the optimized nucleic acid sequence encoding the structurally modified DMAb. Any method for generating a nucleic acid molecule can be used to generate nucleic acid molecules encoding structurally modified DMAbs of the invention. Methods of generating nucleic acid molecules comprising specific nucleotide sequences are generally known in the art.

Partial grafting of DMAbs

In one embodiment, a structurally modified DMAb is generated by partial grafting. In one embodiment, the method of partial grafting comprises one or more of structurally alignment and comparative modeling of potential modifications. In one embodiment, the parental DMAb VL domain sequence is aligned to the VL domain of one or more validated highly expressed scaffold VL sequences. In one embodiment, comparative modeling is performed on multiple potential partial graft sequences comprising mutations that are predicted through structural super-positioning of the VL domains. In one embodiment, the method of comparative modeling includes at least one of energy minimization analysis, interface analysis, sequence property prediction, and Ramachandran analysis. A candidate partial graft DMAb is one with favorable predicted characteristics (e.g., improved stability).

In one embodiment, a nucleic acid sequence encoding a structurally modified DMAb is generated from the predicted partial graft DMAb amino acid sequence. In one embodiment, a nucleic acid sequence encoding a structurally modified DMAb is further optimized, and a nucleic acid molecule is generated comprising the optimized nucleic acid sequence encoding the structurally modified DMAb.

### Scaffold Modification

In one embodiment, a structurally modified DMAb is generated by scaffold modification. In one embodiment, the method of scaffold modification comprises making one or more specific amino acid changes to the amino acid sequence of a parental DMAb to generate a structurally modified DMAb of the invention. In one embodiment, the specific amino acid changes improve at least one of stability, the heavy and light chain interface, and secretion of the structurally modified DMAb as compared to the parental DMAb. In one embodiment, the specific amino acid changes decrease aggregation of DMAbs based on variable chain interface, Pi interactions, isoelectric point, and Ramachandran analysis of the structurally modified DMAb as compared to the parental DMAb. In one embodiment, one or more specific amino acid changes are made to result in isoelectric point modification, alterations in VH-VL interface interactions or a combination thereof.

In one embodiment, a nucleic acid sequence encoding a structurally modified DMAb is generated from the modified amino acid sequence. In one embodiment, a nucleic acid sequence encoding a structurally modified DMAb is further optimized, and a nucleic acid molecule is generated comprising the optimized nucleic acid sequence encoding the structurally modified DMAb.

### ScFv-Fc Structurally Modified DMAbs

In one embodiment, a parental DMAb showing low expression undergoes one or more rounds of scFv modeling. In various embodiments, ScFv modeling includes at least one of linker modeling, hinge modification modeling, framework modeling, and CDR loop refinement. In various embodiments, modeling is performed on a DMAb sequence in at least one of VH-linker-VL and VL-linker-VH formats. In one embodiment, multiple rounds of modeling are performed with various input sequences until a variant is predicted where the linker does not obscure or minimally impacts the CDRs. For example, in one embodiment, the linker of a predicted variant is predicted to obscure one or more of the CDRs, therefore another round of ScFv modeling is conducted using a new linker sequence. In one embodiment, a new linker may be longer, shorter, or have a different amino acid sequence than the previously modeled linker. In one embodiment, following one or more rounds of ScFv modeling, an analysis is conducted on the predicted structure to determine whether the predicted DMAb is a candidate for further development. For example, in one embodiment, a RMSD analysis of the region near the linker fusion sites is performed. In one embodiment, a predicted DMAb with minimal obscurity of the CDRs by the linker is selected for as a candidate DMAb. In one embodiment, a predicted DMAb with a low predicted RMSD near the linker fusion sites is selected as a candidate DMAb.

In one embodiment, a nucleic acid molecule encoding a candidate variant is optimized. In one embodiment, optimization is performed on a nucleotide sequence encoding each of the VH, linker and VL sequences individually (i.e., modular optimization of the nucleotide sequence). In one embodiment, optimization is performed on a nucleotide sequence encoding VH-linker-VL as a single nucleotide sequence. In one embodiment, optimization is performed on a nucleotide sequence encoding VL-linker-VH as a single nucleotide sequence.

In one embodiment, a nucleic acid molecule encoding a structurally modified DMAb is generated. In one embodiment, the nucleic acid molecule comprises an optimized nucleic acid sequence encoding a structurally modified DMAb.

In one embodiment, the expression level and antigen binding of the structurally modified DMAb are then compared to that of the parental DMAb. In one embodiment, a structurally modified DMAb having greater expression than the parental DMAb, and still showing antigen binding, is then further tested for in vivo expression and ability to generate an immune response in a subject.

### Method of Generating the Synthetic Antibody

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### Method of Identifying or Screening for the Antibody

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### Method of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### Electroporation

Administration of the composition via electroporation may be accomplished using electroporation devices that can be configured to deliver to a desired tissue of a mammal, a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the composition of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the composition include those provided in co-pending and co-owned U.S. patent application Ser. No. 11/874,072, filed Oct. 17, 2007, which claims the benefit under 35 USC 119 (e) to U.S. Provisional Application Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, US Patents 6, 110, 161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### Method of Treatment

Also provided herein is a method of treating, protecting against, and/or preventing disease in a subject in need thereof by generating a structurally modified DMAb in the subject. The method can include administering the composition to the subject. Administration of the composition to the subject can be done using the method of delivery described above.

Upon generation of the structurally modified DMAb in the subject, the synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The method of delivering the vaccine or vaccination may be provided to induce a therapeutic and prophylactic immune response. The vaccination process may generate in the mammal an immune response against the antigen. The vaccine may be delivered to an individual to modulate the activity of the mammal's immune system and enhance the immune response. The delivery of the vaccine may be the transfection of the consensus antigen as a nucleic acid molecule that is expressed in the cell and delivered to the surface of the cell upon which the immune system recognized and induces a cellular, humoral, or cellular and humoral response. The delivery of the vaccine may be used to induce or elicit and immune response in mammals against the antigen by administering to the mammals the vaccine as discussed above.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The composition can comprise 1 or more, 2 or more, 3 or more, 4 or more, 5 or more, 6 or more, 7 or more, 8 or more, 9 or more, or 10 or more DNA vaccines encoding an antigen. The composition may comprise 1 or more, 2 or more, 3 or more, 4 or more, 5 or more, 6 or more, 7 or more, 8 or more, 9 or more, or 10 or more structurally modified DMAbs or fragments thereof.

The DNA vaccine and the nucleic acid molecule encoding a structurally modified DMAb may be administered at the same time or at different times. In one embodiment, the DNA vaccine and the nucleic acid molecule encoding a structurally modified DMAb are administered simultaneously. In one embodiment, the DNA vaccine is administered before the nucleic acid molecule encoding a structurally modified DMAb. In one embodiment, the nucleic acid molecule encoding a structurally modified DMAb is administered before the DNA vaccine.

In certain embodiments, the DNA vaccine is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the nucleic acid molecule encoding a structurally modified DMAb is administered. In certain embodiments, the DNA vaccine is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the nucleic acid molecule encoding a structurally modified DMAb is administered. In certain embodiments, the DNA vaccine is administered 1 or more months, 2 or more months, 3 or more months, 4 or more months, 5 or more months, 6 or more months, 7 or more months, 8 or more months, 9 or more months, 10 or more months, 11 or more months, or 12 or more months after the nucleic acid molecule encoding a structurally modified DMAb is administered.

In certain embodiments, the nucleic acid molecule encoding a structurally modified DMAb is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, or 14 or more days after the DNA vaccine is administered. In certain embodiments, the nucleic acid molecule encoding a structurally modified DMAb is administered 1 or more weeks, 2 or more weeks, 3 or more weeks, 4 or more weeks, 5 or more weeks, 6 or more weeks, 7 or more weeks, 8 or more weeks, 9 or more weeks, or 10 or more weeks after the DNA vaccine is administered. In certain embodiments, the nucleic acid molecule encoding a structurally modified DMAb is administered 1 or more months, 2 or more months, 3 or more months, 4 or more months, 5 or more months, 6 or more months, 7 or more months, 8 or more months, 9 or more months, 10 or more months, 11 or more months, or 12 or more months after the DNA vaccine is administered.

In certain embodiments, the nucleic acid molecule encoding a structurally modified DMAb and DNA vaccine are administered once. In certain embodiments, the nucleic acid molecule encoding a structurally modified DMAb and/or the DNA vaccine are administered more than once. In certain embodiments, administration of the nucleic acid molecule encoding a structurally modified DMAb and DNA vaccine provides a persistent and systemic immune response.

Use in Combination with Antibiotics

The present invention also provides a method of treating, protecting against, and/or preventing disease in a subject in need thereof by administering a combination of the structurally modified DMAb and a therapeutic antibiotic agent.

The structurally modified DMAb and an antibiotic agent may be administered using any suitable method such that a combination of the structurally modified DMAb and antibiotic agent are both present in the subject. In one embodiment, the method may comprise administration of a first composition comprising a nucleic acid molecule encoding a structurally modified DMAb of the invention by any of the methods described in detail above and administration of a second composition comprising an antibiotic agent less than 1, less than 2, less than 3, less than 4, less than 5, less than 6, less than 7, less than 8, less than 9 or less than 10 days following administration of the synthetic antibody. In one embodiment, the method may comprise administration of a first composition comprising a nucleic acid molecule encoding a structurally modified DMAb of the invention by any of the methods described in detail above and administration of a second composition comprising an antibiotic agent more than 1, more than 2, more than 3, more than 4, more than 5, more than 6, more than 7, more than 8, more than 9 or more than 10 days following administration of the synthetic antibody. In one embodiment, the method may comprise administration of a first composition comprising an antibiotic agent and administration of a second composition comprising a nucleic acid molecule encoding a structurally modified DMAb of the invention by any of the methods described in detail above less than 1, less than 2, less than 3, less than 4, less than 5, less than 6, less than 7, less than 8, less than 9 or less than 10 days following administration of the antibiotic agent. In one embodiment, the method may comprise administration of a first composition comprising an antibiotic agent and administration of a second composition comprising a nucleic acid molecule encoding a structurally modified DMAb of the invention by any of the methods described in detail above more than 1, more than 2, more than 3, more than 4, more than 5, more than 6, more than 7, more than 8, more than 9 or more than 10 days following administration of the antibiotic agent. In one embodiment, the method may comprise administration of a first composition comprising a nucleic acid molecule encoding a structurally modified DMAb of the invention by any of the methods described in detail above and a second composition comprising an antibiotic agent concurrently. In one embodiment, the method may comprise administration of a single composition comprising a nucleic acid molecule encoding a structurally modified DMAb of the invention and an antibiotic agent.

Non-limiting examples of antibiotics that can be used in combination with the synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

The present invention has multiple aspects, illustrated by the following non-limiting examples.

### EXAMPLES

The present invention is further illustrated in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Grafting and Scaffold Modification

DNA vector-encoded monoclonal antibodies (DMAbs) offer a means to generate in vivo mAbs by using electroporation (EP) to transfect skeletal muscle. In previous studies, it has been demonstrated that DMAbs can achieve high serum levels and shown protection comparable to purified mAbs in flu and pseudomonas murine challenge models. Working toward clinical application, efforts have been focused on further increasing the in vivo expression levels of DMAbs through formulation, administration, nucleotide and amino acid optimization. Multiple DMAb antibody modification strategies have been developed employing framework grafting to increase the in vivo expression levels of DMAbs without sacrificing the biology of the original mAb clone.

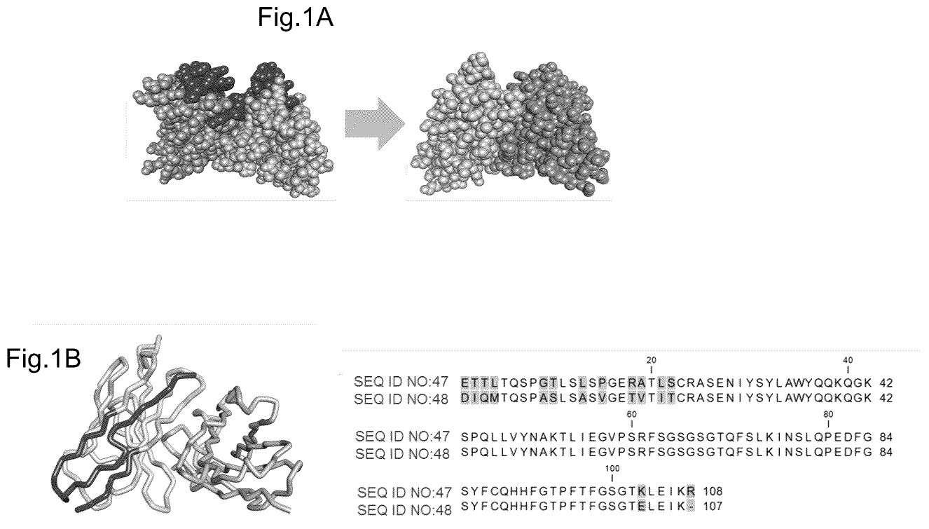

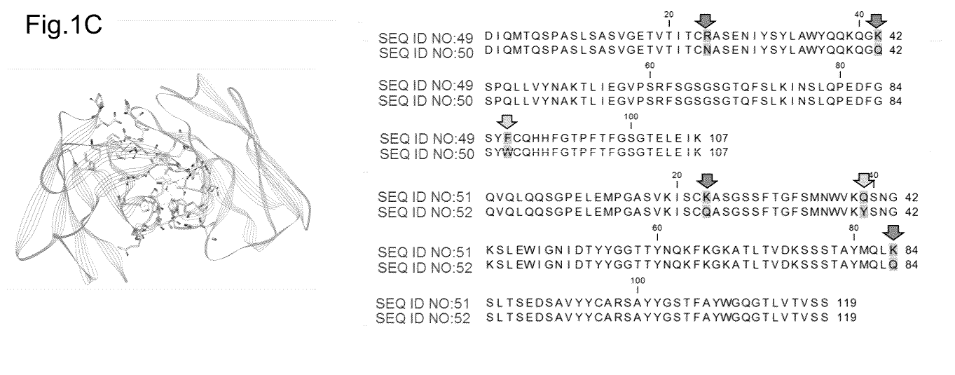

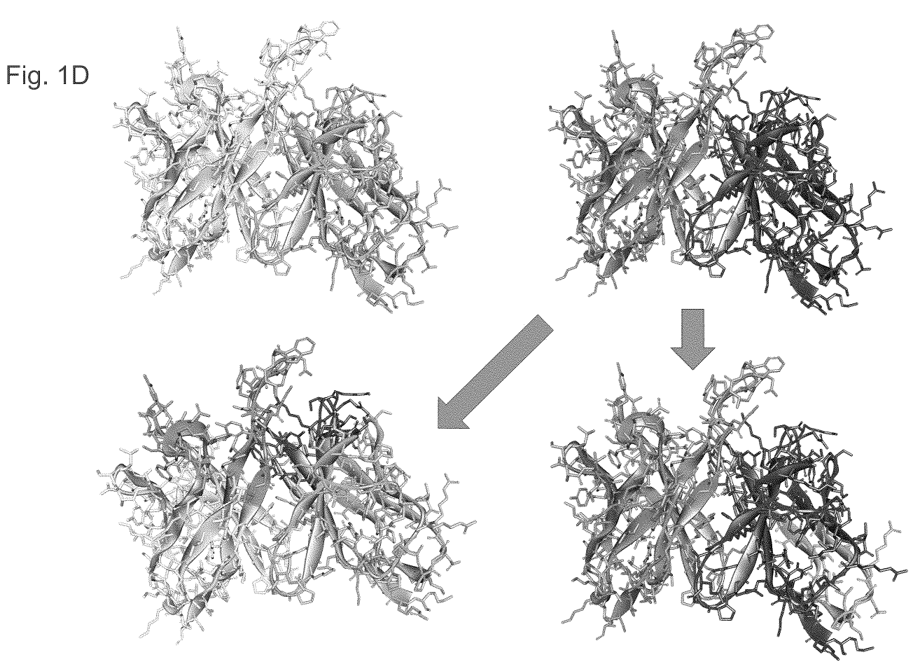

FIG. 1 provides an overview of multiple strategies used to generate optimized DMABs. Strategies include full scaffold grafting, partial scaffold grafting and scaffold modification (multiple point mutations).

Full scaffold grafting includes moving CDRs from one Fv region (V_{H} or V_{L}) with undesirable properties onto the framework of a second Fv which has desirable properties. The method is somewhat similar to that employed in antibody humanization. A limited selection of Fv's is available, making ideal CDR placement challenging. Expression can be impacted positively, but binding can be negatively impacted if the scaffold is not fully compatible.

Partial scaffold grafting includes altering selected regions of the scaffold, with the goal of increasing fold stability. Regions too close to the CDRs are avoided to minimize CDR perturbation. Generally, changes are made within the first 20 residues at the N terminus of the variable light chain to mimic the sequence of a DMAb having high expression.

Scaffold modification includes making a predicted series of multiple mutations to increase stabilizing interactions at the V_{H}-V_{L} interface or to favorably alter isoelectric point. Generally about 3 to 4 individual amino acid changes are made to increase stability

The methods are now described

BALB/c mice (n=8) were administrated 100 μg of DNA-plasmid encoding a DMAb in one treatment site through intramuscular delivery followed by electroporation (IM-EP). Serum levels of DMAb were quantified by ELISA at day 7. Binding of serum DMAbs was assessed by ELISA at day 7.

Partial grafts, full grafts and scaffold modification were performed on BDBV223 and Z5D2 antibodies as detailed in Table 1.

TABLE 1
Engineered DMAbs

| DMAb type | Name | Description |
|---|---|---|
| original | pGX9228 | Ebola BDBV223 |
| original | pGX9224 | Ebola Z5D2 |
| partial graft | pGX9292 | Ebola Z5D2 partial graft |
| full graft | pGX9293 | Ebola Z5D2 graft on MERSYTE_1 |
| full graft | pGX9294 | Ebola Z5D2 graft on MERSYTE_2 |
| full graft | pGX9295 | Ebola Z5D2 graft on V2L2 |
| partial graft | pGX9297 | Ebola BDBV223 partial graft |
| full graft | pGX9298 | Ebola BDBV223 graft on MERSYTE |
| full graft | pGX9299 | Ebola BDBV223 graft on V2L2 |

The results are now described

Multiple constructs were generated and screened for in vivo expression by ELISA. The partial graft method consists of replacing a portion of the variable light chain framework region from a poor expresser with that of a higher expressing DMAb. The new partial graft constructs showed increases approximately a log higher than the original DMAbs, while maintaining binding.

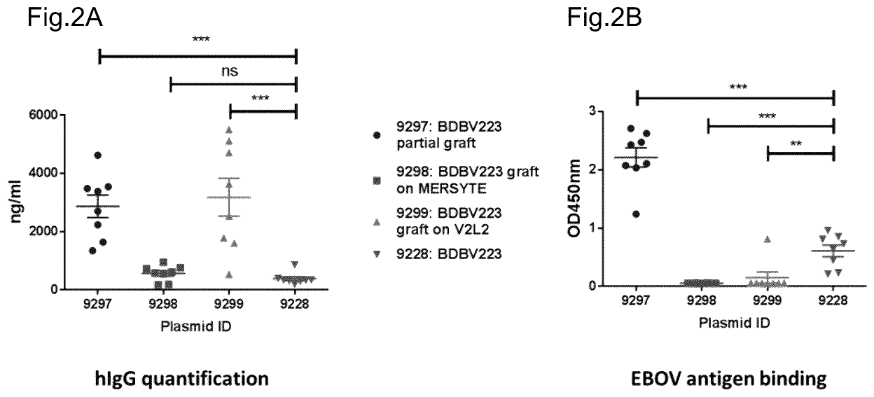

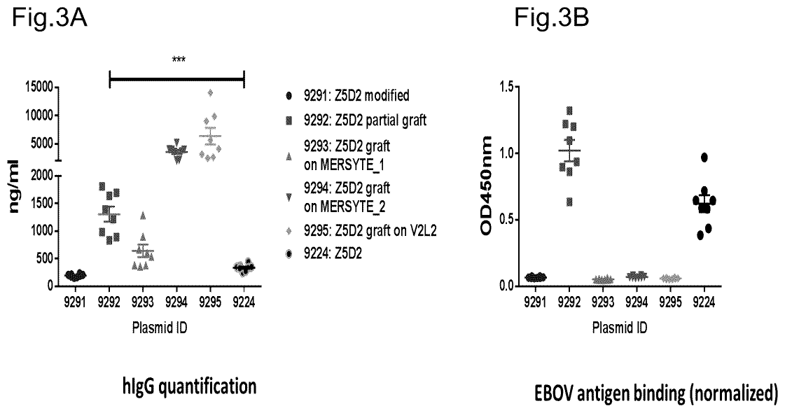

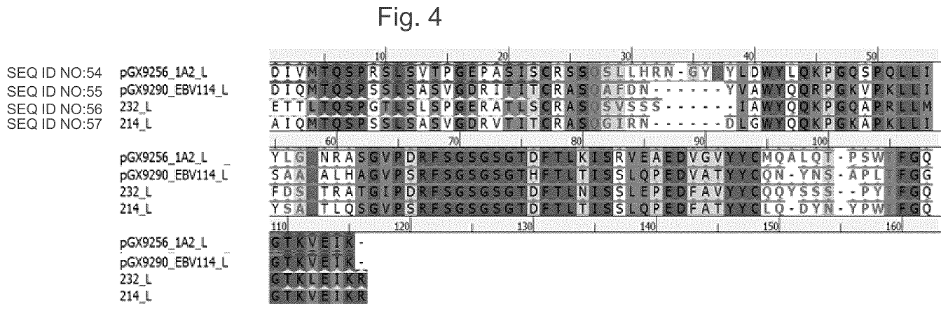

Partial grafting of BDBV223 resulted in an optimized antibody with enhanced expression and maintained binding (FIG. 2). In contrast, a full graft of BDBV223 onto V2L2 enhanced expression but resulted in loss of antigen binding (FIG. 2). Similar results were seen with the Z5D2 antibody. Partial grafting of Z5D2 resulted in an optimized antibody with enhanced expression and maintained binding whereas full grafting of Z5D2 resulted in loss of antigen binding (FIG. 3). FIG. 4 provides a summary of the effects of full grafting, partial grafting and scaffold modification on expression and antigen binding of Structurally modified DMAbs modified from three different parental DMAb sequences.

### Example 2: Partial Graft Design

Two high-expressing DMABs (pGX9232 and pGX9214) were aligned to DMAB light chains of pGX9256 (1A2) and pGX9290 (EBV114) (FIG. 4).

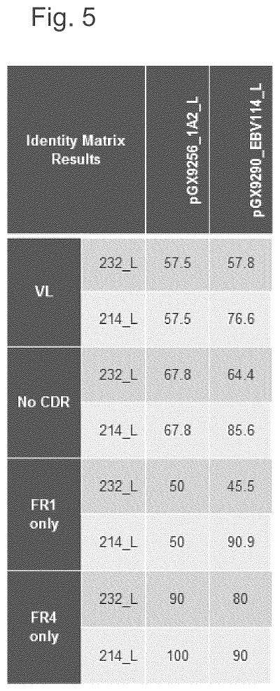

Identity matrices with and without CDRs were calculated. Results are provided in FIG. 5. Alignment matrices were also generated using only FR1 (up to first Cys residue) and FR4. The pattern from the FR1 matrix pattern is identical to the VL and No CDR matrices. The FR4 matrix, based on only 10 residues (excluding terminal R), gives a different pattern but is highly similar overall. Without being bound by any particular theory, it is hypothesized that a higher similar score is better. Sequences for the engineered partial grafts are:
>pGX9256_(232)_L (SEQ ID NO:58)
>pGX9290_(232)_L (SEQ ID NO:59)
>pGX9256_(214)_L (SEQ ID NO:60)
>pGX9290_(214)_L (SEQ ID NO:61)

### Example 3: scFv-Fc Conversion

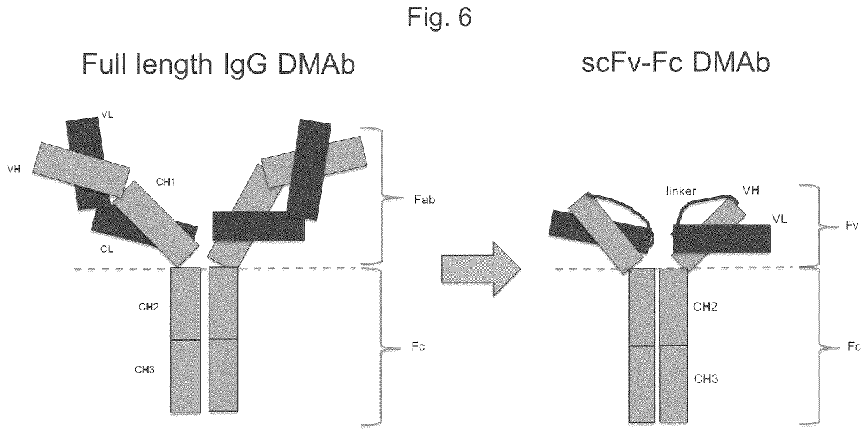

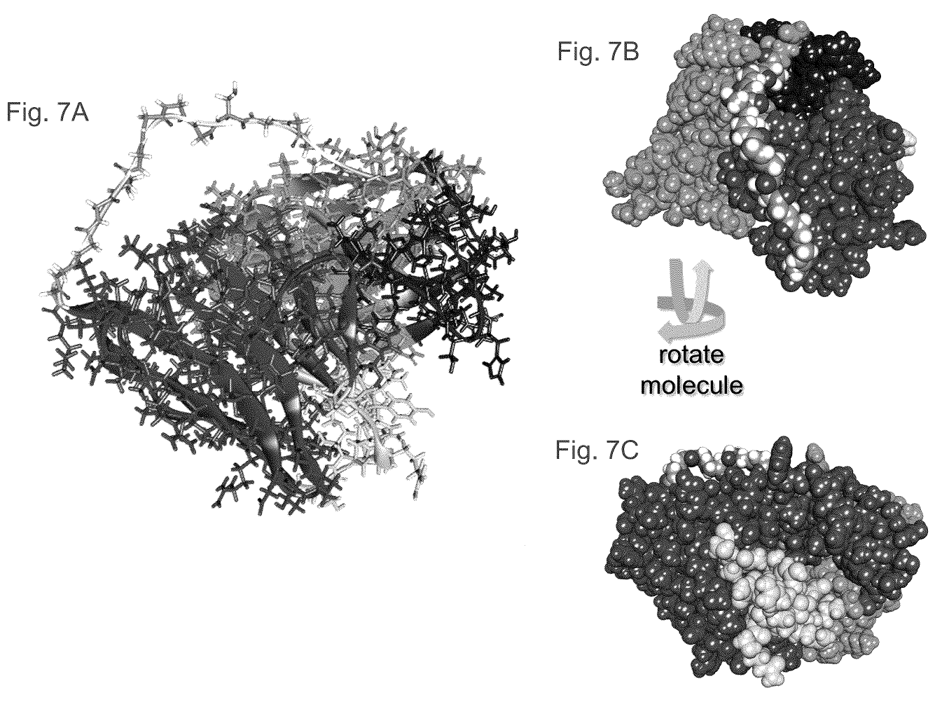

Single chain Fv-Fc (scFv-Fc) conversion is the removal of CH1 and CL regions, and the addition of a linker between VH and VL. Conversion promotes heavy chain-light chain pairing and tissue penetration. DMAbs are converted from a full length antibody to scFv-Fc through addition of a linker (as depicted in FIGS. 6 and 7).

The methods are now described

BALB/c mice (n=5) were administrated 100 μg of DNA-plasmid encoding scFv-Fc DMAb in one treatment site through intramuscular delivery followed by electroporation (IM-EP). Serum was collected over the course of 35 days post administration.

293T cells were transfected with DNA-plasmid encoding scFv-Fc DMAb. scFv-Fc DMAbs were purified from cell supernatant using Protein A, and their normalized binding affinity was analyzed by ELISA using an identical antigen from two different Ebola virus outbreak strains, Zaire ebolavirus glycoprotein (GP) from the 1976 Mayinga strain or the 2014 Guinea strain.

TABLE 2
scFv-Fc Engineered DMAbs

| DMAb type | Name | Description |
|---|---|---|
| original | pGX9224 | Ebola Z5D2 |
| original | pGX9225 | Ebola Z1H3 |
| original | pGX9228 | Ebola BDBV223 |
| scFV-Fc | pGX9330 | Ebola Z5D2 scFv-Fc |
| scFV-Fc | pGX9331 | Ebola Z1H3 scFv-Fc |
| scFV-Fc | pGX9332 | Ebola BDBV223 scFv-Fc |

The results are now described

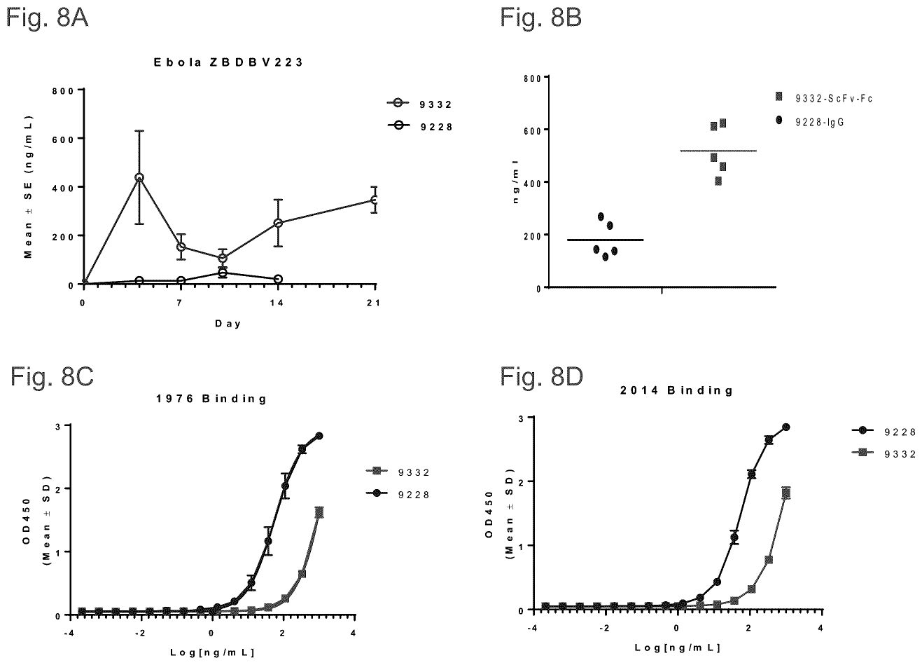

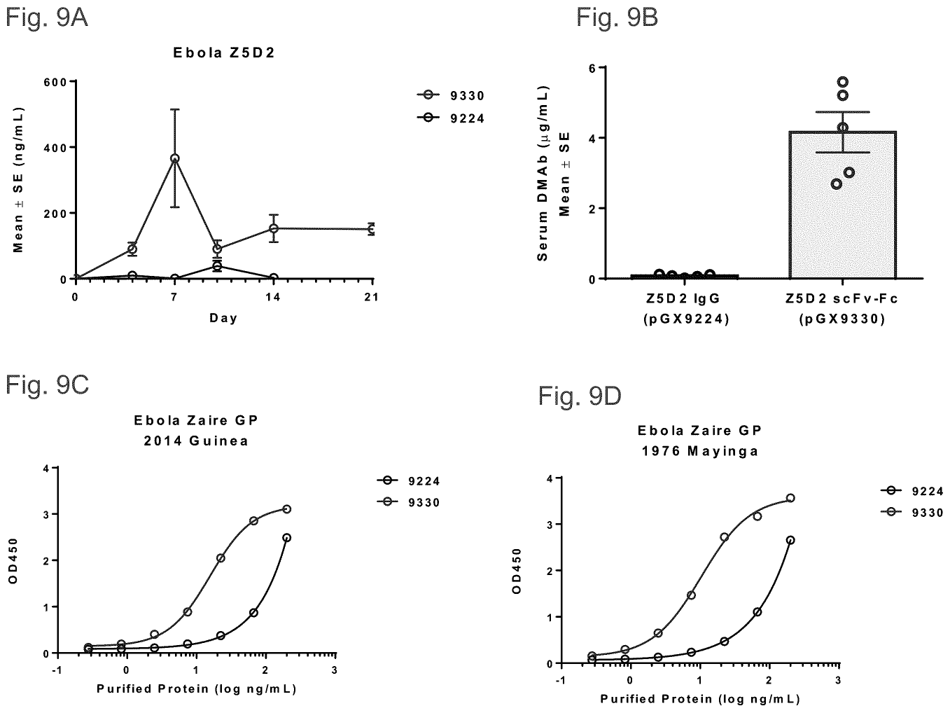

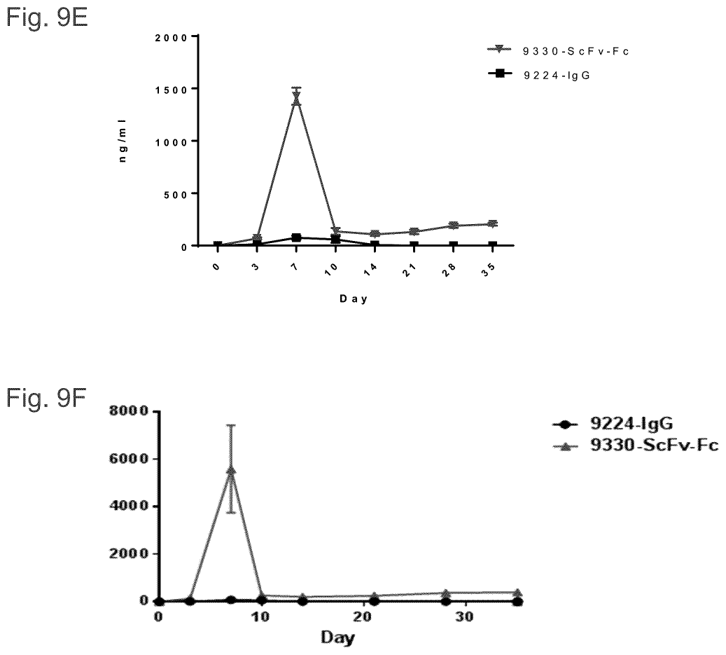

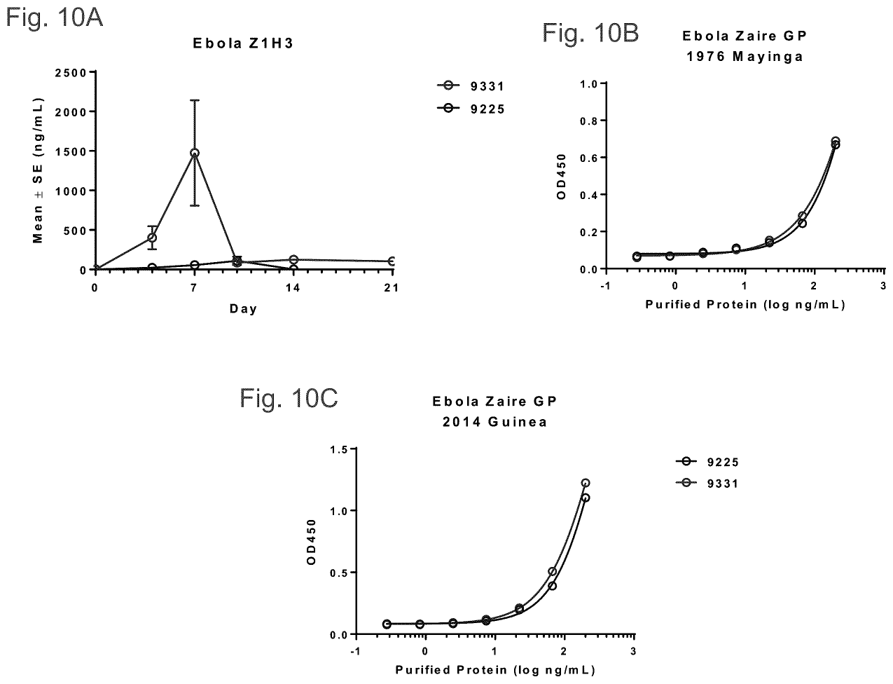

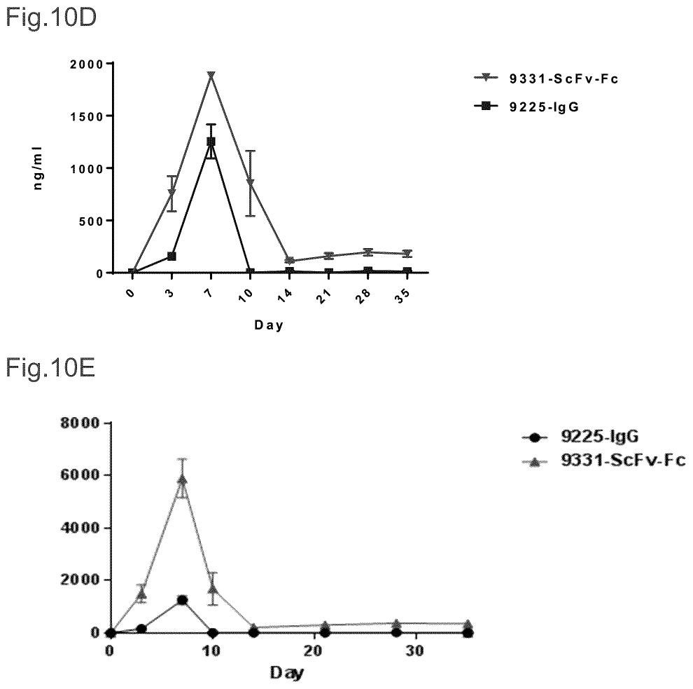

scFv-Fc conversion of BDBV223 resulted in decreased antigen binding (FIG. 8). scFv-Fc conversion of Z5D2 resulted in increased expression and increased antigen binding (FIG. 9). scFv-Fc conversion of Z1H3 resulted in increased expression but had no effect on antigen binding (FIG. 10).

### Example 4: scFv-Fc Design

Several possible engineering approaches exist, but many publications in the area tend to be very system-specific despite claims otherwise. Several new approaches used for design and currently being tested in dMAb space can potentially contribute ideas for scFv-Fc space.

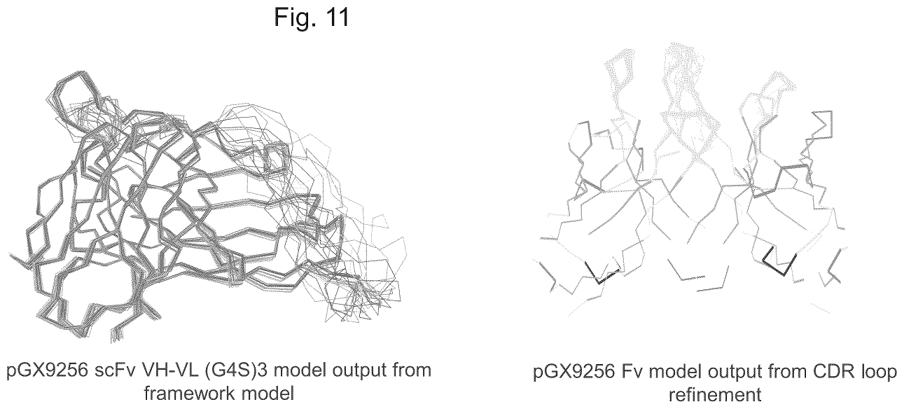

Modeling and analysis was performed to identify structural characteristics of scFv DMAb that are associated with increased binding and/or expression. Modeling was performed on Fv and scFv in both forms (VH-VL and VL-VH) using a (G4S)3 linker. Fifteen sets of models were generated, with multiple models in each set. Scoring methods as well as structural inspection are used to assess model building at each step. FIG. 11 depicts example outputs from different steps in the modeling process which includes framework modelling and CDR loop refinement. The top 20 linker conformations were evaluated. Root-mean-square deviation (RMSD) was generated of the backbone with attention to regions near linkers.

### Linker

The (G4S)3 linker, having a sequence of GGGGSGGGGGGGGS (SEQ ID NO: 53) was selected for inclusion in the studies.

The results are now described

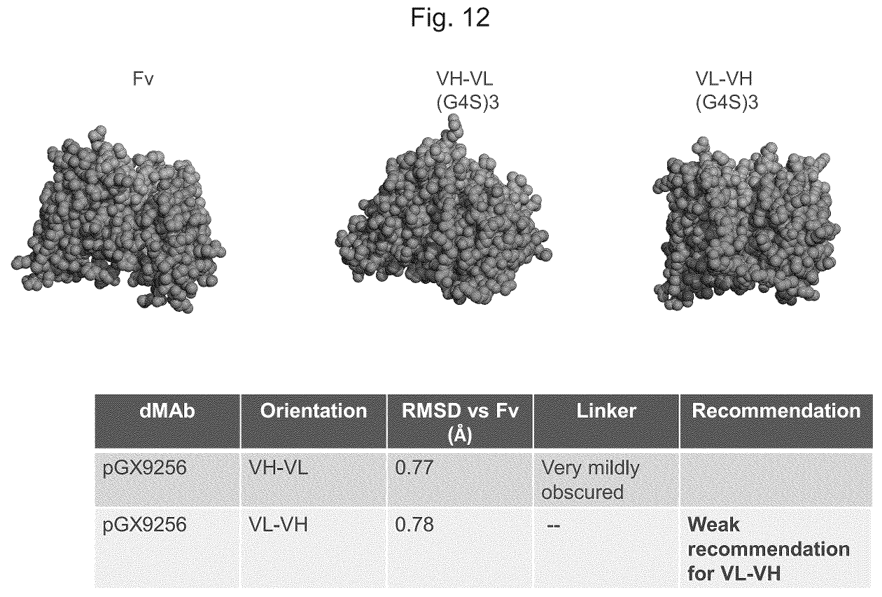

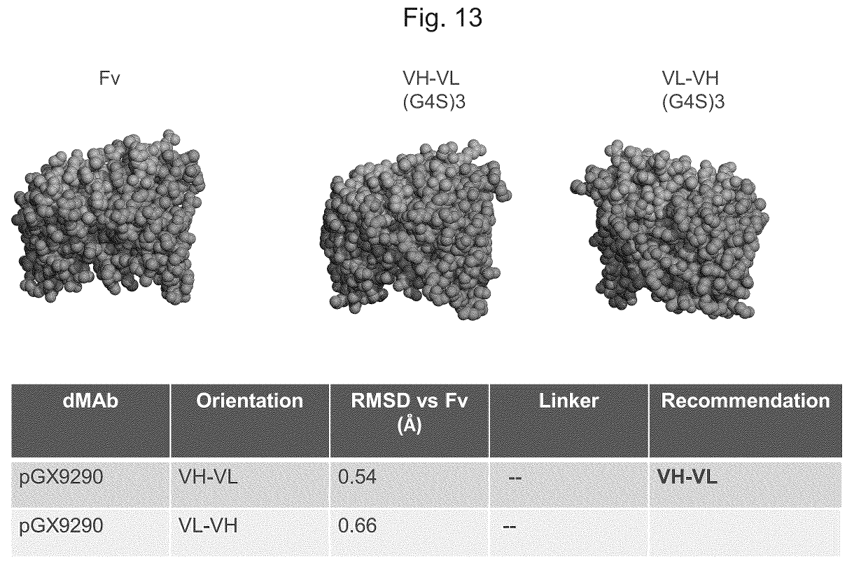

Predictive ScFv-Fc modeling was performed on five different parental DMAbs. Each DMAb was modeled in two orientations: VH-linker-VL and VL-linker-VH. FIG. 12 and FIG. 13 show space filled models of the ScFv-Fc DMAbs that are used to predict whether the linker is likely to obscure or interfere with the CDRs.

Table 3 provides a summary of the decision tree for identifying candidate DMAbs for further development.

| | | Orien- | RMSD vs | | Recommen- |
|---|---|---|---|---|---|
| DMAb | Name | tation | Fv (Å) | Linker | dation |
| 1A2 | pGX9256 | VH-VL | 0.77 | Very mildly | |
| | | | | obscured | |
| 1A2 | pGX9256 | VL-VH | 0.78 | — | Weak |
| | | | | | recommen- |
| | | | | | dation for |
| | | | | | VL-VH |
| EBV114 | pGX9290 | VH-VL | 0.54 | — | VH-VL |
| EBV114 | pGX9290 | VL-VH | 0.66 | — | |

Recommendations based on these results are in Table 3. In several cases, RMSD is within normal error for independently minimized models. In some cases, especially those where no strong signal exists between conformations, there may not be a benefit in choosing one conformation over another (VH-VL and VL-VH). In other cases, modeling indicates that one conformation may be a better candidate than another. RMSD is a simple, consistent measurement for forming a recommendation, however other criteria besides RMSD may also be informative.

Linker evaluation like that used here can detect distortion issues, but it cannot easily detect issues related to V domain association that are dependent on flexibility or intermediates in association and require greater mobility to associate (e.g., ‘entropic considerations’).

### Example 5: Functional Assessment of Structural Reformatting and Protein Engineering Strategies for Therapeutic Gene Transfer of Synthetic DNA-Plasmid Encoding Antibodies Against Ebola Virus Disease (EVD)

Ebola virus disease (EVD) causes severe hemorrhagic fever in humans and is associated with high mortality rate. The Ebola epidemic of 2013-2015 in West Africa was by far the most fatal and the longest lasting in the recent history of emerging diseases. The challenges of such a large outbreak have underscored the need for effective EVD antiviral therapies and vaccines. While a vaccine for EVD was recently found effective in humans, vaccine protection is often not immediate and not beneficial in a therapeutic setting against acute cases of EVD. Passive immunization strategies that require the transfer of monoclonal antibodies (mAb) to confer immediate protective immunity have been used successfully in infectious disease settings including EVD. However, there are conceptual and methodological hurdles associated with antibody administration. This includes their production and optimization in vitro, their dosage in vivo, and finally the costs that are incurred during the manufacturing process. From this standpoint, the in vivo delivery of DNA-plasmid encoding antibodies offers an innovative, safe and cost effective approach to monoclonal antibody (mAb) administration.

In vivo electroporation (EP)-mediated gene delivery in mice of DNA encoding monoclonal antibodies (DMAbs) directed against various pathogens has previously been demonstrated. The current study describes two DMAbs that target Ebola virus glycoprotein (EBOV-GP), EBOMAb-10 and EBOMAb-14; it also describes the functional impact of engineered modifications consisting of reformatting Immunoglobulin (Ig) to single chain antibody (scFv-Fc) and scaffold grafting within the aforementioned DMAbs structural frames. EP-mediated gene transfer of EBOMAb-10 and EBOMAb-14 in their un-modified and modified formats leads to the secretion of functional antibodies in mice serum as assessed by EBOV-GP antigen binding by ELISA and viral neutralization in an EBOV-GP pseudotyped virus assay. These structural changes have disparate effects on EBOV-GP binding in vitro and in live cell expressing surface glycoprotein, as well as on viral neutralization. Taken together, the data described here provide the conceptual framework for the development of synthetic-DNA plasmid encoding monoclonal antibodies (DMAbs) with enhanced therapeutic potency against emerging diseases such as EVD. The study also provides the functional paradigm to assess the structural manipulations of DMAbs, and overall supports further animal testing in vivo to translate DNA-based passive immunization approaches into clinic in a safe and cost efficient manner.

The methods are now described.

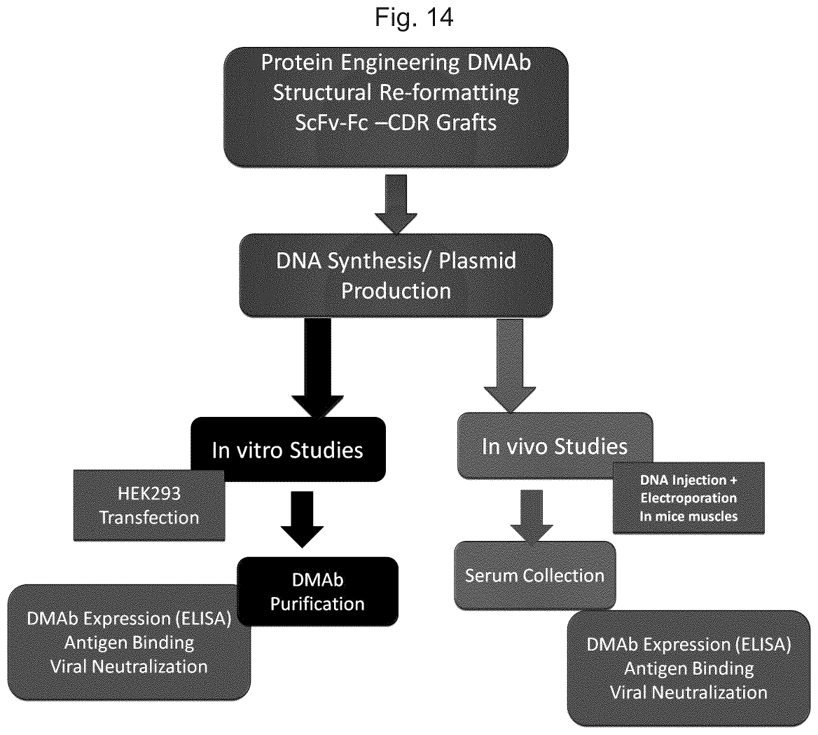

FIG. 14 provides a flow diagram of the methods used in these studies. The DMAb design strategies used for generation of the ScFv-Fc DMAbs include 1) Linker design; 2) VH-VL Orientation; and 3) Hinge-CH2-CH3 choice. Parental DMAbs underwent partial grafting, ScFv-Fc conversion, or a combination of partial grafting and ScFv-Fc conversion.

Two groups of DMAbs were analyzed in these studies. Group1 consists of the EBOMAb-10 DMAbs: EBOMAb-10-IgG (control), EBOMAb-10-2 (ScFv-Fc modified DMAb), EBOMAb-10-3-IgG partial graft and EBOMAb-10-4 ScFv-Fc partial graft. Group2 consists of the EBOMAb-14 DMAbs: EBOMAb-14-IgG (control), EBOMAb-14-2 (ScFv-Fc modified DMAb), EBOMAb-14-3-IgG partial graft and EBOMAb-14-4 ScFv-Fc partial graft. The sequences used in these studies are indicated in Table 4.

TABLE 4

| Name | Description | ASGCT code |
|---|---|---|
| pGX9256 | Ebola 5.6.1A2 | EBOMAb-10 |
| pGX9346 | 1A2_scFv-Fc | EBOMAb-10-2 |
| pGX9356 | 1A2_Full length_partial graft | EBOMAb-10-3 |
| pGX9357 | 1A2_scFv-Fc_partial graft | EBOMAb-10-4 |
| pGX9290 | Ebola EBV114 | EBOMAb-14 |
| pGX9345 | EBV114_scFv-Fc | EBOMAb-14-2 |
| pGX9362 | EBV114_Full length_partial graft | EBOMAb-14-3 |
| pGX9363 | EBV114_scFv-Fc_partial graft | EBOMAb-14-4 |

The results are now described.

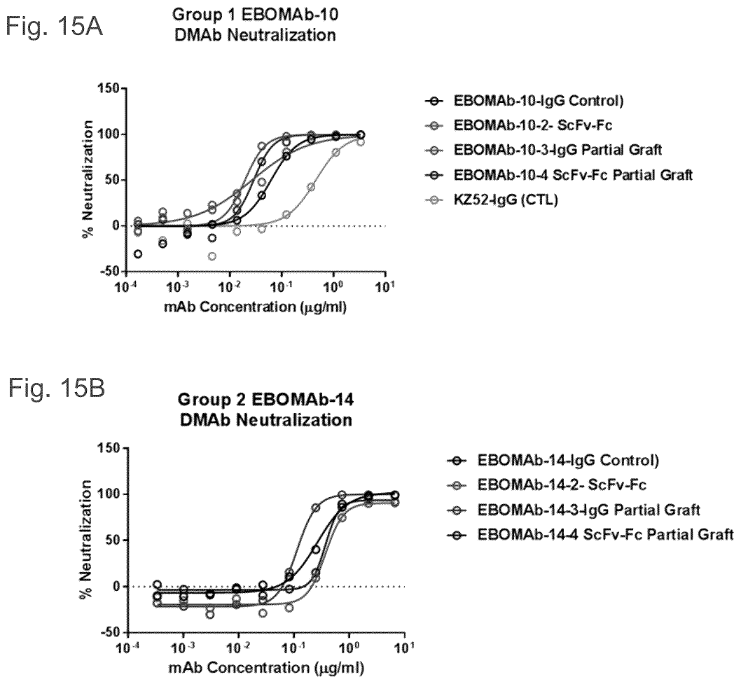

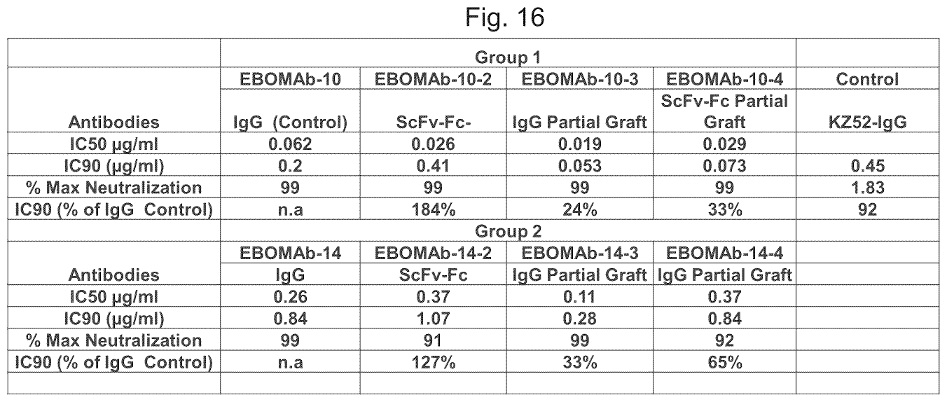

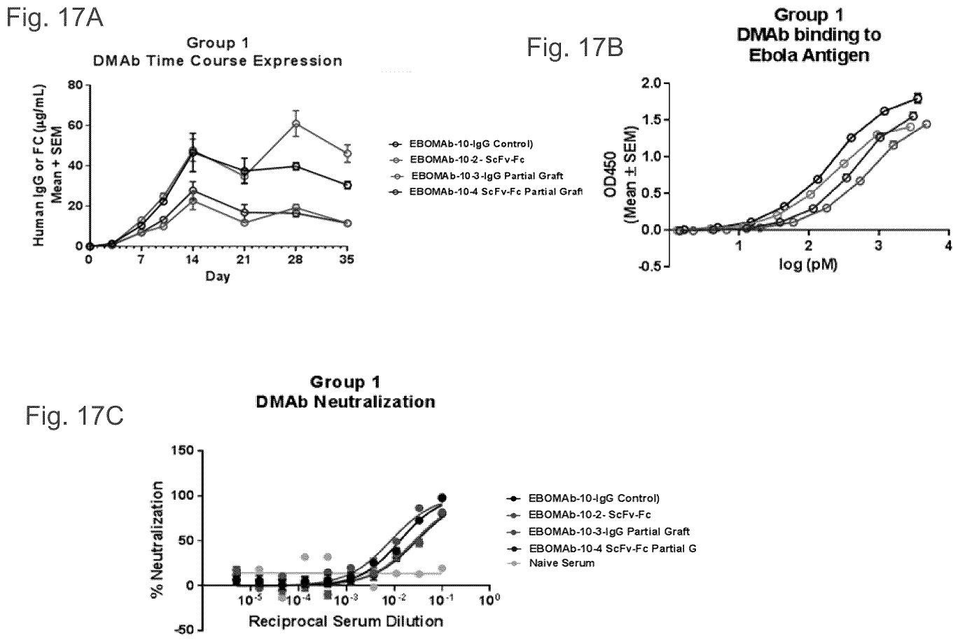

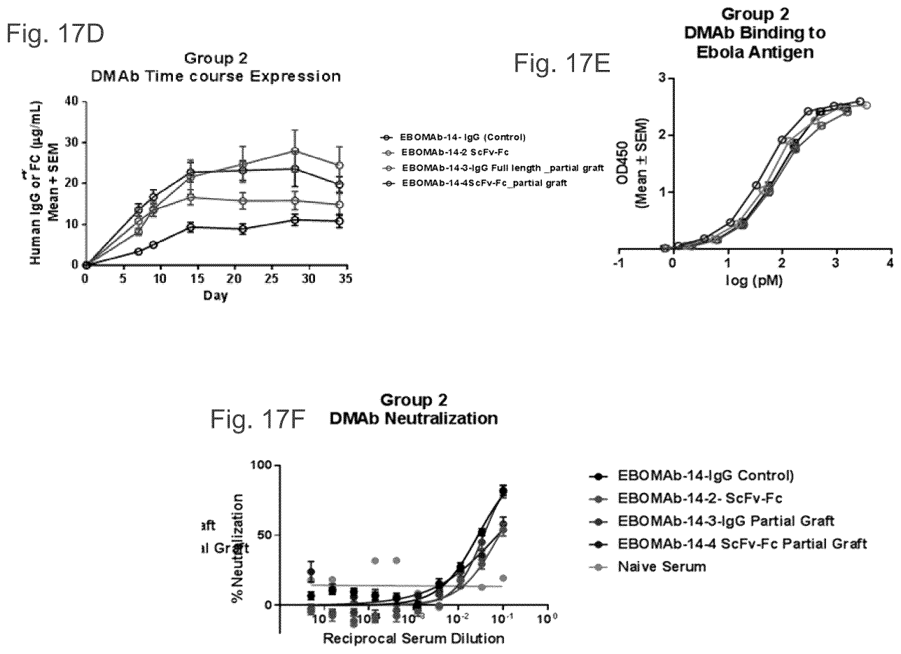

The neutralizing activity of structurally modified DMAbs was evaluated. All structurally reformatted DMAbs were comparable to their parental control DMAb (FIG. 15). An evaluation of the IC50 and IC90 showed that the ScFv-Fc DMAbs had a greater IC90 than that of the control DMAb whereas partial grafting modified DMAbs had a lower IC90 than that of the control (FIG. 16). Group 2 DMAbs showed increased expression as compared to the control DMAb and antigen binding was slightly increased or equivalent to control (FIG. 17).

Structurally modified DMAbs for the most part can be expressed at high level in mice, without substantial loss of function (i.e. antigen binding and inhibitory potency). For EBOMAb10-3 and EBOMAb-14-3 structural modification leads to increase expression in mice, and increase inhibitory potency as calculated through IC50 & IC90

The data provide proof of concept that DMAb is a malleable tool that can be engineered for possibly higher expression and higher therapeutic efficacy

Table 5 provides a listing of example Ebola DMAb plasmid constructs that utilize framework mutations, full grafts, partial grafts, and scFv-Fc conversions.

TABLE 5

| SEQ | Sequence | | |
|---|---|---|---|
| ID NO: | type | Name | Description |
| 1 | Nucleotide | pGX9291 | Ebola Z5D2 modified |
| 2 | Amino acid | pGX9291 | Ebola Z5D2 modified |
| 3 | Nucleotide | pGX9292 | Ebola Z5D2 partial graft |
| 4 | Amino acid | pGX9292 | Ebola Z5D2 partial graft |
| 5 | Nucleotide | pGX9293 | Ebola Z5D2 graft on MERSYTE_1 |
| 6 | Amino acid | pGX9293 | Ebola Z5D2 graft on MERSYTE_1 |
| 7 | Nucleotide | pGX9294 | Ebola Z5D2 graft on MERSYTE_2 |
| 8 | Amino acid | pGX9294 | Ebola Z5D2 graft on MERSYTE_2 |
| 9 | Nucleotide | pGX9295 | Ebola Z5D2 graft on V2L2 |
| 10 | Amino acid | pGX9295 | Ebola Z5D2 graft on V2L2 |
| 11 | Nucleotide | pGX9296 | Ebola ZBDBV223 modified |
| 12 | Amino acid | pGX9296 | Ebola ZBDBV223 modified |
| 13 | Nucleotide | pGX9297 | Ebola ZBDBV223 partial graft |
| 14 | Amino acid | pGX9297 | Ebola ZBDBV223 partial graft |
| 15 | Nucleotide | pGX9298 | Ebola ZBDBV223 graft on |
| | | | MERSYTE |
| 16 | Amino acid | pGX9298 | Ebola ZBDBV223 graft on |
| | | | MERSYTE |
| 17 | Nucleotide | pGX9299 | Ebola ZBDBV223 graft on V2L2 |
| 18 | Amino acid | pGX9299 | Ebola ZBDBV223 graft on V2L2 |
| 19 | Nucleotide | pGX9330 | Ebola Z5D2 scFv-Fc |
| 20 | Amino acid | pGX9330 | Ebola Z5D2 scFv-Fc |
| 21 | Nucleotide | pGX9331 | Ebola Z1H3 scFv-Fc |
| 22 | Amino acid | pGX9331 | Ebola Z1H3 scFv-Fc |
| 23 | Nucleotide | pGX9332 | Ebola ZBDBV223 scFv-Fc |
| 24 | Amino acid | pGX9332 | Ebola ZBDBV223 scFv-Fc |
| 25 | Nucleotide | pGX9345 | EBV114_scFv-Fc |
| 26 | Amino acid | pGX9345 | EBV114_scFv-Fc |
| 27 | Nucleotide | pGX9346 | 1A2_scFv-Fc |
| 28 | Amino acid | pGX9346 | 1A2_scFv-Fc |
| 29 | Nucleotide | pGX9356 | 1A2_Full length_partial graft |
| 30 | Amino acid | pGX9356 | 1A2_Full length_partial graft |
| 31 | Nucleotide | pGX9357 | 1A2_scFv-Fc_partial graft |
| 32 | Amino acid | pGX9357 | 1A2_scFv-Fc_partial graft |
| 33 | Nucleotide | pGX9362 | EBV114_Full length_partial graft |
| 34 | Amino acid | pGX9362 | EBV114_Full length_partial graft |
| 35 | Nucleotide | pGX9363 | EBV114_scFv-Fc_partial graft |
| 36 | Amino acid | pGX9363 | EBV114_scFv-Fc_partial graft |
| 37 | Nucleotide | pGX9224 | Ebola Z5D2 |
| 38 | Amino acid | pGX9224 | Ebola Z5D2 |
| 39 | Nucleotide | pGX9225 | Ebola Z1H3 |
| 40 | Amino acid | pGX9225 | Ebola Z1H3 |
| 41 | Nucleotide | pGX9228 | Ebola ZBDBV223 |
| 42 | Amino acid | pGX9228 | Ebola ZBDBV223 |
| 43 | Nucleotide | pGX9256 | 1A2 |
| 44 | Amino acid | pGX9256 | 1A2 |
| 45 | Nucleotide | pGX9290 | EBV114 |
| 46 | Amino acid | pGX9290 | EBV114 |

### Example 6: Exploration of Gene Optimization and scFv-Fc Reformatting as Strategies to Increase In Vivo Expression Levels of DNA-Encoded Monoclonal Antibodies (DMAbs) Against Zika Virus

Two antibody modification strategies were used to generate modified DMAbs targeting the Zika virus: gene optimization and scFv-Fc reformatting (FIG. 1).

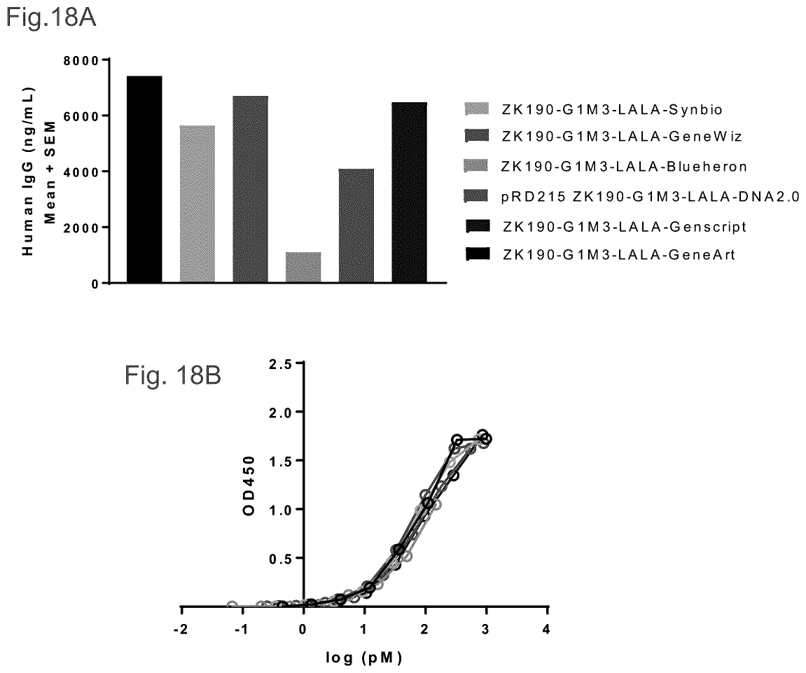

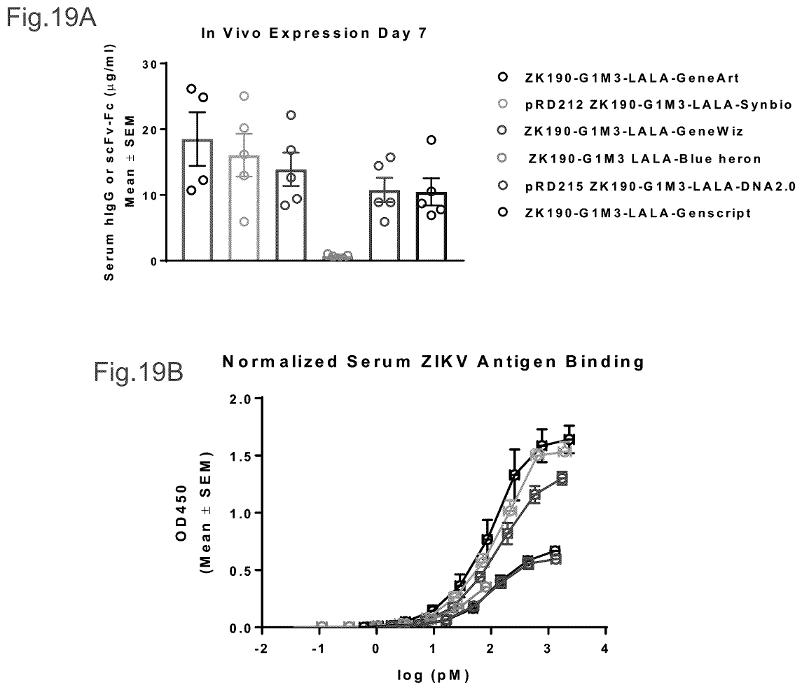

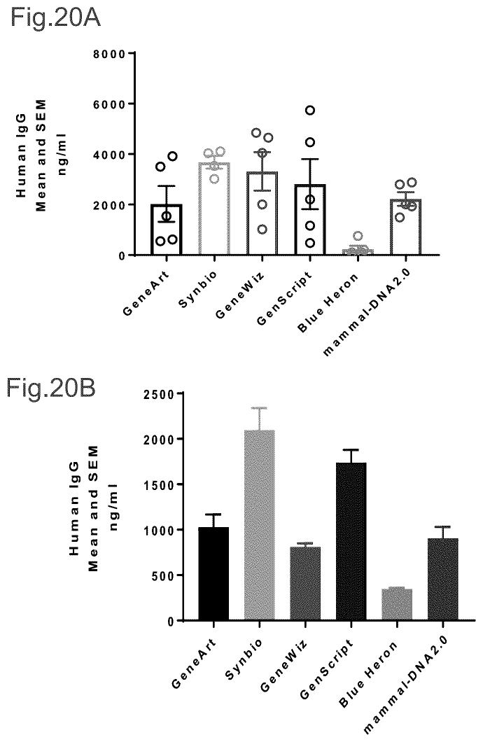

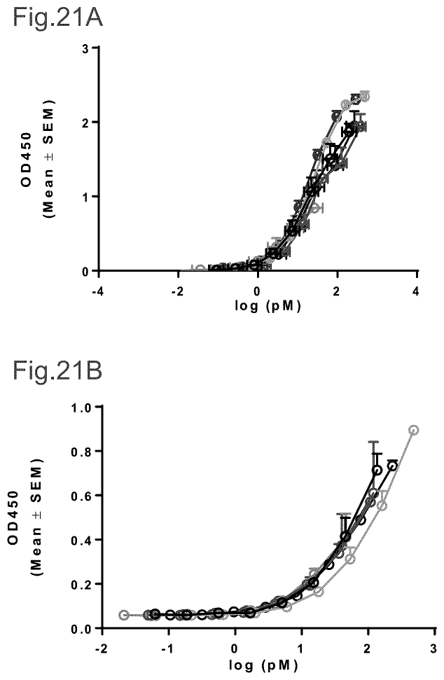

The gene optimization method consisted of selecting two full length Zika DMAb sequences and optimizing via six different algorithms. Multiple parameters affecting transcription and translation, such as codon usage, GC content, cryptic splice sites and mRNA secondary structure are weighted in proprietary multivariate regression algorithms. Much of the data referenced, however, was generated using in vitro expression systems. To find an algorithm most suited to the in vivo expression of the Zika DMAbs, BALB/c mice (n=5) were administered with 100 μg of plasmid DNA in one treatment site through intramuscular delivery followed by electroporation. Serum levels and normalized binding of DMAbs were quantified by ELISA at day 7. For ZKDMAB-1, Algorithm 1 gave the highest expression at 18 μg/ml (FIG. 18 and FIG. 19). For ZKDMAB-2, Algorithm 2 gave the highest expression of 3.5 ug/ml (FIG. 20). Consistently, both DMAbs optimized by Algorithm 6 exhibited the lowest or no expression in vivo. In most cases, binding by ELISA was retained, however several algorithms saw a decrease for ZKDMAB-1, suggesting that protein folding or conformation of the expressed DMAb could have been affected by the nucleotide sequence (FIG. 18, FIG. 19 and FIG. 21).

Additionally, single chain Fv-Fc (scFv-Fc) conversion was tested. ScFv-Fc conversion is the removal of CH1 and CL regions, and the addition of a linker between VH and VL. Conversion promotes heavy chain-light chain pairing and tissue penetration. DMAbs are converted from a full length antibody to scFv-Fc through addition of a linker (as depicted in FIG. 1).

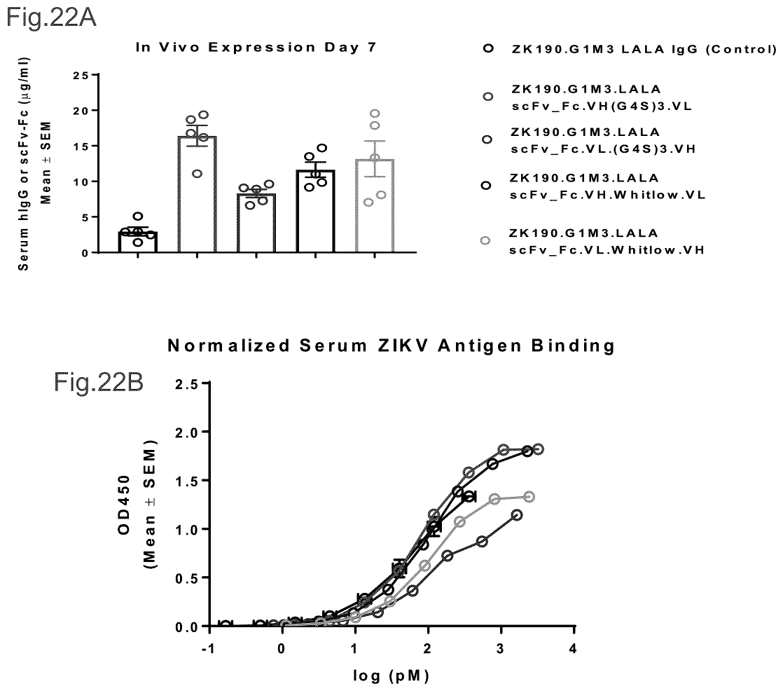

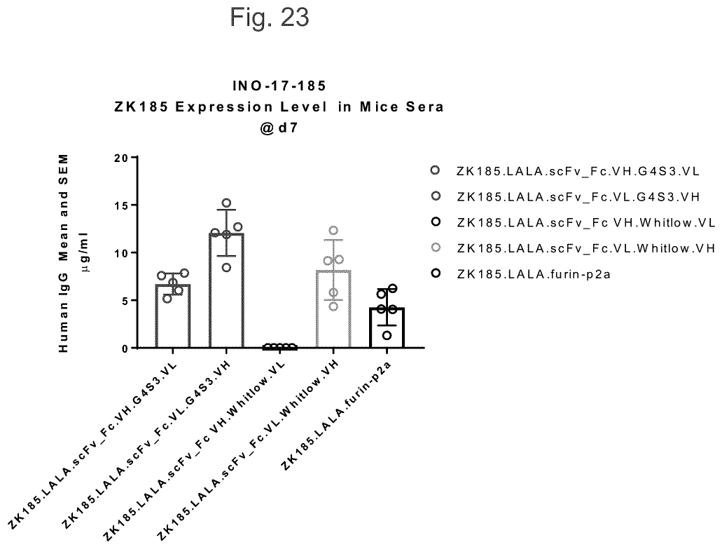

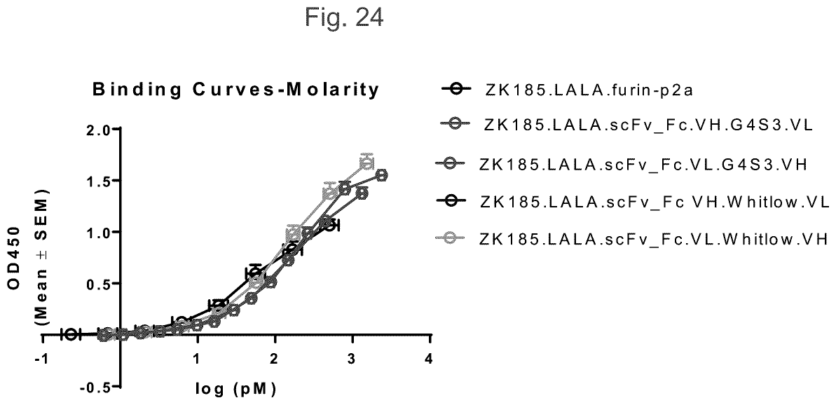

Two Zika DMAbs were chosen, and from them multiple constructs were generated. They differed in their choice of linker molecule and the orientation of the VH-VL. Converting DMAbs from a full length antibody to scFv-Fc resulted in an increase in murine expression of up to 6 fold compared to the original DMAb. For ZKDMAB-1, expression of the four formats tested ranged from 16 μg/ml down to 8 μg/ml and favored the (G4S)3 linker in the VH-VL orientation. ZKDMAB-2 saw highest expression reach 12 μg/ml using the (G4S)3 linker in the VL-VH orientation. Importantly, modifications made to the majority of DMAbs retained antigen binding. Through these changes the in vivo expression levels were increased without sacrificing the biology of the original mAb clone (FIG. 22 through FIG. 24). These data demonstrate the obvious benefit of gene and protein modulation when designing DMAbs for gene therapy applications.

TABLE 6
Engineered anti-ZIKV DMAbs: GO = gene optimization;
190 = ZK190-G1M3-LALA; 185 = ZK185.LALA.furin-p2a

| SEQ | Sequence | DMAb | | |
|---|---|---|---|---|
| ID NO: | Type | Type | Name | Description |
| 63 | Nucleotide | GO | pRD211 | 190-FP2A-mouse-GeneArt |
| 64 | Amino acid | GO | pRD211 | 190-FP2A-mouse-GeneArt |
| 65 | Nucleotide | GO | pRD212 | 190-FP2A-mouse-Synbio |
| 66 | Amino acid | GO | pRD212 | 190-FP2A-mouse-Synbio |
| 67 | Nucleotide | GO | pRD213 | 190-FP2A-mouse-Genewiz |
| 68 | Amino acid | GO | pRD213 | 190-FP2A-mouse-Genewiz |
| 69 | Nucleotide | GO | pRD214 | 190-FP2A-mouse-Blue heron |
| 70 | Amino acid | GO | pRD214 | 190-FP2A-mouse-Blue heron |
| 71 | Nucleotide | GO | pRD215 | 190-FP2A-mammal-DNA2.0 |
| 72 | Amino acid | GO | pRD215 | 190-FP2A-mammal-DNA2.0 |
| 73 | Nucleotide | GO | pRD216 | 190-FP2A-mouse-Genscript |
| 74 | Amino acid | GO | pRD216 | 190-FP2A-mouse-Genscript |
| 75 | Nucleotide | GO | pRD225 | 185-mouse-GeneArt |
| 76 | Amino acid | GO | pRD225 | 185-mouse-GeneArt |
| 77 | Nucleotide | GO | pRD227 | 185-mouse-Synbio |
| 78 | Amino acid | GO | pRD227 | 185-mouse-Synbio |
| 79 | Nucleotide | GO | pRD229 | 185-mouse-GeneWiz |
| 80 | Amino acid | GO | pRD229 | 185-mouse-GeneWiz |
| 81 | Nucleotide | GO | pRD231 | 185-mouse-GenScript |
| 82 | Amino acid | GO | pRD231 | 185-mouse-GenScript |
| 83 | Nucleotide | GO | pRD233 | 185-mouse-Blue Heron |
| 84 | Amino acid | GO | pRD233 | 185-mouse-Blue Heron |
| 85 | Nucleotide | GO | pRD234 | 185-mammal-DNA2.0 |
| 86 | Amino acid | GO | pRD234 | 185-mammal-DNA2.0 |
| 87 | Nucleotide | Parental | pGX9382 | 190 |
| 88 | Amino acid | Parental | pGX9382 | 190 |
| 89 | Nucleotide | scFv_Fc | pGX93100 | 190.scFv_Fc.VH.G4S3.VL |
| 90 | Amino acid | scFv_Fc | pGX93100 | 190.scFv_Fc.VH.G4S3.VL |
| 91 | Nucleotide | scFv_Fc | pGX93101 | 190.scFv_Fc.VL.G4S3.VH |
| 92 | Amino acid | scFv_Fc | pGX93101 | 190.scFv_Fc.VL.G4S3.VH |
| 93 | Nucleotide | scFv_Fc | pGX93102 | 190.scFv_Fc VH.Whitlow.VL |
| 94 | Amino acid | scFv_Fc | pGX93102 | 190.scFv_Fc VH.Whitlow.VL |
| 95 | Nucleotide | scFv_Fc | pGX93103 | 190.scFv_Fc.VL.Whitlow.VH |
| 96 | Amino acid | scFv_Fc | pGX93103 | 190.scFv_Fc.VL.Whitlow.VH |
| 97 | Nucleotide | Parental | pGX93134 | 185 |
| 98 | Amino acid | Parental | pGX93134 | 185 |
| 99 | Nucleotide | scFv_Fc | pGX93129 | 185.scFv_Fc.VH.G4S3.VL |
| 100 | Amino acid | scFv_Fc | pGX93129 | 185.scFv_Fc.VH.G4S3.VL |
| 101 | Nucleotide | scFv_Fc | pGX93130 | 185.scFv_Fc.VL.G4S3.VH |
| 102 | Amino acid | scFv_Fc | pGX93130 | 185.scFv_Fc.VL.G4S3.VH |
| 103 | Nucleotide | scFv_Fc | pGX93131 | 185.scFv_Fc VH.Whitlow.VL |
| 104 | Amino acid | scFv_Fc | pGX93131 | 185.scFv_Fc VH.Whitlow.VL |
| 105 | Nucleotide | scFv_Fc | pGX93132 | 185.scFv_Fc.VL.Whitlow.VH |
| 106 | Amino acid | scFv_Fc | pGX93132 | 185.scFv_Fc.VL.Whitlow.VH |

### Example 7: Evaluation of a Multivalent scFv-Fc DNA-Encoded Monoclonal Antibodies (DMAb) Platform Against Zika Virus (ZIKV) and Dengue Virus (DENV) Infections

This study describes the engineering of two single-chain fragment variable-Fc (scFv-Fcs) DMAbs, Z-DMAb1-sc and D-DMAb1-sc that target ZIKV and DENV, respectively. It also describes the engineering of an additional DMAb that encodes both Z-DMAb1-sc and D-DMAb1-sc in a multivalent bi-directional promoter format (Z/D-DMAb1-sc). Using a murine model, the CELLECTRA®-EP technology was used to deliver intramuscularly in various cocktail combinations Z-DMAb1-sc and D-DMAb1-sc as well as individually formulated multivalent Z/D-DMAb1-sc. EP-mediated gene transfer of each of these scFv-Fc DMAbs leads to the secretion of functional scFv-Fcs in mice serum as assessed by ELISA and viral antigen binding assays. From this observation, higher scFv-Fc expression for Z-DMAb1-sc and D-DMAb1-sc was observed when expressed in the single multivalent bi-directional promoter construct (Z/D-DMAb1-sc) than when the two DNA plasmid constructs were co-formulated in a single preparation or separately delivered at two-individual muscle sites. Furthermore, the effect of these various co-formulations and multivalent combinations of the neutralization phenotype was analyzed. Taken all together these data provide evidence for adopting a multivalent scFv-Fc DMAb platform that may prove more versatile to combat infections by multiple pathogens such as ZIKV and DENV that are prevalent in overlapping endemic zones.

TABLE 7
Engineered anti-DENV DMAbs:

| SEQ | Sequence | DMAb | | |
|---|---|---|---|---|
| ID NO: | Type | type | Name | Description |
| 107 | Nucleotide | scFv_Fc | pGX93141 | DVSF3 LALA |
| | | | | scFv-Fc |
| | | | | VH.G4S3.VL |
| 108 | Amino acid | scFv_Fc | pGX93141 | DVSF3 LALA |
| | | | | scFv-Fc |
| | | | | VH.G4S3.VL |

### Example 8: Functional Characterization of In Vivo Expressed DNA-Based Monoclonal Antibodies (dMAbs) Against Respiratory Syncytial Virus (RSV)

While mAbs have been shown to be effective in providing protection against many infectious diseases their widespread use is limited. The limited in vivo half-life means multiple doses are required to maintain immunity, and the high costs and complexities involved in development, manufacture and cold chain distribution, and lack of suitable dosing methods which can be employed in the field, also hinder their global use. In response, new strategies based on the in vivo delivery of antibody genes are being developed. One such platform is dMAb, a synthetic plasmid DNA-encoded mAb. A DNA sequence encoding for a human mAb is inserted into a plasmid. As depicted in FIG. 1B, the dMAb plasmid is delivered directly to the muscle tissue and in-vivo electroporation enhances cellular uptake by myocytes. The transfected myocytes produce and secrete the mAb. The mAb enters blood circulation and can function systemically. In proof-of-concept studies dMAbs have provided protection against various infectious diseases, including influenza, pseudomonas, Zika and Ebola in pre-clinical animal models. Here will delineate the preclinical development of a dMAb targeting RSV. A RSV-dMAb could be delivered to high-risk populations to provide prophylactic protection against severe complications from RSV-infection across the season. Due to maintained expression one initial dMAb delivery would cover the whole RSV-season, re-delivery might be not necessary.

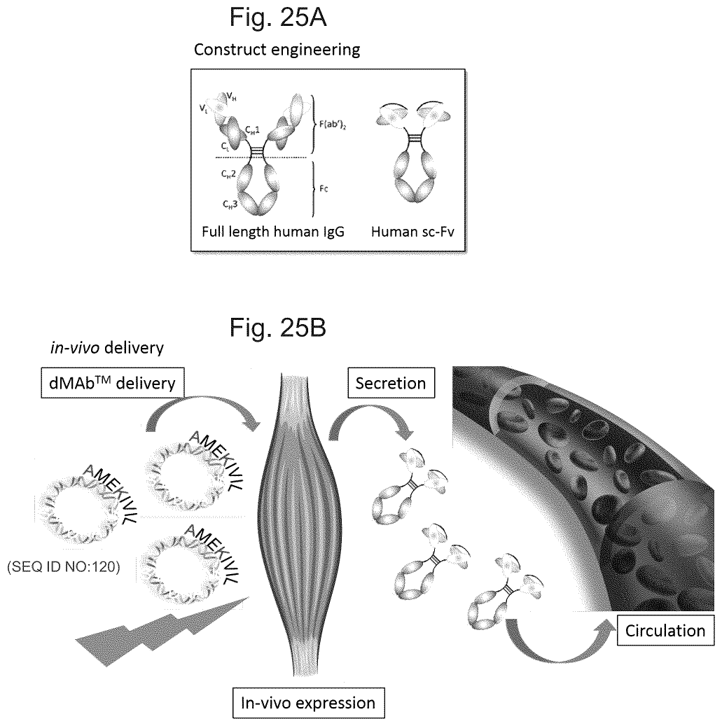

An anti-RSV sc-Fv encoding DNA plasmid was engineered (FIG. 25A) with an improved in-vivo expression profile compared to the full length human IgG. To further enhance systemic expression an optimized delivery protocol was employed. An optimized formulation enhances dispersion of the plasmid DNA through modifications of the extracellular matrix of the target tissue. Electroporation increases cellular uptake of the DNA molecules by target cells. The functionality of the in-vivo expressed human sc-Fv was confirmed from serum of treated mice for binding to the RSV-Fusion protein (RSV-F) antigen and neutralizing live RSV-A virus in-vitro. In addition to serum-level expression, the in-vivo expressed human sc-Fv was also detected in the lung, the location of natural RSV-infection. Dosing and delivery methods were then applied to cotton rats, the standard pre-clinical model for RSV-prophylaxis development.

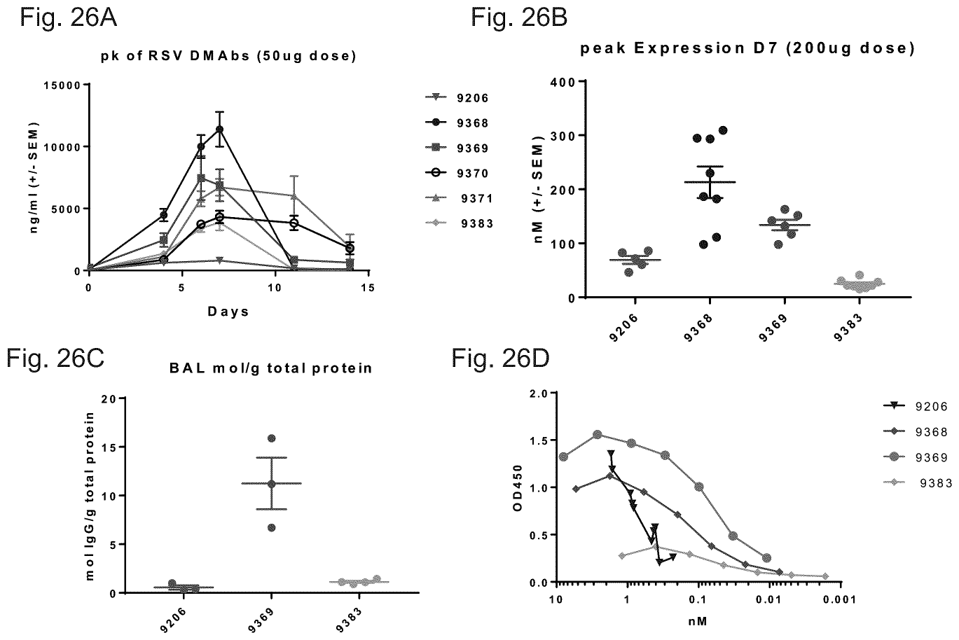

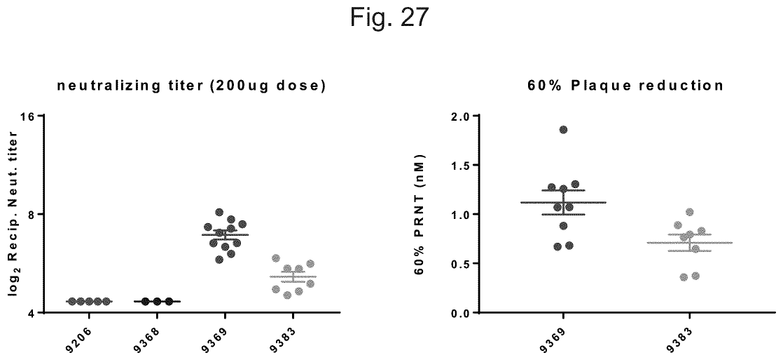

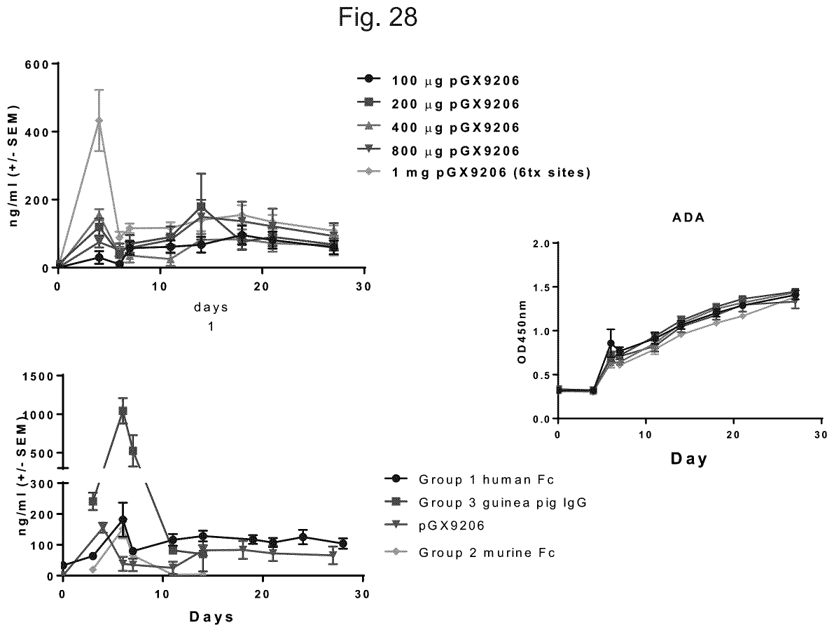

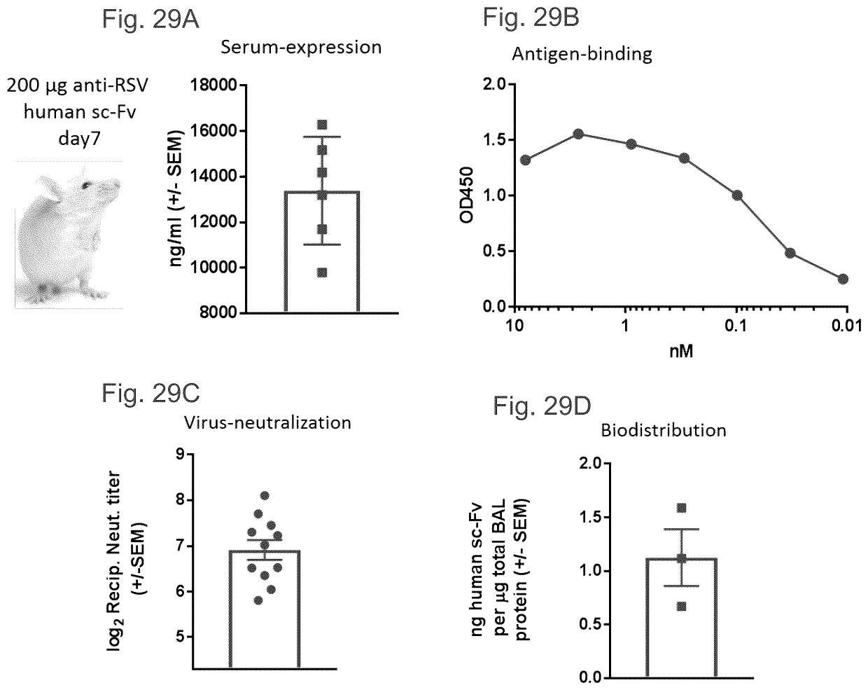

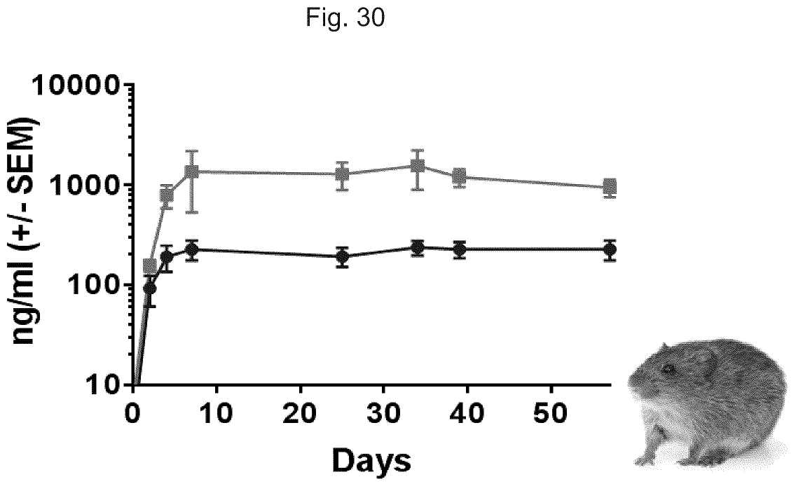

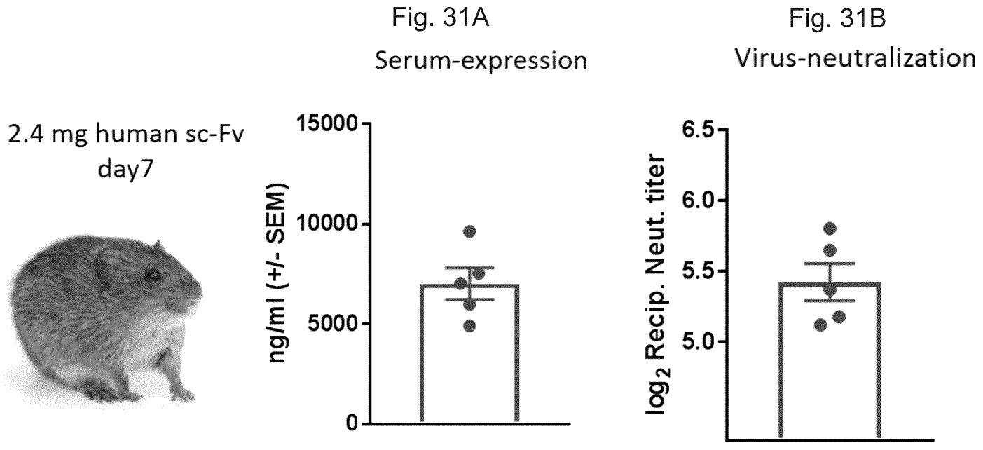

In vivo delivery of this dMAb resulted in robust systemic levels of the antibody in the serum of mice (FIG. 26 and FIG. 29A). Matched levels of recombinant Pavilizumab provide protection from lower respiratory disease after RSV infection. In cotton rats, which is the gold-standard to model human disease following RSV infection, sustained serum-expression of the dMAb was observed up to 60 days after delivery (FIG. 30). The antibody was also detected in lung-lavage samples, demonstrating effective biodistribution (FIG. 26C and FIG. 29D). Furthermore, serum from animals harboring RSV-F dMAb was functionally active in terms of antigen binding and neutralizing live virus (FIG. 27, FIG. 29B, FIG. 29C and FIG. 31B). Experiments are designed to conduct an in-vivo live virus challenge of cotton rats. The cotton rat is considered the model of choice for preclinical development of RSV vaccines because of their high susceptibility to non-adapted human RSV and display of many features of the pathology of infection in humans.

These results suggest that the anti-RSV human sc-Fv dMAb could be an effective alternative to repetitive injections of protein-mAb throughout RSV-season. RSV-dMAb has the potential to overcome some of the hurdles associated with the passive immunization.

TABLE 8
RSV DMAb Plasmids:

| | | Fc/ | Delivery |
|---|---|---|---|
| DMAb | description | conformation | protocol |
| 9206 | Manni's | huIgG | 30 min pre-tx |
| | Motavizumab | | Sigma-HYA |
| 9368 | Palivizumab | huIgG | Co-formulation |
| | | | Intropharma HYA |
| 9369 | Palivizumab | huIgG sc-Fv | Co-formulation |
| | | | Intropharma HYA |
| 9370 | Palivizumab | muIgG | Co-formulation |
| | | | Intropharma HYA |
| 9371 | Palivizumab | muIgG sc-Fv | Co-formulation |
| | | | Intropharma HYA |
| 9283 | ADImab | huIgG | Co-formulation |
| | | | Hylenex |

TABLE 9
Engineered anti-RSV DMAbs:

| SEQ | Sequence | DMAb | |
|---|---|---|---|
| ID NO: | Type | type | Name |
| 110 | Nucleotide | GO | pGX9368 |
| 111 | Amino acid | GO | pGX9368 |
| 112 | Nucleotide | scFv-Fc | pGX9369 |
| 113 | Amino acid | scFv-Fc | pGX9369 |
| 114 | Nucleotide | GO | pGX9370 |
| 115 | Amino acid | GO | pGX9370 |
| 116 | Nucleotide | scFv-Fc | pGX9371 |
| 117 | Amino acid | scFv-Fc | pGX9371 |
| 118 | Nucleotide | GO | pGX9283 |
| 119 | Amino acid | GO | pGX9283 |

### Example 9: A Multivalent scFv-Fc DNA-Encoded Monoclonal Antibodies (dMAb™) Platform Against Zika Virus (ZIKV) and Dengue Virus (DENV) Infections

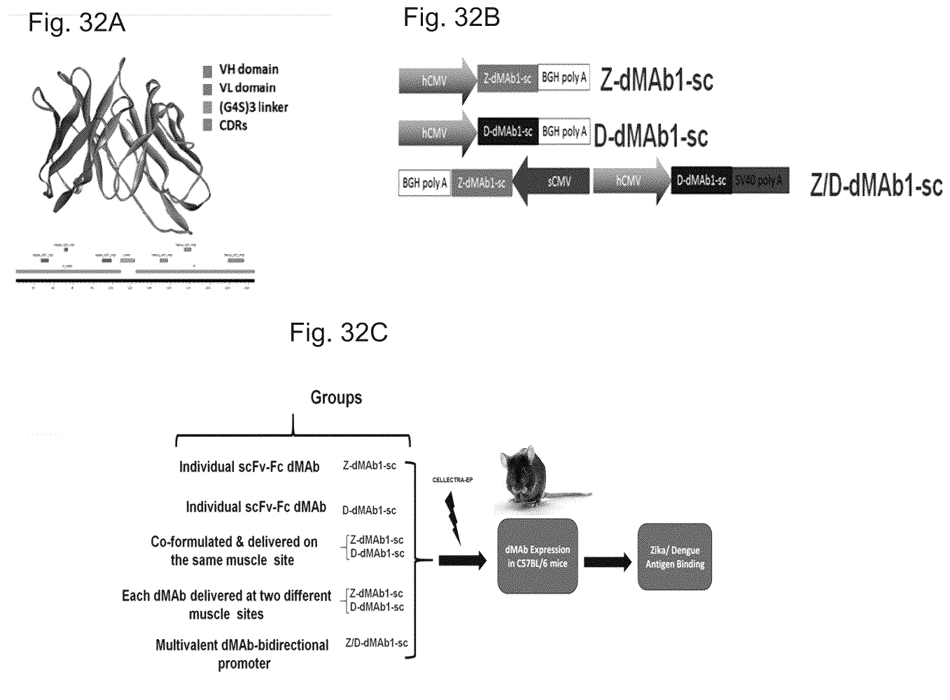

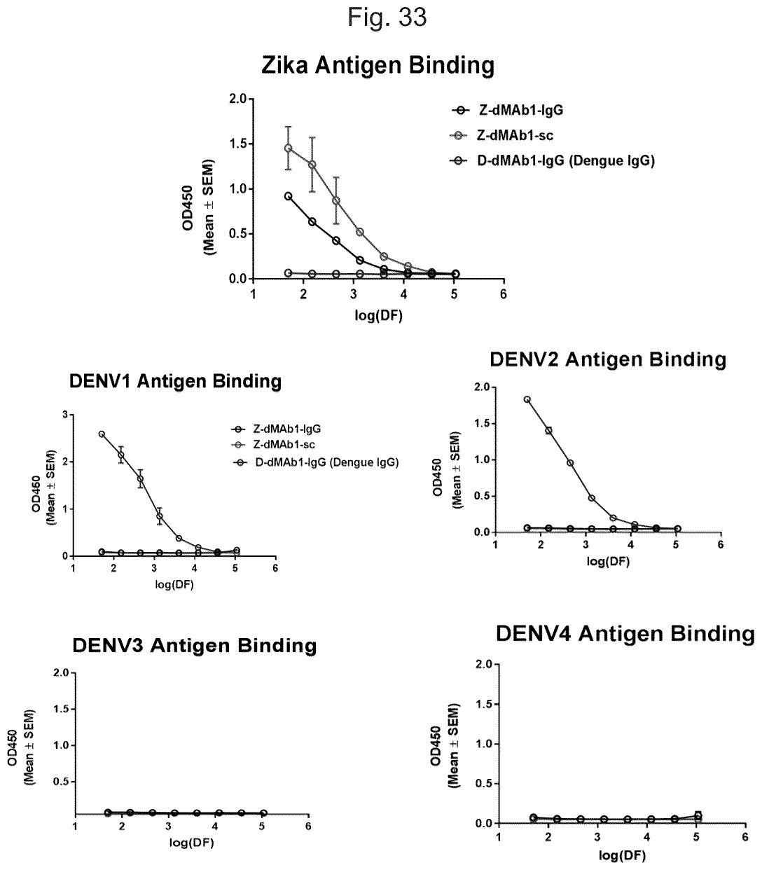

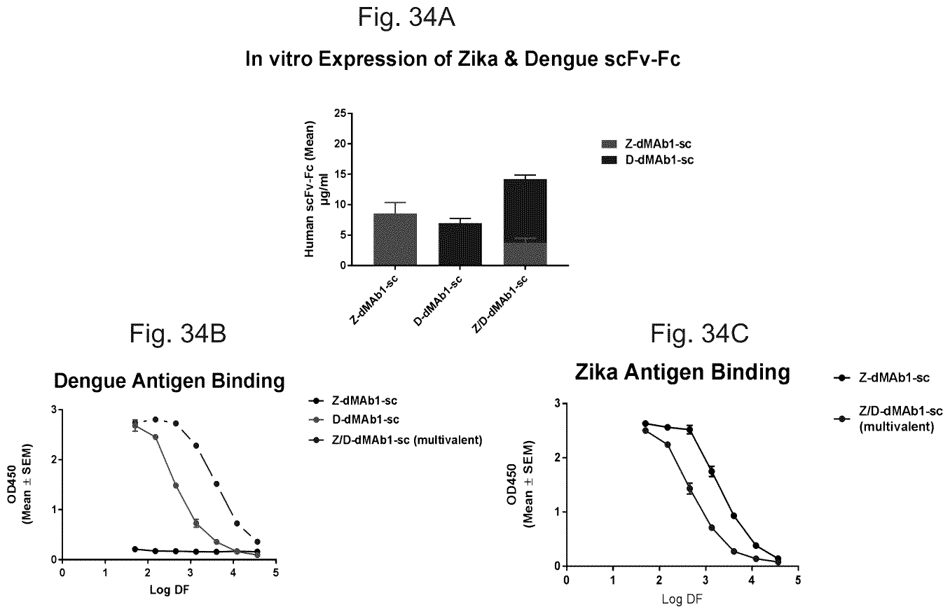

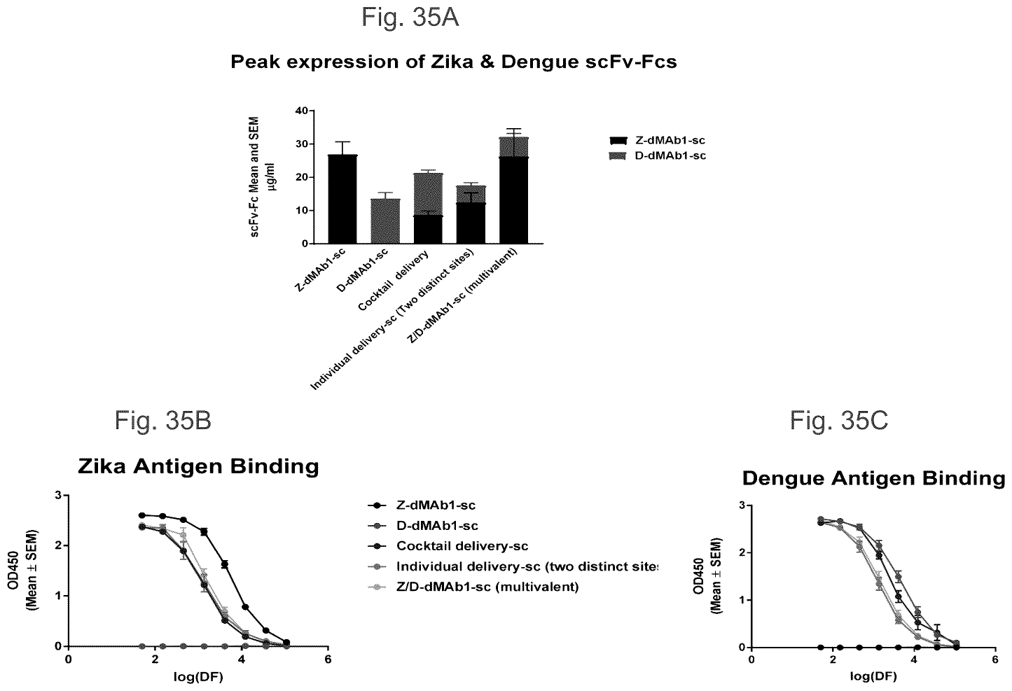

Zika (ZIKV) and Dengue (DENV) viruses are mosquito-borne flavivirus that cause from mild to severe pathologies varying from minor rashes to severe organ failure that could lead to death furthermore, infection by ZIKV specifically during pregnancy is associated with spontaneous abortion or severe developmental defects in newborns, including microcephaly and cognitive impairment that can be individually and societally burdensome. Previously published pre-clinical models have laid the rationale for using neutralizing monoclonal antibodies (mAbs) as basis for therapeutic intervention against ZIKV and DENV infections. While mAbs administration holds great promises as both prophylactic and curative approaches for infectious diseases there are conceptual and methodological impediments associated with the large scale administration of protein mAbs specifically for several millions people potentially at risk of contracting ZIKV or/and DENV infections. An alternative approach for treatment of these diseases is based on expression of plasmid-encoding monoclonal antibody (dMAbs) in skeletal muscles that leads to in vivo production and secretion of mAbs in the serum. In an attempt to design a more versatile dMAb platform, with increased pathogenic coverage, two single-chain fragment variable-Fc (scFv-Fcs) dMAbs were engineered, Z-dMAb1-sc and D-dMAb1-sc that target ZIKV and DENV, respectively (FIG. 32). An additional dMAb that encodes both Z-dMAb1-sc and D-dMAb1-sc in a multivalent bi-directional promoter format (Z/D-dMAb1-sc) was also engineered (FIG. 32 and Table 10). The CELLECTRA®-EP technology was used to deliver intramuscularly in a murine model various cocktail combinations of plasmid DNA encoding Z-dMAb1-sc and D-dMAb1-sc as well as individually formulated multivalent plasmid DNA encoding Z/D-dMAb1-sc dMAb. EP-mediated gene transfer of each of these scFv-Fc dMAbs leads to the secretion of functional scFv-Fcs in mice serum as assessed by ELISA and viral antigen binding assays (FIG. 33). From this observation, disparate scFv-Fc expression was noted for Z-dMAb1-sc and D-dMAb1-sc when expressed in the single multivalent bi-directional promoter construct (Z/D-dMAb1-sc) as compared to when the two DNA plasmid constructs were co-formulated in a single preparation or separately delivered at two-individual muscle sites (FIG. 34). Furthermore, how these various co-formulations and multivalent combinations affect the neutralization phenotype was analyzed (FIG. 35). The multivalent scFv-Fc dMAb platform allows the production of two functional scFv-Fc dMAbs (Z-dMAb1-sc & D-dMAb1-sc). Cocktailing/Mixing scFv-Fc Zika/Dengue dMAb appears to benefit dengue dMAb1-sc expression. Further, molecular engineering of Zika/Dengue dMAb in a bi-directional promoter platform appears to benefit significantly the expression of Dengue dMAb with no significant difference in Zika expression

Taken all together these data provide support for a multivalent scFv-Fc dMAb platform that may prove more adaptable to combat infections by multiple pathogens, such as ZIKV and DENV, that are prevalent in overlapping endemic zones.

TABLE 10
Structurally Modified DMAb sequences

| SEQ | Sequence | DMAb | | |
|---|---|---|---|---|
| ID NO: | Type | type | Name | Description |
| 89 | Nucleotide | scFv_Fc | pGX93100 | 190.scFv_Fc.VH.G4S3.VL |
| 90 | Amino acid | scFv_Fc | pGX93100 | 190.scFv_Fc.VH.G4S3.VL |
| 107 | Nucleotide | scFv_Fc | pGX93141 | DVSF3 LALA scFv-Fc |
| | | | | VH.G4S3.VL |
| 108 | Amino acid | scFv_Fc | pGX93141 | DVSF3 LALA scFv-Fc |
| | | | | VH.G4S3.VL |
| 126 | Nucleotide | Bidirectional | pRD245 | DVSF3 LALA scFv-Fc |
| | | scFv_Fc | | VH.G4S3.VL and |
| | | | | 190.scFv_Fc.VH.G4S3.VL |
| | | | | bidirectional expression |
| | | | | plasmid |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method of generating a nucleic acid sequence encoding a structurally modified DNA encoded antibody (DMAb).

2. The method of claim 1 comprising the steps of:
a) identifying one or more CDR region of a first DMAb,
b) identifying one or more CDR region of a second DMAb,
c) substituting the amino acid sequence of one or more CDR region of a second DMAb with the amino acid sequence of one or more CDR region of a first DMAb to generate an amino acid sequence of a structurally modified DMAb,
d) generating a nucleic acid sequence encoding a structurally modified DMAb, and
e) optimizing the nucleic acid sequence.

3. The method of claim 1 comprising the steps of:
a) performing sequence alignment of an amino acid sequence or a fragment of an amino acid sequence of a first DMAb with the same from one or more additional DMAb sequences,
b) substituting one or more amino acid residues of the amino acid sequence of the first DMAb with one or more amino acid residues of the amino acid sequence of one or more additional DMAb sequences, wherein the one or more amino acid residues are not residues in a CDR region, to generate an amino acid sequence of a structurally modified DMAb, and
d) generating a nucleic acid sequence encoding a structurally modified DMAb.

4. The method of claim 3 further comprising optimizing the nucleic acid sequence.

5. The method of claim 1 comprising the steps of:
a) identifying amino acid residues of a DMAb that are predicted to participate in interaction at an interface of a variable heavy chain and variable light chain domain,
b) making one or more amino acid substitutions of the identified amino acid residues, wherein the one or more substitutions is predicted to alter at least one of an isoelectric point and a surface charge at the interface, to generate an amino acid sequence of a structurally modified DMAb, and
d) generating a nucleic acid sequence encoding a structurally modified DMAb.

6. The method of claim 5 further comprising optimizing the nucleic acid sequence.

7. The method of claim 1 comprising the steps of:
a) removing an amino acid sequence for a CH and CL domain from an amino acid sequence of a DMAb,
b) adding an amino acid sequence of a linker between an amino acid sequence for a VH and VL domain to an amino acid sequence of a DMAb to generate an amino acid sequence of a structurally modified DMAb,
c) performing at least one round of modeling on the generated amino acid sequence, and
d) generating a nucleotide sequence encoding a structurally modified DMAb.

8. The method of claim 7 further comprising optimizing the nucleic acid sequence.

9. The method of claim 7, wherein the method of modeling comprises at least one of linker modeling, hinge modification modeling, framework modeling, and CDR loop refinement.

10. The method of claim 7, wherein the linker is C-terminally linked to the VH domain and N-terminally linked to the VL domain.

11. The method of claim 7, wherein the linker is N-terminally linked to the VH domain and C-terminally linked to the VL domain.

12. The method of claim 2 further comprising the steps of:
a) removing an amino acid sequence for a CH and CL domain from an amino acid sequence of a DMAb,
b) adding an amino acid sequence of a linker between an amino acid sequence for a VH and VL domain to an amino acid sequence of a DMAb to generate an amino acid sequence of a structurally modified DMAb, and
c) performing at least one round of modeling on the generated amino acid sequence.

13. A structurally modified DMAb encoded by the nucleic acid molecule generated according to the method of claim 1.

14. A structurally modified DMAb comprising at least one selected from the group consisting of:
a) one or more CDR region from a first DMAb and one or more constant region from a second DMAb,
b) one or more amino acid substitutions, and
c) a CH1, CH2, hinge, VH, linker and VL domain,
wherein the structurally modified DMAb exhibits at least one of higher in vivo expression and higher binding compared to a corresponding DMAb not so modified.

15. The structurally modified DMAb of claim 14, wherein the structurally modified DMAb exhibits at least the same or higher antigen binding specificity compared to the specificity of the structurally modified DMAb prior to having been so modified.

16.-17. (canceled)

18. A composition comprising a nucleic acid molecule comprising at least one nucleotide sequence encoding a structurally modified DMAb of claim 13.

19. The composition of claim 18, wherein at least one structurally modified DMAb comprises an amino acid sequence selected from the group consisting of:
a) an amino acid sequence having at least 90% over an entire length of the encoded sequence to an amino acid sequence selected from the group consisting of: SEQ ID NO: 2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO:18, SEQ ID NO:20, SEQ ID NO:22, SEQ ID NO: 24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO: 46, SEQ ID NO:64, SEQ ID NO:66, SEQ ID NO:68, SEQ ID NO: 70, SEQ ID NO:72, SEQ ID NO:74, SEQ ID NO:76, SEQ ID NO: 78, SEQ ID NO:80, SEQ ID NO:82, SEQ ID NO: 84, SEQ ID NO: 86, SEQ ID NO:88, SEQ ID NO:90, SEQ ID NO: 92, SEQ ID NO:94, SEQ ID NO:96, SEQ ID NO:98, SEQ ID NO: 100, SEQ ID NO:102, SEQ ID NO:104, SEQ ID NO: 106, SEQ ID NO: 108, SEQ ID NO: 111, SEQ ID NO: 113, SEQ ID NO:115 and SEQ ID NO: 119; and
b) a fragment comprising at least 60% of an amino acid sequence selected from the group consisting of: SEQ ID NO:2, SEQ ID NO:4, SEQ ID NO:6, SEQ ID NO:8, SEQ ID NO:10, SEQ ID NO: 12, SEQ ID NO: 14, SEQ ID NO: 16, SEQ ID NO: 18, SEQ ID NO: 20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO: 42, SEQ ID NO:44, SEQ ID NO:46, SEQ ID NO:64, SEQ ID NO:66, SEQ ID NO:68, SEQ ID NO:70, SEQ ID NO: 72, SEQ ID NO: 74, SEQ ID NO: 76, SEQ ID NO:78, SEQ ID NO: 80, SEQ ID NO:82, SEQ ID NO: 84, SEQ ID NO: 86, SEQ ID NO:88, SEQ ID NO:90, SEQ ID NO:92, SEQ ID NO:94, SEQ ID NO:96, SEQ ID NO:98, SEQ ID NO: 100, SEQ ID NO: 102, SEQ ID NO: 104, SEQ ID NO: 106, SEQ ID NO: 108, SEQ ID NO: 111, SEQ ID NO: 113, SEQ ID NO:115 and SEQ ID NO:119.

20. The composition of claim 18, wherein the nucleic acid molecule comprises at least two nucleotide sequences wherein each nucleotide sequence encodes a structurally modified DMAb.

21. A method of treating a subject having a disease or disorder comprising administering at least one composition of claim 18 to a subject in need thereof.


SEQUENCE LISTING (128 sequences)

SEQ ID NO: 1 - DNA, 2193 bp, synthetic construct
  misc_feature 1..2193: note = Chemically Synthesized, pGX9291, Ebola Z5D2 modified
  source 1..2193: mol_type = other DNA
atggactggacctggagaatcctgttcctggtggcagcagcaaccggaacacacgcagaggtgcagctgcaggagagcggaccaggcctggtgcggcccagccagtccctgtctctgacctgcacagtgaccggctacagcatcacatccgattatgcctggaactggatcaggcagttccctggctacaagatcgagtggctgggctatatcacaaacaccggcagcaccggctttaatccatctctgaagagccggatctccatcacaagagacacctccaagaatcagttctttctgcagctgatctctgtgaccacagaggatacagcaacctaccactgcgccagaggcctggcatattggggacagggcacactggtgaccgtgagctccgcctctaccaagggaccaagcgtgtttccactggcaccttctagcaagagcacatccggcggcaccgccgccctgggatgtctggtgaaggactacttccctgagccagtgaccgtgtcttggaacagcggcgccctgacatccggagtgcacacctttccagccgtgctgcagtcctctggcctgtacagcctgagctccgtggtgaccgtgccctctagctccctgggcacacagacctatatctgcaacgtgaatcacaagccctctaatacaaaggtggacaagaaggtggagcctaagagctgtgataagacacacacctgccctccctgtccagcacctgagctgctgggcggcccaagcgtgttcctgtttccacccaagcccaaggacaccctgatgatctccaggacacctgaggtgacctgcgtggtggtggacgtgtctcacgaggaccccgaggtgaagttcaactggtacgtggatggcgtggaggtgcacaatgccaagaccaagccacgggaggagcagtacaactccacctatagagtggtgtctgtgctgacagtgctgcaccaggactggctgaacggcaaggagtataagtgcaaggtgtccaataaggccctgccagcccccatcgagaagaccatcagcaaggcaaagggacagccaagggagccacaggtgtacacactgcctccaagccgcgacgagctgaccaagaaccaggtgtccctgacatgtctggtgaagggcttctatccatctgatatcgccgtggagtgggagagcaatggccagcccgagaacaattacaagaccacaccccctgtgctggactccgatggctctttctttctgtattccaagctgaccgtggataagtctcgctggcagcagggcaacgtgttttcttgcagcgtgatgcacgaggccctgcacaatcactacacccagaagtccctgtctctgagccctggcaagaggggaaggaagcggagatccggctctggagccacaaacttctccctgctgaagcaggcaggcgacgtggaggagaatcctggaccaatggtgctgcagacccaggtgtttatcagcctgctgctgtggatctccggcgcctacggcgatgtggtgctgacacagacccctctgacactgagcgtgaccatcggccagccagccagcatctcctgtaactctagccagtctctgctggacagcgatggcaagacctacctgaattggctgctgcagaggcctggccagtccccaaagcgcctgatctatctggtgtccaagctggactctggcgtgacagatgagttcaccggctctggcagcggcacagactttaccctgaagatcagccgcgtggaggccgaggatctgggcatctactattgctggcagggcacacactctcccttcacctttggcagcggcacaaagctggagatcaagaccgtggccgcccctagcgtgttcatctttccaccctccgacgagcagctgaagagcggcacagcctccgtggtgtgcctgctgaacaatttctacccccgggaggccaaggtgcagtggaaggtggataacgccctgcagagcggcaattcccaggagtctgtgaccgagcaggacagcaaggattccacatattccctgtctaacacactgaccctgtccaaggccgactacgagaagcacaaggtgtatgcatgcgaggtgacccaccagggcctgtcctctcctgtgacaaagagctttaatcggggcgagtgttgataa

SEQ ID NO: 2 - protein, 729 aa, synthetic construct
  REGION 1..729: note = Chemically Synthesized, pGX9291, Ebola Z5D2 modified
MDWTWRILFLVAAATGTHAEVQLQESGPGLVRPSQSLSLTCTVTGYSITSDYAWNWIRQFPGYKIEWLGYITNTGSTGFNPSLKSRISITRDTSKNQFFLQLISVTTEDTATYHCARGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVLTQTPLTLSVTIGQPASISCNSSQSLLDSDGKTYLNWLLQRPGQSPKRLIYLVSKLDSGVTDEFTGSGSGTDFTLKISRVEAEDLGIYYCWQGTHSPFTFGSGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 3 - DNA, 2196 bp, synthetic construct
  misc_feature 1..2196: note = Chemically Synthesized, pGX9292, Ebola Z5D2 partial graft
  source 1..2196: mol_type = other DNA
atggactggacctggagaatcctgttcctggtggcagcagcaaccggaacacacgcagaggtgcagctgcaggagtccggaccaggcctggtgcgccctagccagtccctgtctctgacctgcacagtgaccggctacagcatcacatccgattatgcctggaactggatcagacagttccctggcaataagctggagtggctgggctacatcacaaacaccggcagcaccggctttaatccatctctgaagagccggatctccatcacaagagacacctccaagaaccagttctttctgcagctgatctctgtgaccacagaggatacagcaacctaccactgcgccagaggcctggcatattggggacagggcacactggtgaccgtgagctccgcctctaccaagggaccaagcgtgtttccactggcaccttctagcaagagcacatccggcggcaccgccgccctgggatgtctggtgaaggactacttccctgagccagtgaccgtgtcttggaacagcggcgccctgacatccggagtgcacacctttccagccgtgctgcagtcctctggcctgtacagcctgagctccgtggtgaccgtgccttctagctccctgggcacacagacctatatctgcaacgtgaatcacaagccctctaatacaaaggtggacaagaaggtggagcctaagagctgtgataagacacacacctgccctccctgtccagcacctgagctgctgggcggcccttccgtgttcctgtttccacccaagccaaaggacaccctgatgatctccaggacacctgaggtgacctgcgtggtggtggacgtgtctcacgaggaccccgaggtgaagttcaactggtacgtggatggcgtggaggtgcacaatgccaagaccaagccacgggaggagcagtacaattccacctatagagtggtgtctgtgctgacagtgctgcaccaggattggctgaacggcaaggagtataagtgcaaggtgagcaataaggccctgccagcccccatcgagaagaccatctccaaggcaaagggacagccaagggagccacaggtgtacacactgcctccaagccgcgacgagctgactaagaaccaggtgtccctgacatgtctggtgaagggcttctatccatctgatatcgccgtggagtgggagagcaatggccagcccgagaacaattacaagaccacaccccctgtgctggactccgatggctctttctttctgtattccaagctgaccgtggacaagtctcgctggcagcagggcaacgtgttttcttgcagcgtgatgcacgaggccctgcacaatcactacacccagaagtccctgtctctgagccctggcaagaggggaaggaagcggagatccggctctggagccacaaacttcagcctgctgaagcaggccggcgatgtggaggagaatcctggcccaatggtgctgcagacccaggtgtttatcagcctgctgctgtggatctccggcgcctatggcgagacaaccctgacacagtctccaggcaccctgagcctgtccccaggagagagggccaccctgagctgtaagtctagccagtctctgctggacagcgatggcaagacatacctgaactggctgctgcagaggcctggacagtccccaaagcgcctgatctatctggtgtccaagctggactctggcgtgacagatcggttcaccggctctggcagcggcacagactttaccctgaagatcagcagagtggaggccgaggatctgggcgtgtactattgctggcagggcacacactctccattcacctttggcagcggcacaaagctggagatcaagagaaccgtggccgcccccagcgtgttcatctttccaccctccgacgagcagctgaagagcggcacagcctccgtggtgtgcctgctgaacaatttctaccccagggaggccaaggtgcagtggaaggtggataacgccctgcagagcggcaattcccaggagtctgtgaccgagcaggacagcaaggattccacatattccctgtctaacacactgaccctgtccaaggccgactacgagaagcacaaggtgtatgcatgcgaggtgacccaccagggcctgtcctctcctgtgacaaagagctttaatcggggcgagtgttgataa

SEQ ID NO: 4 - protein, 730 aa, synthetic construct
  REGION 1..730: note = Chemically Synthesized, pGX9292, Ebola Z5D2 partial graft
MDWTWRILFLVAAATGTHAEVQLQESGPGLVRPSQSLSLTCTVTGYSITSDYAWNWIRQFPGNKLEWLGYITNTGSTGFNPSLKSRISITRDTSKNQFFLQLISVTTEDTATYHCARGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCKSSQSLLDSDGKTYLNWLLQRPGQSPKRLIYLVSKLDSGVTDRFTGSGSGTDFTLKISRVEAEDLGVYYCWQGTHSPFTFGSGTKLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 5 - DNA, 2196 bp, synthetic construct
  misc_feature 1..2196: note = Chemically Synthesized, pGX9293, Ebola Z5D2 graft on MERSYTE_1
  source 1..2196: mol_type = other DNA
atggactggacctggagaatcctgttcctggtggcagcagcaaccggaacacacgcacaggtgcagctggtgcagtctggagccgaggtgaagaagccaggcagctccgtgaaggtgtcctgcaaggcctccggctactctatcacaagcgattatgccatcagctgggtgcggcaggcaccaggacagggcctggagtggatgggcggcatcaccaacacaggctccaccaattacgcccagaagttccagggcagagtgaccatcacagccgacacctccacatctaccgcctatatggagctgtctagcctgcggagcgaggataccgccgtgtactattgcgccagaggcctggcatactggggacagggcaccacagtgaccgtgtcctctgcctccacaaagggaccaagcgtgttcccactggcacctagctccaagagcacatccggcggcaccgccgccctgggatgtctggtgaaggactatttccctgagccagtgaccgtgtcctggaactctggcgccctgacaagcggagtgcacacctttccagccgtgctgcagtctagcggcctgtactccctgtcctctgtggtgaccgtgcctagctcctctctgggcacacagacctatatctgcaacgtgaatcacaagccttccaatacaaaggtggacaagaaggtggagccaaagtcttgtgataagacacacacctgccctccctgtccagcacctgagctgctgggcggcccaagcgtgttcctgtttccacccaagcccaaggacacactgtacatcacccgggagccagaggtgacctgcgtggtggtggacgtgtcccacgaggaccccgaggtgaagttcaactggtacgtggatggcgtggaggtgcacaatgccaagaccaagcctcgggaggagcagtacaactctacctatagagtggtgagcgtgctgacagtgctgcaccaggactggctgaacggcaaggagtacaagtgcaaggtgagcaataaggccctgccagcccccatcgagaagaccatctccaaggcaaagggacagccaagggagccacaggtgtacacactgcctccaagccgcgacgagctgaccaagaaccaggtgtccctgacatgtctggtgaagggcttctatccctccgatatcgccgtggagtgggagtctaatggccagcctgagaacaattacaagaccacaccccctgtgctggacagcgatggctccttctttctgtatagcaagctgaccgtggacaagtcccggtggcagcagggcaacgtgttttcttgcagcgtgatgcacgaggccctgcacaatcactacacccagaagtccctgtctctgagcccaggcaagaggggaaggaagcggagatccggctctggagccacaaacttcagcctgctgaagcaggccggcgatgtggaggagaatcctggcccaatggtgctgcagacccaggtgtttatctccctgctgctgtggatctctggcgcctacggcgagacaaccctgacacagtccccaggcaccctgagcctgtcccctggagagagggccaccctgtcttgtagagcctctcagagcctgctggacagcgatggcaagacatacatcgcctggtatcagcagaagcctggccaggccccaagactgctgatgttcctggtgagcacaagggccaccggcatccctgaccgcttctccggctctggcagcggcacagacttcaccctgaacatcagctccctggagcctgaggactttgccgtgtactattgctggcagggcacacacagcccattcacctttggccagggcacaaagctggagatcaagaggaccgtggccgcccctagcgtgttcatctttccacccagcgacgagcagctgaagtctggcacagccagcgtggtgtgcctgctgaacaatttctacccacgcgaggccaaggtgcagtggaaggtggataacgccctgcagtctggcaatagccaggagtccgtgaccgagcaggactctaaggatagcacatattccctgtctagcacactgaccctgagcaaggccgattacgagaagcacaaggtgtatgcatgcgaggtgacccaccagggcctgaggagccccgtgacaaagtcctttaatcgcggcgagtgttgataa

SEQ ID NO: 6 - protein, 730 aa, synthetic construct
  REGION 1..730: note = Chemically Synthesized, pGX9293, Ebola Z5D2 graft on MERSYTE_1
MDWTWRILFLVAAATGTHAQVQLVQSGAEVKKPGSSVKVSCKASGYSITSDYAISWVRQAPGQGLEWMGGITNTGSTNYAQKFQGRVTITADTSTSTAYMELSSLRSEDTAVYYCARGLAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCRASQSLLDSDGKTYIAWYQQKPGQAPRLLMFLVSTRATGIPDRFSGSGSGTDFTLNISSLEPEDFAVYYCWQGTHSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 7 - DNA, 2196 bp, synthetic construct
  misc_feature 1..2196: note = Chemically Synthesized, pGX9294, Ebola Z5D2 graft on MERSYTE_2
  source 1..2196: mol_type = other DNA
atggactggacctggagaatcctgttcctggtggcagcagcaaccggaacacacgcacaggtgcagctggtgcagtctggagccgaggtgaagaagccaggcagctccgtgaaggtgtcctgcaaggcctccggctactctatcacaagcgattatgcctggagctgggtgcggcaggcaccaggacagggcctggagtggatgggcggcatcaccaacacaggctccaccaattacgcccagaagttccagggcagagtgaccatcacagccgacacctccacatctaccgcctatatggagctgtctagcctgcggagcgaggataccgccgtgtactattgcgccagaggcctggcatactggggacagggcaccacagtgaccgtgtcctctgcctccacaaagggaccaagcgtgttcccactggcacctagctccaagagcacatccggcggcaccgccgccctgggatgtctggtgaaggactatttccctgagccagtgaccgtgtcctggaactctggcgccctgacaagcggagtgcacacctttccagccgtgctgcagtctagcggcctgtactccctgtcctctgtggtgaccgtgcctagctcctctctgggcacacagacctatatctgcaacgtgaatcacaagccttccaatacaaaggtggacaagaaggtggagccaaagtcttgtgataagacacacacctgccctccctgtccagcacctgagctgctgggcggcccaagcgtgttcctgtttccacccaagcccaaggacacactgtacatcacccgggagccagaggtgacctgcgtggtggtggacgtgtcccacgaggaccccgaggtgaagttcaactggtacgtggatggcgtggaggtgcacaatgccaagaccaagcctcgggaggagcagtacaactctacctatagagtggtgagcgtgctgacagtgctgcaccaggactggctgaacggcaaggagtacaagtgcaaggtgagcaataaggccctgccagcccccatcgagaagaccatctccaaggcaaagggacagccaagggagccacaggtgtacacactgcctccaagccgcgacgagctgaccaagaaccaggtgtccctgacatgtctggtgaagggcttctatccctccgatatcgccgtggagtgggagtctaatggccagcctgagaacaattacaagaccacaccccctgtgctggacagcgatggctccttctttctgtatagcaagctgaccgtggacaagtcccggtggcagcagggcaacgtgttttcttgcagcgtgatgcacgaggccctgcacaatcactacacccagaagtccctgtctctgagcccaggcaagaggggaaggaagcggagatccggctctggagccacaaacttcagcctgctgaagcaggccggcgatgtggaggagaatcctggcccaatggtgctgcagacccaggtgtttatctccctgctgctgtggatctctggcgcctacggcgagacaaccctgacacagtccccaggcaccctgagcctgtcccctggagagagggccaccctgtcttgtagagcctctcagagcctgctggacagcgatggcaagacatacatcgcctggtatcagcagaagcctggccaggccccaagactgctgatgttcctggtgagcacaagggccaccggcatccctgaccgcttctccggctctggcagcggcacagacttcaccctgaacatcagctccctggagcctgaggactttgccgtgtactattgctggcagggcacacacagcccattcacctttggccagggcacaaagctggagatcaagaggaccgtggccgcccctagcgtgttcatctttccacccagcgacgagcagctgaagtctggcacagccagcgtggtgtgcctgctgaacaatttctacccacgcgaggccaaggtgcagtggaaggtggataacgccctgcagtctggcaatagccaggagtccgtgaccgagcaggactctaaggatagcacatattccctgtctagcacactgaccctgagcaaggccgattacgagaagcacaaggtgtatgcatgcgaggtgacccaccagggcctgaggagccccgtgacaaagtcctttaatcgcggcgagtgttgataa

SEQ ID NO: 8 - protein, 730 aa, synthetic construct
  REGION 1..730: note = Chemically Synthesized, pGX9294, Ebola Z5D2 graft on MERSYTE_2
MDWTWRILFLVAAATGTHAQVQLVQSGAEVKKPGSSVKVSCKASGYSITSDYAWSWVRQAPGQGLEWMGGITNTGSTNYAQKFQGRVTITADTSTSTAYMELSSLRSEDTAVYYCARGLAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCRASQSLLDSDGKTYIAWYQQKPGQAPRLLMFLVSTRATGIPDRFSGSGSGTDFTLNISSLEPEDFAVYYCWQGTHSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 9 - DNA, 2196 bp, synthetic construct
  misc_feature 1..2196: note = Chemically Synthesized, pGX9295, Ebola Z5D2 graft on V2L2
  source 1..2196: mol_type = other DNA
atggactggacctggagaatcctgttcctggtggcagcagcaaccggaacacacgcagaggtgcagctgctggagagcggcggcggcctggtgcagcccggcggctctctgaggctgagctgcgccgcctctggctacagcatcacctccgattatgcaatgaactgggtgcgccaggcacctggcaagggcctggagtgggtgtctgccatcaccaatacaggcagcaccggctacacagacgatgtgaagggccggttcacaatctccagagacaactctaagaataccctgtatctgcagatgaactccctgagggccgaggataccgccgtgtactattgcgcccgcggcctggcatactggggacagggcaccacagtgacagtgagctccgcctctaccaagggaccaagcgtgtttccactggcaccttctagcaagtctaccagcggcggcacagccgccctgggatgtctggtgaaggactatttccctgagccagtgaccgtgtcttggaacagcggcgccctgaccagcggagtgcacacatttcctgccgtgctgcagtcctctggcctgtactccctgagctccgtggtgaccgtgccatctagctccctgggcacccagacatatatctgcaacgtgaatcacaagccaagcaatacaaaggtggacaagaaggtggagcccaagtcctgtgataagacccacacatgccctccctgtccagcacctgagctgctgggcggcccaagcgtgttcctgtttccacccaagcctaaggacaccctgatgatctctcggacccccgaggtgacatgcgtggtggtggacgtgagccacgaggaccccgaggtgaagtttaactggtacgtggatggcgtggaggtgcacaatgccaagaccaagccccgggaggagcagtacaactccacatatagagtggtgtctgtgctgaccgtgctgcaccaggactggctgaacggcaaggagtataagtgcaaggtgtccaataaggccctgccagcccccatcgagaagacaatctctaaggcaaagggacagccacgggagccacaggtgtacaccctgcctccatccagagacgagctgacaaagaaccaggtgtctctgacctgtctggtgaagggcttctatccttctgatatcgccgtggagtgggagagcaatggccagccagagaacaattacaagaccacaccccctgtgctggactccgatggctctttctttctgtattccaagctgaccgtggataagtctaggtggcagcagggcaacgtgttttcctgctctgtgatgcacgaggccctgcacaatcactacacccagaagagcctgtccctgtctcctggcaagaggggaaggaagcggagaagcggctccggagccacaaacttcagcctgctgaagcaggcaggcgacgtggaggagaatcctggaccaatggtgctgcagacccaggtgtttatcagcctgctgctgtggatctccggagcatacggagccatccagatgacacagagcccttctagcctgtctgccagcgtgggcgatagggtgaccatcacatgtcgcgcctcccagtctctgctggactccgatggcaagacctacctgggctggtatcagcagaagcctggcaaggccccaaagctgctgatctacctggtgagcacactgcagtccggagtgccatctcgcttcagcggctccggctctggaaccgactttaccctgacaatctcctctctgcagccagaggatttcgccacatactattgctggcagggcacccactcccccttcacatttggccagggcaccaaggtggagatcaagaggaccgtggcagcaccaagcgtgttcatctttccaccctccgacgagcagctgaagagcggcacagcctccgtggtgtgcctgctgaacaacttctaccccagagaggccaaggtgcagtggaaggtggataacgccctgcagagcggcaattcccaggagtctgtgacagagcaggacagcaaggattccacctatagcctgtccaacaccctgacactgagcaaggccgactacgagaagcacaaggtgtatgcctgcgaggtgacacaccagggcctgagctcccccgtgaccaagtccttcaatagaggcgagtgttgataa

SEQ ID NO: 10 - protein, 730 aa, synthetic construct
  REGION 1..730: note = Chemically Synthesized, pGX9295, Ebola Z5D2 graft on V2L2
MDWTWRILFLVAAATGTHAEVQLLESGGGLVQPGGSLRLSCAASGYSITSDYAMNWVRQAPGKGLEWVSAITNTGSTGYTDDVKGRFTISRDNSKNTLYLQMNSLRAEDTAVYYCARGLAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGAIQMTQSPSSLSASVGDRVTITCRASQSLLDSDGKTYLGWYQQKPGKAPKLLIYLVSTLQSGVPSRFSGSGSGTDFTLTISSLQPEDFATYYCWQGTHSPFTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 11 - DNA, 2208 bp, synthetic construct
  misc_feature 1..2208: note = Chemically Synthesized, pGX9296, Ebola ZBDBV223 modified
  source 1..2208: mol_type = other DNA
atggattggacctggagaatcctgttcctggtggcagcagcaacaggaacccacgcacaggtgcagctgcagcagtggggcgccggcctgctgaagccaagcgagacactgtccctgacatgcgccgtgtacggcggcagctttaccacaacctattggaattggatcaggcagccccctggcaagggcctggagtggatcggcgaggtgaactactctggcaacgccaattataacccaagcctgcagggaagggtggcaatctctgtggacacaagcaagaatcagttctctctgcagctgaacagcgtgaccgccgccgatacagccatctactattgcaccagccggatcagaagccacatcgcctactcctggaagggctgggtgtggggcaagggcacaaccgtgaccgtgagctccgcctccacaaagggaccaagcgtgttcccactggcaccctctagcaagagcacctccggcggcacagccgccctgggctgtctggtgaaggattatttccccgagcctgtgaccgtgagctggaattccggcgccctgaccagcggagtgcacacatttccagccgtgctgcagtcctctggcctgtactccctgagctccgtggtgaccgtgccctctagctccctgggcacacagacctatatctgcaatgtgaaccacaagccctctaacacaaaggtggacaagaaggtggagcctaagagctgtgataagacacacacctgcccaccctgtccagcaccagagctgctgggcggccctagcgtgttcctgtttcctccaaagccaaaggataccctgatgatctcccgcacccctgaggtgacatgcgtggtggtggacgtgtctcacgaggaccccgaggtgaagttcaattggtacgtggacggcgtggaggtgcacaacgccaagaccaagcctagggaggagcagtacaatagcacctatcgcgtggtgtccgtgctgacagtgctgcaccaggactggctgaatggcaaggagtataagtgcaaggtgagcaacaaggccctgcctgccccaatcgagaagaccatctccaaggcaaagggacagcctcgggagccacaggtgtacacactgccccctagcagagacgagctgaccaagaatcaggtgtccctgacatgtctggtgaagggcttctatccatccgatatcgccgtggagtgggagtctaacggccagcccgagaacaattacaagacaaccccacccgtgctggactctgatggcagcttctttctgtattccaagctgaccgtggataagtctcggtggcagcagggcaacgtgttcagctgcagcgtgatgcacgaggccctgcacaaccactacacccagaagtccctgtctctgagcccaggcaagcggggcagaaagcggagatccggctctggcgccacaaatttcagcctgctgaagcaggcaggcgacgtggaggagaacccaggacctatggtgctgcagacccaggtgtttatctctctgctgctgtggatcagcggcgcctacggcgagatcgtgatgacccagagcccaggcacactgagcctgtccccaggagagcaggccaccctgtcctgtagggcctctcagagcgtgccccgcaactacatcggctggtggcagcagacaccaggacaggcccctcggctgctgatctatggagcctctagccgggccgcaggattccctgacagattttccggctctggcagcggcaccgatttcacactgaccatcacagacctggagcccgaggattttgccatgtactattgccaccagtacgacagactgccttataccttcggccagggcacaaagctggagatcaagaccgtggccgcccctagcgtgttcatctttcctccatccgatgagcagctgaagagcggcacagcctccgtggtgtgcctgctgaacaacttctacccaagggaggccaaggtgcagtggaaggtggacaatgccctgcagtccggcaactctcaggagagcgtgaccgagcaggactccaaggattctacatattccctgtctaataccctgacactgagcaaggccgactacgagaagcacaaggtgtatgcatgcgaggtgacccaccagggcctgtcctctcctgtgacaaagtccttcaacagaggcgagtgttgataa

SEQ ID NO: 12 - protein, 734 aa, synthetic construct
  REGION 1..734: note = Chemically Synthesized, pGX9296, Ebola ZBDBV223 modified
MDWTWRILFLVAAATGTHAQVQLQQWGAGLLKPSETLSLTCAVYGGSFTTTYWNWIRQPPGKGLEWIGEVNYSGNANYNPSLQGRVAISVDTSKNQFSLQLNSVTAADTAIYYCTSRIRSHIAYSWKGWVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVMTQSPGTLSLSPGEQATLSCRASQSVPRNYIGWWQQTPGQAPRLLIYGASSRAAGFPDRFSGSGSGTDFTLTITDLEPEDFAMYYCHQYDRLPYTFGQGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 13 - DNA, 2211 bp, synthetic construct
  misc_feature 1..2211: note = Chemically Synthesized, pGX9297, Ebola ZBDBV223 partial graft
  source 1..2211: mol_type = other DNA
atggactggacctggagaatcctgttcctggtggcagcagcaacaggaacccacgcacaggtgcagctgcagcagtggggcgccggcctgctgaagccaagcgagacactgtccctgacatgcgccgtgtacggcggcagctttaccacaacctattggaattggatcagacagccccctggcaagggcctggagtggatcggcgaggtgaactactctggcaacgccaattataaccccagcctgaagggcagggtggccatctctgtggacacaagcaagaatcagttctctctgcgcctgaacagcgtgaccgcagcagatacagccatctactattgcaccagccggatcagaagccacatcgcctactcctggaagggcgacgtgtggggcaagggcacaaccgtgaccgtgagctccgcctccacaaagggaccaagcgtgttcccactggcaccctctagcaagagcacctccggcggcacagccgccctgggctgtctggtgaaggattatttccccgagcctgtgaccgtgagctggaattccggcgccctgaccagcggagtgcacacatttccagccgtgctgcagtcctctggcctgtactccctgagctccgtggtgaccgtgccctctagctccctgggcacacagacctatatctgcaatgtgaaccacaagccctctaacacaaaggtggacaagaaggtggagcctaagagctgtgataagacacacacctgcccaccctgtccagcaccagagctgctgggcggccctagcgtgttcctgtttcctccaaagccaaaggacaccctgatgatctcccggacccctgaggtgacatgcgtggtggtggacgtgtctcacgaggaccccgaggtgaagttcaattggtacgtggatggcgtggaggtgcacaacgccaagaccaagcctagggaggagcagtacaatagcacctatcgcgtggtgtccgtgctgacagtgctgcaccaggattggctgaatggcaaggagtataagtgcaaggtgagcaacaaggccctgcctgccccaatcgagaagaccatctccaaggcaaagggacagcctcgggagccacaggtgtacacactgccccctagcagagacgagctgaccaagaatcaggtgtccctgacatgtctggtgaagggcttctatccatccgatatcgccgtggagtgggagtctaacggccagcccgagaacaattacaagacaaccccacccgtgctggactctgatggcagcttctttctgtattccaagctgaccgtggacaagtctagatggcagcagggcaacgtgttcagctgcagcgtgatgcacgaggccctgcacaaccactacacccagaagtccctgtctctgagcccaggcaagcggggcagaaagcggagatccggctctggcgccacaaatttcagcctgctgaagcaggcaggcgatgtggaggagaacccaggacctatggtgctgcagacccaggtgtttatctctctgctgctgtggatcagcggagcatacggagagacaaccctgacccagagcccaggcacactgagcctgtccccaggagagagggccaccctgtcctgtagggcatctcagagcgtgccacggaactacatcggatggttccagcagaagccaggacaggcccctagactgctgatctatggagcctctagccgggccgcaggattccctgacagattttccggctctggcagcggcaccgatttcacactgaccatcacaaggctggagcccgaggactttgccatgtactattgccaccagtacgataggctgccttataccttcggccagggcacaaagctggagatcaagaggaccgtggcagcacctagcgtgttcatctttcctccatccgacgagcagctgaagagcggcacagcctccgtggtgtgcctgctgaacaacttctacccacgcgaggccaaggtgcagtggaaggtggataatgccctgcagtccggcaactctcaggagagcgtgaccgagcaggactccaaggattctacatattccctgtctaataccctgacactgagcaaggccgactacgagaagcacaaggtgtatgcatgcgaggtgacccaccagggcctgtcctctcctgtgacaaagtcctttaacaggggcgagtgttgataa

SEQ ID NO: 14 - protein, 735 aa, synthetic construct
  REGION 1..735: note = Chemically Synthesized, pGX9297, Ebola ZBDBV223 partial graft
MDWTWRILFLVAAATGTHAQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 15 - DNA, 2211 bp, synthetic construct
  misc_feature 1..2211: note = Chemically Synthesized, pGX9298, Ebola ZBDBV223 graft on MERSYTE
  source 1..2211: mol_type = other DNA
atggactggacctggagaatcctgttcctggtggcagcagcaacaggaacccacgcacaggtgcagctggtgcagagcggagccgaggtgaagaagccaggcagctccgtgaaggtgtcctgcaaggccagcggcggctccttcaccacaacctacatctcttgggtgcgccaggcaccaggacagggcctggagtggatgggcggcgtgaactacagcggcaacgccaattatgcccagaagtttcagggccgggtgacaatcaccgccgacacaagcacctccacagcctacatggagctgtctagcctgagaagcgaggataccgccgtgtactattgcaccagccggatcagaagccacatcgcctattcctggaagggcgacgtgtggggacagggaacaaccgtgaccgtgtcctctgcctccacaaagggaccaagcgtgttcccactggcacctagctccaagtctaccagcggcggcacagccgccctgggatgtctggtgaaggattacttccctgagccagtgaccgtgtcttggaacagcggcgccctgaccagcggagtgcacacatttcccgccgtgctgcagtctagcggcctgtactccctgtcctctgtggtgaccgtgcctagctcctctctgggcacccagacatatatctgcaacgtgaatcacaagccttctaatacaaaggtggacaagaaggtggagccaaagagctgtgataagacccacacatgccctccctgtccagcacctgagctgctgggcggcccaagcgtgttcctgtttccacccaagcccaaggacaccctgtacatcacacgggagccagaggtgacctgcgtggtggtggacgtgtcccacgaggaccccgaggtgaagttcaactggtacgtggatggcgtggaggtgcacaatgccaagaccaagccaagggaggagcagtacaattctacctatcgcgtggtgagcgtgctgacagtgctgcaccaggattggctgaacggcaaggagtacaagtgcaaggtgtccaataaggccctgccagcccccatcgagaagaccatctctaaggcaaagggacagccaagggagccacaggtgtacacactgcctccaagccgcgacgagctgaccaagaaccaggtgtccctgacatgtctggtgaagggcttctatccctccgatatcgccgtggagtgggagtctaatggccagcctgagaacaattacaagacaacccctcccgtgctggactccgatggctctttctttctgtatagcaagctgaccgtggacaagtccagatggcagcagggcaacgtgttttcctgctctgtgatgcacgaggccctgcacaatcactacacccagaagagcctgtccctgtctcctggcaagaggggaagaaagcggagaagcggctccggcgccacaaacttcagcctgctgaagcaggccggcgatgtggaggagaatcctggcccaatggtgctgcagacccaggtgtttatctccctgctgctgtggatctctggcgcctatggagagacaaccctgacccagagcccaggcacactgtctctgagccctggagagagggccaccctgtcctgtagggcatcccagtctgtgccacggaactacatcgcctggtatcagcagaagcctggccaggccccaagactgctgatgttcggagccagctcccgggccgcaggcatccctgacagattcagcggctccggctctggaaccgacttcaccctgaacatctctagcctggagcctgaggacttcgccgtgtactattgccaccagtacgataggctgccatatacctttggccagggcacaaagctggagatcaagaggaccgtggcagcacctagcgtgttcatctttccacccagcgacgagcagctgaagtctggcacagccagcgtggtgtgcctgctgaacaatttctacccaagggaggccaaggtgcagtggaaggtggataacgccctgcagagcggcaattcccaggagtctgtgaccgagcaggacagcaaggattccacatattctctgtcctctaccctgacactgtccaaggccgactacgagaagcacaaggtgtatgcatgcgaggtgacccaccagggcctgcggagccccgtgacaaagtcctttaacagaggcgagtgttgataa

SEQ ID NO: 16 - protein, 735 aa, synthetic construct
  REGION 1..735: note = Chemically Synthesized, pGX9298, Ebola ZBDBV223 graft on MERSYTE
MDWTWRILFLVAAATGTHAQVQLVQSGAEVKKPGSSVKVSCKASGGSFTTTYISWVRQAPGQGLEWMGGVNYSGNANYAQKFQGRVTITADTSTSTAYMELSSLRSEDTAVYYCTSRIRSHIAYSWKGDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGETTLTQSPGTLSLSPGERATLSCRASQSVPRNYIAWYQQKPGQAPRLLMFGASSRAAGIPDRFSGSGSGTDFTLNISSLEPEDFAVYYCHQYDRLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 17 - DNA, 2211 bp, synthetic construct
  misc_feature 1..2211: note = Chemically Synthesized, pGX9299, Ebola ZBDBV223 graft on V2L2
  source 1..2211: mol_type = other DNA
atggactggacctggagaatcctgttcctggtggcagcagcaacaggaacccacgcagaggtgcagctgctggagagcggcggcggcctggtgcagccaggcggctccctgaggctgtcttgcgcagcatctggcggcagcttcaccacaacctacatgaactgggtgcgccaggcaccaggcaagggcctggagtgggtgagcgccgtgaactattccggcaatgcctactatacagacgatgtgaagggccggtttaccatcagcgtggacacatccaagaacaccctgtacctgcagatgaattccctgagagccgaggatacagccgtgtactattgcaccagccggatcagaagccacatcgcctatagctggaagggcgacgtgtggggacagggaacaaccgtgacagtgagctccgcctctaccaagggcccaagcgtgtttcccctggccccttctagcaagtctacaagcggcggcaccgccgccctgggatgtctggtgaaggattacttccctgagccagtgaccgtgtcctggaactctggcgccctgacatccggcgtgcacacctttcctgccgtgctgcagtcctctggcctgtacagcctgagctccgtggtgaccgtgccatctagctccctgggcacacagacctatatctgcaacgtgaatcacaagccatccaatacaaaggtggacaagaaggtggagcccaagtcttgtgataagacacacacctgccctccctgtccagcacctgagctgctgggcggcccaagcgtgttcctgtttccacccaagcctaaggacaccctgatgatcagcaggacacccgaggtgacctgcgtggtggtggacgtgtcccacgaggaccccgaggtgaagttcaactggtacgtggatggcgtggaggtgcacaatgccaagaccaagcctcgggaggagcagtacaactccacatatagagtggtgtctgtgctgaccgtgctgcaccaggactggctgaacggcaaggagtataagtgcaaggtgtctaataaggccctgccagcccccatcgagaagacaatcagcaaggcaaagggacagccaagggagccacaggtgtacaccctgcctccatctcgcgacgagctgacaaagaaccaggtgagcctgacctgtctggtgaagggcttctatcctagcgatatcgccgtggagtgggagtccaatggccagccagagaacaattacaagacaacccctcccgtgctggacagcgatggctccttctttctgtattctaagctgaccgtggataagagccgctggcagcagggcaacgtgttttcctgctctgtgatgcacgaggccctgcacaatcactacacccagaagagcctgtccctgtctccaggcaagaggggaagaaagcggagaagcggctccggagcaaccaacttctccctgctgaagcaggcaggcgacgtggaggagaatcctggaccaatggtgctgcagacccaggtgtttatcagcctgctgctgtggatctccggagcatacggagccatccagatgacacagagcccctctagcctgtctgccagcgtgggcgatagggtgacaatcacctgtcgcgcctcccagtctgtgcctaggaattacctgggctggtatcagcagaagcctggcaaggccccaaagctgctgatctacggagcatccacactgcagtctggagtgcctagccgcttcagcggctccggctctggaaccgactttacactgaccatctcctctctgcagccagaggacttcgccacatactattgccaccagtacgatcggctgccctatacatttggccagggcaccaaggtggagatcaagagaaccgtggccgccccttccgtgttcatctttccaccctctgacgagcagctgaagtccggcacagcctctgtggtgtgcctgctgaacaatttctacccacgggaggccaaggtgcagtggaaggtggataacgccctgcagtctggcaatagccaggagtccgtgacagagcaggactctaaggatagcacctatagcctgtccaacacactgaccctgtccaaggccgattacgagaagcacaaggtgtatgcctgcgaggtgacacaccagggcctgagctcccccgtgaccaagagctttaatagaggcgagtgttgataa

SEQ ID NO: 18 - protein, 735 aa, synthetic construct
  REGION 1..735: note = Chemically Synthesized, pGX9299, Ebola ZBDBV223 graft on V2L2
MDWTWRILFLVAAATGTHAEVQLLESGGGLVQPGGSLRLSCAASGGSFTTTYMNWVRQAPGKGLEWVSAVNYSGNAYYTDDVKGRFTISVDTSKNTLYLQMNSLRAEDTAVYYCTSRIRSHIAYSWKGDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGAIQMTQSPSSLSASVGDRVTITCRASQSVPRNYLGWYQQKPGKAPKLLIYGASTLQSGVPSRFSGSGSGTDFTLTISSLQPEDFATYYCHQYDRLPYTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 19 - DNA, 1476 bp, synthetic construct
  misc_feature 1..1476: note = Chemically Synthesized, pGX9330, Ebola Z5D2 scFv-Fc
  source 1..1476: mol_type = other DNA
atggtgctgcagacccaggtgttcatttccctgctgctgtggattagcggagcctatggagatgtcgtgctgacccagaccccactgaccctgagcgtgaccatcggccagccagcctccatctcttgcaagtccagccagtccctgctggattctgacggcaagacctacctgaattggctgctgcagagacctggccagagccctaagcggctgatctatctggtgtctaagctggattctggcgtgaccgatagattcaccggctccggctctggcaccgactttaccctgaagatctccagggtggaggccgaggatctgggcgtgtattactgttggcagggcacccactccccctttacattcggcagcggcacaaagctggagatcaagggcggcggcggctccggcggcggcggctctggcggcggcggctctgaggtgcagctgcaggagagcggcccaggcctggtgcgcccatcccagagcctgtctctgacatgtaccgtgacaggctacagcatcacatccgattacgcctggaactggatcagacagttcccaggcaacaagctggagtggctgggctatatcaccaacaccggctctacaggcttcaacccttccctgaagtccagaatctctatcacccgcgatacatccaagaaccagttctttctgcagctgatcagcgtgaccacagaggataccgccacatatcactgtgccagaggcctggcctactggggccagggcaccctggtgaccgtgtcctctgagcccaagtcctgtgataagacccacacctgcccaccttgtcccgccccagagctgctgggcggccccagcgtgttcctgtttcctccaaagcccaaggacaccctgatgatcagcaggacaccagaggtgacatgcgtggtggtggacgtgtctcacgaggacccagaggtgaagttcaactggtacgtggatggcgtggaggtgcacaacgccaagaccaagccacgggaggagcagtacaacagcacctacagagtggtgtccgtgctgaccgtgctgcaccaggattggctgaacggcaaggagtacaagtgtaaggtgtctaacaaggccctgcccgccccaatcgagaagaccatctccaaggccaagggccagcctagggagccacaggtgtatacactgccaccctctagagatgagctgacaaagaatcaggtgtccctgacatgcctggtgaagggcttctatccatccgacatcgccgtggagtgggagagcaatggccagccagagaataactacaagaccacaccacctgtgctggattccgacggctctttcttcctgtactccaagctgaccgtggataagtccaggtggcagcagggcaacgtgttttcttgcagcgtcatgcacgaagcactgcacaaccattacactcagaagagcctgtcactgtcaccttgataa

SEQ ID NO: 20 - protein, 490 aa, synthetic construct
  REGION 1..490: note = Chemically Synthesized, pGX9330, Ebola Z5D2 scFv-Fc
MVLQTQVFISLLLWISGAYGDVVLTQTPLTLSVTIGQPASISCKSSQSLLDSDGKTYLNWLLQRPGQSPKRLIYLVSKLDSGVTDRFTGSGSGTDFTLKISRVEAEDLGVYYCWQGTHSPFTFGSGTKLEIKGGGGSGGGGSGGGGSEVQLQESGPGLVRPSQSLSLTCTVTGYSITSDYAWNWIRQFPGNKLEWLGYITNTGSTGFNPSLKSRISITRDTSKNQFFLQLISVTTEDTATYHCARGLAYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - DNA, 1488 bp, synthetic construct
  misc_feature 1..1488: note = Chemically Synthesized, pGX9331, Ebola Z1H3 scFv-Fc
  source 1..1488: mol_type = other DNA
atggactggacctggagaatcctgttcctggtcgccgccgctactgggactcacgccgaagtgcagctgcagcagagtggagccgagctggtgaagccaggcgccagcgtgaagctgtcctgcacagcctccggctttaacatcaaggatacctacatccactgggtgaagcagggccctgagcagggcctggagtggatcggcaggatcgatcccgccaacggcaatacaaagtacgatccaaagtttcagggcaaggccaccatcacagccgacacctctagcaatacagcctatctgcagctgtctggcctgacatccgaggatacagccgtgtactattgtgcccgggagtctagaatcagcacaatgctgaccacaggctactttgactactggggccagggcaccaccctgaccgtgagcagcggcggcggcggctctggcggcggcggctccggcggcggcggcagccagatcgtgctgacccagtccccagccatcatgagcgcctccccaggcgagaaggtgaccatgacctgctccgcctctagctccgtgtcttatatgtactggtatcagcagaagccaggctcctctccaagactgctgatctatgataccagcaatctggcctctggcgtgccagtgagattctctggcagcggctccggcaccagctactccctgacaatcagcaggatggaggccgaggatgccgccacatactattgccagcagtggtcttcctatccctacacctttggcggcggcacaaagctggagatcaaggagcctaagtcctgcgataagacccacacctgtcctccctgcccagcccccgagctgctgggcggcccatccgtgtttctgttccctccaaagcccaaggataccctgatgatctcccggacaccagaggtgacatgcgtggtggtggatgtgagccacgaggacccagaggtgaagttcaactggtacgtggatggcgtggaggtgcacaatgccaagaccaagccaagagaggagcagtacaacagcacatacagggtggtgtctgtgctgacagtgctgcaccaggactggctgaatggcaaggagtataagtgcaaggtgtccaataaggccctgcctgcccctatcgagaagaccatctccaaggccaagggccagcccagggagccacaggtgtataccctgcctcccagccgggatgagctgacaaagaaccaggtgtccctgacatgtctggtgaagggcttctaccctagcgatatcgccgtggagtgggagtccaacggccagccagagaacaattataagacaaccccacctgtgctggattccgacggcagcttctttctgtattccaagctgacagtggacaagagcaggtggcagcagggcaacgtgtttagctgctccgtcatgcacgaagccctgcacaaccactacacccagaagtcactgagcctgtcaccttgataa

SEQ ID NO: 22 - protein, 494 aa, synthetic construct
  REGION 1..494: note = Chemically Synthesized, pGX9331, Ebola Z1H3 scFv-Fc
MDWTWRILFLVAAATGTHAEVQLQQSGAELVKPGASVKLSCTASGFNIKDTYIHWVKQGPEQGLEWIGRIDPANGNTKYDPKFQGKATITADTSSNTAYLQLSGLTSEDTAVYYCARESRISTMLTTGYFDYWGQGTTLTVSSGGGGSGGGGSGGGGSQIVLTQSPAIMSASPGEKVTMTCSASSSVSYMYWYQQKPGSSPRLLIYDTSNLASGVPVRFSGSGSGTSYSLTISRMEAEDAATYYCQQWSSYPYTFGGGTKLEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - DNA, 1488 bp, synthetic construct
  misc_feature 1..1488: note = Chemically Synthesized, pGX9332, Ebola ZBDBV223 scFv-Fc
  source 1..1488: mol_type = other DNA
atggattggacttggaggattctgtttctggtcgccgccgctacagggactcacgctcaggtgcagctgcagcagtggggagccgggctgctgaagccatctgagaccctgtctctgacatgcgccgtgtacggcggctccttcacaaccacctactggaactggatcagacagccacccggcaagggcctggagtggatcggcgaggtgaactacagcggcaatgccaactacaatccctccctgaagggcagggtggccatctccgtggatacatccaagaaccagttttccctgcggctgaatagcgtgacagccgccgacaccgccatctattactgcacaagccggatcagaagccacatcgcctactcttggaagggcgacgtgtggggcaagggcacaaccgtgacagtgtcctctggcggcggcggctccggcggcggcggcagcggcggcggcggcagcgagatcgtgatgacccagtccccaggcacactgagcctgtccccaggcgagagagccaccctgagctgtagggcctctcagtccgtgccaaggaactatatcggctggtttcagcagaagccaggccaggcccctagactgctgatctatggcgccagctccagagccgccggcttccccgataggttttctggctccggctctggcaccgacttcacactgaccatcacaagactggagccagaggactttgccatgtattactgccaccagtatgatcggctgccatacaccttcggccagggcaccaagctggagatcaaggagccaaagagctgtgacaagacccacacctgccctccctgcccagccccagagctgctgggcggcccatccgtgttcctgttcccaccaaagcccaaggacaccctgatgatctcccgcacaccagaggtgacctgcgtggtggtggacgtgtctcacgaggacccagaggtgaagtttaactggtacgtggacggcgtggaggtgcacaacgccaagacaaagcccagagaggagcagtacaattccacatacagagtggtgtctgtgctgacagtgctgcaccaggattggctgaacggcaaggagtataagtgcaaggtgtctaacaaggccctgcccgccccaatcgagaagacaatcagcaaggccaagggccagccaagagagccccaggtgtataccctgcctccatctagagacgagctgaccaagaatcaggtgtccctgacatgtctggtgaagggcttctaccctagcgacatcgccgtggagtgggagtccaatggccagccagagaataactacaagaccacccctccagtgctggattctgacggcagctttttcctgtattctaagctgaccgtggacaagagcagatggcagcagggcaacgtgttttcctgcagcgtcatgcacgaagccctgcacaaccattacacccagaagagcctgtcactgagcccctgataa

SEQ ID NO: 24 - protein, 494 aa, synthetic construct
  REGION 1..494: note = Chemically Synthesized, pGX9332, Ebola ZBDBV223 scFv-Fc
MDWTWRILFLVAAATGTHAQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSGGGGSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 25 - DNA, 1482 bp, synthetic construct
  misc_feature 1..1482: note = Chemically Synthesized, pGX9345, EBV114_scFv-Fc
  source 1..1482: mol_type = other DNA
atggactggacctggagaatcctgttcctggtcgccgccgctactgggactcacgccgaagtgcagctggtcgaatctgggggaggcctgatccagccaggcggctccctgaggctgtcttgcgccgcctctggcttcgccctgagaatgtacgatatgcactgggtgcggcagacaatcgacaagagactggagtgggtgtctgccgtgggcccaagcggcgacacctattacgccgactctgtgaagggccggttcgccgtgtctagagagaatgccaagaactccctgagcctgcagatgaacagcctgacagccggcgacaccgccatctattactgcgtgagaagcgacaggggcgtggccggcctgttcgattcctggggccagggcatcctggtgacagtgtccagcggcggcggcggctctggcggcggcggctccggcggcggcggcagcgatatccagatgacccagagcccctcctccctgagcgcctccgtgggcgacagaatcacaatcacctgtagagcctcccaggcctttgataattacgtggcctggtatcagcagcggccaggcaaggtgccaaagctgctgatcagcgccgcctctgccctgcacgccggcgtgccaagcagattctctggctctggctccggcacccacttcacactgaccatctctagcctgcagcccgaggatgtggccacctattactgccagaactacaattccgccccactgacatttggcggcggcacaaaggtggagatcaaggagccaaagtcttgcgacaagacccacacctgtccaccctgccccgccccagagctgctgggcggcccttccgtgttcctgtttcccccaaagcctaaggataccctgatgatctccaggacacccgaggtgacatgcgtggtggtggatgtgagccacgaggacccagaggtgaagttcaattggtacgtggacggcgtggaggtgcacaatgccaagacaaagcctagagaggagcagtataactccacctatagggtggtgtccgtgctgacagtgctgcaccaggattggctgaacggcaaggagtacaagtgcaaggtgtccaataaggccctgcccgcccctatcgagaagacaatctctaaggccaagggccagccaagagagccacaggtgtacaccctgccaccctccagagacgagctgacaaagaaccaggtgtccctgacatgtctggtgaagggcttctatccctccgatatcgccgtggagtgggagtctaatggccagcccgagaacaattacaagacaaccccacctgtgctggactccgacggcagctttttcctgtactccaagctgaccgtggataagtcccggtggcagcagggcaacgtgttttcctgctccgtgatgcacgaagcactgcacaaccactacactcagaaaagcctgtccctgtcacctggcaaatgataa

SEQ ID NO: 26 - protein, 492 aa, synthetic construct
  REGION 1..492: note = Chemically Synthesized, pGX9345, EBV114_scFv-Fc
MDWTWRILFLVAAATGTHAEVQLVESGGGLIQPGGSLRLSCAASGFALRMYDMHWVRQTIDKRLEWVSAVGPSGDTYYADSVKGRFAVSRENAKNSLSLQMNSLTAGDTAIYYCVRSDRGVAGLFDSWGQGILVTVSSGGGGSGGGGSGGGGSDIQMTQSPSSLSASVGDRITITCRASQAFDNYVAWYQQRPGKVPKLLISAASALHAGVPSRFSGSGSGTHFTLTISSLQPEDVATYYCQNYNSAPLTFGGGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1512 bp, synthetic construct
  misc_feature 1..1512: note = Chemically Synthesized, pGX9346, 1A2_scFv-Fc
  source 1..1512: mol_type = other DNA
atggtgctgcagacccaggtgttcatctctctgctgctgtggatctctggcgcctatggcgatatcgtgatgacccagtcccctagaagcctgtccgtgacacccggcgagcctgccagcatctcctgcagatcctctcagagcctgctgcacaggaatggctacaactacctggactggtatctgcagaagcctggccagtcccctcagctgctgatctatctgggctctaatcgcgccagcggcgtgcctgacaggttctccggcagcggctctggcaccgacttcacactgaagatcagcagagtggaggccgaggacgtgggcgtgtactattgtatgcaggccctgcagaccccttcctggacattcggccagggcaccaaggtggagatcaagggcggcggcggctccggcggcggcggctctggcggcggcggctccgaggtgcagctggtggagtctggcggcggcctgatccagccaggcggctccctgagactgtcttgcgccgcctctggcttcgccgtgaggtccaactacctgtcttgggtgcgccaggcccctggcaagggcctggagtgggtgtctctgatctactccggcggcctgacagcctacgccgattccgtggagggccggttcacaatctccagagacaattccaagaacaccctgtacctgcagatgaactccctgagagtggaggatacagccctgtattactgtgccagagtggccagctccgccggcacattctattacggcatggacgtgtggggccagggcacaaccgtgaccgtgtcttccgagcctaagtcctgtgacaagacccacacctgtccaccctgtcctgccccagagctgctgggcggcccatccgtgttcctgttccctccaaagcccaaggatacactgatgatctctaggacccctgaggtgacctgcgtggtggtggatgtgagccacgaggaccctgaggtgaagtttaactggtacgtggatggcgtggaggtgcacaatgccaagacaaagccaagagaggagcagtacaattctacatacagagtggtgtctgtgctgacagtgctgcaccaggactggctgaatggcaaggagtacaagtgcaaggtgtccaacaaggccctgccagcccctatcgagaagaccatctccaaggccaagggccagccccgggagccacaggtgtataccctgccaccaagcagagatgagctgaccaagaatcaggtgtccctgacatgtctggtgaagggcttctatcccagcgatatcgccgtggagtgggagagcaacggccagcctgagaataactacaagaccacaccacctgtgctggattccgatggcagcttctttctgtattccaagctgaccgtggacaagtccagatggcagcagggcaacgtgttcagctgctccgtgatgcacgaggccctgcacaatcactacacccagaagtctctgtccctgtctcctggcaagtgataa

SEQ ID NO: 28 - protein, 502 aa, synthetic construct
  REGION 1..502: note = Chemically Synthesized, pGX9346, 1A2_scFv-Fc
MVLQTQVFISLLLWISGAYGDIVMTQSPRSLSVTPGEPASISCRSSQSLLHRNGYNYLDWYLQKPGQSPQLLIYLGSNRASGVPDRFSGSGSGTDFTLKISRVEAEDVGVYYCMQALQTPSWTFGQGTKVEIKGGGGSGGGGSGGGGSEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 2226 bp, synthetic construct
  misc_feature 1..2226: note = Chemically Synthesized, pGX9356, 1A2_Full length_partial graft
  source 1..2226: mol_type = other DNA
atggactggacctggagaatcctgttcctggtggcagcagcaaccggaacacacgcagaggtgcagctggtggagagcggcggcggcctgatccagcccggcggctctctgaggctgagctgcgcagcatccggatttgccgtgaggtctaactacctgagctgggtgcgccaggcacctggcaagggcctggagtgggtgtccctgatctactctggcggcctgaccgcatatgcagactccgtggagggccggttcaccatcagcagagataactccaagaatacactgtatctgcagatgaatagcctgagggtggaggacaccgccctgtactattgcgcccgcgtggccagctccgccggcacattctactatggcatggacgtgtggggccagggcaccacagtgaccgtgtctagcgccagcacaaagggaccatccgtgtttccactggcaccttcctctaagtccacctctggcggcacagccgccctgggctgtctggtgaaggactacttccctgagccagtgaccgtgtcttggaacagcggcgccctgacctctggagtgcacacatttccagccgtgctgcagagctccggcctgtacagcctgtctagcgtggtgaccgtgccctcctctagcctgggcacccagacatatatctgcaacgtgaatcacaagccatctaatacaaaggtggacaagaaggtggagcccaagagctgtgataagacccacacatgccctccctgtccagcacctgagctgctgggcggcccaagcgtgttcctgtttccacccaagcctaaggacaccctgatgatctccaggacccccgaggtgacatgcgtggtggtggacgtgtctcacgaggaccccgaggtgaagtttaactggtacgtggatggcgtggaggtgcacaatgccaagaccaagccccgggaggagcagtacaactctacctatagagtggtgagcgtgctgacagtgctgcaccaggattggctgaacggcaaggagtataagtgcaaggtgagcaataaggccctgccagcccccatcgagaagaccatctccaaggcaaagggacagccacgggagccacaggtgtacacactgcctccaagcagagacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtatagcaagctgaccgtggacaagtcccggtggcagcagggcaacgtgtttagctgctccgtgatgcacgaggccctgcacaatcactacacccagaagtctctgagcctgtcccctggcaagaggggaaggaagcggagatctggcagcggagccacaaacttctccctgctgaagcaggccggcgatgtggaggagaatcctggcccaatggtgctgcagacccaggtgtttatctccctgctgctgtggatctctggcgcctacggcgccatccagatgacacagagcccatcctctctgtccgcctctgtgggcgacagggtgaccatcacatgtcgcagctcccagtccctgctgcacagaaacggctacaattatctggattggtacctgcagaagcctggccagagcccacagctgctgatctatctgggctctaacagggcaagcggagtgccagacagattcagcggctccggctctggaaccgacttcaccctgaagatctcccgggtggaggcagaggacgtgggcgtgtactattgcatgcaggccctgcagacccctagctggacattcggccagggcaccaaggtggagatcaagagaacagtggccgccccaagcgtgttcatctttccacccagcgacgagcagctgaagtccggcaccgcctctgtggtgtgcctgctgaacaacttctaccctagggaggccaaggtgcagtggaaggtggataacgccctgcagagcggcaattcccaggagtctgtgaccgagcaggacagcaaggattccacatatagcctgtccaacaccctgacactgtccaaggccgattacgagaagcacaaggtgtatgcctgcgaggtgacccaccagggcctgtctagcccagtgacaaagagcttcaatcgcggcgagtgttgataa

SEQ ID NO: 30 - protein, 740 aa, synthetic construct
  REGION 1..740: note = Chemically Synthesized, pGX9356, 1A2_Full length_partial graft
MDWTWRILFLVAAATGTHAEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGAIQMTQSPSSLSASVGDRVTITCRSSQSLLHRNGYNYLDWYLQKPGQSPQLLIYLGSNRASGVPDRFSGSGSGTDFTLKISRVEAEDVGVYYCMQALQTPSWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 31 - DNA, 1512 bp, synthetic construct
  misc_feature 1..1512: note = Chemically Synthesized, pGX9357, 1A2_scFv-Fc_partial graft
  source 1..1512: mol_type = other DNA
atggtgctgcagacccaggtgttcatctctctgctgctgtggatctctggcgcctatggcgctatccagatgacccagtccccttcaagcctgtccgcctctgtgggcgacagggtgacaatcacctgcagatcctctcagagcctgctgcacaggaatggctacaactacctggactggtatctgcagaagcctggccagtcccctcagctgctgatctatctgggctctaatcgcgccagcggcgtgcctgacaggttctccggcagcggctctggcaccgacttcacactgaagatcagcagagtggaggccgaggacgtgggcgtgtactattgtatgcaggccctgcagaccccttcctggacattcggccagggcaccaaggtggagatcaagggcggcggcggctccggcggcggcggctctggcggcggcggctccgaggtgcagctggtggagtctggcggcggcctgatccagccaggcggctccctgagactgtcttgcgccgcctctggcttcgccgtgaggtccaactacctgtcttgggtgcgccaggcccctggcaagggcctggagtgggtgtctctgatctactccggcggcctgacagcctacgccgattccgtggagggccggttcacaatctccagagacaattccaagaacaccctgtacctgcagatgaactccctgagagtggaggatacagccctgtattactgtgccagagtggccagctccgccggcacattctattacggcatggacgtgtggggccagggcacaaccgtgaccgtgtcttccgagcctaagtcctgtgacaagacccacacctgtccaccctgtcctgccccagagctgctgggcggcccatccgtgttcctgttccctccaaagcccaaggatacactgatgatctctaggacccctgaggtgacctgcgtggtggtggatgtgagccacgaggaccctgaggtgaagtttaactggtacgtggatggcgtggaggtgcacaatgccaagacaaagccaagagaggagcagtacaattctacatacagagtggtgtctgtgctgacagtgctgcaccaggactggctgaatggcaaggagtacaagtgcaaggtgtccaacaaggccctgccagcccctatcgagaagaccatctccaaggccaagggccagccccgggagccacaggtgtataccctgccaccaagcagagatgagctgaccaagaatcaggtgtccctgacatgtctggtgaagggcttctatcccagcgatatcgccgtggagtgggagagcaacggccagcctgagaataactacaagaccacaccacctgtgctggattccgatggcagcttctttctgtattccaagctgaccgtggacaagtccagatggcagcagggcaacgtgttcagctgctccgtgatgcacgaggccctgcacaatcactacacccagaagtctctgtccctgtctcctggcaagtgataa

SEQ ID NO: 32 - protein, 502 aa, synthetic construct
  REGION 1..502: note = Chemically Synthesized, pGX9357, 1A2_scFv-Fc_partial graft
MVLQTQVFISLLLWISGAYGAIQMTQSPSSLSASVGDRVTITCRSSQSLLHRNGYNYLDWYLQKPGQSPQLLIYLGSNRASGVPDRFSGSGSGTDFTLKISRVEAEDVGVYYCMQALQTPSWTFGQGTKVEIKGGGGSGGGGSGGGGSEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 2199 bp, synthetic construct
  misc_feature 1..2199: note = Chemically Synthesized, pGX9362, EBV114_ Full length _partial graft
  source 1..2199: mol_type = other DNA
atggactggacctggagaatcctgttcctggtggcagcagcaaccggaacacacgcagaggtgcagctggtggagagcggcggcggcctgatccagccaggcggctccctgaggctgtcttgcgcagcaagcggatttgcactgaggatgtacgacatgcactgggtgcggcagaccatcgataagagactggagtgggtgagcgccgtgggaccatccggcgacacatactatgccgatagcgtgaagggcaggttcgccgtgtcccgcgagaacgccaagaattctctgagcctgcagatgaacagcctgaccgccggcgacacagccatctactattgcgtgcggtccgacagaggcgtggcaggcctgttcgattcttggggccagggcatcctggtgaccgtgagctccgcctctacaaagggaccaagcgtgtttccactggcaccttctagcaagtccacctctggcggcacagccgccctgggctgtctggtgaaggattacttccctgagccagtgaccgtgtcttggaacagcggcgccctgacctctggagtgcacacatttcctgccgtgctgcagtcctctggcctgtacagcctgagctccgtggtgaccgtgccatctagctccctgggcacccagacatatatctgcaacgtgaatcacaagccatctaatacaaaggtggacaagaaggtggagcccaagagctgtgataagacccacacatgccctccctgtccagcacctgagctgctgggcggcccatccgtgttcctgtttccacccaagcctaaggacaccctgatgatctccaggacccccgaggtgacatgcgtggtggtggacgtgtctcacgaggaccccgaggtgaagttcaactggtacgtggatggcgtggaggtgcacaatgccaagaccaagccccgggaggagcagtacaattccacctatagagtggtgtctgtgctgacagtgctgcaccaggactggctgaacggcaaggagtataagtgcaaggtgagcaataaggccctgccagcccccatcgagaagaccatctccaaggcaaagggacagccacgggagccacaggtgtacacactgcctccaagcagagacgagctgaccaagaaccaggtgtccctgacatgtctggtgaagggcttctatccttctgatatcgccgtggagtgggagagcaatggccagccagagaacaattacaagaccacaccccctgtgctggactccgatggctctttctttctgtattctaagctgaccgtggacaagagccgctggcagcagggcaacgtgtttagctgctccgtgatgcacgaggccctgcacaatcactacacccagaagtctctgagcctgtcccctggcaagaggggaaggaagcggagatctggcagcggagccacaaacttcagcctgctgaagcaggccggcgatgtggaggagaatcctggcccaatggtgctgcagacccaggtgtttatcagcctgctgctgtggatctccggcgcctatggcgccatccagatgacacagtccccttctagcctgtccgcctctgtgggcgacagggtgaccatcacatgtcgcgccagccaggccttcgataactacgtggcctggtatcagcagaggcctggcaaggtgccaaagctgctgatcagcgccgcatccgccctgcacgcaggagtgccatcccgcttcagcggctccggctctggaacccactttaccctgacaatctcctctctgcagccagaggacgtggccacatactattgccagaactacaatagcgcacccctgaccttcgggggcgggacaaaggtggagatcaagaggaccgtggcagcaccatccgtgttcatctttccaccctctgacgagcagctgaagtccggcacagcctctgtggtgtgcctgctgaacaacttctaccctagagaggccaaggtgcagtggaaggtggataacgccctgcagtctggcaatagccaggagtccgtgaccgagcaggactctaaggatagcacatatagcctgtccaacaccctgacactgtccaaggccgattacgagaagcacaaggtgtatgcatgcgaggtgacccaccagggcctgagctccccagtgacaaagagctttaatagaggcgagtgttgataa

SEQ ID NO: 34 - protein, 731 aa, synthetic construct
  REGION 1..731: note = Chemically Synthesized, pGX9362, EBV114_ Full length _partial graft
MDWTWRILFLVAAATGTHAEVQLVESGGGLIQPGGSLRLSCAASGFALRMYDMHWVRQTIDKRLEWVSAVGPSGDTYYADSVKGRFAVSRENAKNSLSLQMNSLTAGDTAIYYCVRSDRGVAGLFDSWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGAIQMTQSPSSLSASVGDRVTITCRASQAFDNYVAWYQQRPGKVPKLLISAASALHAGVPSRFSGSGSGTHFTLTISSLQPEDVATYYCQNYNSAPLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 35 - DNA, 1485 bp, synthetic construct
  misc_feature 1..1485: note = Chemically Synthesized, pGX9363, EBV114_scFv-Fc_partial graft
  source 1..1485: mol_type = other DNA
atggtgctgcagactcaggtgtttatttcactgctgctgtggatttcaggagcctatggagctattcagatgactcagagcccaagcagcctgtctgcctccgtgggcgatagagtgacaatcacctgtagagccagccaggccttcgacaattacgtggcctggtaccagcagagacccggcaaggtgccaaagctgctgatcagcgccgcctccgccctgcacgccggcgtgccttctcgcttttccggcagcggcagcggcacccactttaccctgacaatctccagcctgcagcctgaggatgtggccacctactattgtcagaattacaactctgccccactgaccttcggcggcggcaccaaggtggagatcaagggcggcggcggcagcggcggcggcggctccggcggcggcggctctgaggtgcagctggtggagtctggcggcggcctgatccagcccggcggctctctgaggctgtcctgcgccgcctccggctttgccctgcggatgtatgacatgcactgggtgcgccagacaatcgacaagagactggagtgggtgtctgccgtgggcccttccggcgatacctactatgccgatagcgtgaagggcagatttgccgtgagcagagagaacgccaagaactctctgagcctgcagatgaacagcctgaccgccggcgatacagccatctactattgcgtgagaagcgacagaggcgtggccggcctgttcgactcttggggccagggcatcctggtgacagtgtccagcgagcctaagtcttgcgataagacccacacctgtcccccttgcccagcccctgagctgctgggcggccccagcgtgttcctgttcccacctaagcctaaggatacactgatgatctccaggacaccagaggtgacatgcgtggtggtggacgtgtcccacgaggaccctgaggtgaagttcaactggtacgtggatggcgtggaggtgcacaacgccaagacaaagcccagggaggagcagtacaacagcacctatagagtggtgtccgtgctgacagtgctgcaccaggattggctgaatggcaaggagtacaagtgcaaggtgtccaacaaggccctgcccgcccctatcgagaagaccatctccaaggccaagggccagcctagagagccccaggtgtataccctgcctccctctagggatgagctgacaaagaaccaggtgtccctgacctgtctggtgaagggcttctatccaagcgatatcgccgtggagtgggagtctaacggccagcccgagaataactacaagacaacccctccagtgctggacagcgatggctccttctttctgtacagcaagctgaccgtggacaagtccaggtggcagcagggcaacgtgtttagctgttccgtgatgcacgaagccctgcacaaccactacactcagaagtcactgtcactgtctcctgggaagtgataa

SEQ ID NO: 36 - protein, 493 aa, synthetic construct
  REGION 1..493: note = Chemically Synthesized, pGX9363, EBV114_scFv-Fc_partial graft
MVLQTQVFISLLLWISGAYGAIQMTQSPSSLSASVGDRVTITCRASQAFDNYVAWYQQRPGKVPKLLISAASALHAGVPSRFSGSGSGTHFTLTISSLQPEDVATYYCQNYNSAPLTFGGGTKVEIKGGGGSGGGGSGGGGSEVQLVESGGGLIQPGGSLRLSCAASGFALRMYDMHWVRQTIDKRLEWVSAVGPSGDTYYADSVKGRFAVSRENAKNSLSLQMNSLTAGDTAIYYCVRSDRGVAGLFDSWGQGILVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 2193 bp, synthetic construct
  misc_feature 1..2193: note = Chemically Synthesized, pGX9224, Ebola Z5D2
  source 1..2193: mol_type = other DNA
atggattggacttggaggattctgtttctggtcgccgccgctactgggacacacgccgaggtgcagctgcaggagagtgggcctggactggtgcgacccagccagtccctgtctctgacatgcactgtgaccggctacagtatcacatcagactatgcctggaactggattcgccagttcccaggcaataagctggaatggctgggatacatcacaaacactggcagcaccgggtttaatcccagcctgaagtcccgaatctctattacaagggacacttctaaaaaccagttctttctgcagctgattagtgtgaccacagaggataccgcaacataccactgcgcccggggactggcttattggggacaggggaccctggtcacagtgagctccgctagtacaaaggggccttcagtgttccccctggcaccttctagtaaaagtacatcaggcggaactgccgctctgggctgtctggtgaaggattacttccctgagccagtcaccgtgagttggaactcaggagcactgacctccggggtccatacatttcccgccgtgctgcagtcaagcggcctgtactctctgtcctctgtggtcactgtgcctagttcaagcctgggaactcagacctatatctgcaacgtgaatcacaagcctagcaataccaaagtcgacaagaaagtggaaccaaagagctgtgataaaacacatacttgcccaccttgtccagcacctgagctgctgggaggaccaagcgtgttcctgtttccacccaagcctaaagacacactgatgatctcccgcaccccagaagtcacatgtgtggtcgtggacgtgtctcacgaggaccccgaagtcaagttcaactggtacgtggatggcgtcgaggtgcataatgctaagaccaaaccacgcgaggaacagtacaacagcacatatcgagtcgtgtccgtcctgactgtgctgcaccaggactggctgaacggcaaggagtataagtgcaaagtgagcaataaggctctgccagcacccatcgagaaaacaattagcaaggcaaaaggacagccaagggaaccccaggtgtacactctgcctccatccagagacgagctgactaagaaccaggtctctctgacctgtctggtgaaaggattctatcccagcgatatcgccgtggagtgggaatccaatgggcagcctgaaaacaattacaagactaccccccctgtgctggacagcgatgggtccttctttctgtattccaagctgaccgtggataaatctcggtggcagcagggcaacgtctttagctgctccgtgatgcatgaggccctgcacaatcattacacacagaagtctctgagtctgtcacctggcaagcggggacgcaaaaggagaagcggctccggagcaactaacttcagcctgctgaaacaggccggggacgtggaggaaaatcctggcccaatggtcctgcagacccaggtgtttatctctctgctgctgtggattagtggggcctatggcgatgtcgtgctgacccagacaccactgactctgagcgtgaccatcggacagcccgcttctattagttgtaagtcctctcagtctctgctggacagtgatggcaaaacctacctgaactggctgctgcagagacctggacagtccccaaagcggctgatctatctggtctcaaaactggacagcggcgtgacagatcggttcactgggtcaggcagcggaactgactttaccctgaagatttctcgcgtcgaggctgaagatctgggagtgtactattgctggcaggggactcactcacctttcacctttgggagcggcacaaagctggaaatcaaaaccgtcgcagccccaagtgtgttcatttttccaccctcagacgagcagctgaagtccgggacagcatctgtcgtgtgtctgctgaacaatttctaccctagggaggctaaggtccagtggaaagtggataacgcactgcagtctggcaatagtcaggagtcagtgaccgaacaggacagcaaggattccacatattccctgtctaacactctgaccctgagcaaagccgactacgagaagcacaaagtctatgcttgcgaagtgactcatcaggggctgagttcaccagtgaccaagagctttaatagaggcgagtgttgataa

SEQ ID NO: 38 - protein, 729 aa, synthetic construct
  REGION 1..729: note = Chemically Synthesized, pGX9224, Ebola Z5D2
MDWTWRILFLVAAATGTHAEVQLQESGPGLVRPSQSLSLTCTVTGYSITSDYAWNWIRQFPGNKLEWLGYITNTGSTGFNPSLKSRISITRDTSKNQFFLQLISVTTEDTATYHCARGLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDVVLTQTPLTLSVTIGQPASISCKSSQSLLDSDGKTYLNWLLQRPGQSPKRLIYLVSKLDSGVTDRFTGSGSGTDFTLKISRVEAEDLGVYYCWQGTHSPFTFGSGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 39 - DNA, 2208 bp, synthetic construct
  misc_feature 1..2208: note = Chemically Synthesized, pGX9225, Ebola Z1H3
  source 1..2208: mol_type = other DNA
atggattggacatggagaatcctgttcctggtcgccgccgctactgggacacacgccgaagtgcagctgcagcagagtggagccgagctggtcaaacctggggcatccgtgaagctgtcttgcactgccagtggcttcaacatcaaagacacctacattcactgggtgaagcagggcccagagcagggactggaatggatcggacggattgacccagcaaacgggaataccaagtatgatcccaaatttcaggggaaggcaaccatcacagccgacacaagctccaatactgcctacctgcagctgtcaggcctgaccagcgaggatacagccgtgtactattgcgccagggagagcaggatttccaccatgctgaccacaggatatttcgactactggggacaggggactaccctgaccgtctctagtgccagcacaaaagggccttccgtgtttcccctggctccttcaagcaagtctactagtggaggaaccgcagctctgggatgtctggtgaaggattacttccctgagccagtcaccgtgtcttggaacagtggggctctgacctccggcgtccacacatttccagcagtgctgcagtcctctgggctgtattctctgagttcagtggtcaccgtgcccagctcctctctgggcactcagacctacatctgcaacgtgaatcacaaaccctccaatacaaaggtcgacaagaaagtggaacctaaatcttgtgataagacacatacttgcccaccttgtccagcacctgagctgctgggaggacctagcgtgttcctgtttccacccaagccaaaagacaccctgatgatcagccgcacacctgaagtcacttgtgtggtcgtggacgtgtcccacgaggaccccgaagtcaagttcaactggtacgtggatggcgtcgaggtgcataatgccaagacaaaacccagagaggaacagtataactctacataccgggtcgtgagtgtcctgactgtgctgcaccaggattggctgaacggcaaggagtacaagtgcaaagtgtctaacaaggccctgccagctcccatcgagaagaccattagcaaggccaaaggacagccacgcgaaccccaggtgtatacactgcctcccagcagggacgagctgactaaaaaccaggtcagcctgacctgtctggtgaagggcttctacccaagcgatatcgctgtggagtgggaatccaatggacagcccgaaaacaattataagacaactccccctgtgctggactcagatggcagcttctttctgtacagtaaactgaccgtggacaagtcaagatggcagcagggaaacgtcttttcatgcagcgtgatgcatgaggccctgcacaatcattatactcagaagtccctgtctctgagtccagggaaacggggccgcaagaggagatcaggcagcggagctacaaacttcagcctgctgaagcaggcaggcgatgtggaggaaaatcctggaccaatggtcctgcagactcaggtgtttatctcactgctgctgtggattagcggagcctacggacagatcgtgctgacccagtccccagctattatgtccgcatctcctggcgagaaagtgaccatgacatgttccgctagttcaagcgtgtcttacatgtattggtaccagcagaagcctggcagcagcccaaggctgctgatctatgacacctccaacctggcttctggggtccccgtgagattcagtgggtcaggcagcggaacttcctactctctgaccatttcccggatggaggcagaagatgcagccacatactattgccagcagtggagttcatatccctacacatttggagggggcactaaactggaaatcaagacagtcgctgcaccttctgtgttcatttttccacccagtgacgagcagctgaagagtggcactgcctcagtcgtgtgtctgctgaacaatttctatccccgcgaggccaaagtccagtggaaggtggataacgctctgcagtccggcaattctcaggagagtgtgaccgaacaggactcaaaagatagcacatacagtctgtcaaacactctgaccctgagcaaggcagactatgagaagcacaaagtctacgcctgcgaagtgacacatcagggactgagctcccctgtgactaagtcctttaatcgaggggagtgttgataa

SEQ ID NO: 40 - protein, 734 aa, synthetic construct
  REGION 1..734: note = Chemically Synthesized, pGX9225, Ebola Z1H3
MDWTWRILFLVAAATGTHAEVQLQQSGAELVKPGASVKLSCTASGFNIKDTYIHWVKQGPEQGLEWIGRIDPANGNTKYDPKFQGKATITADTSSNTAYLQLSGLTSEDTAVYYCARESRISTMLTTGYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGQIVLTQSPAIMSASPGEKVTMTCSASSSVSYMYWYQQKPGSSPRLLIYDTSNLASGVPVRFSGSGSGTSYSLTISRMEAEDAATYYCQQWSSYPYTFGGGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 41 - DNA, 2208 bp, synthetic construct
  misc_feature 1..2208: note = Chemically Synthesized, pGX9228, Ebola ZBDBV223
  source 1..2208: mol_type = other DNA
atggattggacttggagaattctgtttctggtggccgccgctacaggaactcacgctcaggtgcagctgcagcagtggggagccgggctgctgaagccaagcgagacactgtccctgacttgcgccgtgtacggcggaagcttcaccacaacttattggaattggatcaggcagccccctggcaagggactggagtggattggggaagtgaactacagcggcaacgctaattataacccctccctgaagggccgagtcgcaatctctgtggacactagtaaaaatcagtttagtctgaggctgaactcagtgaccgccgctgatacagcaatctactattgcaccagcaggatcaggagccacattgcctactcctggaagggagacgtgtgggggaaaggcaccacagtcaccgtgagctccgccagcacaaagggaccatccgtgttcccactggctccctctagtaaaagtacttcagggggcaccgcagccctgggatgtctggtgaaggattacttccccgagcctgtcaccgtgtcctggaattctggcgcactgacctctggagtccatacatttccagccgtgctgcagtcaagcgggctgtacagcctgtcctctgtggtcaccgtccccagttcaagcctgggcacacagacttatatctgcaatgtgaaccacaagccctctaacacaaaagtcgacaagaaagtggaacctaagagctgtgataaaacccatacatgcccaccctgtccagcaccagagctgctgggagggcctagcgtgttcctgtttcctccaaagccaaaagacacactgatgattagcaggacacctgaagtcacttgcgtggtcgtggacgtgtcccacgaggaccccgaagtcaagtttaattggtacgtggatggcgtcgaggtgcataacgctaagaccaaacctagggaggaacagtacaactctacatatagagtcgtgagtgtcctgactgtgctgcaccaggactggctgaatgggaaggagtataagtgcaaagtgtctaacaaggcactgcctgccccaatcgagaaaactattagcaaggctaaaggccagcctcgggaaccacaggtgtacaccctgccccctagccgcgacgagctgactaagaatcaggtctccctgacctgtctggtgaaaggcttctatccatctgatatcgcagtggagtgggaaagtaacggacagcccgaaaacaattacaagactaccccacccgtcctggacagtgatggctcattctttctgtattccaagctgaccgtggacaaatctagatggcagcagggaaatgtctttagctgctccgtgatgcacgaggccctgcacaaccattacactcagaagtctctgagtctgtcaccagggaagcgaggcaggaaaaggagaagcggcagcggggcaaccaatttctctctgctgaaacaggccggagatgtggaggaaaaccccgggcctatggtcctgcagacacaggtgtttatctcactgctgctgtggattagcggagcctacggggaaatcgtgatgactcagagcccaggcaccctgtctctgagtcccggagagagagctacactgtcctgtcgggcatcacagagcgtgcccagaaattacatcggatggttccagcagaagccaggacaggcccctcggctgctgatctacggagcttcctctcgcgctgcagggttccctgaccgattttccggctctggaagtgggaccgatttcactctgaccatcacacgcctggagcccgaagactttgccatgtactattgccaccagtacgatcgactgccttatacattcggccagggaactaagctggaaatcaaaacagtcgccgctcctagcgtgttcatctttcctccatcagacgagcagctgaagtccggaactgcttctgtggtgtgcctgctgaacaacttctacccacgcgaagctaaggtccagtggaaagtggataatgcactgcagagcggcaactcccaggagtctgtgaccgaacaggacagtaaggattcaacatattcactgagcaacactctgaccctgtccaaagccgactacgagaagcataaagtgtatgcttgcgaggtcacccaccaggggctgtcatctccagtcactaagtccttcaatagaggcgaatgttgataa

SEQ ID NO: 42 - protein, 734 aa, synthetic construct
  REGION 1..734: note = Chemically Synthesized, pGX9228, Ebola ZBDBV223
MDWTWRILFLVAAATGTHAQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 43 - DNA, 2223 bp, synthetic construct
  misc_feature 1..2223: note = Chemically Synthesized, pGX9256, 1A2
  source 1..2223: mol_type = other DNA
atggactggacctggagaatcctgttcctggtggcagcagcaaccggaacacacgcagaggtgcagctggtggagagcggcggcggcctgatccagccaggcggcagcctgaggctgtcctgcgcagcatctggatttgccgtgaggagcaactacctgtcctgggtgagacaggcaccaggcaagggactggagtgggtgtctctgatctacagcggcggcctgaccgcatatgcagacagcgtggagggcaggttcaccatctccagagataactctaagaatacactgtatctgcagatgaattccctgcgggtggaggacaccgccctgtactattgcgcccgcgtggccagctccgccggcacattctactatggcatggacgtgtggggccagggcaccacagtgaccgtgtctagcgcctccacaaagggaccaagcgtgttcccactggcaccttcctctaagtccacctctggcggcacagccgccctgggctgtctggtgaaggattacttccctgagccagtgaccgtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacacactgatgatctctaggaccccagaggtgacatgcgtggtggtggacgtgagccacgaggaccccgaggtgaagtttaactggtacgtggatggcgtggaggtgcacaatgccaagaccaagccaagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccaagggagccacaggtgtacacactgcctccatccagagacgagctgaccaagaaccaggtgtctctgacatgtctggtgaagggcttctatccctctgatatcgccgtggagtgggagagcaatggccagcctgagaacaattacaagaccacaccccctgtgctggactccgatggctctttctttctgtattccaagctgaccgtggataagtctcggtggcagcagggcaacgtgtttagctgctccgtgatgcacgaggccctgcacaatcactacacccagaagtctctgagcctgtcccctggcaagaggggaaggaagaggagatctggcagcggcgccacaaacttcagcctgctgaagcaggcaggcgacgtggaggagaatcctggaccaatggtgctgcagacccaggtgtttatctctctgctgctgtggatcagcggcgcctacggcgatatcgtgatgacccagtcccctcgctccctgtctgtgacacctggcgagccagccagcatctcctgtcggtcctctcagtctctgctgcaccgcaacggctacaattatctggactggtacctgcagaagcccggccagtcccctcagctgctgatctatctgggcagcaacagggcatccggagtgccagaccgcttctctggcagcggctccggaaccgacttcaccctgaagatcagcagggtggaggccgaggatgtgggcgtgtactattgcatgcaggccctgcagaccccctcctggacattcggccagggcaccaaggtggagatcaagacagtggccgcccctagcgtgttcatctttccaccctccgacgagcagctgaagtctggcaccgccagcgtggtgtgcctgctgaacaacttctaccccagagaggccaaggtgcagtggaaggtggataacgccctgcagagcggcaattcccaggagtctgtgaccgagcaggacagcaaggattccacatattctctgagctccaccctgacactgagcaaggccgactacgagaagcacaaggtgtatgcctgcgaggtgacccaccagggcctgtctagccctgtgacaaagtccttcaacagaggcgagtgttgataa

SEQ ID NO: 44 - protein, 739 aa, synthetic construct
  REGION 1..739: note = Chemically Synthesized, pGX9256, 1A2
MDWTWRILFLVAAATGTHAEVQLVESGGGLIQPGGSLRLSCAASGFAVRSNYLSWVRQAPGKGLEWVSLIYSGGLTAYADSVEGRFTISRDNSKNTLYLQMNSLRVEDTALYYCARVASSAGTFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPRSLSVTPGEPASISCRSSQSLLHRNGYNYLDWYLQKPGQSPQLLIYLGSNRASGVPDRFSGSGSGTDFTLKISRVEAEDVGVYYCMQALQTPSWTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 2196 bp, synthetic construct
  misc_feature 1..2196: note = Chemically Synthesized, pGX9290, EBV114
  source 1..2196: mol_type = other DNA
atggactggacatggagaatcctgttcctggtcgccgccgctactgggactcacgccgaggtgcagctggtcgaaagtggagggggactgatccagccaggcggaagcctgcgactgtcctgcgcagcttctggattcgcactgcgcatgtacgacatgcactgggtgcgccagacaattgataagcgactggaatgggtcagcgccgtgggaccatccggggacacttactatgctgattctgtgaaggggagatttgctgtcagtcgggagaacgcaaaaaattctctgagtctgcagatgaactctctgaccgcaggcgacacagccatctactattgcgtgcgatccgacaggggcgtcgcaggactgttcgattcttggggccagggaattctggtcacagtgagctccgcctctactaagggaccaagcgtgttcccactggctccttctagtaaatcaactagcgggggcaccgcagccctgggatgtctggtgaaggattacttccctgagccagtcaccgtgagttggaactcaggcgcactgaccagcggagtgcatacatttcctgccgtcctgcagtcaagcggcctgtactccctgtcctctgtggtcacagtgccaagttcaagcctgggaactcagacctatatctgcaacgtgaatcacaagccatctaatactaaagtcgacaagaaagtggaacccaagagctgtgataaaacacatacttgccctccctgtccagcacctgagctgctgggagggccaagcgtgttcctgtttccacccaagcctaaagacaccctgatgattagccggacacccgaagtgacttgcgtggtcgtggacgtgagccacgaggaccccgaagtgaagttcaactggtacgtggatggcgtcgaggtgcataatgctaagacaaaaccccgggaggaacagtacaattcaacttatcgcgtcgtgagcgtcctgaccgtgctgcaccaggactggctgaacggaaaggagtataagtgcaaagtgtctaataaggcactgccagcccccatcgagaaaaccattagcaaggccaaagggcagccaagggaaccccaggtgtacacactgcctccaagtagagacgagctgaccaagaaccaggtgtccctgacatgtctggtcaaaggattctatccttcagatatcgctgtggagtgggaaagcaatgggcagccagaaaacaattacaagaccacaccccctgtgctggacagcgatggcagcttcttcctgtatagtaagctgaccgtggataaatcaaggtggcagcaggggaacgtcttttcctgctctgtgatgcatgaggccctgcacaatcattacacccagaagagtctgtcactgagccctgggaagcgaggacgaaaaaggagatccgggtctggcgccacaaacttcagcctgctgaagcaggctggggacgtggaggaaaatcctggcccaatggtcctgcagacccaggtgtttatctccctgctgctgtggatttctggagcatatggggatatccagatgacacagtctccttcctctctgagtgcttcagtgggcgacaggatcaccattacatgtagagctagccaggcattcgataactacgtggcctggtatcagcagcggcctgggaaggtgccaaaactgctgatctctgctgcaagtgccctgcacgctggagtgccaagccgcttcagcggcagcgggtctggaactcacttcaccctgaccattagttcactgcagccagaggacgtggctacctactattgccagaactacaattccgcacccctgactttcggcggagggaccaaggtcgaaatcaaaactgtggccgctcccagcgtcttcatttttccaccctccgacgagcagctgaagagtggcaccgcctcagtggtgtgcctgctgaacaacttctaccctagagaagcaaaggtccagtggaaagtggataacgccctgcagtcaggaaatagccaggagtccgtgacagaacaggactctaaggatagtacttattcactgagctccacactgactctgtccaaagctgactacgagaagcacaaagtgtatgcatgcgaagtgacccaccagggactgagcagccccgtgaccaagagctttaatagaggagaatgttgataa

SEQ ID NO: 46 - protein, 730 aa, synthetic construct
  REGION 1..730: note = Chemically Synthesized, pGX9290, EBV114
MDWTWRILFLVAAATGTHAEVQLVESGGGLIQPGGSLRLSCAASGFALRMYDMHWVRQTIDKRLEWVSAVGPSGDTYYADSVKGRFAVSRENAKNSLSLQMNSLTAGDTAIYYCVRSDRGVAGLFDSWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRITITCRASQAFDNYVAWYQQRPGKVPKLLISAASALHAGVPSRFSGSGSGTHFTLTISSLQPEDVATYYCQNYNSAPLTFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 108 aa, synthetic construct
  REGION 1..108: note = Chemically Synthesized
ETTLTQSPGTLSLSPGERATLSCRASENIYSYLAWYQQKQGKSPQLLVYNAKTLIEGVPSRFSGSGSGTQFSLKINSLQPEDFGSYFCQHHFGTPFTFGSGTKLEIKR

SEQ ID NO: 48 - protein, 107 aa, synthetic construct
  REGION 1..107: note = Chemically Synthesized
DIQMTQSPASLSASVGETVTITCRASENIYSYLAWYQQKQGKSPQLLVYNAKTLIEGVPSRFSGSGSGTQFSLKINSLQPEDFGSYFCQHHFGTPFTFGSGTELEIK

SEQ ID NO: 49 - protein, 107 aa, synthetic construct; identical to SEQ ID NO: 48
  REGION 1..107: note = Chemically Synthesized

SEQ ID NO: 50 - protein, 107 aa, synthetic construct
  REGION 1..107: note = Chemically Synthesized
DIQMTQSPASLSASVGETVTITCNASENIYSYLAWYQQKQGQSPQLLVYNAKTLIEGVPSRFSGSGSGTQFSLKINSLQPEDFGSYWCQHHFGTPFTFGSGTELEIK

SEQ ID NO: 51 - protein, 119 aa, synthetic construct
  REGION 1..119: note = Chemically Synthesized
QVQLQQSGPELEMPGASVKISCKASGSSFTGFSMNWVKQSNGKSLEWIGNIDTYYGGTTYNQKFKGKATLTVDKSSSTAYMQLKSLTSEDSAVYYCARSAYYGSTFAYWGQGTLVTVSS

SEQ ID NO: 52 - protein, 119 aa, synthetic construct
  REGION 1..119: note = Chemically Synthesized
QVQLQQSGPELEMPGASVKISCQASGSSFTGFSMNWVKYSNGKSLEWIGNIDTYYGGTTYNQKFKGKATLTVDKSSSTAYMQLQSLTSEDSAVYYCARSAYYGSTFAYWGQGTLVTVSS

SEQ ID NO: 53 - protein, 15 aa, synthetic construct
  REGION 1..15: note = Chemically Synthesized
GGGGSGGGGSGGGGS

SEQ ID NO: 54 - protein, 113 aa, synthetic construct
  REGION 1..113: note = Chemically Synthesized
DIVMTQSPRSLSVTPGEPASISCRASQSLLHRNGYNYLDWYLQKPGQSPQLLIYLGSNRASGVPDRFSGSGSGTDFTLKISRVEAEDVGVYYCMQALQTPSWTFGQGTKVEIK

SEQ ID NO: 55 - protein, 107 aa, synthetic construct [sequence omitted]

SEQ ID NO: 56 - protein, 108 aa, synthetic construct
  REGION 1..108: note = Chemically Synthesized
ETTLTQSPGTLSLSPGERATLSCRASQSVSSSIAWYQQKPGQAPRLLMFDSSTRATGIPDRFSGSGSGTDFTLNISSLEPEDFAVYYCQQYSSSPYTFGQGTKLEIKR

SEQ ID NO: 57 - protein, 108 aa, synthetic construct
  REGION 1..108: note = Chemically Synthesized
AIQMTQSPSSLSASVGDRVTITCRASQGIRNDLGWYQQKPGKAPKLLIYSASTLQSGVPSRFSGSGSGTDFTLTISSLQPEDFATYYCLQDYNYPWTFGQGTKVEIKR

SEQ ID NO: 58 - protein, 114 aa, synthetic construct
  REGION 1..114: note = Chemically Synthesized
ETTLTQSPGTLSLSPGERATLSCRSSQSLLHRNGYNYLDWYLQKPGQSPQLLIYLGSNRASGVPDRFSGSGSGTDFTLKISRVEAEDVGVYYCMQALQTPSWTFGQGTKLEIKR

SEQ ID NO: 59 - protein, 108 aa, synthetic construct
  REGION 1..108: note = Chemically Synthesized
ETTLTQSPGTLSLSPGERATLSCRASQAFDNYVAWYQQRPGKVPKLLISAASALHAGVPSRFSGSGSGTHFTLTISSLQPEDVATYYCQNYNSAPLTFGGGTKLEIKR

SEQ ID NO: 60 - protein, 114 aa, synthetic construct
  REGION 1..114: note = Chemically Synthesized
AIQMTQSPSSLSASVGDRVTITCRSSQSLLHRNGYNYLDWYLQKPGQSPQLLIYLGSNRASGVPDRFSGSGSGTDFTLKISRVEAEDVGVYYCMQALQTPSWTFGQGTKVEIKR

SEQ ID NO: 61 - protein, 108 aa, synthetic construct
  REGION 1..108: note = Chemically Synthesized
AIQMTQSPSSLSASVGDRVTITCRASQAFDNYVAWYQQRPGKVPKLLISAASALHAGVPSRFSGSGSGTHFTLTISSLQPEDVATYYCQNYNSAPLTFGGGTKVEIKR

SEQ ID NO: 62 - protein, 47 aa, synthetic construct
  REGION 1..47: note = Chemically Synthesized
DAEFRHDSGYEVHHQKLVFFAEDVGSNKGAIIGLMVGGVVIATVIVI

SEQ ID NO: 63 - DNA, 2235 bp, synthetic construct
  misc_feature 1..2235: note = Chemically Synthesized, pRD211, 190-FP2A-mouse-GeneArt
  source 1..2235: mol_type = other DNA
atggattggacttggagaatcctgttcctggtggccgctgctacaggcacacatgctcaggtgcagctggttgaaagcggaggcggagttgtgcagcctggcagatctctgagactgtcttgtgccgccagcggcttcaccttctctaagtacggaatgcactgggtccgacaggcccctggaaaaggactggaatgggtcgccgtgatcagctacgagggcagcaacaagtactacgccgacagcgtgaagggcagattcaccatcagcagagacaacagcaagaacaccctgtacctgcagatgaacagcctgagagccgaggacaccgccgtgtactactgtgctaagagcggcacccagtactacgacaccaccggctacgagtacagaggcctggaatactttggctactggggccagggaaccctggtcacagttagctctgctagcacaaagggccccagcgttttcccactggctcctagctctaagagcacatctggcggaacagctgctctgggctgtctggtcaaggactactttcctgagcctgtgaccgtgtcctggaactctggtgctctgacaagcggcgtgcacacatttccagctgtgctgcagtctagcggcctgtactctctgtctagcgtcgtgacagtgcctagcagctctctgggaacccagacctacatctgcaacgtgaaccacaagcctagcaacaccaaggtggacaagagagtggaacccaagagctgcgacaagacccacacctgtcctccatgtcctgctccagaagctgcaggcggaccctctgtgttcctgtttcctccaaagcctaaggacaccctgatgatcagcagaacccctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcctgctcctatcgaaaagaccatctccaaggctaagggccagcctagggaaccccaggtttacacactgcctccaagcagagaagagatgaccaagaaccaggtgtccctgacctgcctcgtgaagggattctacccttccgatatcgccgtggaatgggagtctaacggccagccagagaacaactacaagacaacccctcctgtgctggacagcgacggctcattcttcctgtacagcaagctgaccgtggacaagtccagatggcagcagggcaacgtgttcagctgttctgtgatgcacgaggccctgcacaaccactacacccagaagtctctgtctctgagccccggcaagcgcggcagaaagagaagatctggaagcggcgccaccaacttcagtctgctgaaacaggctggcgacgtggaagagaaccctggacctatggtgctgcagacccaggtgttcattagcctgctgctgtggatctctggcgcctatggcgagatcgtgctgacccagtctcctggcacattgagcctgtctccaggcgagagagccacactgagctgtagagcctctcagagcgtgtcctctagctacctggcctggtatcagcaaaagagaggccaggctcctagactgctgatctacgacgcctcttccagagccacaggcatccctgatagattcagcggctctggcagcggcaccgacttcacactgaccatctctagactggaacccgaggatttcgctgtgtactattgccagcagtacggcagatccagatggacctttggacagggcacaaaggtggaaatcaagagaaccgtggccgctcctagcgtgttcatctttccaccaagcgacgagcagctgaagtctggcacagcttctgtcgtgtgcctgctgaacaacttctaccccagagaagccaaggtgcagtggaaggtcgacaacgctctgcagtccggcaacagccaagagagcgtgacagagcaggactccaaggacagcacatactccctgagcagcaccctgacactgagcaaggccgactacgaaaagcacaaggtgtacgcctgcgaagtgacacaccagggactgagcagccctgtgaccaagtctttcaacagaggcgagtgctgatga

SEQ ID NO: 64 - protein, 743 aa, synthetic construct
  REGION 1..743: note = Chemically Synthesized, pRD211, 190-FP2A-mouse-GeneArt
MDWTWRILFLVAAATGTHAQVQLVESGGGVVQPGRSLRLSCAASGFTFSKYGMHWVRQAPGKGLEWVAVISYEGSNKYYADSVKGRFTISRDNSKNTLYLQMNSLRAEDTAVYYCAKSGTQYYDTTGYEYRGLEYFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGERATLSCRASQSVSSSYLAWYQQKRGQAPRLLIYDASSRATGIPDRFSGSGSGTDFTLTISRLEPEDFAVYYCQQYGRSRWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - DNA, 2235 bp, synthetic construct
  misc_feature 1..2235: note = Chemically Synthesized, pRD212, 190-FP2A-mouse-Synbio
  source 1..2235: mol_type = other DNA
atggactggacttggagaatcctgttcctggtggcagcagctacaggaacacacgctcaggtgcagctggtggaatcaggaggaggagtggtgcagccaggcagaagcctgagactgtcttgcgccgctagcggcttcacattcagcaagtacggcatgcattgggtccgacaggctccaggaaagggactggagtgggtggccgtgattagctacgagggcagcaacaagtactacgccgacagcgtgaagggcaggttcaccatcagccgggacaacagcaagaacaccctgtacctgcagatgaacagcctgagggccgaggataccgccgtgtactattgcgccaagagcggaacccagtactacgacaccaccggctacgagtacaggggactggagtacttcggctattggggccagggaacactggtgaccgtgtctagcgctagcaccaagggacctagcgtgttccctctggccccttctagcaagtctaccagcggaggaacagccgctctgggttgtctggtgaaggactacttcccagagcccgtgaccgtgtcttggaatagcggagctctgaccagcggagtgcacacattcccagcagtgctgcagagcagcggactgtactctctgagcagcgtggtgaccgtgccttcttctagcctgggcacccagacctacatctgcaacgtgaaccacaagcccagcaacaccaaggtggacaagagggtggagcccaagtcttgcgacaagacccacacctgtcccccttgtccagctccagaagcagcaggaggacctagcgtgttcctgttccctcccaagcccaaggacaccctgatgatcagccggaccccagaagtgacttgcgtggtggtggacgtgtctcacgaggaccccgaggtcaagttcaattggtacgtggacggagtggaggtgcacaacgctaagaccaagcccagggaggagcagtacaacagcacctacagggtggtgtccgtgctgacagtgctgcaccaggattggctgaacggcaaggagtacaagtgcaaggtgtccaacaaggctctgccagcccccatcgagaagaccatcagcaaggctaagggacagcctagggaacctcaggtgtacaccctgcctcctagcagggaggagatgaccaagaaccaggtgtccctgacttgcctcgtgaagggcttctaccctagcgacatcgccgtggagtgggaatctaacggccagccagagaacaactacaagaccacccccccagtgctggacagcgacggcagcttcttcctgtacagcaagctgaccgtggacaagagccgttggcagcagggcaacgtgttctcttgcagcgtgatgcacgaggccctgcacaaccactacacccagaagagcctgagcctgagcccaggaaagaggggcagaaagagaagaagcggaagcggcgctaccaacttcagcctgctgaagcaggccggagacgtggaggagaatccaggacccatggtgctgcagacccaggtgttcatcagcctcctcctgtggatcagcggagcttacggcgagatcgtgctgacacagagcccaggaaccctgtctctgtctccaggagagagagccaccctgtcttgcagagctagccagagcgtgtctagcagctacctggcttggtaccagcagaagaggggccaggctcctagactgctgatctacgacgccagctctagagccaccggaatccccgacagattcagcggaagcggaagcggaaccgacttcaccctgaccatcagcagactggagccagaggacttcgccgtctactactgccagcagtacggcagatctcgttggaccttcggacagggaaccaaggtggagatcaagcggaccgtggcagcccctagcgtgttcatcttccctcctagcgacgagcagctgaagagcggaacagctagcgtcgtctgcctgctgaacaacttctaccccagggaggccaaggtccagtggaaggtcgataacgccctgcagagcggaaactctcaggagagcgtgaccgagcaggactctaaggacagcacctacagcctgagcagcacactgaccctgagcaaggccgactacgagaagcacaaggtgtacgcttgcgaagtgacccaccagggactgtctagccccgtgaccaagagcttcaaccggggcgagtgttgataa

SEQ ID NO: 66 - protein, 743 aa, synthetic construct; identical to SEQ ID NO: 64
  REGION 1..743: note = Chemically Synthesized, pRD212, 190-FP2A-mouse-Synbio

SEQ ID NO: 67 - DNA, 2235 bp, synthetic construct
  misc_feature 1..2235: note = Chemically Synthesized, pRD213, 190-FP2A-mouse-Genewiz
  source 1..2235: mol_type = other DNA
atggactggacctggagaatcctgttcctcgtcgctgccgctacaggcacccatgcccaggtgcagctggtggaaagcggcggaggcgtcgtgcagcctggaaggagcctgagactgagctgcgccgcctccggcttcaccttctccaaatacggcatgcactgggtgaggcaggcccccggcaagggcctggagtgggtcgctgtgatcagctacgaaggcagcaacaagtactacgccgacagcgtcaagggcaggttcaccatctccagagacaactccaagaatacactgtacctgcagatgaacagcctgagggctgaggacaccgctgtgtactactgcgccaagagcggcacccagtactatgataccaccggctatgagtacaggggcctggaatacttcggatattggggccagggcaccctggtgacagtgagctccgctagcacaaagggacccagcgtgttccctctcgctcccagctccaagtccacaagcggcggaacagccgctctgggctgcctcgtgaaagactatttccctgagcccgtgacagtcagctggaattccggcgccctgacaagcggcgtccataccttccctgccgtcctccaaagctccggcctgtactccctgtcctccgtggtcacagtgcccagctcctccctgggaacccagacctacatttgcaacgtcaatcacaagcccagcaacaccaaggtcgacaaaagggtcgaacccaagagctgcgacaaaacccacacctgtcctccctgccctgctcctgaagccgctggaggacctagcgtgttcctctttcctcctaagcccaaggacaccctgatgatctccaggacccctgaggtgacctgtgtggtcgtggacgtgagccacgaagatcccgaggtcaagttcaactggtacgtggacggcgtcgaagtgcacaatgccaaaaccaagcccagagaggagcaatacaactccacctacagagtggtgtccgtgctcacagtgctgcaccaggactggctgaatggcaaagagtacaagtgtaaagtcagcaataaggctctgcccgctcctattgagaaaaccatcagcaaggctaaaggacagcccagggagccccaggtgtacacactgcctcccagcagggaggagatgacaaagaatcaggtgtccctgacatgcctggtgaagggattctacccctccgatatcgctgtcgagtgggagtccaacggccaacccgaaaacaactataagaccacaccccccgtcctcgattccgacggctccttctttctgtattccaagctgaccgtggacaagtccaggtggcagcagggcaacgtcttctcctgtagcgtgatgcacgaggctctgcacaatcactacacccagaaaagcctgtccctgagccctggcaagagaggcaggaagagaagatccggaagcggagccacaaacttctccctgctgaagcaggccggagacgtcgaagagaaccctggccccatggtcctgcagacacaggtgttcatttccctgctgctgtggatctccggagcctacggcgagatcgtcctcacccagagccctggcacactgtccctgagccctggcgagagagccaccctgtcctgtagagccagccaaagcgtctccagcagctatctggcctggtaccagcagaagaggggccaagctcccagactcctgatctatgacgcttcctccagggctaccggcatccccgataggtttagcggcagcggcagcggcaccgatttcaccctcacaatctccaggctggagcctgaggactttgctgtgtattactgccagcagtacggcaggagcaggtggaccttcggacagggaaccaaggtggagatcaaaaggaccgtcgctgctcccagcgtcttcatcttcccccccagcgatgagcagctgaagtccggcacagctagcgtcgtgtgcctcctgaacaatttctatcccagagaggccaaagtccagtggaaggtggataacgccctgcagagcggaaacagccaagaatccgtcaccgaacaggacagcaaggacagcacctacagcctctcctccacactcaccctcagcaaagccgactacgagaagcacaaggtgtacgcttgcgaggtgacccaccagggcctgagctcccctgtgaccaagagcttcaataggggcgagtgctgataa

SEQ ID NO: 68 - protein, 743 aa, synthetic construct; identical to SEQ ID NO: 64
  REGION 1..743: note = Chemically Synthesized, pRD213, 190-FP2A-mouse-Genewiz

SEQ ID NO: 69 - DNA, 2235 bp, synthetic construct
  misc_feature 1..2235: note = Chemically Synthesized, pRD214, 190-FP2A-mouse-Blue heron
  source 1..2235: mol_type = other DNA
atggactggacctggcgaattctgttcttggttgcagcagcgactggtacacacgctcaggtccagctggtcgaatctgggggcggcgtggtacagccagggagatcactgaggctgagttgcgctgcatccggcttcaccttctccaaatacggcatgcattgggtgcggcaagctcccgggaaaggcttggaatgggtggccgtaatttcatacgaagggtctaacaagtattacgccgattctgtcaagggccgctttacaataagccgcgataatagcaaaaacacgctttacctgcagatgaactctctgagagcagaggacactgctgtgtattactgtgctaaatctgggacgcagtactatgatacaactggatacgagtacagaggcctcgaatacttcggatactggggtcagggaacactggtcacggttagtagcgcctccactaagggtccttcagtcttccccctggcacccagtagtaaatccacctccggcggtactgcagctcttggttgcctcgtgaaagactacttccctgaaccagtgaccgtctcctggaactccggtgcactcaccagcggcgtacatacattcccagctgtgttgcaaagttctggtctgtatagcttgtcttccgtggtgactgtccccagctccagcctggggacccaaacttacatttgtaatgtcaaccacaagccgtctaatacaaaggtggacaagcgggttgagccaaagagctgcgacaaaacgcacacctgcccaccttgccccgccccagaagccgcggggggaccctcagtattcctgttcccccccaagcccaaggacactttgatgattagtcgcactcctgaggtcacctgcgtagtagtcgacgtgagccatgaagatcctgaagtgaagtttaactggtacgtcgacggagtggaggtccataacgccaaaaccaagccgcgcgaagaacaatacaactcaacctacagggtggtctctgtgcttacagtcctccatcaggactggctcaacggcaaagaatacaagtgtaaggtctccaacaaggcgctgcccgcgcccatcgagaagacaatcagcaaggccaaaggccagcccagggagccccaggtgtatactctgccacccagtcgagaggagatgacaaaaaatcaagtgtccctcacatgcttggtaaagggtttctatccgtctgacatagccgtggagtgggagtctaatggccaaccagaaaacaactataagactacacctcccgtccttgactctgacggcagcttttttctttactccaaacttacagtcgataagagtcggtggcagcaaggtaatgtgttcagctgcagcgttatgcacgaagcgctgcacaatcactacacgcagaagtccttgagtctgtctcccggaaaacggggccgcaagcggcgcagtggctcaggggccacaaatttctcactcctcaaacaggctggcgacgtggaggaaaaccccgggccaatggtgctgcagacccaagtcttcataagcttgttgctttggatttccggggcctacggagaaatcgttctcactcagtctccaggcacactctcactgtcacctggagagcgcgctaccctcagttgcagagcaagtcaaagcgtgagctcttcctaccttgcttggtaccaacagaagagaggtcaggccccgcgcttgctgatctatgatgcttcatcccgggcgactggtatccccgaccgattctccggctccggcagcggtaccgattttactttgactattagccgccttgaacctgaggacttcgccgtctactactgtcagcagtacggaagaagccgatggacattcgggcagggtaccaaggtagagatcaagcggactgtggcagccccatctgtgtttatcttccctcctagcgatgaacaacttaagtccggaactgcctccgtagtatgccttctgaacaatttttatccacgcgaggcaaaggtgcagtggaaagtggacaacgcccttcagtctggcaattcccaggaaagcgtcacagaacaggatagcaaggactcaacctactccctctccagtactctgacattgtccaaagccgattatgaaaagcacaaagtgtatgcctgcgaagtaacacaccaagggctgtcatctccagtaaccaagagcttcaaccgcggtgagtgttgatga

SEQ ID NO: 70 - protein, 743 aa, synthetic construct; identical to SEQ ID NO: 64
  REGION 1..743: note = Chemically Synthesized, pRD214, 190-FP2A-mouse-Blue heron

SEQ ID NO: 71 - DNA, 2235 bp, synthetic construct
  misc_feature 1..2235: note = Chemically Synthesized, pRD215, 190-FP2A-mammal-DNA2.0
  source 1..2235: mol_type = other DNA
atggactggacctggcgcatcctgtttctcgtcgctgccgccactggtacccatgcccaggtgcaactggtggagtccggtggcggagtggtgcaaccgggtcgcagcctgagactctcatgcgccgcctcaggcttcaccttctcgaagtacgggatgcactgggtcagacaggctcccgggaaaggcctggaatgggtggccgtcatctcgtacgagggctcaaacaagtactatgccgattccgtgaagggacggttcaccatctcccgggacaacagcaagaacaccctgtacctccaaatgaacagcctgagggccgaggacactgcagtgtactactgcgccaagtcgggcactcagtactacgacaccacgggatacgagtacaggggactcgaatacttcggatactggggccagggcacactcgtgaccgtcagctccgcctccactaagggaccttcagtgttccctctggcgccatcctccaagtcgaccagcggtggaactgcagccctgggatgtctcgtgaaggactacttccctgaacctgtcaccgtgtcctggaacagcggagcactgacctccggggtccacactttccccgcggtgttgcagagctccggcctgtactccctgtcgtccgtcgtgacagtgccgtccagctcgttggggacccagacttacatctgcaacgtgaaccacaagccgtccaacactaaagtggacaaacgggtggaacccaagtcgtgtgacaagactcatacttgtccgccatgtcccgctcctgaagcggcgggaggaccttccgtgttcctcttccccccgaagcctaaggacaccctcatgattagcaggaccccggaagtgacctgtgtcgtggtcgacgtgtcacacgaggaccccgaagtgaagtttaattggtacgtggacggcgtcgaagtgcacaacgccaagaccaagccacgcgaagaacagtacaactccacctaccgggtggtgtccgtgcttactgtgctccatcaggactggctcaacggaaaggagtacaagtgcaaagtgtccaacaaagcgctcccggctcctattgaaaagacgatttccaaggccaagggacagcctcgggaacctcaggtctacaccctgcctccttcgcgcgaggagatgaccaagaatcaggtgtccctgacttgcctggtcaagggattctatccatccgacatcgccgtggagtgggagtccaacggccagccggaaaacaactacaagaccaccccacccgtcctggacagcgacgggtccttcttcctgtactcaaagctgactgtggataagtcccgctggcaacagggcaacgtgttctcctgctccgtgatgcacgaggccctgcacaaccactacacccagaagtcgcttagcctctcgccgggcaaacgaggaagaaagcgccggtccggatcaggagcgactaacttctccctcctgaagcaggccggggatgtggaggaaaatccaggacccatggtgctgcaaacccaggtgttcatcagcctcctcctgtggatctccggagcctacggcgaaattgtgctgacccagtcccctgggaccctgtccctgtcccccggagagagagccaccctttcctgccgggcatcgcagagcgtcagctccagctatctggcctggtaccagcaaaagcgcggacaagctccgagactgctgatctacgatgcctcatcacgcgccaccggaatcccggataggttctcaggatcgggctccggtactgatttcaccctgaccatcagccggctggaacctgaggacttcgctgtgtattactgccagcagtacggccggtccagatggactttcggacagggcactaaggtcgaaatcaagcggactgtcgctgcaccgagcgtgttcattttcccaccctccgacgagcagcttaagagcggaactgcttccgtggtgtgccttctgaacaatttctacccccgggaagccaaggtccagtggaaggtcgacaacgccctgcaatccgggaactcccaagaatccgtcaccgaacaggactccaaggacagcacctactccctctcatctaccctgaccctgtctaaggccgactacgagaagcataaggtctacgcctgcgaagtgacacaccaaggcctttcttcccccgtgaccaaatccttcaaccgcggagagtgctgataa

SEQ ID NO: 72 - protein, 743 aa, synthetic construct; identical to SEQ ID NO: 64
  REGION 1..743: note = Chemically Synthesized, pRD215, 190-FP2A-mammal-DNA2.0

SEQ ID NO: 73 - DNA, 2235 bp, synthetic construct
  misc_feature 1..2235: note = Chemically Synthesized, pRD216, 190-FP2A-mouse-Genscript
  source 1..2235: mol_type = other DNA
atggactggacttggcgcatcctctttctcgtcgcagccgcaactggaactcacgctcaggtgcagctcgtcgaatcagggggaggggtggtgcagccaggcaggagcctgaggctgagctgcgctgcttccggcttcaccttttccaagtacggaatgcactgggtgaggcaggctcctggcaagggactggagtgggtggctgtgatctcttacgagggcagcaacaagtactacgccgatagcgtgaagggaaggttcaccatctccagagacaactctaagaacacactgtacctgcagatgaactccctgagggccgaggataccgccgtgtactactgcgctaagtctggcacacagtactacgacaccacaggatacgagtacagaggcctggagtacttcggatactggggccagggaaccctggtgacagtgagctccgcctctacaaagggccccagcgtgtttcccctggctccttctagcaagagcacctccggcggaacagccgctctgggatgtctggtgaaggactacttccctgagccagtgaccgtgtcctggaactctggcgccctgacctctggagtgcacacatttcctgctgtgctgcagtcctctggcctgtacagcctgagctccgtggtgaccgtgccatctagctccctgggaacccagacatacatctgcaacgtgaaccacaagccatccaacacaaaggtggacaagagggtggagcccaagtcttgtgataagacccacacatgccctccctgtccagctcctgaggctgctggcggaccatccgtgttcctgtttccacccaagcctaaggatacactgatgatcagcagaacccccgaggtgacatgcgtggtggtggacgtgtcccacgaggaccccgaggtgaagttcaactggtacgtggacggcgtggaggtgcacaacgccaagaccaagcctagggaggagcagtacaactccacctacagagtggtgtctgtgctgacagtgctgcaccaggattggctgaacggcaaggagtacaagtgcaaggtgtctaacaaggccctgccagctcccatcgagaagaccatcagcaaggctaagggacagccacgggagccacaggtgtacacactgcctccatctcgcgaggagatgaccaagaaccaggtgagcctgacatgtctggtgaagggcttctaccctagcgatatcgctgtggagtgggagtccaacggacagccagagaacaactacaagaccacaccccctgtgctggacagcgatggctccttctttctgtactctaagctgaccgtggacaagagccggtggcagcagggaaacgtgttttcttgcagcgtgatgcacgaggccctgcacaaccactacacccagaagtccctgtctctgagccctggaaagaggggaaggaagaggagatccggctctggagccacaaacttctccctgctgaagcaggctggcgacgtggaggagaaccctggaccaatggtgctgcagacccaggtgtttatcagcctgctgctgtggatctccggagcctacggcgagatcgtgctgacccagagccctggcacactgagcctgtccccaggagagagggccaccctgtcctgtagagcttctcagagcgtgtctagctcctacctggcttggtaccagcagaagaggggacaggctccacgcctgctgatctacgacgcctctagccgggctaccggaatccccgatcgcttctccggctctggaagcggcacagactttaccctgacaatctcccggctggagccagaggatttcgccgtgtactactgccagcagtacggcaggagcagatggacctttggacagggcacaaaggtggagatcaagcgcaccgtggccgctccatccgtgttcatctttccaccctctgatgagcagctgaagagcggcacagcttccgtggtgtgcctgctgaacaacttctaccccagggaggccaaggtgcagtggaaggtggacaacgctctgcagtctggcaacagccaggagtccgtgaccgagcaggactctaaggatagcacatactccctgtcctctaccctgacactgagcaaggccgactacgagaagcacaaggtgtacgcttgcgaagtgacccaccaggggctgagcagtccagtgacaaagtccttcaatagaggggaatgctgataa

SEQ ID NO: 74 - protein, 743 aa, synthetic construct; identical to SEQ ID NO: 64
  REGION 1..743: note = Chemically Synthesized, pRD216, 190-FP2A-mouse-Genscript

SEQ ID NO: 75 - DNA, 2211 bp, synthetic construct
  misc_feature 1..2211: note = Chemically Synthesized, pRD225, 185-mouse-GeneArt
  source 1..2211: mol_type = other DNA
atggactggacttggagaatcctgttcctggtggccgctgctacaggcacacatgctgaagtgcagctggtgcagtctggcgccgaagtgaagaaacctggcgagagcctgagaatcagctgcaaaggcagcggctacagcttcaccagctactggatcacctgggtccgacagatgcctggcaaaggcctggaatggatggccaagttcgaccctagcgacagccagaccaactacagccctagctttcagggccacgtgaccatcagcgtggacaagagcatcagcacagcctacctgcagtggtctagcctgaaggccagcgacaccgccatgtactactgcgccagaagatactgcagcagcagctcctgctacgtggacaactggggacagggcaccctggtcacaatcttctctgcctctacaaagggccccagcgtgttccctctggctcctagctctaagagcacatctggcggaacagctgctctgggctgtctggtcaaggactactttcctgagcctgtgaccgtgtcctggaactctggtgctctgacaagcggcgtgcacacatttccagcagtgctgcagtctagcggcctgtactctctgtctagcgtggtcacagtgcctagcagcagcctgggaacccagacctacatctgcaacgtgaaccacaagcctagcaacaccaaggtcgacaagaaggtggaacccaagagctgcgacaagacccacacctgtcctccatgtcctgctccagaagctgctggcggaccctctgtgttcctgtttcctccaaagcctaaggacaccctgatgatcagcagaacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggctaagggccagccaagagaaccccaggtgtacacactgcctccaagcagggacgagctgaccaagaatcaggtgtccctgacctgcctcgtgaagggcttctacccttccgatatcgccgtggaatgggagtctaacggacagcccgagaacaactacaagacaacccctcctgtgctggacagcgacggctcattcttcctgtacagcaagctgacagtggacaagtccagatggcagcagggcaacgtgttcagctgttctgtgatgcacgaggccctgcacaaccactacacacagaagtccctgtctctgagccccggcaagaggggcagaaagagaagatctggcagcggcgccacaaacttcagtctgctgaaacaggctggcgacgtggaagagaatcccggacctatggtgctgcagacacaggtgttcatcagcctgctgctgtggatctctggcgcctacggaagcagctatgagctgacacagcctcctagcgtgtccgtgtctcctggccagaccgccagaatcacatgtagcggagatgccctgcctaacaagttcgcctactggtacaggcagaagtccggacaggctcccgtgctggtcatctacgaggacaacaagaggcctagcggcatccctgagagattcagcggctctagctctggcaccatggccacactgacaatcagtggcgctcaggtggaagatgaggccgactaccactgttacagcaccgacagcagctctaaccctctgggagtgtttggcggcggaacaaagcttacagtgctgggccaacctaaggctgccccttctgtgacactgttcccacctagctctgaggaactgcaggctaacaaggccacactcgtgtgcctgatcagcgatttctaccctggcgctgtgacagtggcttggaaggctgatagctctcctgtgaaggccggcgtcgagacaacaacacctagcaagcagagcaacaacaaatacgccgccagctcctatctgagcctgacacctgagcagtggaagtcccacagatcctacagctgccaagtgacccacgagggcagcaccgtggaaaagactgtggctcctaccgagtgctcctgatga

SEQ ID NO: 76 - protein, 735 aa, synthetic construct
  REGION 1..735: note = Chemically Synthesized, pRD225, 185-mouse-GeneArt
MDWTWRILFLVAAATGTHAEVQLVQSGAEVKKPGESLRISCKGSGYSFTSYWITWVRQMPGKGLEWMAKFDPSDSQTNYSPSFQGHVTISVDKSISTAYLQWSSLKASDTAMYYCARRYCSSSSCYVDNWGQGTLVTIFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGSSYELTQPPSVSVSPGQTARITCSGDALPNKFAYWYRQKSGQAPVLVIYEDNKRPSGIPERFSGSSSGTMATLTISGAQVEDEADYHCYSTDSSSNPLGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 77 - DNA, 2211 bp, synthetic construct
  misc_feature 1..2211: note = Chemically Synthesized, pRD227, 185-mouse-Synbio
  source 1..2211: mol_type = other DNA
atggactggacttggagaatcctgttcctggtggcagccgctacaggaacacacgcagaagtgcagctggtgcagagcggagcagaagtgaagaagccaggcgagagcctgcggatctcttgcaagggaagcggctacagcttcaccagctactggatcacttgggtgcgccagatgccaggcaagggactggagtggatggccaagttcgaccctagcgacagccagaccaactacagccctagcttccagggccacgtgacaatcagcgtggacaagagcatcagcaccgcctacctccagtggtctagcctgaaggccagcgataccgccatgtactattgcgcccggcggtattgcagcagcagctcttgctacgtggacaattggggccagggaacactggtgaccatcttcagcgccagcaccaagggacctagcgtgtttcctctggccccttctagcaagagcacaagcggaggaacagccgctctgggctgtctggtgaaagactacttccccgagccagtgaccgtgtcttggaactcaggagccctgacaagcggagtgcacacatttccagccgtgctgcagagcagcggactgtactctctgagcagcgtggtgaccgtgccttcttcttctctgggcacccagacctacatctgcaacgtgaaccacaagcccagcaacaccaaggtggacaagaaggtggagcccaagtcttgcgacaagacccacacttgccccccttgtccagctccagaagcagcaggaggacctagcgtgttcctgttccctcctaagcccaaggacaccctgatgatcagccggaccccagaagtgacttgcgtggtggtggacgtgtcccacgaagaccccgaggtcaagttcaattggtacgtggacggagtggaggtgcacaacgctaagaccaagcccagggaggagcagtacaacagcacctacagggtggtgtccgtgctgacagtgctgcaccaggattggctgaacggcaaggagtacaagtgcaaggtgtccaacaaggccctgccagctcccatcgagaagaccatcagcaaggccaagggacagcctagagagcctcaggtgtacaccctgcctccttctagggacgagctgaccaagaaccaggtgtccctgacttgcctcgtgaagggcttctaccccagcgacatcgcagtggagtgggaaagcaacggtcagccagagaacaactacaagaccacccccccagtgctggacagcgacggcagcttcttcctgtacagcaagctgaccgtggacaaaagccgctggcagcagggcaacgtgttctcttgcagcgtgatgcacgaggccctgcacaaccactacacccagaagagcctgtctctgagcccaggcaagaggggcagaaagagaagaagcggcagcggagccaccaacttcagcctgctgaagcaggcaggagacgtggaggagaacccaggacctatggtgctgcagacccaggtgttcatcagcctcctgctgtggatcagcggagcttacggaagcagctacgagctgacacagcctcctagcgtgtccgtgtctccaggacagaccgccagaatcacttgtagcggcgacgccctgcctaacaagttcgcctattggtaccggcagaagagcggacaggctccagtgctggtcatctacgaggacaacaagaggcctagcggcatcccagagaggttcagcggatctagcagcggcacaatggccacactgaccatcagcggagctcaggtggaggacgaggccgactaccattgctacagcaccgacagcagctctaacccactgggcgtgttcggaggaggaacaaagctgaccgtgctgggacagcctaaggcagctcctagcgtgacactgttccctccttctagcgaggagctgcaggctaacaaggccacactcgtctgcctgatcagcgacttctatccaggcgccgtgacagtggcttggaaggccgatagcagcccagtgaaagccggagtggagacaaccacccctagcaagcagagcaacaacaagtacgccgccagcagctacctgagcctgacaccagagcagtggaagagccacaggagctactcttgccaggtcacccacgagggaagcacagtggagaagacagtggcccctacagagtgctcctgataa

SEQ ID NO: 78 - protein, 735 aa, synthetic construct; identical to SEQ ID NO: 76
  REGION 1..735: note = Chemically Synthesized, pRD227, 185-mouse-Synbio

SEQ ID NO: 79 - DNA, 2211 bp, synthetic construct
  misc_feature 1..2211: note = Chemically Synthesized, pRD229, 185-mouse-GeneWiz
  source 1..2211: mol_type = other DNA
atggactggacatggagaatcctcttcctggtggccgccgctaccggcacacatgctgaggtgcagctggtgcagtccggcgctgaagtgaagaaacccggagagagcctgagaatctcctgtaaaggctccggctacagcttcacctcctactggattacctgggtgagacagatgcccggcaaaggactggagtggatggccaagttcgatccctccgacagccagaccaactacagccccagctttcagggacatgtgaccatcagcgtggataagagcatctccaccgcttatctgcagtggagcagcctcaaggccagcgataccgccatgtactactgcgccaggaggtattgctccagctccagctgctacgtcgacaactggggccagggaacactggtcacaatcttctccgcctccaccaagggcccctccgtgtttcctctggctcctagcagcaagtccacctccggaggaacagctgccctcggctgcctcgtgaaagactacttccccgagcccgtgaccgtgtcctggaactccggagccctgacctccggagtccataccttccctgccgtgctccagagcagcggcctctacagcctgagctccgtggtgaccgtcccttccagcagcctgggcacccagacatatatctgcaacgtgaaccataagcctagcaacacaaaggtggacaagaaggtcgaacccaagagctgtgacaagacccacacctgtcctccttgtcccgcccctgaagctgctggcggacctagcgtgttcctgttccctcctaagcccaaggacaccctcatgatctccagaacccctgaagtgacctgcgtggtcgtggacgtgagccacgaggaccccgaggtcaagttcaattggtatgtggacggcgtcgaggtgcacaatgccaagaccaaacccagagaggagcaatacaacagcacctacagagtggtgtccgtgctgacagtgctgcaccaggattggctcaatggcaaggagtacaaatgcaaagtgtccaacaaggccctgcccgctcccatcgaaaagacaatcagcaaggccaagggccagcccagggaacctcaggtctataccctccctcccagcagggatgagctcaccaagaaccaagtgtccctgacctgtctggtcaaaggattctacccctccgatattgctgtcgagtgggagagcaacggccagcccgaaaacaactacaagacaacccctcctgtgctggatagcgacggttctttcttcctgtactccaagctcacagtggacaaatccaggtggcagcagggcaacgtgttctcctgctccgtcatgcatgaggccctgcacaaccactatacccagaagtccctgtccctgagccccggaaaaagaggcagaaaaagaaggtccggctccggcgccacaaacttctccctgctgaagcaggctggcgacgtggaggagaaccccggccctatggtcctccagacccaggtgtttattagcctgctgctgtggatcagcggcgcctatggcagctcctacgaactgacacagccccctagcgtgagcgtgagccctggacagaccgccagaattacctgcagcggcgatgccctgcccaacaagtttgcttactggtacaggcagaaatccggccaagcccctgtgctggtcatttacgaggacaacaagaggcccagcggcattcctgagaggttcagcggcagcagcagcggaaccatggccaccctgacaatcagcggcgcccaagtggaagacgaggctgactaccactgttacagcacagactcctcctccaatcctctcggcgtgttcggcggcggcacaaaactgacagtgctcggacagcctaaggccgcccctagcgtgacactgtttcctccttcctccgaggaactgcaggccaacaaggccacactggtgtgtctgatctccgacttctaccccggcgctgtgaccgtggcttggaaggccgattccagccccgtgaaggctggcgtcgagaccacaacccccagcaagcagagcaacaacaagtatgctgcctcctcctacctgtccctcacacccgaacagtggaagagccataggtcctacagctgccaggtgacacacgaaggcagcacagtggaaaagaccgtggcccctaccgaatgctcctgataa

SEQ ID NO: 80 - protein, 735 aa, synthetic construct; identical to SEQ ID NO: 76
  REGION 1..735: note = Chemically Synthesized, pRD229, 185-mouse-GeneWiz

SEQ ID NO: 81 - DNA, 2211 bp, synthetic construct
  misc_feature 1..2211: note = Chemically Synthesized, pRD231, 185-mouse-GenScript
  source 1..2211: mol_type = other DNA
atggattggacatggaggattctgtttctggtcgccgccgctactggaactcacgccgaagtgcagctggtgcagtcaggagccgaagtgaagaagccaggcgagagcctgcggatctcctgtaagggaagcggctactcctttacatcttactggatcacctgggtgcgccagatgccaggaaagggcctggagtggatggccaagttcgacccttctgatagccagacaaactactccccatcttttcagggccacgtgacaatcagcgtggacaagagcatctccaccgcctacctgcagtggagctccctgaaggcctccgataccgctatgtactactgcgctaggagatactgttctagctcctcttgctacgtggacaactggggacagggcacactggtgaccatcttctctgccagcacaaagggacccagcgtgtttccactggctcccagctccaagtccacatctggcggaaccgccgctctgggatgtctggtgaaggattacttccccgagcctgtgaccgtgagctggaactccggagccctgacaagcggcgtgcacacctttcccgctgtgctgcagtctagcggactgtactccctgtcctctgtggtgacagtgcctagctcctctctgggcacacagacctacatctgtaacgtgaaccacaagccttccaacaccaaggtggacaagaaggtggagccaaagtcttgcgataagacacacacctgccctccctgtccagctccagaggctgctggcggaccatccgtgttcctgtttccacccaagcctaaggacaccctgatgatcagcaggacaccagaggtgacctgcgtggtggtggacgtgtcccacgaggaccccgaggtgaagttcaactggtacgtggatggcgtggaggtgcacaacgccaagacaaagccaagggaggagcagtacaactctacatacagagtggtgagcgtgctgaccgtgctgcaccaggactggctgaacggaaaggagtacaagtgtaaggtgtctaacaaggccctgcctgctccaatcgagaagacaatcagcaaggctaagggacagcctcgggagccacaggtgtacaccctgcctccatctcgcgacgagctgacaaagaaccaggtgagcctgacctgcctggtgaagggcttctacccctccgatatcgctgtggagtgggagtctaacggccagcctgagaacaactacaagaccacaccccctgtgctggactctgatggaagcttctttctgtacagcaagctgaccgtggacaagtccagatggcagcagggcaacgtgttcagctgttccgtgatgcacgaggccctgcacaaccactacacacagaagtctctgagcctgtcccctggaaagaggggaagaaagcggcgctctggaagcggagccaccaactttagcctgctgaagcaggctggagatgtggaggagaaccccggccctatggtgctgcagacacaggtgttcatctccctgctgctgtggatctctggagcctacggcagctcctacgagctgacccagccaccctccgtgtctgtgagccctggacagacagctaggatcacctgctctggcgacgccctgccaaacaagtttgcttactggtacagacagaagtccggacaggcccccgtgctggtcatctacgaggataacaagcggccctctggcatccctgagaggttcagcggatctagctccggcacaatggctacactgaccatctccggagctcaggtggaggacgaggctgattaccactgttactctaccgactctagctccaaccctctgggagtgttcggcggaggcacaaagctgaccgtgctgggacagccaaaggctgctccaagcgtgaccctgtttcctccatctagcgaggagctgcaggccaacaaggctacactggtgtgcctgatctccgacttctaccctggagctgtgaccgtggcttggaaggctgattcctctccagtgaaggctggcgtggagacaacaaccccctccaagcagtctaacaacaagtacgccgctagctcctacctgagcctgaccccagagcagtggaagtcccaccggtcctactcttgccaggtcactcacgaaggaagcactgtggaaaaaactgtcgcccctaccgaatgttcttgataa

SEQ ID NO: 82 - protein, 735 aa, synthetic construct; identical to SEQ ID NO: 76
  REGION 1..735: note = Chemically Synthesized, pRD231, 185-mouse-GenScript

SEQ ID NO: 83 - DNA, 2211 bp, synthetic construct
  misc_feature 1..2211: note = Chemically Synthesized, pRD233, 185-mouse-Blue Heron
  source 1..2211: mol_type = other DNA
atggactggacctggaggatactgtttctggtggctgccgcaaccggaacacatgccgaagtacagctggttcagtctggagcggaggtcaagaagcccggggaaagtctgaggatctcttgcaaggggtcaggttatagtttcacaagttattggatcacttgggtcagacaaatgccggggaagggactggagtggatggctaagtttgatccctccgattctcagactaattactccccgtcatttcagggtcatgtgactatcagcgtcgataaatctatttcaactgcctacctgcaatggagcagcttgaaggcttccgataccgccatgtattactgtgcacggaggtattgttccagctcctcttgttacgtggataactgggggcaggggacattggtaacgattttttcagcttctacaaagggcccctctgtctttcctctcgctccttctagcaaatccacttctgggggaaccgctgcactgggatgcctcgtgaaagactacttccccgaacccgtgaccgtgtcatggaactcaggtgcccttaccagcggagtccacacgttcccggctgtgcttcagagttccggtctgtattccctctcctctgtggtgacggtgccctccagctcactgggaacgcagacatatatctgtaacgtgaaccacaaaccctccaataccaaggtcgacaagaaggtggagccgaaatcctgtgataagacccatacctgtcctccatgccccgccccagaagcagctggaggaccctctgtgttcttgtttccccccaaaccgaaagatactttgatgatctcccggaccccagaagtcacgtgtgtagtggtcgatgttagtcatgaagaccccgaagtgaaattcaactggtacgtagacggcgtggaggtccataatgctaagacgaagccacgagaagaacagtataattccacctaccgggttgtcagcgtgcttaccgttttgcatcaggattggttgaatggaaaggaatacaaatgcaaggtgtccaataaggccctgcccgctcctatcgagaagacaattagtaaggcaaaaggccaacctcgcgagccccaggtgtataccctccctccctccagggacgaactgacaaagaaccaagtgagcctgacatgtctggtcaagggattttatccttcagatatcgctgtggagtgggaaagcaatggccagccagaaaacaactacaaaacaactccgcccgtcctggactctgacggttcctttttcctgtactctaagctgactgtggataagtcaagatggcagcaggggaatgtgttttcttgtagcgttatgcacgaggctctgcacaaccattatacgcagaagagtttgagcctgagtcctggtaaaaggggccggaaacgcaggtctggatctggggctactaacttcagccttttgaaacaagccggggatgtggaggaaaacccagggcccatggtcctgcagactcaggtttttatcagtctgctgctctggatttcaggcgcttacggtagcagctacgagctgacccagcccccttcagtgtccgtctcaccaggacagaccgcgaggatcacttgcagtggggatgccctgccgaataagtttgcgtattggtacagacaaaagtccggtcaggcaccagtcctggtgatttatgaagacaacaagcgaccaagcggcatccctgagcgcttctccggttccagcagcgggaccatggccacactgacaatcagtggggcccaggtcgaggacgaggccgactaccactgctattctaccgatagctcttcaaatccattgggagtgtttggcggtggaacaaaactcaccgtgctggggcagccaaaggcagctccaagtgtcactctttttccacctagcagtgaagagctccaggccaacaaagcaaccctggtgtgtcttatcagcgatttttaccctggggcggtgacagtggcctggaaagccgattccagccccgtgaaagccggtgtcgaaaccactactcctagcaagcagagcaacaataaatatgccgcgagttcctacctgagccttactccagagcagtggaagtcacaccggtcctatagttgtcaggttacacacgaaggaagtactgttgaaaagactgttgctccgacagaatgcagctgataa

SEQ ID NO: 84 - protein, 735 aa, synthetic construct; identical to SEQ ID NO: 76
  REGION 1..735: note = Chemically Synthesized, pRD233, 185-mouse-Blue Heron

SEQ ID NO: 85 - DNA, 2211 bp, synthetic construct
  misc_feature 1..2211: note = Chemically Synthesized, pRD234, 185-mammal-DNA2.0
  source 1..2211: mol_type = other DNA
atggactggacttggcggattttgttcctggtggcggcggctactggaactcatgcagaagtgcagcttgtgcagtccggcgctgaagtcaagaagcctggagagtccctgagaatcagctgcaagggcagcggctactccttcacctcgtactggatcacttgggtcagacaaatgccgggaaagggactggaatggatggctaaattcgacccgtcggacagccagactaactacagcccgtcgttccaaggacatgtcaccatctccgtggacaaatcgatcagcaccgcgtacctccagtggagctcactcaaagcatccgacaccgcgatgtactactgcgcccgccgctactgctcgtcctcgtcctgctacgtggacaattggggacagggtactcttgtgacaatcttctccgcctccaccaagggcccctcagtgttccccctggcaccatcctctaagtccacctccggaggcaccgccgccttgggttgcctggtcaaggactacttcccggaacctgtgaccgtgtcctggaacagcggggcactgacctccggcgtgcatacttttcccgccgtcctgcaatcttccggcctgtactcactgtcatcagtggtcaccgtgccctcgtcctccctgggcacccagacttacatctgtaacgtgaaccataagccctccaacaccaaagtggacaagaaagtggagccaaagtcgtgtgacaagactcacacttgcccgccgtgcccggccccggaagccgccggtggaccgagcgtgtttctcttcccacccaagccgaaggataccctgatgatttcgcggacccctgaagtgacctgtgtggtggtcgacgtgtcccacgaggaccccgaagtcaagttcaattggtacgtggacggtgtcgaagtgcacaacgccaagacgaagcctcgcgaggaacagtacaacagcacttaccgggtcgtgtccgtcctcaccgtgctgcaccaagattggctcaacgggaaggagtacaagtgcaaagtgtcaaataaggccctgccggccccgattgaaaagaccattagcaaggccaagggacagcctagggaacctcaagtgtacacgctgcccccgtcgcgggacgagctgaccaagaaccaagtgtcgctgacttgccttgtgaagggattctacccgtccgacattgccgtggagtgggagtccaacggacagcccgaaaacaactacaagaccacccctcctgtgctggactctgatggatcattcttcctttactcgaaactcaccgtggacaagtcccgctggcaacagggaaacgtgttcagctgctccgtgatgcacgaggccctgcacaaccactatacccagaagtccctgtcactgagccccgggaagcgcggcaggaagcggagaagcggaagcggagctacaaacttttcgctgctgaagcaggccggcgatgtggaagaaaacccagggcctatggtgctgcagactcaagtgtttatctccctgctcctgtggatctccggagcgtatgggtccagctacgagctcacccagcctccttccgtgtcggtgtcaccaggacagaccgcccggattacctgttccggggatgccctccccaacaagttcgcctactggtaccgccagaagtccggccaggctccagtgcttgtgatctacgaggacaacaagcggccttctggcatccccgagcggttctccgggtcctcctccggcaccatggcaaccctgaccatctcgggagctcaggtcgaagatgaagccgactatcactgctactcgaccgatagctcctcaaacccgttgggggtctttggcggaggaaccaagctgactgtgctgggacagccgaaggccgcgccgtccgtcaccctgttcccgccgagcagcgaggaactccaggccaacaaggccactctcgtgtgcctgatttccgacttctaccctggtgccgtgactgtggcgtggaaggccgactcgtcgccggtcaaggccggcgtggagactaccaccccgtcaaaacagagcaacaataagtacgccgcctcctcctatttgtcactgactcccgagcagtggaagtcccaccggtcctactcatgccaagtcacccatgaagggtccaccgtggaaaagactgtggcccccactgagtgttcgtaatga

SEQ ID NO: 86 - protein, 735 aa, synthetic construct; identical to SEQ ID NO: 76
  REGION 1..735: note = Chemically Synthesized, pRD234, 185-mammal-DNA2.0

SEQ ID NO: 87 - DNA, 2235 bp, synthetic construct
  misc_feature 1..2235: note = Chemically Synthesized, pGX9382, 190
  source 1..2235: mol_type = other DNA
atggattggacctggagaatcctgttcctggtggcagcagcaaccggaacacacgcacaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccgcctccacaaagggaccaagcgtgttcccactggcaccttctagcaagtctaccagcggcggcacagccgccctgggatgtctggtgaaggattacttccctgagccagtgaccgtgagctggaactccggcgccctgacctccggagtgcacacatttcctgccgtgctgcagtcctctggcctgtactctctgagctccgtggtgaccgtgccatctagctccctgggcacccagacatatatctgcaacgtgaatcacaagccaagcaatacaaaggtggacaagagggtggagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagaggggaaggaagaggagaagcggctccggcgccacaaacttcagcctgctgaagcaggcaggcgacgtggaggagaatcctggaccaatggtgctgcagacccaggtgtttatctctctgctgctgtggatcagcggagcatacggagagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaagaggaccgtggcagcaccaagcgtgttcatctttccaccctccgatgagcagctgaagtctggcacagccagcgtggtgtgcctgctgaacaatttctaccccagggaggccaaggtgcagtggaaggtggacaacgccctgcagtccggcaattctcaggagagcgtgaccgagcaggactccaaggattctacatatagcctgtcctctaccctgacactgtccaaggccgactacgagaagcacaaggtgtatgcatgcgaggtgacccaccagggcctgagctccccagtgacaaagagctttaaccgcggcgagtgttgataa

SEQ ID NO: 88 - protein, 743 aa, synthetic construct; identical to SEQ ID NO: 64
  REGION 1..743: note = Chemically Synthesized, pGX9382, 190

SEQ ID NO: 89 - DNA, 1518 bp, synthetic construct
  misc_feature 1..1518: note = Chemically Synthesized, pGX93100, 190.scFv_Fc.VH.G4S3.VL
  source 1..1518: mol_type = other DNA
atggattggacctggagaatcctgttcctggtggcagcagcaaccggaacacacgcacaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccggcggcggcggctccggcggcggcggcagcggcggcggcggcagcgagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaaggagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagtgataa

SEQ ID NO: 90 - protein, 504 aa, synthetic construct
  REGION 1..504: note = Chemically Synthesized, pGX93100, 190.scFv_Fc.VH.G4S3.VL
MDWTWRILFLVAAATGTHAQVQLVESGGGVVQPGRSLRLSCAASGFTFSKYGMHWVRQAPGKGLEWVAVISYEGSNKYYADSVKGRFTISRDNSKNTLYLQMNSLRAEDTAVYYCAKSGTQYYDTTGYEYRGLEYFGYWGQGTLVTVSSGGGGSGGGGSGGGGSEIVLTQSPGTLSLSPGERATLSCRASQSVSSSYLAWYQQKRGQAPRLLIYDASSRATGIPDRFSGSGSGTDFTLTISRLEPEDFAVYYCQQYGRSRWTFGQGTKVEIKEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1521 bp, synthetic construct
  misc_feature 1..1521: note = Chemically Synthesized, pGX93101, 190.scFv_Fc.VL.G4S3.VH
  source 1..1521: mol_type = other DNA
atggtgctgcagacccaggtgtttatctctctgctgctgtggatcagcggagcatacggagagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaagggcggcggcggctccggcggcggcggcagcggcggcggcggcagccaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccgagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagtgataa

SEQ ID NO: 92 - protein, 505 aa, synthetic construct
  REGION 1..505: note = Chemically Synthesized, pGX93101, 190.scFv_Fc.VL.G4S3.VH
MVLQTQVFISLLLWISGAYGEIVLTQSPGTLSLSPGERATLSCRASQSVSSSYLAWYQQKRGQAPRLLIYDASSRATGIPDRFSGSGSGTDFTLTISRLEPEDFAVYYCQQYGRSRWTFGQGTKVEIKGGGGSGGGGSGGGGSQVQLVESGGGVVQPGRSLRLSCAASGFTFSKYGMHWVRQAPGKGLEWVAVISYEGSNKYYADSVKGRFTISRDNSKNTLYLQMNSLRAEDTAVYYCAKSGTQYYDTTGYEYRGLEYFGYWGQGTLVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1527 bp, synthetic construct
  misc_feature 1..1527: note = Chemically Synthesized, pGX93102, 190.scFv_Fc VH.Whitlow.VL
  source 1..1527: mol_type = other DNA
atggattggacctggagaatcctgttcctggtggcagcagcaaccggaacacacgcacaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccggcagcacctctggctccggcaagcccggctctggcgagggcagcaccaagggcgagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaaggagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagtgataa

SEQ ID NO: 94 - protein, 507 aa, synthetic construct
  REGION 1..507: note = Chemically Synthesized, pGX93102, 190.scFv_Fc VH.Whitlow.VL
MDWTWRILFLVAAATGTHAQVQLVESGGGVVQPGRSLRLSCAASGFTFSKYGMHWVRQAPGKGLEWVAVISYEGSNKYYADSVKGRFTISRDNSKNTLYLQMNSLRAEDTAVYYCAKSGTQYYDTTGYEYRGLEYFGYWGQGTLVTVSSGSTSGSGKPGSGEGSTKGEIVLTQSPGTLSLSPGERATLSCRASQSVSSSYLAWYQQKRGQAPRLLIYDASSRATGIPDRFSGSGSGTDFTLTISRLEPEDFAVYYCQQYGRSRWTFGQGTKVEIKEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1530 bp, synthetic construct
  misc_feature 1..1530: note = Chemically Synthesized, pGX93103, 190.scFv_Fc.VL.Whitlow.VH
  source 1..1530: mol_type = other DNA
atggtgctgcagacccaggtgtttatctctctgctgctgtggatcagcggagcatacggagagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaagggcagcacctctggctccggcaagcccggctctggcgagggcagcaccaagggccaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccgagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagtgataa

SEQ ID NO: 96 - protein, 508 aa, synthetic construct
  REGION 1..508: note = Chemically Synthesized, pGX93103, 190.scFv_Fc.VL.Whitlow.VH
MVLQTQVFISLLLWISGAYGEIVLTQSPGTLSLSPGERATLSCRASQSVSSSYLAWYQQKRGQAPRLLIYDASSRATGIPDRFSGSGSGTDFTLTISRLEPEDFAVYYCQQYGRSRWTFGQGTKVEIKGSTSGSGKPGSGEGSTKGQVQLVESGGGVVQPGRSLRLSCAASGFTFSKYGMHWVRQAPGKGLEWVAVISYEGSNKYYADSVKGRFTISRDNSKNTLYLQMNSLRAEDTAVYYCAKSGTQYYDTTGYEYRGLEYFGYWGQGTLVTVSSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 2211 bp, synthetic construct
  misc_feature 1..2211: note = Chemically Synthesized, pGX93134, 185
  source 1..2211: mol_type = other DNA
atggactggacttggagaatcctgttcctggtcgccgcagcaaccgggacccacgccgaggtgcagctggtgcagagcggggcagaagtgaagaagccaggcgagagcctgcggatctcctgtaagggcagcggctactcctttacatcttattggatcacctgggtgcgccagatgccaggcaagggcctggagtggatggccaagttcgacccttctgatagccagacaaactactccccatcttttcagggccacgtgacaatcagcgtggacaagagcatctccaccgcctatctgcagtggagctccctgaaggcctccgataccgccatgtactattgcgccaggagatactgttctagctcctcttgctatgtggacaattggggccagggcacactggtgaccatcttctctgccagcacaaagggaccaagcgtgtttccactggcacccagctccaagtccacatctggcggcaccgccgccctgggatgtctggtgaaggattacttccccgagcctgtgaccgtgagctggaactccggcgccctgacaagcggagtgcacacctttccagccgtgctgcagtctagcggcctgtactccctgtcctctgtggtgacagtgcctagctcctctctgggcacacagacctatatctgtaacgtgaatcacaagccttccaataccaaggtggacaagaaggtggagccaaagtcttgcgataagacacacacctgccctccctgtccagcaccagaggcagccggcggaccatccgtgttcctgtttccacccaagcctaaggacaccctgatgatcagcaggacaccagaggtgacctgcgtggtggtggacgtgtcccacgaggaccccgaggtgaagttcaactggtacgtggatggcgtggaggtgcacaatgccaagacaaagccaagggaggagcagtacaactctacatatagagtggtgagcgtgctgaccgtgctgcaccaggactggctgaacggcaaggagtataagtgtaaggtgtctaataaggccctgcctgccccaatcgagaagacaatcagcaaggcaaagggacagcctcgggagccacaggtgtacaccctgcctccatctcgcgacgagctgacaaagaaccaggtgagcctgacctgcctggtgaagggcttttatccctccgatatcgccgtggagtgggagtctaatggccagcctgagaacaattacaagaccacaccccctgtgctggactctgatggcagcttctttctgtatagcaagctgaccgtggacaagtcccggtggcagcagggcaacgtgttcagctgttccgtgatgcacgaggccctgcacaatcactacacacagaagtctctgagcctgtcccctggcaagaggggaagaaagcggcgctctggcagcggagcaaccaactttagcctgctgaagcaggcaggcgatgtggaggagaatccaggacctatggtgctgcagacacaggtgttcatctccctgctgctgtggatctctggcgcctacggcagctcctatgagctgacccagccaccctccgtgtctgtgagccctggccagacagcaaggatcacctgcagcggcgacgcactgccaaacaagtttgcctactggtatagacagaagtccggacaggcacccgtgctggtcatctacgaggataataagcggccctctggcatccctgagaggttcagcggctctagctccggcacaatggccacactgaccatctccggagcacaggtggaggacgaggcagattaccactgttattctaccgactctagctccaaccctctgggcgtgttcggcggcggaacaaagctgaccgtgctgggacagccaaaggcagcaccaagcgtgaccctgtttcctccatctagcgaggagctgcaggccaataaggccacactggtgtgcctgatctccgacttctaccctggagcagtgaccgtggcatggaaggccgattcctctccagtgaaggccggcgtggagacaacaaccccctccaagcagtctaacaataagtacgccgccagctcctatctgagcctgaccccagagcagtggaagtcccacaggtcctattcttgtcaggtcacccatgaaggctcaacagtggagaaaacagtcgcccctacagaatgctcatgataa

SEQ ID NO: 98 - protein, 735 aa, synthetic construct; identical to SEQ ID NO: 76
  REGION 1..735: note = Chemically Synthesized, pGX93134, 185

SEQ ID NO: 99 - DNA, 1497 bp, synthetic construct
  misc_feature 1..1497: note = Chemically Synthesized, pGX93129, 185.scFv_Fc.VH.G4S3.VL
  source 1..1497: mol_type = other DNA
atggattggacatggagaatcctgttcctggtcgccgccgcaactgggacccacgccgaagtgcagctggtgcagtctggagccgaagtgaagaagccaggcgagtctctgagaatcagctgcaagggcagcggctactctttcacatcctactggatcacctgggtgcgccagatgccaggcaagggcctggagtggatggccaagttcgacccatctgatagccagacaaactacagcccctccttccagggccacgtgacaatctccgtggacaagtccatcagcacagcctatctgcagtggtcttccctgaaggcctctgacaccgccatgtactattgcgcccggagatattgcagctcttccagctgttacgtggacaactggggccagggcaccctggtgacaatcttctctggcggcggcggctctggcggcggcggcagcggcggcggcggctccagctcctatgagctgacccagcccccatccgtgagcgtgtctccaggccagacagcccggatcacatgtagcggcgacgccctgcctaataagtttgcctactggtacaggcagaagagcggccaggccccagtgctggtaatctacgaggacaacaagcgcccatctggcatccccgagagattttctggctcttccagcggcaccatggccaccctgacaatctccggcgcccaggtggaggatgaggccgattatcactgctacagcacagattccagctccaatcccctgggcgtgtttggcggcggcaccaagctgaccgtgctggagccaaagagctccgataagacccacacctgtccaccttgccccgccccagaggccgccggcggcccaagcgtgttcctgttcccacctaagcccaaggacaccctgatgatctccagaacccctgaggtgacatgcgtggtggtggacgtgtctcacgaggacccagaggtgaagttcaactggtatgtggacggcgtggaggtgcacaacgccaagacaaagcctagagaggagcagtacaacagcacctacagagtggtgtctgtgctgacagtgctgcaccaggactggctgaacggcaaggagtacaagtgtaaggtgtccaataaggccctgccagcccctatcgagaagaccatctctaaggccaagggccagccaagagagccacaggtgtataccctgccaccctctagagacgagctgacaaagaaccaggtgtctctgacatgtctggtgaagggcttctacccctctgatatcgccgtggagtgggagtctaatggccagccagagaataactacaagaccacaccacctgtgctggactccgatggcagctttttcctgtacagcaagctgacagtggataagtccagatggcagcagggcaacgtgtttagctgctccgtgatgcacgaagcactgcacaaccactacacccagaagtccctgagcctgtcacccggcaagtgataa

SEQ ID NO: 100 - protein, 497 aa, synthetic construct
  REGION 1..497: note = Chemically Synthesized, pGX93129, 185.scFv_Fc.VH.G4S3.VL
MDWTWRILFLVAAATGTHAEVQLVQSGAEVKKPGESLRISCKGSGYSFTSYWITWVRQMPGKGLEWMAKFDPSDSQTNYSPSFQGHVTISVDKSISTAYLQWSSLKASDTAMYYCARRYCSSSSCYVDNWGQGTLVTIFSGGGGSGGGGSGGGGSSSYELTQPPSVSVSPGQTARITCSGDALPNKFAYWYRQKSGQAPVLVIYEDNKRPSGIPERFSGSSSGTMATLTISGAQVEDEADYHCYSTDSSSNPLGVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - DNA, 1500 bp, synthetic construct
  misc_feature 1..1500: note = Chemically Synthesized, pGX93130, 185.scFv_Fc.VL.G4S3.VH
  source 1..1500: mol_type = other DNA
atggtcctgcagactcaggtgtttatttcactgctgctgtggatttccggcgcttatgggtcatcttacgaactgactcagcctccttccgtgagcgtgtctccaggccagacagccagaatcacatgctccggcgacgccctgccaaacaagtttgcctactggtataggcagaagtccggccaggccccagtgctggtaatctatgaggacaacaagcggccaagcggcatcccagagcggtttagcggctccagctctggcaccatggccaccctgacaatctccggcgcccaggtggaggatgaggccgattatcactgttactccacagatagctcctccaatccactgggcgtgttcggcggcggcaccaagctgaccgtgctgggcggcggcggctccggcggcggcggctctggcggcggcggctccgaggtgcagctggtgcagtctggcgccgaggtgaagaagccaggcgagtccctgcgcatcagctgcaagggctctggctatagcttcacaagctactggatcacctgggtgcggcagatgccaggcaagggcctggagtggatggccaagtttgacccctctgatagccagaccaattactctcccagcttccagggccacgtgacaatcagcgtggacaagtccatcagcacagcctacctgcagtggtctagcctgaaggccagcgatacagccatgtattattgcgcccggagatactgttccagctctagctgttacgtggacaattggggccagggcacactggtgacaatcttttccgagccaaagtcttccgataagacccacacctgtccaccctgtcctgccccagaggccgccggcggccccagcgtgttcctgtttcctccaaagcccaaggacaccctgatgatctccagaacaccagaggtgacctgcgtggtggtggacgtgtctcacgaggacccagaggtgaagtttaactggtacgtggacggcgtggaggtgcacaacgccaagacaaagcctagagaggagcagtacaatagcacctacagagtggtgtctgtgctgacagtgctgcaccaggattggctgaacggcaaggagtacaagtgtaaggtgtccaataaggccctgcccgcccctatcgagaagaccatctctaaggccaagggccagcctagagagccacaggtgtatacactgcccccatccagggacgagctgaccaagaaccaggtgtccctgacatgtctggtgaagggcttttaccctagcgatatcgccgtggagtgggagtctaacggccagcctgagaataactacaagaccacaccacctgtgctggactctgacggcagctttttcctgtacagcaagctgacagtggacaagagcaggtggcagcagggcaacgtgttcagctgctccgtgatgcacgaagcactgcacaaccactacactcagaaatccctgtcactgtcccctggaaaatgataa

SEQ ID NO: 102 - protein, 498 aa, synthetic construct
  REGION 1..498: note = Chemically Synthesized, pGX93130, 185.scFv_Fc.VL.G4S3.VH
MVLQTQVFISLLLWISGAYGSSYELTQPPSVSVSPGQTARITCSGDALPNKFAYWYRQKSGQAPVLVIYEDNKRPSGIPERFSGSSSGTMATLTISGAQVEDEADYHCYSTDSSSNPLGVFGGGTKLTVLGGGGSGGGGSGGGGSEVQLVQSGAEVKKPGESLRISCKGSGYSFTSYWITWVRQMPGKGLEWMAKFDPSDSQTNYSPSFQGHVTISVDKSISTAYLQWSSLKASDTAMYYCARRYCSSSSCYVDNWGQGTLVTIFSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - DNA, 1506 bp, synthetic construct
  misc_feature 1..1506: note = Chemically Synthesized, pGX93131, 185.scFv_Fc VH.Whitlow.VL
  source 1..1506: mol_type = other DNA
atggactggacatggagaatcctgttcctggtcgccgccgctaccgggacccacgcagaagtgcagctggtgcagagtggagccgaagtgaagaagccaggcgagtctctgagaatcagctgcaagggcagcggctattcctttacaagctactggatcacctgggtgcggcagatgcccggcaagggcctggagtggatggccaagttcgatccatccgatagccagaccaattacagcccttctttccagggccacgtgacaatctccgtggacaagtccatcagcacagcctacctgcagtggtcctctctgaaggccagcgacaccgccatgtactactgtgcccggagatattgctcctcttccagctgctatgtggacaattggggccagggcaccctggtgaccatcttcagcggctctacaagcggctctggcaagccaggctctggcgagggctctaccaagggctccagctatgagctgacccagcctccatccgtgagcgtgtccccaggccagacagccagaatcacctgtagcggcgacgccctgcctaacaagtttgcctactggtataggcagaagtctggccaggcccctgtgctggtaatctacgaggataacaagaggccatccggcatccctgagagattttctggctcctctagcggcaccatggccaccctgacaatctctggcgcccaggtggaggatgaggccgactaccactgctactccacagactcttccagcaaccctctgggcgtgttcggcggcggcaccaagctgacagtgctggagcccaagagctctgacaagacccacacctgccctccatgtccagccccagaggccgccggcggcccaagcgtgttcctgtttccacctaagccaaaggacaccctgatgatctctcggaccccagaggtgacatgcgtggtggtggacgtgtcccacgaggaccctgaggtgaagtttaattggtatgtggatggcgtggaggtgcacaatgccaagaccaagccacgggaggagcagtataacagcacctacagagtggtgtctgtgctgaccgtgctgcaccaggactggctgaacggcaaggagtacaagtgtaaggtgtccaacaaggccctgcccgcccctatcgagaagaccatctctaaggccaagggccagccaagagagccacaggtgtataccctgccaccctccagagatgagctgacaaagaatcaggtgtctctgacctgtctggtgaagggcttctacccaagcgatatcgccgtggagtgggagtctaacggccagcctgagaacaactataagaccacacctcccgtgctggattccgacggctctttcttcctgtacagcaagctgaccgtggataagagcagatggcagcagggcaacgtgttctcctgtagcgtgatgcacgaagcactgcacaaccattacacccagaagtccctgagcctgagccctggcaaatgataa

SEQ ID NO: 104 - protein, 500 aa, synthetic construct
  REGION 1..500: note = Chemically Synthesized, pGX93131, 185.scFv_Fc VH.Whitlow.VL
MDWTWRILFLVAAATGTHAEVQLVQSGAEVKKPGESLRISCKGSGYSFTSYWITWVRQMPGKGLEWMAKFDPSDSQTNYSPSFQGHVTISVDKSISTAYLQWSSLKASDTAMYYCARRYCSSSSCYVDNWGQGTLVTIFSGSTSGSGKPGSGEGSTKGSSYELTQPPSVSVSPGQTARITCSGDALPNKFAYWYRQKSGQAPVLVIYEDNKRPSGIPERFSGSSSGTMATLTISGAQVEDEADYHCYSTDSSSNPLGVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 1509 bp, synthetic construct
  misc_feature 1..1509: note = Chemically Synthesized, pGX93132, 185.scFv_Fc.VL.Whitlow.VH
  source 1..1509: mol_type = other DNA
atggtcctgcagactcaggtgtttatttcactgctgctgtggatttccggggcatacgggtcctcatacgaactgactcagcctcccagcgtgtccgtgtctcctggccagaccgccaggatcacctgcagcggcgatgccctgccaaacaagttcgcctattggtacaggcagaagtccggccaggccccagtgctggtaatctacgaggacaataagcggccttctggcatccctgagaggttcagcggctcttcctctggcacaatggccacactgaccatctctggcgcccaggtggaggatgaggccgactaccactgctacagcaccgattcctctagcaatcctctgggcgtgtttggcggcggcaccaagctgacagtgctgggctccacctctggctccggcaagccaggctctggcgagggctctacaaagggcgaggtgcagctggtgcagtccggcgccgaggtgaagaagcctggcgagtctctgcggatcagctgcaagggcagcggctactccttcacctcttactggatcacctgggtgcggcagatgccaggcaagggcctggagtggatggccaagttcgaccctagcgacagccagaccaactacagcccttcttttcagggccacgtgacaatcagcgtggataagtctatctccaccgcctatctgcagtggagctccctgaaggcctctgataccgccatgtattactgcgccagacgctactgttctagcagctcctgctatgtggacaactggggccagggcacactggtgaccatcttcagcgagcctaagagctccgataagacccacacctgccctccctgcccagcccccgaggccgccggcggcccatccgtgtttctgttccctccaaagcccaaggataccctgatgatcagccgcacccctgaggtgacatgcgtggtggtggacgtgtctcacgaggacccagaggtgaagttcaactggtacgtggatggcgtggaggtgcacaatgccaagaccaagcccagagaggagcagtacaattccacctacagagtggtgtctgtgctgaccgtgctgcaccaggattggctgaacggcaaggagtacaagtgtaaggtgtccaataaggccctgccagcccccatcgagaagaccatcagcaaggccaagggccagccaagagagcctcaggtgtataccctgcccccatctcgggacgagctgaccaagaaccaggtgtccctgacatgtctggtgaagggcttttatccatccgacatcgccgtggagtgggagagcaatggccagcccgagaacaattacaagacaacccctccagtgctggatagcgacggctcctttttcctgtattccaagctgaccgtggataagtccagatggcagcagggcaacgtgttctcctgctctgtgatgcacgaagccctgcacaaccattacacccagaaatccctgtccctgtcccccggaaaatgataa

SEQ ID NO: 106 - protein, 501 aa, synthetic construct
  REGION 1..501: note = Chemically Synthesized, pGX93132, 185.scFv_Fc.VL.Whitlow.VH
MVLQTQVFISLLLWISGAYGSSYELTQPPSVSVSPGQTARITCSGDALPNKFAYWYRQKSGQAPVLVIYEDNKRPSGIPERFSGSSSGTMATLTISGAQVEDEADYHCYSTDSSSNPLGVFGGGTKLTVLGSTSGSGKPGSGEGSTKGEVQLVQSGAEVKKPGESLRISCKGSGYSFTSYWITWVRQMPGKGLEWMAKFDPSDSQTNYSPSFQGHVTISVDKSISTAYLQWSSLKASDTAMYYCARRYCSSSSCYVDNWGQGTLVTIFSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1497 bp, synthetic construct
  misc_feature 1..1497: note = Chemically Synthesized, pGX93141, DVSF3 LALA scFv-Fc VH.G4S3.VL
  source 1..1497: mol_type = other DNA
atggactggacttggagaatcctgtttctggtcgcagcagccactggaacccacgccgaggtgcagctggtcgagagcgggggaggcctggtgcagccaggcagatccctgaggctgtcttgtgccgcctctggcttcacatttgatgactacgccatgttctgggtgcggcaggccccaggcaagggcctggagtggatctccggcatcagctggaactctgccaccatcggctatgccgactccgtgaagggcaggttcaccatctcccgggacaatgccaagaagtctctggacctgcagatgaacagcctgagacccgacgataccgccctgtactattgtgccaagggcggcccaagaggcctgcagctgctgtcctcttgggtggattactggggccagggcaccctggtgacagtgtcttccggcggcggcggctctggcggcggcggcagcggcggcggcggctccgatatccagatgacccagtctccctcttccctgagcgcctccgtgggcgatcgggtgacaatcacctgcagagccagccaggatatccggagatacctgaactggtaccagcagaggcctggcagagtgccacagctgctgatctacaccacatccaccctgcagtctggcgtgccaagcagattttccggcagcggctccgtgacagacttcaccctgacaatctccagcctgcagccagaggatttcggcacatattactgccagcagagctactccccacctcacacctttggccagggcacaaagctggagatcaaggagcccaagtcttccgacaagacccacacctgtccaccttgtcccgccccagaggccgccggcggccctagcgtgtttctgttccctccaaagcctaaggataccctgatgatctccagaaccccagaggtgacatgcgtggtggtggacgtgtctcacgaggaccccgaggtgaagtttaactggtacgtggatggcgtggaggtgcacaatgccaagaccaagccaagggaggagcagtacaacagcacctacagagtggtgtccgtgctgacagtgctgcaccaggactggctgaatggcaaggagtataagtgcaaggtgtccaacaaggccctgccagcccccatcgagaagaccatcagcaaggccaagggccagcctagggagccacaggtgtacaccctgccaccctccagagacgagctgacaaagaatcaggtgtctctgacatgcctggtgaagggcttctacccttccgacatcgccgtggagtgggagtctaacggccagcccgagaacaattacaagaccacaccacctgtgctggactccgatggcagcttcttcctgtatagcaagctgaccgtggataagtctagatggcagcagggcaacgtgttctcctgttccgtgatgcacgaagcactgcacaaccactacactcagaagagcctgtccctgtcccctggaaaatgataa

SEQ ID NO: 108 - protein, 497 aa, synthetic construct
  REGION 1..497: note = Chemically Synthesized, pGX93141, DVSF3 LALA scFv-Fc VH.G4S3.VL
MDWTWRILFLVAAATGTHAEVQLVESGGGLVQPGRSLRLSCAASGFTFDDYAMFWVRQAPGKGLEWISGISWNSATIGYADSVKGRFTISRDNAKKSLDLQMNSLRPDDTALYYCAKGGPRGLQLLSSWVDYWGQGTLVTVSSGGGGSGGGGSGGGGSDIQMTQSPSSLSASVGDRVTITCRASQDIRRYLNWYQQRPGRVPQLLIYTTSTLQSGVPSRFSGSGSVTDFTLTISSLQPEDFGTYYCQQSYSPPHTFGQGTKLEIKEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 18 aa, synthetic construct
  REGION 1..18: note = Chemically Synthesized
GSTSGSGKPGSGEGSTKG

SEQ ID NO: 110 - DNA, 2199 bp, synthetic construct
  misc_feature 1..2199: note = Chemically Synthesized, gene optimized anti-RSV, pGX9368
  source 1..2199: mol_type = other DNA
atggactggacatggagaatcctgttcctggtggcagcagcaaccggaacacacgcacaggtgaccctgagagagtccggaccagccctggtgaagccaacccagacactgaccctgacatgcaccttctccggcttttctctgagcacctccggcatgtctgtgggatggatcaggcagccccctggcaaggccctggagtggctggccgacatctggtgggacgataagaaggattacaaccctagcctgaagtcccgcctgacaatcagcaaggacacctccaagaaccaggtggtgctgaaggtgacaaatatggacccagccgatacagccacctactattgcgcccggagcatgatcaccaattggtatttcgacgtgtggggcgccggaaccacagtgacagtgagctccgcctccaccaagggaccaagcgtgttcccactggcaccctctagcaagtctacaagcggcggcaccgccgccctgggatgtctggtgaaggactacttccccgagcctgtgaccgtgtcttggaacagcggcgccctgacatccggagtgcacacctttccagccgtgctgcagtcctctggcctgtacagcctgagctccgtggtgacagtgccctctagctccctgggcacacagacctatatctgcaacgtgaatcacaagccctctaataccaaggtggacaagaaggtggagcctaagagctgtgataagacacacacctgcccaccctgtccagcaccagagctgctgggcggccctagcgtgttcctgtttcctccaaagccaaaggacaccctgatgatctccagaacacctgaggtgacctgcgtggtggtggacgtgtctcacgaggaccccgaggtgaagttcaactggtacgtggatggcgtggaggtgcacaatgccaagaccaagcctcgggaggagcagtacaacagcacatatagagtggtgtccgtgctgaccgtgctgcaccaggattggctgaacggcaaggagtataagtgcaaggtgtccaataaggccctgcctgccccaatcgagaagacaatcagcaaggccaagggccagcctagggagccacaggtgtacaccctgccccctagccgcgacgagctgacaaagaaccaggtgtccctgacctgtctggtgaagggcttctatccatctgatatcgccgtggagtgggagagcaatggccagcccgagaacaattacaagaccacaccacccgtgctggactccgatggctctttctttctgtattccaagctgaccgtggacaagtctaggtggcagcagggcaacgtgttttcctgttctgtgatgcacgaggccctgcacaatcactacacacagaagagcctgtccctgtctccaggcaagaggggaaggaagcggagaagcggctccggagcaaccaacttctccctgctgaagcaggcaggcgatgtggaggagaatccaggacctatggtgctgcagacccaggtgtttatctctctgctgctgtggatcagcggcgcctacggcgacatccagatgacacagtctccaagcaccctgtccgcctctgtgggcgatagggtgacaatcacctgcaagtgtcagctgagcgtgggctacatgcactggtatcagcagaagcccggcaaggcccctaagctgctgatctacgacacctctaagctggcaagcggagtgccctcccgcttcagcggctccggctctggaacagagtttacactgaccatctctagcctgcagcccgacgatttcgccacctactattgctttcagggcagcggctatcccttcaccttcggcggcggcaccaagctggagatcaagcggacagtggccgcccccagcgtgttcatctttcctccatccgacgagcagctgaagtctggcaccgccagcgtggtgtgcctgctgaacaatttctaccctagagaggccaaggtgcagtggaaggtggataacgccctgcagagcggcaattcccaggagtctgtgacagagcaggacagcaaggattccacctattctctgtcctctacactgaccctgtccaaggccgattacgagaagcacaaggtgtatgcctgcgaggtgacacaccagggcctgagctcccctgtgaccaagagctttaacagaggcgagtgttgataa

SEQ ID NO: 111 - protein, 731 aa, synthetic construct
  REGION 1..731: note = Chemically Synthesized, gene optimized anti-RSV, pGX9368
MDWTWRILFLVAAATGTHAQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - DNA, 1485 bp, synthetic construct
  misc_feature 1..1485: note = Chemically Synthesized, scFv-Fc anti-RSV, pGX9369
  source 1..1485: mol_type = other DNA
atggtgctgcagacccaggtgtttatttcactgctgctgtggatttcaggagcctacggggacattcagatgacccagagcccttcaacactgtccgccagcgtgggcgacagagtgacaatcacctgtaagtgccagctgagcgtgggctatatgcactggtatcagcagaagcctggcaaggccccaaagctgctgatctatgacaccagcaagctggcctctggcgtgccatccagattctccggctctggcagcggcaccgagtttacactgaccatctccagcctgcagccagatgacttcgccacctactattgcttccagggcagcggctatcccttcacctttggcggcggcacaaagctggagatcaagggcggcggcggctccggcggcggcggctctggcggcggcggctctcaggtgaccctgagagagtccggcccagccctggtgaagccaacccagaccctgacactgacatgcaccttctccggcttcagcctgtccaccagcggcatgtccgtgggctggatcaggcagcccccaggcaaggccctggagtggctggccgatatctggtgggacgataagaaggactacaacccctccctgaagagcagactgaccatcagcaaggataccagcaagaaccaggtggtgctgaaggtgacaaatatggacccagccgataccgccacatactactgtgccagatccatgatcacaaattggtacttcgacgtgtggggcgccggcacaaccgtgacagtgagctctgagccaaagtcctgcgacaagacccacacctgtcctccttgtccagcccccgagctgctgggcggcccaagcgtgttcctgtttccccctaagccaaaggataccctgatgatctccagaaccccagaggtgacatgcgtggtggtggacgtgagccacgaggaccccgaggtgaagttcaattggtacgtggatggcgtggaggtgcacaatgccaagaccaagccaagagaggagcagtataactctacatatcgcgtggtgtccgtgctgacagtgctgcaccaggactggctgaatggcaaggagtacaagtgcaaggtgtccaataaggccctgccagcccctatcgagaagacaatctccaaggccaagggccagcccagagagccacaggtgtatacactgccaccctccagagatgagctgacaaagaatcaggtgtccctgacatgtctggtgaagggcttttatccctccgatatcgccgtggagtgggagtctaatggccagcccgagaataactataagacaacccctccagtgctggactccgatggctcctttttcctgtattccaagctgaccgtggataagagcaggtggcagcagggcaacgtgttctcttgttccgtgatgcacgaagcactgcacaaccactacacccagaagtcactgtcactgtcaccaggaaaatgataa

SEQ ID NO: 113 - protein, 493 aa, synthetic construct
  REGION 1..493: note = Chemically Synthesized, scFv-Fc anti-RSV, pGX9369
MVLQTQVFISLLLWISGAYGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKGGGGSGGGGSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - DNA, 2199 bp, synthetic construct
  misc_feature 1..2199: note = Chemically Synthesized, gene optimized anti-RSV, pGX9370
  source 1..2199: mol_type = other DNA
atggactggacctggagaatcctgttcctggtggcagcagcaaccggaacacacgcacaggtgacactgagggagagcggacctgccctggtgaagccaacccagacactgaccctgacatgcaccttctctggcttttccctgtctaccagcggcatgagcgtgggatggatcaggcagccccctggcaaggccctggagtggctggccgacatctggtgggacgataagaaggattacaacccctctctgaagagccgcctgaccatcagcaaggatacatccaagaaccaggtggtgctgaaggtgaccaatatggaccctgccgatacagccacctactattgtgcccggagcatgatcaccaattggtactttgacgtgtggggcgccggcaccacagtgacagtgagctccgccaagaccacagccccttccgtgtatcctctggccccagtgtgcggcgataccacaggctctagcgtgaccctgggctgtctggtgaagggctacttcccagagcccgtgacactgacctggaactccggctctctgtcctctggcgtgcacacctttccagccgtgctgcagagcgacctgtacacactgagctcctctgtgacagtgaccagctccacctggccaagccagtccatcacatgcaacgtggcccaccccgcctctagcaccaaggtggataagaagatcgagcccagaggccctacaatcaagccctgtccaccctgcaagtgtcctgccccaaatctgctgggcggcccttccgtgttcatctttcctccaaagatcaaggacgtgctgatgatctctctgagccctatcgtgacctgcgtggtggtggacgtgtccgaggacgatccagatgtgcagatctcttggttcgtgaacaatgtggaggtgcacaccgcccagacacagacccaccgggaggattataacagcacactgagagtggtgtccgccctgccaatccagcaccaggactggatgagcggcaaggagtttaagtgcaaggtgaacaataaggatctgcccgcccctatcgagcggaccatctccaagcccaagggctctgtgagagcccctcaggtgtacgtgctgccccctccagaggaggagatgaccaagaagcaggtgacactgacctgtatggtgacagacttcatgcctgaggatatctacgtggagtggaccaacaatggcaagacagagctgaactataagaataccgagccagtgctggactccgatggctcttactttatgtatagcaagctgagggtggagaagaagaactgggtggagcgcaattcctattcttgtagcgtggtgcacgagggcctgcacaatcaccacaccacaaagtccttctctagaaccccaggcaagaggggaaggaagcggagaagcggctccggagccacaaacttttccctgctgaagcaggcaggcgacgtggaggagaatccaggacccatggtgctgcagacccaggtgttcatctctctgctgctgtggatcagcggcgcctacggcgacatccagatgacccagtctcccagcacactgtccgcctctgtgggcgatcgggtgacaatcacctgcaagtgtcagctgtccgtgggctacatgcactggtatcagcagaagccaggcaaggcccccaagctgctgatctatgacacctctaagctggccagcggcgtgccttccagattcagcggctccggctctggcaccgagtttacactgaccatctcctctctgcagccagacgatttcgccacatactattgctttcagggcagcggataccccttcaccttcggcggcggcacaaagctggagatcaagagggccgatgccgccccaaccgtgtccatcttccctcccagcagcgagcagctgacatctggcggcgccagcgtggtgtgcttcctgaacaacttctaccccaaggacatcaacgtgaagtggaagatcgatggcagcgagcgccagaacggcgtgctgaattcctggaccgaccaggatagcaaggactccacatactctatgtctagcacactgaccctgacaaaggatgagtacgagcggcacaattcctatacctgcgaggccacacacaagaccagcacatcccctatcgtgaagtcttttaacagaaatgagtgttgataa

SEQ ID NO: 115 - protein, 731 aa, synthetic construct
  REGION 1..731: note = Chemically Synthesized, gene optimized anti-RSV, pGX9370
MDWTWRILFLVAAATGTHAQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRGRKRRSGSGATNFSLLKQAGDVEENPGPMVLQTQVFISLLLWISGAYGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 116 - DNA, 1488 bp, synthetic construct
  misc_feature 1..1488: note = Chemically Synthesized, scFv-Fc anti-RSV, pGX9371
  source 1..1488: mol_type = other DNA
atggtgctgcagactcaggtgtttatttcactgctgctgtggatttccggcgcttacggcgacattcagatgacccagagcccctccacactgagcgcctccgtgggcgacagagtgacaatcacatgcaagtgtcagctgtctgtgggctatatgcactggtatcagcagaagcccggcaaggccccaaagctgctgatctatgacacctctaagctggcctctggcgtgccaagcagattctccggcagcggctccggcaccgagttcaccctgacaatctcctctctgcagccagacgatttcgccacatactactgctttcagggctccggctacccattcacatttggcggcggcacaaagctggagatcaagggcggcggcggctccggcggcggcggctctggcggcggcggctctcaggtgacactgcgggagtccggcccagccctggtgaagccaacccagacactgacactgacctgtacattttccggcttctctctgtccaccagcggcatgagcgtgggctggatcagacagccccctggcaaggccctggagtggctggccgatatctggtgggacgataagaaggactacaatccttccctgaagtctagactgaccatctccaaggatacctccaagaatcaggtggtgctgaaggtgaccaacatggaccctgccgatacagccacctattactgcgccagaagcatgatcaccaactggtactttgacgtgtggggcgccggcacaaccgtgacagtgtcttccgagcctagaggcccaaccatcaagccatgcccaccctgtaagtgtcccgccccaaacctgctgggcggcccatccgtgttcatctttccccctaagatcaaggacgtgctgatgatcagcctgagcccaatcgtgacatgcgtggtggtggacgtgtccgaggatgacccagatgtgcagatctcttggttcgtgaataacgtggaggtgcacaccgcccagacccagacccacagagaggattacaattccacactgagagtggtgtccgccctgcctatccagcaccaggattggatgagcggcaaggagtttaagtgcaaggtgaacaataaggacctgcccgccccaatcgagagaaccatctccaagccaaagggctctgtgagggccccacaggtgtacgtgctgcctcctccagaggaggagatgacaaagaagcaggtgacactgacctgcatggtgaccgacttcatgcccgaggacatctacgtggagtggacaaacaatggcaagacagagctgaactataagaacaccgagccagtgctggattccgacggctcttacttcatgtactccaagctgagagtggagaagaagaactgggtggagcggaatagctactcctgttccgtggtccacgaagggctgcataaccaccacaccactaagtcattttcaagaaccccaggcaaatgataa

SEQ ID NO: 117 - protein, 494 aa, synthetic construct
  REGION 1..494: note = Chemically Synthesized, scFv-Fc anti-RSV, pGX9371
MVLQTQVFISLLLWISGAYGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKGGGGSGGGGSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 118 - DNA, 2220 bp, synthetic construct
  misc_feature 1..2220: note = Chemically Synthesized, gene optimized anti-RSV, pGX9283
  source 1..2220: mol_type = other DNA
atggactggacttggagaatcctgttcctggtcgccgccgcaactgggactcatgctcaggtgcagctggtgcagagcggggcagaggtgaagaagccaggcagctccgtgaaggtgtcttgcaaggcaagcggcggctctctgagcacctacggcatccactgggtgaggcaggcaccaggacagggcctggagtgggtgggcggcgtgatgaccgtgtacggcaagaccacatatggccagaacttccagggcagggtgacaatcgccgtggaccgctctaccaatacagcctacatggagctgtctagcctgaccagcgacgataccggcacatactattgcgccaccgactcttactacgtgtggacaggcagctatccccctccattcgatctgtggggccagggcaccctggtgacagtgtcctctgcctctacaaagggaccaagcgtgtttccactggcacctagctccaagtccacctctggcggcacagccgccctgggctgtctggtgaaggattacttccctgagccagtgaccgtgtcctggaactctggcgccctgaccagcggagtgcacacatttcccgccgtgctgcagtctagcggcctgtactccctgtcctctgtggtgaccgtgcctagctcctctctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacaccctgatgatctccagaaccccagaggtgacatgcgtggtggtggacgtgtctcacgaggaccccgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatcccgcgacgagctgaccaagaaccaggtgtctctgacatgtctggtgaagggcttctatccctctgatatcgccgtggagtgggagagcaatggccagcctgagaacaattacaagaccacaccccctgtgctggacagcgatggctccttctttctgtattccaagctgaccgtggacaagtctcggtggcagcagggcaacgtgtttagctgctccgtgatgcacgaggccctgcacaatcactacacccagaagtctctgagcctgtccccaggcaagaggggaagaaagcggagatctggcagcggcgccacaaacttcagcctgctgaagcaggccggcgatgtggaggagaatcctggcccaatggtgctgcagacccaggtgtttatcagcctgctgctgtggatctccggagcatacggagagatcgtgctgacccagacaccaggaacccagtccctgtctcctggacagtccgccacactgtcttgtagagccagccactccgtgggcaatgactacctggcctggtatcagcagaagcctggacagagcccacggctgctgatccacggagcatacaggagggactccggcatccctgatagattcatcggctctggcagcggcaccgactttaccctgacaatcgatagcctggagcctgacgattgcgccgtgtactattgtcagcagtatggctcctggccactgaccttcggcggcggcacaaaggtggacatcaagaggaccgtggccgcccctagcgtgttcatctttccaccctccgatgagcagctgaagagcggcacagcctccgtggtgtgcctgctgaacaacttctacccacgcgaggccaaggtgcagtggaaggtggacaacgccctgcagtctggcaatagccaggagtccgtgaccgagcaggactctaaggatagcacatattccctgagctccaccctgacactgtccaaggccgattacgagaagcacaaggtgtatgcctgtgaggtcacccaccagggactgtcttcacccgtcacaaaatccttcaataggggagaatgctgataa

SEQ ID NO: 119 - protein, 738 aa, synthetic construct
  REGION 1..738: note = Chemically Synthesized, gene optimized anti-RSV, pGX9283
MDWTWRILFLVAAATGTHAQVQLVQSGAEVKKPGSSVKVSCKASGGSLSTYGIHWVRQAPGQGLEWVGGVMTVYGKTTYGQNFQGRVTIAVDRSTNTAYMELSSLTSDDTGTYYCATDSYYVWTGSYPPPFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQTPGTQSLSPGQSATLSCRASHSVGNDYLAWYQQKPGQSPRLLIHGAYRRDSGIPDRFIGSGSGTDFTLTIDSLEPDDCAVYYCQQYGSWPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 8 aa, synthetic construct
  REGION 1..8: note = Chemically Synthesized
AMEKIVIL

SEQ ID NO: 121 - DNA, 5153 bp, synthetic construct
  misc_feature 1..5153: note = Chemically Synthesized, pGX9368 full plasmid sequence
  source 1..5153: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacatggagaatcctgttcctggtggcagcagcaaccggaacacacgcacaggtgaccctgagagagtccggaccagccctggtgaagccaacccagacactgaccctgacatgcaccttctccggcttttctctgagcacctccggcatgtctgtgggatggatcaggcagccccctggcaaggccctggagtggctggccgacatctggtgggacgataagaaggattacaaccctagcctgaagtcccgcctgacaatcagcaaggacacctccaagaaccaggtggtgctgaaggtgacaaatatggacccagccgatacagccacctactattgcgcccggagcatgatcaccaattggtatttcgacgtgtggggcgccggaaccacagtgacagtgagctccgcctccaccaagggaccaagcgtgttcccactggcaccctctagcaagtctacaagcggcggcaccgccgccctgggatgtctggtgaaggactacttccccgagcctgtgaccgtgtcttggaacagcggcgccctgacatccggagtgcacacctttccagccgtgctgcagtcctctggcctgtacagcctgagctccgtggtgacagtgccctctagctccctgggcacacagacctatatctgcaacgtgaatcacaagccctctaataccaaggtggacaagaaggtggagcctaagagctgtgataagacacacacctgcccaccctgtccagcaccagagctgctgggcggccctagcgtgttcctgtttcctccaaagccaaaggacaccctgatgatctccagaacacctgaggtgacctgcgtggtggtggacgtgtctcacgaggaccccgaggtgaagttcaactggtacgtggatggcgtggaggtgcacaatgccaagaccaagcctcgggaggagcagtacaacagcacatatagagtggtgtccgtgctgaccgtgctgcaccaggattggctgaacggcaaggagtataagtgcaaggtgtccaataaggccctgcctgccccaatcgagaagacaatcagcaaggccaagggccagcctagggagccacaggtgtacaccctgccccctagccgcgacgagctgacaaagaaccaggtgtccctgacctgtctggtgaagggcttctatccatctgatatcgccgtggagtgggagagcaatggccagcccgagaacaattacaagaccacaccacccgtgctggactccgatggctctttctttctgtattccaagctgaccgtggacaagtctaggtggcagcagggcaacgtgttttcctgttctgtgatgcacgaggccctgcacaatcactacacacagaagagcctgtccctgtctccaggcaagaggggaaggaagcggagaagcggctccggagcaaccaacttctccctgctgaagcaggcaggcgatgtggaggagaatccaggacctatggtgctgcagacccaggtgtttatctctctgctgctgtggatcagcggcgcctacggcgacatccagatgacacagtctccaagcaccctgtccgcctctgtgggcgatagggtgacaatcacctgcaagtgtcagctgagcgtgggctacatgcactggtatcagcagaagcccggcaaggcccctaagctgctgatctacgacacctctaagctggcaagcggagtgccctcccgcttcagcggctccggctctggaacagagtttacactgaccatctctagcctgcagcccgacgatttcgccacctactattgctttcagggcagcggctatcccttcaccttcggcggcggcaccaagctggagatcaagcggacagtggccgcccccagcgtgttcatctttcctccatccgacgagcagctgaagtctggcaccgccagcgtggtgtgcctgctgaacaatttctaccctagagaggccaaggtgcagtggaaggtggataacgccctgcagagcggcaattcccaggagtctgtgacagagcaggacagcaaggattccacctattctctgtcctctacactgaccctgtccaaggccgattacgagaagcacaaggtgtatgcctgcgaggtgacacaccagggcctgagctcccctgtgaccaagagctttaacagaggcgagtgttgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 122 - DNA, 4439 bp, synthetic construct
  misc_feature 1..4439: note = Chemically Synthesized, pGX9369 full plasmid sequence
  source 1..4439: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggtgctgcagacccaggtgtttatttcactgctgctgtggatttcaggagcctacggggacattcagatgacccagagcccttcaacactgtccgccagcgtgggcgacagagtgacaatcacctgtaagtgccagctgagcgtgggctatatgcactggtatcagcagaagcctggcaaggccccaaagctgctgatctatgacaccagcaagctggcctctggcgtgccatccagattctccggctctggcagcggcaccgagtttacactgaccatctccagcctgcagccagatgacttcgccacctactattgcttccagggcagcggctatcccttcacctttggcggcggcacaaagctggagatcaagggcggcggcggctccggcggcggcggctctggcggcggcggctctcaggtgaccctgagagagtccggcccagccctggtgaagccaacccagaccctgacactgacatgcaccttctccggcttcagcctgtccaccagcggcatgtccgtgggctggatcaggcagcccccaggcaaggccctggagtggctggccgatatctggtgggacgataagaaggactacaacccctccctgaagagcagactgaccatcagcaaggataccagcaagaaccaggtggtgctgaaggtgacaaatatggacccagccgataccgccacatactactgtgccagatccatgatcacaaattggtacttcgacgtgtggggcgccggcacaaccgtgacagtgagctctgagccaaagtcctgcgacaagacccacacctgtcctccttgtccagcccccgagctgctgggcggcccaagcgtgttcctgtttccccctaagccaaaggataccctgatgatctccagaaccccagaggtgacatgcgtggtggtggacgtgagccacgaggaccccgaggtgaagttcaattggtacgtggatggcgtggaggtgcacaatgccaagaccaagccaagagaggagcagtataactctacatatcgcgtggtgtccgtgctgacagtgctgcaccaggactggctgaatggcaaggagtacaagtgcaaggtgtccaataaggccctgccagcccctatcgagaagacaatctccaaggccaagggccagcccagagagccacaggtgtatacactgccaccctccagagatgagctgacaaagaatcaggtgtccctgacatgtctggtgaagggcttttatccctccgatatcgccgtggagtgggagtctaatggccagcccgagaataactataagacaacccctccagtgctggactccgatggctcctttttcctgtattccaagctgaccgtggataagagcaggtggcagcagggcaacgtgttctcttgttccgtgatgcacgaagcactgcacaaccactacacccagaagtcactgtcactgtcaccaggaaaatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 123 - DNA, 5153 bp, synthetic construct
  misc_feature 1..5153: note = Chemically Synthesized, pGX9370 full plasmid sequence
  source 1..5153: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacctggagaatcctgttcctggtggcagcagcaaccggaacacacgcacaggtgacactgagggagagcggacctgccctggtgaagccaacccagacactgaccctgacatgcaccttctctggcttttccctgtctaccagcggcatgagcgtgggatggatcaggcagccccctggcaaggccctggagtggctggccgacatctggtgggacgataagaaggattacaacccctctctgaagagccgcctgaccatcagcaaggatacatccaagaaccaggtggtgctgaaggtgaccaatatggaccctgccgatacagccacctactattgtgcccggagcatgatcaccaattggtactttgacgtgtggggcgccggcaccacagtgacagtgagctccgccaagaccacagccccttccgtgtatcctctggccccagtgtgcggcgataccacaggctctagcgtgaccctgggctgtctggtgaagggctacttcccagagcccgtgacactgacctggaactccggctctctgtcctctggcgtgcacacctttccagccgtgctgcagagcgacctgtacacactgagctcctctgtgacagtgaccagctccacctggccaagccagtccatcacatgcaacgtggcccaccccgcctctagcaccaaggtggataagaagatcgagcccagaggccctacaatcaagccctgtccaccctgcaagtgtcctgccccaaatctgctgggcggcccttccgtgttcatctttcctccaaagatcaaggacgtgctgatgatctctctgagccctatcgtgacctgcgtggtggtggacgtgtccgaggacgatccagatgtgcagatctcttggttcgtgaacaatgtggaggtgcacaccgcccagacacagacccaccgggaggattataacagcacactgagagtggtgtccgccctgccaatccagcaccaggactggatgagcggcaaggagtttaagtgcaaggtgaacaataaggatctgcccgcccctatcgagcggaccatctccaagcccaagggctctgtgagagcccctcaggtgtacgtgctgccccctccagaggaggagatgaccaagaagcaggtgacactgacctgtatggtgacagacttcatgcctgaggatatctacgtggagtggaccaacaatggcaagacagagctgaactataagaataccgagccagtgctggactccgatggctcttactttatgtatagcaagctgagggtggagaagaagaactgggtggagcgcaattcctattcttgtagcgtggtgcacgagggcctgcacaatcaccacaccacaaagtccttctctagaaccccaggcaagaggggaaggaagcggagaagcggctccggagccacaaacttttccctgctgaagcaggcaggcgacgtggaggagaatccaggacccatggtgctgcagacccaggtgttcatctctctgctgctgtggatcagcggcgcctacggcgacatccagatgacccagtctcccagcacactgtccgcctctgtgggcgatcgggtgacaatcacctgcaagtgtcagctgtccgtgggctacatgcactggtatcagcagaagccaggcaaggcccccaagctgctgatctatgacacctctaagctggccagcggcgtgccttccagattcagcggctccggctctggcaccgagtttacactgaccatctcctctctgcagccagacgatttcgccacatactattgctttcagggcagcggataccccttcaccttcggcggcggcacaaagctggagatcaagagggccgatgccgccccaaccgtgtccatcttccctcccagcagcgagcagctgacatctggcggcgccagcgtggtgtgcttcctgaacaacttctaccccaaggacatcaacgtgaagtggaagatcgatggcagcgagcgccagaacggcgtgctgaattcctggaccgaccaggatagcaaggactccacatactctatgtctagcacactgaccctgacaaaggatgagtacgagcggcacaattcctatacctgcgaggccacacacaagaccagcacatcccctatcgtgaagtcttttaacagaaatgagtgttgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 124 - DNA, 4442 bp, synthetic construct
  misc_feature 1..4442: note = Chemically Synthesized, pGX371 full plasmid sequence
  source 1..4442: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggtgctgcagactcaggtgtttatttcactgctgctgtggatttccggcgcttacggcgacattcagatgacccagagcccctccacactgagcgcctccgtgggcgacagagtgacaatcacatgcaagtgtcagctgtctgtgggctatatgcactggtatcagcagaagcccggcaaggccccaaagctgctgatctatgacacctctaagctggcctctggcgtgccaagcagattctccggcagcggctccggcaccgagttcaccctgacaatctcctctctgcagccagacgatttcgccacatactactgctttcagggctccggctacccattcacatttggcggcggcacaaagctggagatcaagggcggcggcggctccggcggcggcggctctggcggcggcggctctcaggtgacactgcgggagtccggcccagccctggtgaagccaacccagacactgacactgacctgtacattttccggcttctctctgtccaccagcggcatgagcgtgggctggatcagacagccccctggcaaggccctggagtggctggccgatatctggtgggacgataagaaggactacaatccttccctgaagtctagactgaccatctccaaggatacctccaagaatcaggtggtgctgaaggtgaccaacatggaccctgccgatacagccacctattactgcgccagaagcatgatcaccaactggtactttgacgtgtggggcgccggcacaaccgtgacagtgtcttccgagcctagaggcccaaccatcaagccatgcccaccctgtaagtgtcccgccccaaacctgctgggcggcccatccgtgttcatctttccccctaagatcaaggacgtgctgatgatcagcctgagcccaatcgtgacatgcgtggtggtggacgtgtccgaggatgacccagatgtgcagatctcttggttcgtgaataacgtggaggtgcacaccgcccagacccagacccacagagaggattacaattccacactgagagtggtgtccgccctgcctatccagcaccaggattggatgagcggcaaggagtttaagtgcaaggtgaacaataaggacctgcccgccccaatcgagagaaccatctccaagccaaagggctctgtgagggccccacaggtgtacgtgctgcctcctccagaggaggagatgacaaagaagcaggtgacactgacctgcatggtgaccgacttcatgcccgaggacatctacgtggagtggacaaacaatggcaagacagagctgaactataagaacaccgagccagtgctggattccgacggctcttacttcatgtactccaagctgagagtggagaagaagaactgggtggagcggaatagctactcctgttccgtggtccacgaagggctgcataaccaccacaccactaagtcattttcaagaaccccaggcaaatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 125 - DNA, 5174 bp, synthetic construct
  misc_feature 1..5174: note = Chemically Synthesized, pGX9283 full plasmid sequence
  source 1..5174: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacttggagaatcctgttcctggtcgccgccgcaactgggactcatgctcaggtgcagctggtgcagagcggggcagaggtgaagaagccaggcagctccgtgaaggtgtcttgcaaggcaagcggcggctctctgagcacctacggcatccactgggtgaggcaggcaccaggacagggcctggagtgggtgggcggcgtgatgaccgtgtacggcaagaccacatatggccagaacttccagggcagggtgacaatcgccgtggaccgctctaccaatacagcctacatggagctgtctagcctgaccagcgacgataccggcacatactattgcgccaccgactcttactacgtgtggacaggcagctatccccctccattcgatctgtggggccagggcaccctggtgacagtgtcctctgcctctacaaagggaccaagcgtgtttccactggcacctagctccaagtccacctctggcggcacagccgccctgggctgtctggtgaaggattacttccctgagccagtgaccgtgtcctggaactctggcgccctgaccagcggagtgcacacatttcccgccgtgctgcagtctagcggcctgtactccctgtcctctgtggtgaccgtgcctagctcctctctgggcacccagacatatatctgcaacgtgaatcacaagcctagcaatacaaaggtggacaagaaggtggagccaaagtcctgtgataagacccacacatgccctccctgtccagcacctgagctgctgggcggcccaagcgtgttcctgtttccacccaagcccaaggacaccctgatgatctccagaaccccagaggtgacatgcgtggtggtggacgtgtctcacgaggaccccgaggtgaagtttaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgagcaataaggccctgccagcccccatcgagaagaccatctccaaggcaaagggacagccaagggagccacaggtgtacacactgcctccatcccgcgacgagctgaccaagaaccaggtgtctctgacatgtctggtgaagggcttctatccctctgatatcgccgtggagtgggagagcaatggccagcctgagaacaattacaagaccacaccccctgtgctggacagcgatggctccttctttctgtattccaagctgaccgtggacaagtctcggtggcagcagggcaacgtgtttagctgctccgtgatgcacgaggccctgcacaatcactacacccagaagtctctgagcctgtccccaggcaagaggggaagaaagcggagatctggcagcggcgccacaaacttcagcctgctgaagcaggccggcgatgtggaggagaatcctggcccaatggtgctgcagacccaggtgtttatcagcctgctgctgtggatctccggagcatacggagagatcgtgctgacccagacaccaggaacccagtccctgtctcctggacagtccgccacactgtcttgtagagccagccactccgtgggcaatgactacctggcctggtatcagcagaagcctggacagagcccacggctgctgatccacggagcatacaggagggactccggcatccctgatagattcatcggctctggcagcggcaccgactttaccctgacaatcgatagcctggagcctgacgattgcgccgtgtactattgtcagcagtatggctcctggccactgaccttcggcggcggcacaaaggtggacatcaagaggaccgtggccgcccctagcgtgttcatctttccaccctccgatgagcagctgaagagcggcacagcctccgtggtgtgcctgctgaacaacttctacccacgcgaggccaaggtgcagtggaaggtggacaacgccctgcagtctggcaatagccaggagtccgtgaccgagcaggactctaaggatagcacatattccctgagctccaccctgacactgtccaaggccgattacgagaagcacaaggtgtatgcctgtgaggtcacccaccagggactgtcttcacccgtcacaaaatccttcaataggggagaatgctgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 126 - DNA, 6990 bp, synthetic construct
  misc_feature 1..6990: note = Chemically Synthesized, pRD245 full plasmid sequence
  source 1..6990: mol_type = other DNA
gctgcttcgcgatgtacgggccagatatacgcttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcagtcagtcacttgccaggtgacagtgacagtgacttctgagtgtaatggttgtgcagtgcctcgtgcatcacgctgcaggagaacacgttgccctgctgccaccggctcttatccactgtcagcttgctgtacaggaaaaaggagccatcagaatccagcacgggtggtgtggtcttgtagttgttctctggctggccattgctctcccactccacggcgatgtcgctagggtaaaagcccttcaccagacaggtcagagacacctggttcttggtcagctcatctctgcttggaggcagggtatacacctgaggctctcttggctggcccttggccttggagatggtcttctcgataggggctggcagggccttattggacaccttgcacttgtactccttgccgttcagccaatcctggtgcagcactgtcagcacggacaccactctgtaggtggagttgtactgctcctctctgggctttgtcttggcattgtgcacctccacgccgtccacgtaccagttgaacttcacctcggggtcctcgtggctcacatccaccaccacgcaggtcacctctggtgttctggagatcatcagggtgtccttaggctttggtggaaacaggaacacggatgggccgccggcggcctctggggcggggcatggaggacatgtgtgggtcttatcgctggacttaggctccttgatctccagctttgtgccctggccaaaggtgtgaggtggggaatagctctgctggcaatagtaggtgccgaagtcctctggctgcaggctagagatggtcagtgtgaagtctgtcacagagccgctgccagaaaaccgggatggcacgccagactgcagggtgcttgtggtgtagatcagcagctgaggcactctgcctggtctctgctggtaccagttcaggtagcgtctgatgtcctggctggctctgcaggtgattgtcaccctgtcgcccacgctggcggacagagaagatggggactgggtcatctggatgtcggagccgccgccgccgctgccgccgccgccagagccgccgccgccggaagacacggtcaccagtgtgccctggccccaatagtccacccaggagctcagcagctgcaggcctctggggccgcccttggcgcagtagtacagggcggtatcgtcaggtctcagggagttcatctgcaggtccagggacttcttggcgttatccctgctgatggtgaatctgcccttcacggagtcggcgtagccgattgtggcggagttccaagagatgccgctgatccactccaggcccttgcctggggcctgccgcacccaaaacatggcgtaatcgtcgaatgtaaagccgctggcggcacaggacagtctcaggcttctgcctggctgcaccagcccgcctccgctttcgaccagctggacctctgcgtgagtgccggtggctgctgcgaccaggaacaggatgcgccaagtccaatccatggtggcggctccgagctctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgtagcttaatcggccgccagcttatcggtaccgttatcagatcggcttcgaggggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccacatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaagtacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaacgggaggtctatataagcaatgctcgtttagggaaccgccattctgcctggggacgtcggaggagcagctcggagccgccaccatggactggacttggcgcattctgtttctggtcgcagccgctactggaacccacgctcaggtgcagctggtcgaatcaggaggaggggtggtgcagccaggcagatccctgagactgtcttgcgccgccagcggcttcacattctccaagtacggcatgcactgggtgcggcaggcccccggcaagggcctggagtgggtggccgtgatctcttacgagggcagcaacaagtactatgccgactctgtgaagggcagattcacaatcagcagagacaatagcaagaacaccctgtacctgcagatgaactccctgagagccgaggacaccgccgtgtactattgcgccaagtccggcacccagtactatgacaccacaggctacgagtatagaggcctggagtactttggctattggggccagggcacactggtgacagtgtcctctggcggcggcggctccggcggcggcggcagcggcggcggcggctctgagatcgtgctgacacagagcccaggcacactgtccctgagcccaggcgagagagccacactgtcctgtagagcctctcagtccgtgagctcctcttacctggcctggtatcagcagaagagaggccaggcccccagactgctgatctatgacgcctccagcagagccacaggcatccctgatcggttctctggcagcggctccggcaccgatttcaccctgacaatctctagactggagcctgaggactttgccgtgtactactgccagcagtatggcagatctcggtggacatttggccagggcaccaaggtggagatcaaggagccaaagagctgtgacaagacccacacctgtccaccctgcccagcccctgaggccgccggcggccctagcgtgttcctgttcccacctaagcccaaggacaccctgatgatcagccggacacctgaggtgacctgcgtggtggtggacgtgagccacgaggaccctgaggtgaagttcaactggtatgtggatggcgtggaggtgcacaacgccaagacaaagcccagagaggagcagtacaattccacctacagagtggtgtccgtgctgaccgtgctgcaccaggactggctgaatggcaaggagtacaagtgcaaggtgtccaacaaggccctgcccgccccaatcgagaagaccatcagcaaggccaagggccagcccagagagccacaggtgtataccctgcctccatccagagaggagatgaccaagaatcaggtgtctctgacctgtctggtgaagggcttctatccaagcgatatcgccgtggagtgggagtccaatggccagcctgagaataattacaagaccacaccccctgtgctggatagcgatggctccttctttctgtactccaagctgaccgtggacaagtccagatggcagcagggcaacgtgttttcctgctctgtgatgcatgaagccctgcataaccattacacccagaaatccctgtccctgagccccggaaaatgactgactgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 127 - DNA, 4472 bp, synthetic construct
  misc_feature 1..4472: note = Chemically Synthesized, pGX 93100 full plasmid sequence
  source 1..4472: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacctggagaatcctgttcctggtggcagcagcaaccggaacacacgcacaggtgcagctggtggagagcggcggcggcgtggtgcagccaggccggtccctgaggctgtcttgcgcagcaagcggcttcacctttagcaagtacggaatgcactgggtgagacaggcacctggcaagggcctggagtgggtggccgtgatctcctatgagggctctaacaagtactatgccgattccgtgaagggcaggtttaccatcagcagagacaactccaagaatacactgtacctgcagatgaatagcctgagggccgaggataccgccgtgtactattgcgccaagtccggcacacagtactatgacaccacaggctacgagtatagaggcctggagtacttcggctattggggccagggcaccctggtgacagtgagctccggcggcggcggctccggcggcggcggcagcggcggcggcggcagcgagatcgtgctgacccagtctcctggcacactgtctctgagcccaggagagagggccaccctgagctgtagagcctcccagagcgtgagcagcagctacctggcctggtatcagcagaagaggggacaggccccacgcctgctgatctacgacgcctctagccgggccaccggcatccccgatcgcttcagcggctccggctctggcacagactttaccctgacaatctcccggctggagcctgaggatttcgccgtgtactattgccagcagtatggcaggagcagatggacctttggccagggcacaaaggtggagatcaaggagcccaagtcctgtgataagacccacacatgccctccctgtccagcacctgaggcagccggcggcccaagcgtgttcctgtttccacccaagcctaaggatacactgatgatctctagaacccccgaggtgacatgcgtggtggtggacgtgagccacgaggaccccgaggtgaagttcaactggtacgtggacggcgtggaggtgcacaatgccaagaccaagcccagggaggagcagtacaacagcacctatagagtggtgtccgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctccatcccgcgaggagatgaccaagaaccaggtgtctctgacatgtctggtgaagggcttctatccttctgatatcgccgtggagtgggagagcaatggccagccagagaacaattacaagaccacaccccctgtgctggactctgatggcagcttctttctgtattccaagctgaccgtggacaagtctaggtggcagcagggcaacgtgttttcctgctctgtgatgcacgaggccctgcacaatcactacacccagaagagcctgtccctgtctcctggcaagtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 128 - DNA, 4451 bp, synthetic construct
  misc_feature 1..4451: note = Chemically Synthesized, pGX 93141 full plasmid sequence
  source 1..4451: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacttggagaatcctgtttctggtcgcagcagccactggaacccacgccgaggtgcagctggtcgagagcgggggaggcctggtgcagccaggcagatccctgaggctgtcttgtgccgcctctggcttcacatttgatgactacgccatgttctgggtgcggcaggccccaggcaagggcctggagtggatctccggcatcagctggaactctgccaccatcggctatgccgactccgtgaagggcaggttcaccatctcccgggacaatgccaagaagtctctggacctgcagatgaacagcctgagacccgacgataccgccctgtactattgtgccaagggcggcccaagaggcctgcagctgctgtcctcttgggtggattactggggccagggcaccctggtgacagtgtcttccggcggcggcggctctggcggcggcggcagcggcggcggcggctccgatatccagatgacccagtctccctcttccctgagcgcctccgtgggcgatcgggtgacaatcacctgcagagccagccaggatatccggagatacctgaactggtaccagcagaggcctggcagagtgccacagctgctgatctacaccacatccaccctgcagtctggcgtgccaagcagattttccggcagcggctccgtgacagacttcaccctgacaatctccagcctgcagccagaggatttcggcacatattactgccagcagagctactccccacctcacacctttggccagggcacaaagctggagatcaaggagcccaagtcttccgacaagacccacacctgtccaccttgtcccgccccagaggccgccggcggccctagcgtgtttctgttccctccaaagcctaaggataccctgatgatctccagaaccccagaggtgacatgcgtggtggtggacgtgtctcacgaggaccccgaggtgaagtttaactggtacgtggatggcgtggaggtgcacaatgccaagaccaagccaagggaggagcagtacaacagcacctacagagtggtgtccgtgctgacagtgctgcaccaggactggctgaatggcaaggagtataagtgcaaggtgtccaacaaggccctgccagcccccatcgagaagaccatcagcaaggccaagggccagcctagggagccacaggtgtacaccctgccaccctccagagacgagctgacaaagaatcaggtgtctctgacatgcctggtgaagggcttctacccttccgacatcgccgtggagtgggagtctaacggccagcccgagaacaattacaagaccacaccacctgtgctggactccgatggcagcttcttcctgtatagcaagctgaccgtggataagtctagatggcagcagggcaacgtgttctcctgttccgtgatgcacgaagcactgcacaaccactacactcagaagagcctgtccctgtcccctggaaaatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt